SEQUENCE LISTING (473 sequences)

SEQ ID NO: 1 - DNA, 379 bp, artificial sequence
  synthetic polynucleotide
gaggtgcagctggtgcagtctggggctgaggtgaagaagcctgggtcctcggtgaaggtctcctgcaaggcttctggaggcaccttcagcaactatgctatcagctgggtgcgacaggcccctggacaagggcttgagtggatgggagggatcatccctatctttggtacagcaaactacgcacagaacttccagggcagagtcacgattaccgcggacgaatccatgagcacagcctacatggagctgagcagcctgagatctgaggacacggccgtatattactgtgcgggaggtgggaggtattgtagtggtggtaggtgccactctgcctactctgcctactggggccagggaaccctggtcaccgtctcctcag

SEQ ID NO: 2 - protein, 126 aa, artificial sequence
  synthetic polypeptide
EVQLVQSGAEVKKPGSSVKVSCKASGGTFSNYAISWVRQAPGQGLEWMGGIIPIFGTANYAQNFQGRVTITADESMSTAYMELSSLRSEDTAVYYCAGGGRYCSGGRCHSAYSAYWGQGTLVTVSS

SEQ ID NO: 3 - DNA, 322 bp, artificial sequence
  synthetic polynucleotide
gccatccagttgacccagtctccaggcaccctgtctttgcctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggaccctgaagattttgcagtgtattactgtcagcaatatggtagctcactcactttcggcggagggaccaaagtggatatcaaac

SEQ ID NO: 4 - protein, 107 aa, artificial sequence
  synthetic polypeptide
AIQLTQSPGTLSLPPGERATLSCRASQSVSSSYLAWYQQKPGQAPRLLIYGASSRATGIPDRFSGSGSGTDFTLTISRLDPEDFAVYYCQQYGSSLTFGGGTKVDIK

SEQ ID NO: 5 - protein, 8 aa, artificial sequence
  synthetic polypeptide
GGTFSNYA

SEQ ID NO: 6 - protein, 8 aa, artificial sequence
  synthetic polypeptide
IIPIFGTA

SEQ ID NO: 7 - protein, 19 aa, artificial sequence
  synthetic polypeptide
AGGGRYCSGGRCHSAYSAY

SEQ ID NO: 8 - protein, 7 aa, artificial sequence
  synthetic polypeptide
QSVSSSY

SEQ ID NO: 9 - protein, 3 aa, artificial sequence
  synthetic polypeptide
GAS

SEQ ID NO: 10 - protein, 8 aa, artificial sequence
  synthetic polypeptide
QQYGSSLT

SEQ ID NO: 11 - DNA, 364 bp, artificial sequence
  synthetic polynucleotide
caggtgcagctggtggagtctgggggaggcttggtccaccctggggggtccctgagactctcctgttcagcctctggattcaccttcagtaactatgctatgcactgggtccgccaggctccagggaagggactggaatatgtttcagctattagtagtagtggggatatcacatactacgcggactccgtaaagggcagattcaccatctccagagacaattccaagaactcactgtatcttcaaatgaacagtctgagagctgaggacacggctgtttattactgtgtgaaagatgtaacgaggacctactacgtagtctttgactactggggccagggaaccctggtcaccgtctcctcag

SEQ ID NO: 12 - protein, 121 aa, artificial sequence
  synthetic polypeptide
QVQLVESGGGLVHPGGSLRLSCSASGFTFSNYAMHWVRQAPGKGLEYVSAISSSGDITYYADSVKGRFTISRDNSKNSLYLQMNSLRAEDTAVYYCVKDVTRTYYVVFDYWGQGTLVTVSS

SEQ ID NO: 13 - DNA, 322 bp, artificial sequence
  synthetic polynucleotide
gccatccagttgacccagtctccatcctccctgtctgcatctgtgggagacagagtcaccatcacttgccgggcaagtcagagcattagcagttatttaaattggtatcagcaggaaccagggaaagcccctaaactcctgatctatgctgcatccagtttgcaaggtggggtcccatcaaggttcagtggcagtggatctgggacagatttcactctcaccatcagcagtctgcaacctgaagattttgcaacttactactgtcaacagagttacactaccccgtacacttttggccaggggaccaaagtggatatcaaac

SEQ ID NO: 14 - protein, 107 aa, artificial sequence
  synthetic polypeptide
AIQLTQSPSSLSASVGDRVTITCRASQSISSYLNWYQQEPGKAPKLLIYAASSLQGGVPSRFSGSGSGTDFTLTISSLQPEDFATYYCQQSYTTPYTFGQGTKVDIK

SEQ ID NO: 15 - protein, 8 aa, artificial sequence
  synthetic polypeptide
GFTFSNYA

SEQ ID NO: 16 - protein, 8 aa, artificial sequence
  synthetic polypeptide
ISSSGDIT

SEQ ID NO: 17 - protein, 14 aa, artificial sequence
  synthetic polypeptide
VKDVTRTYYVVFDY

SEQ ID NO: 18 - protein, 6 aa, artificial sequence
  synthetic polypeptide
QSISSY

SEQ ID NO: 19 - protein, 3 aa, artificial sequence
  synthetic polypeptide
AAS

SEQ ID NO: 20 - protein, 9 aa, artificial sequence
  synthetic polypeptide
QQSYTTPYT

SEQ ID NO: 21 - DNA, 358 bp, artificial sequence
  synthetic polynucleotide
caggtgcagctggtggagtctgggggaggcttggtccagcctggggggtccctgagactctcctgtgcagtctctggattcaccgtcagtaggaactacatgagctgggtccgccaggctccagggaaggggctggagtgggtctcacttatttatagcggtggtagcacattctacgcagactccgtgaagggcagattcaccatctccagagacaattccaagaacacgctgtatcttcaaatgaacagcctgagagccgaggacacggctgtgtattactgtgcgagagatctgtttcataggagtggttatcacgactactggggccagggaaccctggtcaccgtctcctcag

SEQ ID NO: 22 - protein, 119 aa, artificial sequence
  synthetic polypeptide
QVQLVESGGGLVQPGGSLRLSCAVSGFTVSRNYMSWVRQAPGKGLEWVSLIYSGGSTFYADSVKGRFTISRDNSKNTLYLQMNSLRAEDTAVYYCARDLFHRSGYHDYWGQGTLVTVSS

SEQ ID NO: 23 - DNA, 319 bp, artificial sequence
  synthetic polynucleotide
gtcatctggatgacccagtctccatcctccctgtctgcatctgtaggagacagagtcaccatcacttgccaggcgagtcaggacattaacaactatttaaattggtatcagcagaaaccagggaaagcccctaagctcctgatcttcgatgcctccaatttggaaacaggggtcccatcaaggttcagtggcagtggatctgggacagattttactttcaccatcagcagcctacagcctgaagatattgcaacatattactgtcaacagtatgataatctccctgccttcggcggagggaccaaagtggatatcaaac

SEQ ID NO: 24 - protein, 106 aa, artificial sequence
  synthetic polypeptide
VIWMTQSPSSLSASVGDRVTITCQASQDINNYLNWYQQKPGKAPKLLIFDASNLETGVPSRFSGSGSGTDFTFTISSLQPEDIATYYCQQYDNLPAFGGGTKVDIK

SEQ ID NO: 25 - protein, 8 aa, artificial sequence
  synthetic polypeptide
GFTVSRNY

SEQ ID NO: 26 - protein, 7 aa, artificial sequence
  synthetic polypeptide
IYSGGST

SEQ ID NO: 27 - protein, 13 aa, artificial sequence
  synthetic polypeptide
ARDLFHRSGYHDY

SEQ ID NO: 28 - protein, 6 aa, artificial sequence
  synthetic polypeptide
QDINNY

SEQ ID NO: 29 - protein, 3 aa, artificial sequence
  synthetic polypeptide
DAS

SEQ ID NO: 30 - protein, 8 aa, artificial sequence
  synthetic polypeptide
QQYDNLPA

SEQ ID NO: 31 - DNA, 361 bp, artificial sequence
  synthetic polynucleotide
gaagtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcggttgtatggtatgatggaagcaagaaatactatgcagactccgtgaagggccgattcaccatctccagagacaattccaagaacaccctgtatctgcaaatgaacagcctgagagtcgaggacacggctgtgtattactgcgcgagagattttgcggtgggggaggagatcgctgactcctggggccagggaaccctggtcaccgtctcctcag

SEQ ID NO: 32 - protein, 120 aa, artificial sequence
  synthetic polypeptide
EVQLVESGGGVVQPGRSLRLSCAASGFTFSNYGMHWVRQAPGKGLEWVAVVWYDGSKKYYADSVKGRFTISRDNSKNTLYLQMNSLRVEDTAVYYCARDFAVGEEIADSWGQGTLVTVSS

SEQ ID NO: 33 - DNA, 325 bp, artificial sequence
  synthetic polynucleotide
tcctatgagctgactcagccaccctcggtgtcagtgtccccaggacaaacggccaggatcacctgctctggagatgcattgccaaaaaaatatgcttattggtaccagcagaagtcaggccaggcccctgtactggtcatctatgaggacagcaaacgaccctccgggatccctgagagattctctgggtccagctcagggacaatggccaccttgactatcagtggggcccaggtggaggatgaaggtgactactactgttactcaagagacagcagtggtgatcattgggtgttcggcgcagggaccaagctgaccgtcctag

SEQ ID NO: 34 - protein, 108 aa, artificial sequence
  synthetic polypeptide
SYELTQPPSVSVSPGQTARITCSGDALPKKYAYWYQQKSGQAPVLVIYEDSKRPSGIPERFSGSSSGTMATLTISGAQVEDEGDYYCYSRDSSGDHWVFGAGTKLTVL

SEQ ID NO: 35 - protein, 8 aa, artificial sequence
  synthetic polypeptide
GFTFSNYG

SEQ ID NO: 36 - protein, 8 aa, artificial sequence
  synthetic polypeptide
VWYDGSKK

SEQ ID NO: 37 - protein, 13 aa, artificial sequence
  synthetic polypeptide
ARDFAVGEEIADS

SEQ ID NO: 38 - protein, 6 aa, artificial sequence
  synthetic polypeptide
ALPKKY

SEQ ID NO: 39 - protein, 3 aa, artificial sequence
  synthetic polypeptide
EDS

SEQ ID NO: 40 - protein, 11 aa, artificial sequence
  synthetic polypeptide
YSRDSSGDHWV

SEQ ID NO: 41 - DNA, 363 bp, artificial sequence
  synthetic polynucleotide
caggtgcagctggtggagtctgggggaggcgtggtccagcctgggaggtccctgagactctcctgtgcagcctctggattcaccttcagtacctatgctatgcactgggtccgccaggctccaggcaaggggctggagtgggtggctgttctttcatatgatggaagcaataaatactacgcagactccgtgaagggccgattcaccatctccagagacaattccaagaacacgctgtatctgcaaatgaacagcctgagagctgaggacacggctgtgtattactgtgcgaaagggggctcgtacgcgtactactactacatggacgtctggggcaaagggaccacggtcaccgtctcctca

SEQ ID NO: 42 - protein, 121 aa, artificial sequence
  synthetic polypeptide
QVQLVESGGGVVQPGRSLRLSCAASGFTFSTYAMHWVRQAPGKGLEWVAVLSYDGSNKYYADSVKGRFTISRDNSKNTLYLQMNSLRAEDTAVYYCAKGGSYAYYYYMDVWGKGTTVTVSS

SEQ ID NO: 43 - DNA, 322 bp, artificial sequence
  synthetic polynucleotide
gacatccagttgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaggtggatctgggacagattttactttcaccatcaccagcctgcagcctgaagatattgcaacatattactgtcaacagtatgataatctcccgctcactttcggcggagggaccaaagtggatatcaaac

SEQ ID NO: 44 - protein, 107 aa, artificial sequence
  synthetic polypeptide
DIQLTQSPSSLSASVGDRVTITCQASQDISNYLNWYQQKPGKAPKLLIYDASNLETGVPSRFSGGGSGTDFTFTITSLQPEDIATYYCQQYDNLPLTFGGGTKVDIK

SEQ ID NO: 45 - protein, 8 aa, artificial sequence
  synthetic polypeptide
GFTFSTYA

SEQ ID NO: 46 - protein, 8 aa, artificial sequence
  synthetic polypeptide
LSYDGSNK

SEQ ID NO: 47 - protein, 14 aa, artificial sequence
  synthetic polypeptide
AKGGSYAYYYYMDV

SEQ ID NO: 48 - protein, 6 aa, artificial sequence
  synthetic polypeptide
QDISNY

SEQ ID NO: 49 - protein, 3 aa, artificial sequence
  synthetic polypeptide
DAS

SEQ ID NO: 50 - protein, 9 aa, artificial sequence
  synthetic polypeptide
QQYDNLPLT

SEQ ID NO: 51 - DNA, 376 bp, artificial sequence
  synthetic polynucleotide
gttcagctggtgcaggagtcgggcccaggactggtgaagccttcggagaccctgtccctcacctgcactgtctctggtggctccgtcagtagtggtagttactactggagctggatccggcagcccccagggaagggactggagtggattgggtatatgtatttcagtgggagcaccaactataatccctccctcaagagtcgagtcaccatatcattagccacgtccaagaaccagttctccctgaagctgagctctgtgaccgctgcggacacggccgtctattactgtgcgagaggggattacgatttttggagtggtccccccggtcgggtggacgtctggggcaaagggaccacggtcaccgtctcctcag

SEQ ID NO: 52 - protein, 125 aa, artificial sequence
  synthetic polypeptide
VQLVQESGPGLVKPSETLSLTCTVSGGSVSSGSYYWSWIRQPPGKGLEWIGYMYFSGSTNYNPSLKSRVTISLATSKNQFSLKLSSVTAADTAVYYCARGDYDFWSGPPGRVDVWGKGTTVTVSS

SEQ ID NO: 53 - DNA, 325 bp, artificial sequence
  synthetic polynucleotide
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcactatggtagttcacccgtaacttttggccaggggaccaaagtggatatcaaac

SEQ ID NO: 54 - protein, 108 aa, artificial sequence
  synthetic polypeptide
EIVMTQSPGTLSLSPGERATLSCRASQSVSSSYLAWYQQKPGQAPRLLIYGASSRATGIPDRFSGSGSGTDFTLTISRLEPEDFAVYYCQHYGSSPVTFGQGTKVDIK

SEQ ID NO: 55 - protein, 10 aa, artificial sequence
  synthetic polypeptide
GGSVSSGSYY

SEQ ID NO: 56 - protein, 7 aa, artificial sequence
  synthetic polypeptide
MYFSGST

SEQ ID NO: 57 - protein, 17 aa, artificial sequence
  synthetic polypeptide
ARGDYDFWSGPPGRVDV

SEQ ID NO: 58 - protein, 7 aa, artificial sequence
  synthetic polypeptide
QSVSSSY

SEQ ID NO: 59 - protein, 3 aa, artificial sequence
  synthetic polypeptide
GAS

SEQ ID NO: 60 - protein, 9 aa, artificial sequence
  synthetic polypeptide
QHYGSSPVT

SEQ ID NO: 61 - DNA, 367 bp, artificial sequence
  synthetic polynucleotide
caggtgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagatgagcagcctgagatccgaggacacggccgtgtattactgtgcggcaccggcctgtggtaccagctgctctgatgcctttgatatctggggccaagggacaatggtcaccgtctcttcag

SEQ ID NO: 62 - protein, 122 aa, artificial sequence
  synthetic polypeptide
QVQLVQSGPEVKKPGTSVKVSCKASGFTFTSSAVQWVRQARGQRLEWIGWIVVGSGNTNYAQKFQERVTITRDMSTSTAYMEMSSLRSEDTAVYYCAAPACGTSCSDAFDIWGQGTMVTVSS

SEQ ID NO: 63 - DNA, 325 bp, artificial sequence
  synthetic polynucleotide
gacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttggagtgtattactgtcagcagtatggtagctcaccgtggacgttcggccaagggaccaaggtggaaatcaaac

SEQ ID NO: 64 - protein, 108 aa, artificial sequence
  synthetic polypeptide
DIQMTQSPGTLSLSPGERATLSCRASQSVSSSYLAWYQQKPGQAPRLLIYGASSRATGIPDRFSGSGSGTDFTLTISRLEPEDFGVYYCQQYGSSPWTFGQGTKVEIK

SEQ ID NO: 65 - protein, 8 aa, artificial sequence
  synthetic polypeptide
GFTFTSSA

SEQ ID NO: 66 - protein, 8 aa, artificial sequence
  synthetic polypeptide
IVVGSGNT

SEQ ID NO: 67 - protein, 15 aa, artificial sequence
  synthetic polypeptide
AAPACGTSCSDAFDI

SEQ ID NO: 68 - protein, 7 aa, artificial sequence
  synthetic polypeptide
QSVSSSY

SEQ ID NO: 69 - protein, 3 aa, artificial sequence
  synthetic polypeptide
GAS

SEQ ID NO: 70 - protein, 9 aa, artificial sequence
  synthetic polypeptide
QQYGSSPWT

SEQ ID NO: 71 - DNA, 382 bp, artificial sequence
  synthetic polynucleotide
caggtgcagctggtggagtctgggggaggcttggtacagcctggcaggtccctgagactctcctgtgcagcctctggattcacctttgatgattatgccatgcactgggtccggcaacctccagggaagggcctggagtgggtctcaggtgtcagttggaacagtggtaccataggctatgcggactctgtgaagggccgattcatcatctccagagacaacgccaagaactccctgtatctgcaaatgaacagtctgaaagctgaggacacggccttgtattactgtgcaagagaagtgggggggacttttggagtccttatttcacgcgaggggggacttgattactggggccagggaaccctggtcaccgtctcctcag

SEQ ID NO: 72 - protein, 127 aa, artificial sequence
  synthetic polypeptide
QVQLVESGGGLVQPGRSLRLSCAASGFTFDDYAMHWVRQPPGKGLEWVSGVSWNSGTIGYADSVKGRFIISRDNAKNSLYLQMNSLKAEDTALYYCAREVGGTFGVLISREGGLDYWGQGTLVTVSS

SEQ ID NO: 73 - DNA, 325 bp, artificial sequence
  synthetic polynucleotide
tcctatgagctgacacagccaccctcggtgtcagtggccccaggacagacggccagaattacctgtgggggaaacaccattggaagtaaaagtgtgcactggtaccagcagagaccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcgggtggtattcggcggagggaccaagctgaccgtcctag

SEQ ID NO: 74 - protein, 108 aa, artificial sequence
  synthetic polypeptide
SYELTQPPSVSVAPGQTARITCGGNTIGSKSVHWYQQRPGQAPVLVVYDDSDRPSGIPERFSGSNSGNTATLTISRVEAGDEADYYCQVWDSSSDRVVFGGGTKLTVL

SEQ ID NO: 75 - protein, 8 aa, artificial sequence
  synthetic polypeptide
GFTFDDYA

SEQ ID NO: 76 - protein, 8 aa, artificial sequence
  synthetic polypeptide
VSWNSGTI

SEQ ID NO: 77 - protein, 20 aa, artificial sequence
  synthetic polypeptide
AREVGGTFGVLISREGGLDY

SEQ ID NO: 78 - protein, 6 aa, artificial sequence
  synthetic polypeptide
TIGSKS

SEQ ID NO: 79 - protein, 3 aa, artificial sequence
  synthetic polypeptide
DDS

SEQ ID NO: 80 - protein, 11 aa, artificial sequence
  synthetic polypeptide
QVWDSSSDRVV

SEQ ID NO: 81 - DNA, 385 bp, artificial sequence
  synthetic polynucleotide
caggtgcagctgcaggagtcgggcccaggactggtgaagccttcggagaccctgtccctcatctgcactgtctctggtggctccgtcagcagtggtaatttctactggagctggatccggcagcccccagggaagggactggagtggattggatctatctattacactgggagccccaactacaacccctccctcaagagtcgagtcaccatatccctagacacgtccaagaaccagttctccctgaagctgagctctgtgaccgctgcggacacggccgtgtattactgtgcgagagagatctattattatgatagaagtggttcttacaactctgatgcttttgatatctggggccaagggacaatggtcaccgtctcttcag

SEQ ID NO: 82 - protein, 128 aa, artificial sequence
  synthetic polypeptide
QVQLQESGPGLVKPSETLSLICTVSGGSVSSGNFYWSWIRQPPGKGLEWIGSIYYTGSPNYNPSLKSRVTISLDTSKNQFSLKLSSVTAADTAVYYCAREIYYYDRSGSYNSDAFDIWGQGTMVTVSS

SEQ ID NO: 83 - DNA, 325 bp, artificial sequence
  synthetic polynucleotide
gatatcgtgatgactcagtctccagccaccctgtctgtgtctccaggggaaagaggcaccctctcctgcagggccagtcagagtgttagcagcaacttagcctggtaccagcagaaaccgggccaggctcccaggctcctcatctatggtgcatccacgagggccactggtatcccagccaggttcagtggcagtgggtctgggacagagttcactctcaccatcagcagcctgcagtctgaagattttgcagtttattactgccagcagtataataactggcctccgctcactttcggcggagggaccaaagtggatatcaaac

SEQ ID NO: 84 - protein, 108 aa, artificial sequence
  synthetic polypeptide
DIVMTQSPATLSVSPGERGTLSCRASQSVSSNLAWYQQKPGQAPRLLIYGASTRATGIPARFSGSGSGTEFTLTISSLQSEDFAVYYCQQYNNWPPLTFGGGTKVDIK

SEQ ID NO: 85 - protein, 10 aa, artificial sequence
  synthetic polypeptide
GGSVSSGNFY

SEQ ID NO: 86 - protein, 7 aa, artificial sequence
  synthetic polypeptide
IYYTGSP

SEQ ID NO: 87 - protein, 20 aa, artificial sequence
  synthetic polypeptide
AREIYYYDRSGSYNSDAFDI

SEQ ID NO: 88 - protein, 6 aa, artificial sequence
  synthetic polypeptide
QSVSSN

SEQ ID NO: 89 - protein, 3 aa, artificial sequence
  synthetic polypeptide
GAS

SEQ ID NO: 90 - protein, 10 aa, artificial sequence
  synthetic polypeptide
QQYNNWPPLT

SEQ ID NO: 91 - DNA, 381 bp, artificial sequence
  synthetic polynucleotide
caggtgcagctggtggagtctgggggaggcgtggttcagcctgggaggtccctgagactctcctgtgcagcctctggattcaccttcaataactatcctttgcactgggtccgccaggctccaggcaaggggccggagtgggtggcagttatttcacaggatggaggcaataaatactacgtagactccgtgaagggccgattcaccatctccagagacaattccaagaacaccctgtatctgcaaatgaacaacctgagagctgaggacacggctctgtattactgtgcgagagatgttgtagtggtggtagctgctaggaaccactactacaacggtatggacgtctggggccaagggaccacggtcaccgtctcctca

SEQ ID NO: 92 - protein, 127 aa, artificial sequence
  synthetic polypeptide
QVQLVESGGGVVQPGRSLRLSCAASGFTFNNYPLHWVRQAPGKGPEWVAVISQDGGNKYYVDSVKGRFTISRDNSKNTLYLQMNNLRAEDTALYYCARDVVVVVAARNHYYNGMDVWGQGTTVTVSS

SEQ ID NO: 93 - DNA, 322 bp, artificial sequence
  synthetic polynucleotide
gacatccagttgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgtatccagtttgcaaagtggggtcccatcaaggttcagcggcagtggatctgggacagatttcactctcaccatcagcagcctgcagcctgaagattttgcaacttactattgtcaacaggctaagagtttccctttcactttcggccctgggaccaaggtggagattaaac

SEQ ID NO: 94 - protein, 107 aa, artificial sequence
  synthetic polypeptide
DIQLTQSPSSVSASVGDRVTITCRASQGISSWLAWYQQKPGKAPKLLIYAVSSLQSGVPSRFSGSGSGTDFTLTISSLQPEDFATYYCQQAKSFPFTFGPGTKVEIK

SEQ ID NO: 95 - protein, 8 aa, artificial sequence
  synthetic polypeptide
GFTFNNYP

SEQ ID NO: 96 - protein, 8 aa, artificial sequence
  synthetic polypeptide
ISQDGGNK

SEQ ID NO: 97 - protein, 20 aa, artificial sequence
  synthetic polypeptide
ARDVVVVVAARNHYYNGMDV

SEQ ID NO: 98 - protein, 6 aa, artificial sequence
  synthetic polypeptide
QGISSW

SEQ ID NO: 99 - protein, 3 aa, artificial sequence
  synthetic polypeptide
AVS

SEQ ID NO: 100 - protein, 9 aa, artificial sequence
  synthetic polypeptide
QQAKSFPFT

SEQ ID NO: 101 - DNA, 381 bp, artificial sequence
  synthetic polynucleotide
cagctgcagctgcaggagtcgggcccaggactggtgaagccttcacagaccctgtccctcacctgcactgtctctggtggctccatcagtagtggtagttataattggacctggatccggcagcccgccgggaagggactggagtggattgggcgtatatataatagtgggagcaccaactacaacccctccctcaagagtcgagtcaccatatcagtagacacgtccaagaaccagttgtccctgaaggtgaggtctgtgaccgccgcagacacggccgtgtattactgtgcgagacattgcagtggtggtacctgctacccgaagtactactacggtatggacgtctggggccaagggaccacggtcaccgtctcctca

SEQ ID NO: 102 - protein, 127 aa, artificial sequence
  synthetic polypeptide
QLQLQESGPGLVKPSQTLSLTCTVSGGSISSGSYNWTWIRQPAGKGLEWIGRIYNSGSTNYNPSLKSRVTISVDTSKNQLSLKVRSVTAADTAVYYCARHCSGGTCYPKYYYGMDVWGQGTTVTVSS

SEQ ID NO: 103 - DNA, 331 bp, artificial sequence
  synthetic polynucleotide
caatctgccctgactcagccaccctcggtgtctgaagcccccaggcagagggtcaccatctcctgttctggaagcagctccaacatcggaaataatgctgtaaactggtaccagcagttcccaggaaaggctcccaaactcctcatctattatgatgatctgctgccctcaggggtctctgaccgattctctggctccaagtctggcacctcagcctccctggccatcagtggggtccagtctgaggatgaggctgattattactgtgcagcatgggatgacagcctgaatgtcgtggtattcggcggagggaccaagctgaccgtcctag

SEQ ID NO: 104 - protein, 110 aa, artificial sequence
  synthetic polypeptide
QSALTQPPSVSEAPRQRVTISCSGSSSNIGNNAVNWYQQFPGKAPKLLIYYDDLLPSGVSDRFSGSKSGTSASLAISGVQSEDEADYYCAAWDDSLNVVVFGGGTKLTVL

SEQ ID NO: 105 - protein, 10 aa, artificial sequence
  synthetic polypeptide
GGSISSGSYN

SEQ ID NO: 106 - protein, 7 aa, artificial sequence
  synthetic polypeptide
IYNSGST

SEQ ID NO: 107 - protein, 19 aa, artificial sequence
  synthetic polypeptide
ARHCSGGTCYPKYYYGMDV

SEQ ID NO: 108 - protein, 8 aa, artificial sequence
  synthetic polypeptide
SSNIGNNA

SEQ ID NO: 109 - protein, 3 aa, artificial sequence
  synthetic polypeptide
YDD

SEQ ID NO: 110 - protein, 11 aa, artificial sequence
  synthetic polypeptide
AAWDDSLNVVV

SEQ ID NO: 111 - DNA, 370 bp, artificial sequence
  synthetic polynucleotide
caggtgcagctggtggagtcgggcccaggactggtgaagccttcggagaccctgtccctcacctgcactgtctctggtggctccatcagcagtaatagttacttctggggctggatccgccagcccccagggacggggctggagtggattgggaatatctattatactgggagcacctactacaacccgtcgttcgagagtcgagtcaccatgtccgtagacacgtcgaagaaccagttctccctgaggctgagctctgtgaccgccgcagacacggctgtgtattactgtgcgagacatgtcagggcctacgactatgatgccccttttgatatctggggccaagggacaatggtcaccgtctcttcag

SEQ ID NO: 112 - protein, 123 aa, artificial sequence
  synthetic polypeptide
QVQLVESGPGLVKPSETLSLTCTVSGGSISSNSYFWGWIRQPPGTGLEWIGNIYYTGSTYYNPSFESRVTMSVDTSKNQFSLRLSSVTAADTAVYYCARHVRAYDYDAPFDIWGQGTMVTVSS

SEQ ID NO: 113 - DNA, 322 bp, artificial sequence
  synthetic polynucleotide
gtcatctggatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacacaattcactctcacaatcagcagcctgcagcctgaagattttgcaacttattactgtctacagattaatagttatccgctcactttcggcggagggaccaaggtggaaatcaaac

SEQ ID NO: 114 - protein, 107 aa, artificial sequence
  synthetic polypeptide
VIWMTQSPSSLSASVGDRVTITCRASQGIRNDLGWYQQKPGKAPKRLIYAASSLQSGVPSRFSGSGSGTQFTLTISSLQPEDFATYYCLQINSYPLTFGGGTKVEIK

SEQ ID NO: 115 - protein, 10 aa, artificial sequence
  synthetic polypeptide
GGSISSNSYF

SEQ ID NO: 116 - protein, 7 aa, artificial sequence
  synthetic polypeptide
IYYTGST

SEQ ID NO: 117 - protein, 15 aa, artificial sequence
  synthetic polypeptide
ARHVRAYDYDAPFDI

SEQ ID NO: 118 - protein, 6 aa, artificial sequence
  synthetic polypeptide
QGIRND

SEQ ID NO: 119 - protein, 3 aa, artificial sequence
  synthetic polypeptide
AAS

SEQ ID NO: 120 - protein, 9 aa, artificial sequence
  synthetic polypeptide
LQINSYPLT

SEQ ID NO: 121 - DNA, 349 bp, artificial sequence
  synthetic polynucleotide
caggtacagctgcagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagttctgtgaccgccgcggacacggctgtgtattactgtgcgagaactgattactatgatagtatagactggggccagggaaccctggtcaccgtctcctcag

SEQ ID NO: 122 - protein, 116 aa, artificial sequence
  synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARTDYYDSIDWGQGTLVTVSS

SEQ ID NO: 123 - DNA, 328 bp, artificial sequence
  synthetic polynucleotide
cagtctgtgctgactcaggagccctcactgactgtgtccccaggagggacagtcactctcacctgtggctccagcactggagctgtcaccagtggtcattatccctactggttccagcagaagcctggccaagtccccaggacactgatttatgatacaaggaacaaacactcctggacccctgcccggttctcaggctccctccttgggggcaaagctgccctgaccctttcgggtgcgcagcctgaggatgaggctgaatattactgcttgctctcctctagtggtgctcgggtgttcggcggagggaccaagctgaccgtcctag

SEQ ID NO: 124 - protein, 109 aa, artificial sequence
  synthetic polypeptide
QSVLTQEPSLTVSPGGTVTLTCGSSTGAVTSGHYPYWFQQKPGQVPRTLIYDTRNKHSWTPARFSGSLLGGKAALTLSGAQPEDEAEYYCLLSSSGARVFGGGTKLTVL

SEQ ID NO: 125 - protein, 8 aa, artificial sequence
  synthetic polypeptide
GGSFSGYY

SEQ ID NO: 126 - protein, 7 aa, artificial sequence
  synthetic polypeptide
INHSGST

SEQ ID NO: 127 - protein, 10 aa, artificial sequence
  synthetic polypeptide
ARTDYYDSID

SEQ ID NO: 128 - protein, 9 aa, artificial sequence
  synthetic polypeptide
TGAVTSGHY

SEQ ID NO: 129 - protein, 3 aa, artificial sequence
  synthetic polypeptide
DTR

SEQ ID NO: 130 - protein, 9 aa, artificial sequence
  synthetic polypeptide
LLSSSGARV

SEQ ID NO: 131 - DNA, 358 bp, artificial sequence
  synthetic polynucleotide
gaggtgcagctggtggagtctgggggaggcttggtacagcctggggggtccctgagactctcctgtgcagcctctggattcaccttcagtacctacgacatccactgggtccgccaagctacaggaaaaggtctggagtgggtctcagctattggtactgctggtgacacatactattcaggctccgtgaagggccgattcaccatctccagagaaaatgccaagaactccttgtatcttcaaatgaacagcctgagagccggggacacggctgtgtattactgtgcaaggggtagtgggacctacttctactactttgactactggggccagggaaccctggtcaccgtctcctcag

SEQ ID NO: 132 - protein, 119 aa, artificial sequence
  synthetic polypeptide
EVQLVESGGGLVQPGGSLRLSCAASGFTFSTYDIHWVRQATGKGLEWVSAIGTAGDTYYSGSVKGRFTISRENAKNSLYLQMNSLRAGDTAVYYCARGSGTYFYYFDYWGQGTLVTVSS

SEQ ID NO: 133 - DNA, 325 bp, artificial sequence
  synthetic polynucleotide
gacatcgtgatgactcagtctccatcctccctgtctgcatctgtaggagacagaatcaccatcacttgccgggcaagtcagagcattaacaactatttaaattggtatcagcagaaaccagggaaagcccctaagctcctgatctatgctgcatcccgtttgcaaactggggtcccatcaaggttcagtggcagtggatctgggacagattccactctcaccatcaacactctgcaacctgaagattttgcaacttactactgtcaacagagttacagtgcccctccgtggacgttcggccaagggaccaaagtggatatcaaac

SEQ ID NO: 134 - protein, 108 aa, artificial sequence
  synthetic polypeptide
DIVMTQSPSSLSASVGDRITITCRASQSINNYLNWYQQKPGKAPKLLIYAASRLQTGVPSRFSGSGSGTDSTLTINTLQPEDFATYYCQQSYSAPPWTFGQGTKVDIK

SEQ ID NO: 135 - protein, 8 aa, artificial sequence
  synthetic polypeptide
GFTFSTYD

SEQ ID NO: 136 - protein, 7 aa, artificial sequence
  synthetic polypeptide
IGTAGDT

SEQ ID NO: 137 - protein, 13 aa, artificial sequence
  synthetic polypeptide
ARGSGTYFYYFDY

SEQ ID NO: 138 - protein, 6 aa, artificial sequence
  synthetic polypeptide
QSINNY

SEQ ID NO: 139 - protein, 3 aa, artificial sequence
  synthetic polypeptide
AAS

SEQ ID NO: 140 - protein, 10 aa, artificial sequence
  synthetic polypeptide
QQSYSAPPWT

SEQ ID NO: 141 - DNA, 366 bp, artificial sequence
  synthetic polynucleotide
caggtgcagctggtggagtcgggcccaggactggtgaagccttcggagaccctgtccctcacctgcactgtctctggtggctcgatcagcagttcttactactggggctggatccgccagcccccagggaaggggctggagtggattgggagtgtctattatagtgggagcacctactacaacccgtccctcaagagtcgagtcaccatatccgtggacacgtccaagaaccagttctccctgaggctgagctctgtgaccgccgcagacacggctgtgtattattgtgcgaggctgatgaccacggaagactactactccggtatggacgtctggggccaagggaccacggtcaccgtctcctca

SEQ ID NO: 142 - protein, 122 aa, artificial sequence
  synthetic polypeptide
QVQLVESGPGLVKPSETLSLTCTVSGGSISSSYYWGWIRQPPGKGLEWIGSVYYSGSTYYNPSLKSRVTISVDTSKNQFSLRLSSVTAADTAVYYCARLMTTEDYYSGMDVWGQGTTVTVSS

SEQ ID NO: 143 - DNA, 322 bp, artificial sequence
  synthetic polynucleotide
gccatccagatgacccagtctccatcctcactgtctgcatctgtaggagacagagtcaccatcacttgtcgggcgagtcagggcattagcgattatttagcctggtttcagcagaaaccagggaaagcccctaagtccctgatctatgctgcatccagtttgcaaagtggggtcccatcaaagttcagcggcggtggatctgggacagatttcactctcaccatcagcagcctgcagcctgaagattttgcaacttattactgccaacagtatcatagttacccgatcaccttcggccaagggacacgactggagattaaac

SEQ ID NO: 144 - protein, 107 aa, artificial sequence
  synthetic polypeptide
AIQMTQSPSSLSASVGDRVTITCRASQGISDYLAWFQQKPGKAPKSLIYAASSLQSGVPSKFSGGGSGTDFTLTISSLQPEDFATYYCQQYHSYPITFGQGTRLEIK

SEQ ID NO: 145 - protein, 9 aa, artificial sequence
  synthetic polypeptide
GGSISSSYY

SEQ ID NO: 146 - protein, 7 aa, artificial sequence
  synthetic polypeptide
VYYSGST

SEQ ID NO: 147 - protein, 15 aa, artificial sequence
  synthetic polypeptide
ARLMTTEDYYSGMDV

SEQ ID NO: 148 - protein, 6 aa, artificial sequence
  synthetic polypeptide
QGISDY

SEQ ID NO: 149 - protein, 3 aa, artificial sequence
  synthetic polypeptide
AAS

SEQ ID NO: 150 - protein, 9 aa, artificial sequence
  synthetic polypeptide
QQYHSYPIT

SEQ ID NO: 151 - DNA, 351 bp, artificial sequence
  synthetic polynucleotide
caggtgcagctggtggagtctggaggaggcttgatccagcctggggggtccctgagactctcctgtgcagcctctggggtcaccgtcagtagcaactacatgagttgggtccgccaggctccagggaaggggctggagtgggtctcaattatttatagtggtggtaccacatactacgcagactccgtgaagggccgattcaccatctccagagactcttccatgaacacgctgtatcttcaaatgaacagcctgagagccgaggacacggccgtgtattactgtgcgagagatctgatggtgtacggtatagacgtctggggccaagggaccacggtcaccgtctcctca

SEQ ID NO: 152 - protein, 117 aa, artificial sequence
  synthetic polypeptide
QVQLVESGGGLIQPGGSLRLSCAASGVTVSSNYMSWVRQAPGKGLEWVSIIYSGGTTYYADSVKGRFTISRDSSMNTLYLQMNSLRAEDTAVYYCARDLMVYGIDVWGQGTTVTVSS

SEQ ID NO: 153 - DNA, 328 bp, artificial sequence
  synthetic polynucleotide
gaaatagtgatgacgcagtctccatcctccctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagatttcactctcaccatcagcagcctgcagcctgaagattttgcaacttattactgtcaacagcttgatagttacccccccgggtacacttttggccaggggaccaaagtggatatcaaac

SEQ ID NO: 154 - protein, 109 aa, artificial sequence
  synthetic polypeptide
EIVMTQSPSSLSASVGDRVTITCRASQGISSYLAWYQQKPGKAPKLLIYAASTLQSGVPSRFSGSGSGTDFTLTISSLQPEDFATYYCQQLDSYPPGYTFGQGTKVDIK

SEQ ID NO: 155 - protein, 8 aa, artificial sequence
  synthetic polypeptide
GVTVSSNY

SEQ ID NO: 156 - protein, 7 aa, artificial sequence
  synthetic polypeptide
IYSGGTT

SEQ ID NO: 157 - protein, 11 aa, artificial sequence
  synthetic polypeptide
ARDLMVYGIDV

SEQ ID NO: 158 - protein, 6 aa, artificial sequence
  synthetic polypeptide
QGISSY

SEQ ID NO: 159 - protein, 3 aa, artificial sequence
  synthetic polypeptide
AAS

SEQ ID NO: 160 - protein, 11 aa, artificial sequence
  synthetic polypeptide
QQLDSYPPGYT

SEQ ID NO: 161 - DNA, 351 bp, artificial sequence
  synthetic polynucleotide
gaggtgcagctgttggagtctggaggagacttgatccagcctggggggtccctgagactctcctgtgcagcctctggggtcaccgtcagtagcaactacatgagctgggtccgccaggctccagggaaggggctggagtgggtctcaattatttatcccggtgggagcacattctacgcagactccgtgaagggccgattcaccatctccagagacaattccaagaacacgctgtatcttcaaatgcacagcctgagagccgaggacacggccgtgtattactgtgcgagagatcttggctcaggggacatggacgtctggggcaaagggaccacggtcaccgtctcctca

SEQ ID NO: 162 - protein, 117 aa, artificial sequence
  synthetic polypeptide
EVQLLESGGDLIQPGGSLRLSCAASGVTVSSNYMSWVRQAPGKGLEWVSIIYPGGSTFYADSVKGRFTISRDNSKNTLYLQMHSLRAEDTAVYYCARDLGSGDMDVWGKGTTVTVSS

SEQ ID NO: 163 - DNA, 322 bp, artificial sequence
  synthetic polynucleotide
gacatcgtgatgactcagtctccatccttcctgtctgcatctgtaggagacagagtcaccatcacttgccgggccagtcagggcattagcagttatttagcctggtatcagcaaaaaccggggaaagcccctaagctcctgatccaagctgcatccactttgcaaagtggggtcccatcaaggttcagcggcagtggatctgggacagaattcactctcacaatcagcagcctgcagcctgaagattttgcaacttattactgtcaacagcttaatagttaccggtacacttttggccaggggaccaaggtggagatcaaac

SEQ ID NO: 164 - protein, 107 aa, artificial sequence
  synthetic polypeptide
DIVMTQSPSFLSASVGDRVTITCRASQGISSYLAWYQQKPGKAPKLLIQAASTLQSGVPSRFSGSGSGTEFTLTISSLQPEDFATYYCQQLNSYRYTFGQGTKVEIK

SEQ ID NO: 165 - protein, 8 aa, artificial sequence
  synthetic polypeptide
GVTVSSNY

SEQ ID NO: 166 - protein, 7 aa, artificial sequence
  synthetic polypeptide
IYPGGST

SEQ ID NO: 167 - protein, 11 aa, artificial sequence
  synthetic polypeptide
ARDLGSGDMDV

SEQ ID NO: 168 - protein, 6 aa, artificial sequence
  synthetic polypeptide
QGISSY

SEQ ID NO: 169 - protein, 3 aa, artificial sequence
  synthetic polypeptide
AAS

SEQ ID NO: 170 - protein, 9 aa, artificial sequence
  synthetic polypeptide
QQLNSYRYT

SEQ ID NO: 171 - DNA, 369 bp, artificial sequence
  synthetic polynucleotide
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcacttatatcatatgatggaggtaatagatactatgcagactccgtgaagggccgattcaccatctccagagacaattccaagaacacgctgtatctgcaaatgaacagactgagagctgaagacacggctatgtattactgtgcgaaagatcgtgatgatgggtgggattggtactacttcatggacgtctggggcaaagggaccacggtcaccgtctcctca

SEQ ID NO: 172 - protein, 123 aa, artificial sequence
  synthetic polypeptide
EVQLVESGGGVVQPGRSLRLSCAASGFTFSSYGMHWVRQAPGKGLEWVALISYDGGNRYYADSVKGRFTISRDNSKNTLYLQMNRLRAEDTAMYYCAKDRDDGWDWYYFMDVWGKGTTVTVSS

SEQ ID NO: 173 - DNA, 322 bp, artificial sequence
  synthetic polynucleotide
gacatccagttgacccagtctccaggcaccctgtctttgtctccaggggaaagagccaccctctcctgcagggccagtcagagtattagcggcaactacttagcctggtaccagcataaacctggccaggctcccagactcctcatctatggtgcatccaccagggccactggcatcccagacaggttcagtggcagtgggtctgggacagacttcactctcaccatcagcagactggagcctgaagattttgcagtgtattactgtcagcagtatggtagctcgtacacttttggccaggggaccaaggtggagatcaaac

SEQ ID NO: 174 - protein, 107 aa, artificial sequence
  synthetic polypeptide
DIQLTQSPGTLSLSPGERATLSCRASQSISGNYLAWYQHKPGQAPRLLIYGASTRATGIPDRFSGSGSGTDFTLTISRLEPEDFAVYYCQQYGSSYTFGQGTKVEIK

SEQ ID NO: 175 - protein, 8 aa, artificial sequence
  synthetic polypeptide
GFTFSSYG

SEQ ID NO: 176 - protein, 8 aa, artificial sequence
  synthetic polypeptide
ISYDGGNR

SEQ ID NO: 177 - protein, 16 aa, artificial sequence
  synthetic polypeptide
AKDRDDGWDWYYFMDV

SEQ ID NO: 178 - protein, 7 aa, artificial sequence
  synthetic polypeptide
QSISGNY

SEQ ID NO: 179 - protein, 3 aa, artificial sequence
  synthetic polypeptide
GAS

SEQ ID NO: 180 - protein, 8 aa, artificial sequence
  synthetic polypeptide
QQYGSSYT

SEQ ID NO: 181 - DNA, 370 bp, artificial sequence
  synthetic polynucleotide
caggttcagctggtgcagtctgggcctgaggtgaagaagcctgggacctcagtgaaggtctcctgcaaggcttctggattcacctttactagctctgctgtgcagtgggtgcgacaggctcgtggacagcgccttgagtggataggatggatcgtcgttggcagtggtaacacaaactacgcacagaagttccaggaaagcgtcaccattaccagggacatgtccacaagcacagcctacatggagctgagcagcctgagatccgaggacacggccgtgtattactgtgcggccccacattgtattggtggtagctgccatgatgcttttgatatctggggccaagggacaatggtcaccgtctcttcag

SEQ ID NO: 182 - protein, 123 aa, artificial sequence
  synthetic polypeptide
QVQLVQSGPEVKKPGTSVKVSCKASGFTFTSSAVQWVRQARGQRLEWIGWIVVGSGNTNYAQKFQESVTITRDMSTSTAYMELSSLRSEDTAVYYCAAPHCIGGSCHDAFDIWGQGTMVTVSS

SEQ ID NO: 183 - DNA, 325 bp, artificial sequence
  synthetic polynucleotide
gatatcgtgatgacccagtctccaggcaccctatctttgtctccaggggaaagagccaccctctcctgcagggccagtcagagtgttagaagcagctacttagcctggtaccagcagaaacctggccaggctcccaggctcctcatctatggtgcatccaggaggggcactggcatcccagacaggttcagtggcagtgggtctgggacagacttcactctcaccatcagcagactggagcctgaagattttgcagtgtattactgtcagcagtatggtagctcaccctggacgttcggccaagggaccaaggtggaaatcaaac

SEQ ID NO: 184 - protein, 108 aa, artificial sequence
  synthetic polypeptide
DIVMTQSPGTLSLSPGERATLSCRASQSVRSSYLAWYQQKPGQAPRLLIYGASRRGTGIPDRFSGSGSGTDFTLTISRLEPEDFAVYYCQQYGSSPWTFGQGTKVEIK

SEQ ID NO: 185 - protein, 8 aa, artificial sequence
  synthetic polypeptide
GFTFTSSA

SEQ ID NO: 186 - protein, 8 aa, artificial sequence
  synthetic polypeptide
IVVGSGNT

SEQ ID NO: 187 - protein, 16 aa, artificial sequence
  synthetic polypeptide
AAPHCIGGSCHDAFDI

SEQ ID NO: 188 - protein, 7 aa, artificial sequence
  synthetic polypeptide
QSVRSSY

SEQ ID NO: 189 - protein, 3 aa, artificial sequence
  synthetic polypeptide
GAS

SEQ ID NO: 190 - protein, 9 aa, artificial sequence
  synthetic polypeptide
QQYGSSPWT

SEQ ID NO: 191 - DNA, 352 bp, artificial sequence
  synthetic polynucleotide
caggtgcagctggtggagtcaggagcagaggtgaaaaagcccggggagtctctgaagatctcctgtaagggttctggatacagctttaccagctactggatcgtctgggtgcgccagatgcccgggaaaggcctggagtggatggggatcatctatcctggtgactctgataccaaatacagtccgtccttccaaggccaggtcagcatctcagccgacaagcccatcagcaccgcctacctgcagtggagcaggctgaaggcctcggacaccgccatgtattactgtgcgagactagggaattggctggtggactactggggccagggaaccctggtcaccgtctcctcag

SEQ ID NO: 192 - protein, 117 aa, artificial sequence
  synthetic polypeptide
QVQLVESGAEVKKPGESLKISCKGSGYSFTSYWIVWVRQMPGKGLEWMGIIYPGDSDTKYSPSFQGQVSISADKPISTAYLQWSRLKASDTAMYYCARLGNWLVDYWGQGTLVTVSS

SEQ ID NO: 193 - DNA, 343 bp, artificial sequence
  synthetic polynucleotide
gatattgtgatgactcagtctcctctctctctgtccgtcacccctggacagccggcctccatctcctgcaagtctagtcagagcctcctgcatagtgatggaaagacctatttgtattggtacctgcagaagccaggccagcctccacagctcctgatgtatgaagtttccaaccggttctctggagtgccagataggttcagtggcagcgggtcagggacagacttcacacttaaaatcagccgggtggagtctgaggatgttggggtttattactgcatgcaaagtatacagcttcctcgcgggatcaccttcggccaagggacacgactggagattaaac

SEQ ID NO: 194 - protein, 114 aa, artificial sequence
  synthetic polypeptide
DIVMTQSPLSLSVTPGQPASISCKSSQSLLHSDGKTYLYWYLQKPGQPPQLLMYEVSNRFSGVPDRFSGSGSGTDFTLKISRVESEDVGVYYCMQSIQLPRGITFGQGTRLEIK

SEQ ID NO: 195 - protein, 8 aa, artificial sequence
  synthetic polypeptide
GYSFTSYW

SEQ ID NO: 196 - protein, 8 aa, artificial sequence
  synthetic polypeptide
IYPGDSDT

SEQ ID NO: 197 - protein, 10 aa, artificial sequence
  synthetic polypeptide
ARLGNWLVDY

SEQ ID NO: 198 - protein, 11 aa, artificial sequence
  synthetic polypeptide
QSLLHSDGKTY

SEQ ID NO: 199 - protein, 3 aa, artificial sequence
  synthetic polypeptide
EVS

SEQ ID NO: 200 - protein, 11 aa, artificial sequence
  synthetic polypeptide
MQSIQLPRGIT

SEQ ID NO: 201 - DNA, 343 bp, artificial sequence
  synthetic polynucleotide
gaggtgcagctggtggagtctggaggaggcttgatccagcctggggggtccctgagactctcctgtgcagcctctgggctcaccgtcagtcgcaattacatgagctgggtccgccaggctccagggaaggggctggagtgggtctcacttatttatagcggtggtagcacatactacgcagactccgtgaagggccgattcaccatctccagagacaattccaagaacacgctgtatcttcaaatgaacagcctgagagccgaggacacggccgtgtattactgtgcgagagatctacgcggagaagtctggggccaagggacaatggtcaccgtctcttcag

SEQ ID NO: 202 - protein, 114 aa, artificial sequence
  synthetic polypeptide
EVQLVESGGGLIQPGGSLRLSCAASGLTVSRNYMSWVRQAPGKGLEWVSLIYSGGSTYYADSVKGRFTISRDNSKNTLYLQMNSLRAEDTAVYYCARDLRGEVWGQGTMVTVSS

SEQ ID NO: 203 - DNA, 322 bp, artificial sequence
  synthetic polynucleotide
gccatccagatgacccagtctccatcctccctgtctgcatctgtaggagacagagtcaccatcacttgccaggcgagtcaggacattagcaactttttaaattggtatcagcagaaaccagggaaagcccctaagctcctgatctacgatgcatccaatttggaaacaggggtcccatcaaggttcagtggaagtggatctgggacagattttactttcaccatcagtagcctgcagcctgaagatattgcaacatattactgtcaccagtatgataatctccctcgaacgttcggccaagggaccaaagtggatatcaaac

SEQ ID NO: 204 - protein, 107 aa, artificial sequence
  synthetic polypeptide
AIQMTQSPSSLSASVGDRVTITCQASQDISNFLNWYQQKPGKAPKLLIYDASNLETGVPSRFSGSGSGTDFTFTISSLQPEDIATYYCHQYDNLPRTFGQGTKVDIK

SEQ ID NO: 205 - protein, 8 aa, artificial sequence
  synthetic polypeptide
GLTVSRNY

SEQ ID NO: 206 - protein, 7 aa, artificial sequence
  synthetic polypeptide
IYSGGST

SEQ ID NO: 207 - protein, 8 aa, artificial sequence
  synthetic polypeptide
ARDLRGEV

SEQ ID NO: 208 - protein, 6 aa, artificial sequence
  synthetic polypeptide
QDISNF

SEQ ID NO: 209 - protein, 3 aa, artificial sequence
  synthetic polypeptide
DAS

SEQ ID NO: 210 - protein, 9 aa, artificial sequence
  synthetic polypeptide
HQYDNLPRT

SEQ ID NO: 211 - DNA, 372 bp, artificial sequence
  synthetic polynucleotide
gaggtgcagctggtggagtctgggggaggcttggtacagcctggggggtccctgagactctcctgtgcagcctctggattcaccttcagtaactacgacatgcactgggtccgccaagctacaggaaaaggtctggagtgggtctcacttattggtactgctggtgacacatactatccagactccgtgaagggccgattcaccatctccagagaaaatgccaagaactccttgtatcttcaaatgaacagcctgagagccggggacacggctgtgtattactgtgcaagagggcaacacactcaaatcggtcactactactactactacatggacgtctggggcaaagggaccacggtcaccgtctcctca

SEQ ID NO: 212 - protein, 124 aa, artificial sequence
  synthetic polypeptide
EVQLVESGGGLVQPGGSLRLSCAASGFTFSNYDMHWVRQATGKGLEWVSLIGTAGDTYYPDSVKGRFTISRENAKNSLYLQMNSLRAGDTAVYYCARGQHTQIGHYYYYYMDVWGKGTTVTVSS

SEQ ID NO: 213 - DNA, 328 bp, artificial sequence
  synthetic polynucleotide
gccatccggatgacccagtctccatcgtccctgtctgcatctgtaggagacagagtcaccatcacttgccgggcaagtcagagcattagcagctatttaaattggtatcagcagaaaccagggaaagcccctaagctcctgatctttgctgcatccagtttgcaaagtggggtcccatcaaggttcagtggcagtggatctgggacagattccactctaaccatcagcagtctgcaacctgaagattttgcaacttactactgtcaacagagttacagtaaccctccggagggcagttttggccaggggaccaaagtggagattaaac

SEQ ID NO: 214 - protein, 109 aa, artificial sequence
  synthetic polypeptide
AIRMTQSPSSLSASVGDRVTITCRASQSISSYLNWYQQKPGKAPKLLIFAASSLQSGVPSRFSGSGSGTDSTLTISSLQPEDFATYYCQQSYSNPPEGSFGQGTKVEIK

SEQ ID NO: 215 - protein, 8 aa, artificial sequence
  synthetic polypeptide
GFTFSNYD

SEQ ID NO: 216 - protein, 7 aa, artificial sequence
  synthetic polypeptide
IGTAGDT

SEQ ID NO: 217 - protein, 18 aa, artificial sequence
  synthetic polypeptide
ARGQHTQIGHYYYYYMDV

SEQ ID NO: 218 - protein, 6 aa, artificial sequence
  synthetic polypeptide
QSISSY

SEQ ID NO: 219 - protein, 3 aa, artificial sequence
  synthetic polypeptide
AAS

SEQ ID NO: 220 - protein, 11 aa, artificial sequence
  synthetic polypeptide
QQSYSNPPEGS

SEQ ID NO: 221 - DNA, 361 bp, artificial sequence
  synthetic polynucleotide
gaagtgcagctggtggagactggaggaggcttgatccagcctggggggtccctgagactctcctgtgcagcctctgggttcaccgtcagtagcaactacatgagctgggtccgccaggctccagggaaggggctggagtgggtctcagttgtttatggcggtggtaccacatactacgcagactccgtgaagggccgattcaccatctccagagacaattccaagaacacgctgtatcttcaaatgaacagcctgagagccgaggacacggccgtatattactgtgcgactgacaatggatacagctatggtttttcatttgactactggggccagggaaccctggtcatcgtctcctcag

SEQ ID NO: 222 - protein, 120 aa, artificial sequence
  synthetic polypeptide
EVQLVETGGGLIQPGGSLRLSCAASGFTVSSNYMSWVRQAPGKGLEWVSVVYGGGTTYYADSVKGRFTISRDNSKNTLYLQMNSLRAEDTAVYYCATDNGYSYGFSFDYWGQGTLVIVSS

SEQ ID NO: 223 - DNA, 331 bp, artificial sequence
  synthetic polynucleotide
cagtctgtgctgactcagcctgcctccatgtctgggtctcctggacagtcgatcaccatctcctgcactggaaccagcagtgatgttgggggttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagtaattgggtgttcggcggagggaccaagctgaccgtcctag

SEQ ID NO: 224 - protein, 110 aa, artificial sequence
  synthetic polypeptide
QSVLTQPASMSGSPGQSITISCTGTSSDVGGYNLVSWYQQHPGKAPKLMIYEGSKRPSGVSNRFSGSKSGNTASLTISGLQAEDEADYYCCSYAGSSNWVFGGGTKLTVL

SEQ ID NO: 225 - protein, 8 aa, artificial sequence
  synthetic polypeptide
GFTVSSNY

SEQ ID NO: 226 - protein, 7 aa, artificial sequence
  synthetic polypeptide
VYGGGTT

SEQ ID NO: 227 - protein, 14 aa, artificial sequence
  synthetic polypeptide
ATDNGYSYGFSFDY

SEQ ID NO: 228 - protein, 9 aa, artificial sequence
  synthetic polypeptide
SSDVGGYNL

SEQ ID NO: 229 - protein, 3 aa, artificial sequence
  synthetic polypeptide
EGS

SEQ ID NO: 230 - protein, 10 aa, artificial sequence
  synthetic polypeptide
CSYAGSSNWV

SEQ ID NO: 231 - DNA, 373 bp, artificial sequence
  synthetic polynucleotide
caggtgcagctggtggagtctggggctgaggtggagaagcctggggcctcagtgaaggtctcctgcaaggcttctggatacaccttcaccggctactatatgcactgggtgcgacaggcccctggacaagggcttgagtggatgggatggatcaaccctatcagtggtggcacaaactatgcacagaagtttcagggcagggtcaccatgaccagggacacgtccatcagcacagcctacatggacctgagcaggctgagatctgacgacacggccgtgtattactgtgcgagaggaacgtattactatgatagtagtggttacatcccatttgactactggggccagggaaccctggtcaccgtctcctcag

SEQ ID NO: 232 - protein, 124 aa, artificial sequence
  synthetic polypeptide
QVQLVESGAEVEKPGASVKVSCKASGYTFTGYYMHWVRQAPGQGLEWMGWINPISGGTNYAQKFQGRVTMTRDTSISTAYMDLSRLRSDDTAVYYCARGTYYYDSSGYIPFDYWGQGTLVTVSS

SEQ ID NO: 233 - DNA, 331 bp, artificial sequence
  synthetic polynucleotide
cagtctgtgctgactcagcctgcctccgtatctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactttggtattcggcggagggaccaagctgaccgtcctag

SEQ ID NO: 234 - protein, 110 aa, artificial sequence
  synthetic polypeptide
QSVLTQPASVSGSPGQSITISCTGTSSDVGSYNLVSWYQQHPGKAPKLMIYEGSKRPSGVSNRFSGSKSGNTASLTISGLQAEDEADYYCCSYAGSSTLVFGGGTKLTVL

SEQ ID NO: 235 - protein, 8 aa, artificial sequence
  synthetic polypeptide
GYTFTGYY

SEQ ID NO: 236 - protein, 8 aa, artificial sequence
  synthetic polypeptide
INPISGGT

SEQ ID NO: 237 - protein, 17 aa, artificial sequence
  synthetic polypeptide
ARGTYYYDSSGYIPFDY

SEQ ID NO: 238 - protein, 9 aa, artificial sequence
  synthetic polypeptide
SSDVGSYNL

SEQ ID NO: 239 - protein, 3 aa, artificial sequence
  synthetic polypeptide
EGS

SEQ ID NO: 240 - protein, 10 aa, artificial sequence
  synthetic polypeptide
CSYAGSSTLV

SEQ ID NO: 241 - DNA, 385 bp, artificial sequence
  synthetic polynucleotide
caggttcagctggtgcagtctgggtctgagttgaagaagcctggggcctcagtgaaggtttcctgcaaggcttctggatacaccttcagtagctatgctatgacttgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagctctgggatattgtagtagtaccagctgctatcccgcttgggctgcttttgatatctggggccaagggacaatggtcaccgtctcttcag

SEQ ID NO: 242 - protein, 128 aa, artificial sequence
  synthetic polypeptide
QVQLVQSGSELKKPGASVKVSCKASGYTFSSYAMTWVRQAPGQGLEWMGWINTNTGNPTYAQGFTGRFVFSLDTSVSTAYLQISSLKAEDTAVYYCARALGYCSSTSCYPAWAAFDIWGQGTMVTVSS

SEQ ID NO: 243 - DNA, 316 bp, artificial sequence
  synthetic polynucleotide
tcctatgagctgactcagccactctcagtgtcagtggccctgggacagacggccagtattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcagcagagcccaagccggggatgaggctgactataactgtcaggtgtgggacagcagcgtggtattcggcggagggaccaagctgaccgtcctag

SEQ ID NO: 244 - protein, 105 aa, artificial sequence
  synthetic polypeptide
SYELTQPLSVSVALGQTASITCGGNNIGSKNVHWYQQKPGQAPVLVIYRDSNRPSGIPERFSGSNSGNTATLTISRAQAGDEADYNCQVWDSSVVFGGGTKLTVL

SEQ ID NO: 245 - protein, 8 aa, artificial sequence
  synthetic polypeptide
GYTFSSYA

SEQ ID NO: 246 - protein, 8 aa, artificial sequence
  synthetic polypeptide
INTNTGNP

SEQ ID NO: 247 - protein, 21 aa, artificial sequence
  synthetic polypeptide
ARALGYCSSTSCYPAWAAFDI

SEQ ID NO: 248 - protein, 6 aa, artificial sequence
  synthetic polypeptide
NIGSKN

SEQ ID NO: 249 - protein, 3 aa, artificial sequence
  synthetic polypeptide
RDS

SEQ ID NO: 250 - protein, 8 aa, artificial sequence
  synthetic polypeptide
QVWDSSVV

SEQ ID NO: 251 - DNA, 358 bp, artificial sequence
  synthetic polynucleotide
gaggtgcagctggtggagtctggaggaggcttgatccagccgggggggtccctgagactctcctgtgcagcctctgggctcaccgtcagtagcaactacatgagttgggtccgccaggctccagggaaggggctggagtgggtctcagttatttatagtggtggtagcacgttctacgcagactccgtgaagggccgattcaccatctccagagacaattccaagaacacgctgtatcttcaaatgaacagcctgggagccgaggacacggccgtgtattactgtgcgagaggagaaggtagtcctggaaactggttcgacccctggggccagggaaccctggtcaccgtctcctcag

SEQ ID NO: 252 - protein, 119 aa, artificial sequence
  synthetic polypeptide
EVQLVESGGGLIQPGGSLRLSCAASGLTVSSNYMSWVRQAPGKGLEWVSVIYSGGSTFYADSVKGRFTISRDNSKNTLYLQMNSLGAEDTAVYYCARGEGSPGNWFDPWGQGTLVTVSS

SEQ ID NO: 253 - DNA, 322 bp, artificial sequence
  synthetic polynucleotide
gatgttgtgatgactcagtctccaggcaccctgtctttgtctccaggggaaagagccaccctctcctgcagggccagtcagagtgttcccagcagctacttagcctggtaccagcagaaacctggccaggctcccaggctcctcatctatggtgcatccaccagggccactggcatcccagacaggttcagtggcagtgggtctgggacagacttcactctcaccatcagcagactggagcctgaggattttgcagtgtattactgtcagcactatgatacctcaccccgtttcggcggagggaccaaagtggatatcaaac

SEQ ID NO: 254 - protein, 107 aa, artificial sequence
  synthetic polypeptide
DVVMTQSPGTLSLSPGERATLSCRASQSVPSSYLAWYQQKPGQAPRLLIYGASTRATGIPDRFSGSGSGTDFTLTISRLEPEDFAVYYCQHYDTSPRFGGGTKVDIK

SEQ ID NO: 255 - protein, 8 aa, artificial sequence
  synthetic polypeptide
GLTVSSNY

SEQ ID NO: 256 - protein, 7 aa, artificial sequence
  synthetic polypeptide
IYSGGST

SEQ ID NO: 257 - protein, 13 aa, artificial sequence
  synthetic polypeptide
ARGEGSPGNWFDP

SEQ ID NO: 258 - protein, 7 aa, artificial sequence
  synthetic polypeptide
QSVPSSY

SEQ ID NO: 259 - protein, 3 aa, artificial sequence
  synthetic polypeptide
GAS

SEQ ID NO: 260 - protein, 8 aa, artificial sequence
  synthetic polypeptide
QHYDTSPR

SEQ ID NO: 261 - DNA, 370 bp, artificial sequence
  synthetic polynucleotide
caggtccagctggtacagtctgggcctgaggtgaagaagcctgggacctcagtgaaggtctcctgcaaggcttctggattcacctttactacctctgctgtgcagtgggtgcgacaggctcgtggacaacgccttgagtggataggatggatcgtcgttggcagtggtaacacaaactacgcacagaagttccaggaaagagtcaccattaccagggacatgtccacaaccacagcctacatggagctgagcagcctgagatccgaggacacggccgtgtatttctgtgcggcgcctcattgtaatagtaccagctgctatgacgcttttgatatctggggccaagggacaatggtcaccgtctcttcag

SEQ ID NO: 262 - protein, 123 aa, artificial sequence
  synthetic polypeptide
QVQLVQSGPEVKKPGTSVKVSCKASGFTFTTSAVQWVRQARGQRLEWIGWIVVGSGNTNYAQKFQERVTITRDMSTTTAYMELSSLRSEDTAVYFCAAPHCNSTSCYDAFDIWGQGTMVTVSS

SEQ ID NO: 263 - DNA, 325 bp, artificial sequence
  synthetic polynucleotide
gacatccagatgacccagtctccaggcaccctgtctttgtctccaggggaaggagccaccctctcctgcagggccagtcagagtgttagcagcagctacttagcctggtaccagcagaaacctggccaggctcccaggctcctcatctatggtgcatctagtggggccactggcatcccagacagattcagtggcagtgggtctgggacagacttcactctcaccatcagcagactggagcctgaagattttgcagtgtattactgtcagcagtatggtagctcaccttacacttttggccaggggaccaaggtggaaatcaaac

SEQ ID NO: 264 - protein, 108 aa, artificial sequence
  synthetic polypeptide
DIQMTQSPGTLSLSPGEGATLSCRASQSVSSSYLAWYQQKPGQAPRLLIYGASSGATGIPDRFSGSGSGTDFTLTISRLEPEDFAVYYCQQYGSSPYTFGQGTKVEIK

SEQ ID NO: 265 - protein, 8 aa, artificial sequence
  synthetic polypeptide
GFTFTTSA

SEQ ID NO: 266 - protein, 8 aa, artificial sequence
  synthetic polypeptide
IVVGSGNT

SEQ ID NO: 267 - protein, 16 aa, artificial sequence
  synthetic polypeptide
AAPHCNSTSCYDAFDI

SEQ ID NO: 268 - protein, 7 aa, artificial sequence
  synthetic polypeptide
QSVSSSY

SEQ ID NO: 269 - protein, 3 aa, artificial sequence
  synthetic polypeptide
GAS

SEQ ID NO: 270 - protein, 9 aa, artificial sequence
  synthetic polypeptide
QQYGSSPYT

SEQ ID NO: 271 - DNA, 352 bp, artificial sequence
  synthetic polynucleotide
caggtgcagctggtggagtctggaggaggcttgatccagcctggggggtccctgagactctcctgtgcagcctctgggctcaccgtcaataggaactacatgagctggatccgccaggctccagggaaggggctggagtgggtctcagttatttatagcggtggtagtacattttacgcagactccgtgaagggccgattcaccatctccagagacaattccaagaacacactgtctcttcaaatgaacagcctgagagccgaggacacggccatttattactgtgcgagagacttctacgagggttcttttgatatctggggccaagggacaatggtcaccgtctcttcag

SEQ ID NO: 272 - protein, 117 aa, artificial sequence
  synthetic polypeptide
QVQLVESGGGLIQPGGSLRLSCAASGLTVNRNYMSWIRQAPGKGLEWVSVIYSGGSTFYADSVKGRFTISRDNSKNTLSLQMNSLRAEDTAIYYCARDFYEGSFDIWGQGTMVTVSS

SEQ ID NO: 273 - DNA, 325 bp, artificial sequence
  synthetic polynucleotide
gccatccagttgacccagtctccttccttcctgtctgcatctataggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcatcttattactgtcaacagcttaatagttaccccgctccggttttcggccctgggaccaaagtggatatcaaac

SEQ ID NO: 274 - protein, 108 aa, artificial sequence
  synthetic polypeptide
AIQLTQSPSFLSASIGDRVTITCRASQGISSYLAWYQQKPGKAPKLLIYAASTLQSGVPSRFSGSGSGTEFTLTISSLQPEDFASYYCQQLNSYPAPVFGPGTKVDIK

SEQ ID NO: 275 - protein, 8 aa, artificial sequence
  synthetic polypeptide
GLTVNRNY

SEQ ID NO: 276 - protein, 7 aa, artificial sequence
  synthetic polypeptide
IYSGGST

SEQ ID NO: 277 - protein, 11 aa, artificial sequence
  synthetic polypeptide
ARDFYEGSFDI

SEQ ID NO: 278 - protein, 6 aa, artificial sequence
  synthetic polypeptide
QGISSY

SEQ ID NO: 279 - protein, 3 aa, artificial sequence
  synthetic polypeptide
AAS

SEQ ID NO: 280 - protein, 10 aa, artificial sequence
  synthetic polypeptide
QQLNSYPAPV

SEQ ID NO: 281 - DNA, 370 bp, artificial sequence
  synthetic polynucleotide
caggtacagctggtgcagtctggggctgaggtgaagaagcctggggcctcagtgaaggtctcctgcaaggcttctggttacatctttatcagatatggtattagctgggtgcgacaggcccctggacaagggcttgagtggatgggatggatcagcgctaacaatggttacacaaactatgcacagaagctccagggcagagtcaccatgaccacagacacatccacgagcacagcctacatggagctgaggagcctgagatctgacgacacggccgtgtattactgtgcgagagatgggggtattttgactggttatctcgactactttgaccactggggccagggaaccctggtcaccgtctcctcag

SEQ ID NO: 282 - protein, 123 aa, artificial sequence
  synthetic polypeptide
QVQLVQSGAEVKKPGASVKVSCKASGYIFIRYGISWVRQAPGQGLEWMGWISANNGYTNYAQKLQGRVTMTTDTSTSTAYMELRSLRSDDTAVYYCARDGGILTGYLDYFDHWGQGTLVTVSS

SEQ ID NO: 283 - DNA, 325 bp, artificial sequence
  synthetic polynucleotide
gacatccagatgacccagtctccatcctccctgtctgcatctgtaggagacagactcaccatcacttgccgggcaagtcagagcattgccagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttaccactgtcaacagagttacagtaccctcggaatcactttcggccctgggaccaaagtggatatcaaac

SEQ ID NO: 284 - protein, 108 aa, artificial sequence
  synthetic polypeptide
DIQMTQSPSSLSASVGDRLTITCRASQSIASYLNWYQQKPGKAPKLLIYAASSLQSGVPSRFSGSGSGTDFTLTISSLQPEDFATYHCQQSYSTLGITFGPGTKVDIK

SEQ ID NO: 285 - protein, 8 aa, artificial sequence
  synthetic polypeptide
GYIFIRYG

SEQ ID NO: 286 - protein, 8 aa, artificial sequence
  synthetic polypeptide
ISANNGYT

SEQ ID NO: 287 - protein, 16 aa, artificial sequence
  synthetic polypeptide
ARDGGILTGYLDYFDH

SEQ ID NO: 288 - protein, 6 aa, artificial sequence
  synthetic polypeptide
QSIASY

SEQ ID NO: 289 - protein, 3 aa, artificial sequence
  synthetic polypeptide
AAS

SEQ ID NO: 290 - protein, 10 aa, artificial sequence
  synthetic polypeptide
QQSYSTLGIT

SEQ ID NO: 291 - DNA, 367 bp, artificial sequence
  synthetic polynucleotide
caggtgcagctggtggagtctgggggaggcttggtccagcctggggggtccctgagactctcctgtgcagcctctggattcccctttagtatctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcacatgaacagcctgagaggcgaggacacggctgtgtattactgtgcgagccgatattacgattttcgaccggaggcttggtttgactactggggccagggaaccctggtcaccgtctcctcag

SEQ ID NO: 292 - protein, 122 aa, artificial sequence
  synthetic polypeptide
QVQLVESGGGLVQPGGSLRLSCAASGFPFSIYWMSWVRQAPGKGLEWVANIKQDGSEKYYVDSVKGRFTISRDNAKNSLYLHMNSLRGEDTAVYYCASRYYDFRPEAWFDYWGQGTLVTVSS

SEQ ID NO: 293 - DNA, 343 bp, artificial sequence
  synthetic polynucleotide
gatattgtgatgacccagactccactctcctcacctgtcacccttggacagccggcctccatctcctgcaggtctagtcaaagcctcgtacacagggatggaaacacctacttgagctggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaagctacacaatttcctcatgggtacacttttggccaggggaccaaggtggagatcaaac

SEQ ID NO: 294 - protein, 114 aa, artificial sequence
  synthetic polypeptide
DIVMTQTPLSSPVTLGQPASISCRSSQSLVHRDGNTYLSWLQQRPGQPPRLLIYKISNRFSGVPDRFSGSGAGTDFTLKISRVEAEDVGVYYCMQATQFPHGYTFGQGTKVEIK

SEQ ID NO: 295 - protein, 8 aa, artificial sequence
  synthetic polypeptide
GFPFSIYW

SEQ ID NO: 296 - protein, 8 aa, artificial sequence
  synthetic polypeptide
IKQDGSEK

SEQ ID NO: 297 - protein, 15 aa, artificial sequence
  synthetic polypeptide
ASRYYDFRPEAWFDY

SEQ ID NO: 298 - protein, 11 aa, artificial sequence
  synthetic polypeptide
QSLVHRDGNTY

SEQ ID NO: 299 - protein, 3 aa, artificial sequence
  synthetic polypeptide
KIS

SEQ ID NO: 300 - protein, 11 aa, artificial sequence
  synthetic polypeptide
MQATQFPHGYT

SEQ ID NO: 301 - DNA, 355 bp, artificial sequence
  synthetic polynucleotide
caggtgcagctgcaggagtctgggggaggcttggtccagcctggggggtccctgagactctcctgttcagcctctggattcaccgtcagtagcaactacatgacctgggtccgccaggctccagggaaggggctggagtgggtctcagttatttatagcggtggtagcacattctacgcagactccgtgaagggcagattcaccatctccagagacaattccaagaacacgctgtatcttcaaatgaacagcctgagagccgaggacaccgctgtgtattactgtgcgagagatctggaagaggccgggggatttgactactggggccagggaaccctggtcaccgtctcctcag

SEQ ID NO: 302 - protein, 118 aa, artificial sequence
  synthetic polypeptide
QVQLQESGGGLVQPGGSLRLSCSASGFTVSSNYMTWVRQAPGKGLEWVSVIYSGGSTFYADSVKGRFTISRDNSKNTLYLQMNSLRAEDTAVYYCARDLEEAGGFDYWGQGTLVTVSS

SEQ ID NO: 303 - DNA, 325 bp, artificial sequence
  synthetic polynucleotide
gaaattgtgttgacgcagtctccaggcaccctgtctttgtctccaggggaaaaagtcaccctctcctgcagggccagtcagagtgttagcagcacctacttagcctggtaccagcagaaacctggccaggctcccaggctcctcatctatggtgcatccagcagggccactggcgtcccagacaggttccgtggcagtgggtctgggacagacttcactctcaccatcagcagactggagcctgaagattttgcagtgtattactgtcagcagtatggtagctcgctgtacacttttggccaggggaccaaagtggatatcaaac

SEQ ID NO: 304 - protein, 108 aa, artificial sequence
  synthetic polypeptide
EIVLTQSPGTLSLSPGEKVTLSCRASQSVSSTYLAWYQQKPGQAPRLLIYGASSRATGVPDRFRGSGSGTDFTLTISRLEPEDFAVYYCQQYGSSLYTFGQGTKVDIK

SEQ ID NO: 305 - protein, 8 aa, artificial sequence
  synthetic polypeptide
GFTVSSNY

SEQ ID NO: 306 - protein, 7 aa, artificial sequence
  synthetic polypeptide
IYSGGST

SEQ ID NO: 307 - protein, 12 aa, artificial sequence
  synthetic polypeptide
ARDLEEAGGFDY

SEQ ID NO: 308 - protein, 7 aa, artificial sequence
  synthetic polypeptide
QSVSSTY

SEQ ID NO: 309 - protein, 3 aa, artificial sequence
  synthetic polypeptide
GAS

SEQ ID NO: 310 - protein, 9 aa, artificial sequence
  synthetic polypeptide
QQYGSSLYT

SEQ ID NO: 311 - DNA, 385 bp, artificial sequence
  synthetic polynucleotide
cagctgcagctgcaggagtcgggcccaggactggtgaagccttcggagaccctgtccctcacctgcactgtctccggtgactccgtcagtaattactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatcaccgggcttcccggtatagcagtggctggtacgaatggtggaactgcttcgacccctggggccagggaaccctggtcaccgtctcctcag

SEQ ID NO: 312 - protein, 128 aa, artificial sequence
  synthetic polypeptide
QLQLQESGPGLVKPSETLSLTCTVSGDSVSNYYWSWIRQPAGKGLEWIGRIYTSGSTNYNPSLKSRVTMSVDTSKNQFSLKLSSVTAADTAVYYCARDHRASRYSSGWYEWWNCFDPWGQGTLVTVSS

SEQ ID NO: 313 - DNA, 325 bp, artificial sequence
  synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcaacagtctgcaacctgaagattttgcaacttactactgtcaacagagttacagtacccccgcgctcactttcggcggagggaccaaagtggatatcaaac

SEQ ID NO: 314 - protein, 108 aa, artificial sequence
  synthetic polypeptide
DIQMTQSPSSLSASVGDRVTITCRASQSISSYLNWYQQKPGKAPKLLIYAASSLQSGVPSRFSGSGSGTDFTLTINSLQPEDFATYYCQQSYSTPALTFGGGTKVDIK

SEQ ID NO: 315 - protein, 8 aa, artificial sequence
  synthetic polypeptide
GDSVSNYY

SEQ ID NO: 316 - protein, 7 aa, artificial sequence
  synthetic polypeptide
IYTSGST

SEQ ID NO: 317 - protein, 22 aa, artificial sequence
  synthetic polypeptide
ARDHRASRYSSGWYEWWNCFDP

SEQ ID NO: 318 - protein, 6 aa, artificial sequence
  synthetic polypeptide
QSISSY

SEQ ID NO: 319 - protein, 3 aa, artificial sequence
  synthetic polypeptide
AAS

SEQ ID NO: 320 - protein, 10 aa, artificial sequence
  synthetic polypeptide
QQSYSTPALT

SEQ ID NO: 321 - DNA, 367 bp, artificial sequence
  synthetic polynucleotide
caggttcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatacacagaagtttcagggcagggtcaccatgaccagggacacgtccatcagcacagcctacatggagctgagcaggctgagatctgacgacacggccgtgtattcctgtgcgagagatatggcgtttagtatggttcggggttcctttgactactggggccagggaaccctggtcaccgtctcctcag

SEQ ID NO: 322 - protein, 122 aa, artificial sequence
  synthetic polypeptide
QVQLVQSGAEVKKPGASVKVSCKASGYTFTGYYMHWVRQAPGQGLEWMGWINPNSGGTNYTQKFQGRVTMTRDTSISTAYMELSRLRSDDTAVYSCARDMAFSMVRGSFDYWGQGTLVTVSS

SEQ ID NO: 323 - DNA, 331 bp, artificial sequence
  synthetic polynucleotide
caggctgtgctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaaccattgggtgttcggcggagggaccaagctgaccgtcctag

SEQ ID NO: 324 - protein, 110 aa, artificial sequence
  synthetic polypeptide
QAVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNHWVFGGGTKLTVL

SEQ ID NO: 325 - protein, 8 aa, artificial sequence
  synthetic polypeptide
GYTFTGYY

SEQ ID NO: 326 - protein, 8 aa, artificial sequence
  synthetic polypeptide
INPNSGGT

SEQ ID NO: 327 - protein, 15 aa, artificial sequence
  synthetic polypeptide
ARDMAFSMVRGSFDY

SEQ ID NO: 328 - protein, 9 aa, artificial sequence
  synthetic polypeptide
SSDVGGYNY

SEQ ID NO: 329 - protein, 3 aa, artificial sequence
  synthetic polypeptide
EVS

SEQ ID NO: 330 - protein, 10 aa, artificial sequence
  synthetic polypeptide
SSYAGSNHWV

SEQ ID NO: 331 - DNA, 358 bp, artificial sequence
  synthetic polynucleotide
caggtgcagctggtgcagtctgggcctgaggtgaagaagcctgggacctcagtgaaggtctcctgcaaggcgtctggattcacccttactagctctgctatgcagtgggtgcgacaggctcgtggacaacgccttgagtggataggatggatcgtcgttggcagtggcaacacaaactacgcacagaagttccaggaaagagtcaccattaccagggacatgtccacaagcacagcctacatggagctgagcagcctgagatccgaggacacggccgtgtattattgtgcggccggccgtggctacaattcggactttgactactggggccagggaaccctggtcaccgtctcctcag

SEQ ID NO: 332 - protein, 119 aa, artificial sequence
  synthetic polypeptide
QVQLVQSGPEVKKPGTSVKVSCKASGFTLTSSAMQWVRQARGQRLEWIGWIVVGSGNTNYAQKFQERVTITRDMSTSTAYMELSSLRSEDTAVYYCAAGRGYNSDFDYWGQGTLVTVSS

SEQ ID NO: 333 - DNA, 325 bp, artificial sequence
  synthetic polynucleotide
gccatccggatgacccagtctccaggcaccctgtctttgtctccaggggaaagagccaccctctcctgcagggccagtcagagtgttagcagcagctacttagcctggtaccagcagagacctggccaggctcccaggctcctcatctatggtacatccagcagggccactggcatcccagacaggttcagtggcagtgggtctgggacagacttcactctcaccatcagcagactggagcctgaagattttgcagtgtattactgtcagcagtatggttactcagtgtacacttttggccaggggaccaaagtggatatcaaac

SEQ ID NO: 334 - protein, 108 aa, artificial sequence
  synthetic polypeptide
AIRMTQSPGTLSLSPGERATLSCRASQSVSSSYLAWYQQRPGQAPRLLIYGTSSRATGIPDRFSGSGSGTDFTLTISRLEPEDFAVYYCQQYGYSVYTFGQGTKVDIK

SEQ ID NO: 335 - protein, 8 aa, artificial sequence
  synthetic polypeptide
GFTLTSSA

SEQ ID NO: 336 - protein, 8 aa, artificial sequence
  synthetic polypeptide
IVVGSGNT

SEQ ID NO: 337 - protein, 12 aa, artificial sequence
  synthetic polypeptide
AAGRGYNSDFDY

SEQ ID NO: 338 - protein, 7 aa, artificial sequence
  synthetic polypeptide
QSVSSSY

SEQ ID NO: 339 - protein, 3 aa, artificial sequence
  synthetic polypeptide
GTS

SEQ ID NO: 340 - protein, 9 aa, artificial sequence
  synthetic polypeptide
QQYGYSVYT

SEQ ID NO: 341 - DNA, 367 bp, artificial sequence
  synthetic polynucleotide
caggtgcagctggtggagtctgaggctgaggtgaagaagcctggggcctcagtgaaggtttcctgcaaggcatctggatacaccttcaccagctactatatgcactgggtgcgacaggcccctggacaagggcttcagtggatgggaataatcaaccctagtgctggtagcacaagctacgcacagaagttccagggcagagtcaccatgaccacggacacgtccacgaccacagtctacatggagctgagcagcctgagatctgaggacacggccgtgtattactgtgcgagagattctgtactagtaccagctgctaatgcttttgatatctggggccaagggacaatggtcaccgtctcttcag

SEQ ID NO: 342 - protein, 122 aa, artificial sequence
  synthetic polypeptide
QVQLVESEAEVKKPGASVKVSCKASGYTFTSYYMHWVRQAPGQGLQWMGIINPSAGSTSYAQKFQGRVTMTTDTSTTTVYMELSSLRSEDTAVYYCARDSVLVPAANAFDIWGQGTMVTVSS

SEQ ID NO: 343 - DNA, 322 bp, artificial sequence
  synthetic polynucleotide
gaaatagtgatgacgcagtctccagccaccctgtctttgtctccaggggaaagagccaccctctcctgcagggccagtcagagtgttagtagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtcgcaactggctattcactttcggccctgggaccaaagtggatatcaaac

SEQ ID NO: 344 - protein, 107 aa, artificial sequence
  synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQRRNWLFTFGPGTKVDIK

SEQ ID NO: 345 - protein, 8 aa, artificial sequence
  synthetic polypeptide
GYTFTSYY

SEQ ID NO: 346 - protein, 8 aa, artificial sequence
  synthetic polypeptide
INPSAGST

SEQ ID NO: 347 - protein, 15 aa, artificial sequence
  synthetic polypeptide
ARDSVLVPAANAFDI

SEQ ID NO: 348 - protein, 6 aa, artificial sequence
  synthetic polypeptide
QSVSSY

SEQ ID NO: 349 - protein, 3 aa, artificial sequence
  synthetic polypeptide
DAS

SEQ ID NO: 350 - protein, 9 aa, artificial sequence
  synthetic polypeptide
QQRRNWLFT

SEQ ID NO: 351 - DNA, 358 bp, artificial sequence
  synthetic polynucleotide
caggtgcagctggtgcagtctggggctgaggtgaagaagcctggggcctcagtgaaggtttcctgcaaggcttctggagacaccttcactagctatactctgcattgggtgcgccaggcccccggacaaaggcttgagtggatgggatggatcaacgctggcaatggttacacaaaatattcacagaagttccagggcagagtcaccattaccagggacacatccgcgagcacagcctacatggagctgagcagcctgagatctgaagacacggctgtgtattactgtgcgaaatgtactatgatagtagactactttgactactggggccagggaaccctggtcaccgtctcctcag

SEQ ID NO: 352 - protein, 119 aa, artificial sequence
  synthetic polypeptide
QVQLVQSGAEVKKPGASVKVSCKASGDTFTSYTLHWVRQAPGQRLEWMGWINAGNGYTKYSQKFQGRVTITRDTSASTAYMELSSLRSEDTAVYYCAKCTMIVDYFDYWGQGTLVTVSS

SEQ ID NO: 353 - DNA, 322 bp, artificial sequence
  synthetic polynucleotide
gccatccggatgacccagtctccttccaccctgtctgcatctgtaggagacagagtcaccatcacttgccgggccagtcagagtattagtggctggttggcctggtatcagcagaaaccagagaaagcccctaagctcctgatctatgatgcctccaatttggaaagtggggtcccatcaaggttcagcggcagtggatctgggacagaattcactctcaccatcaacagcctgcagcctgatgattttgcaacttattactgccaacagtataatagttacccgtggacgttcggccaagggaccaaagtggatatcaaac

SEQ ID NO: 354 - protein, 107 aa, artificial sequence
  synthetic polypeptide
AIRMTQSPSTLSASVGDRVTITCRASQSISGWLAWYQQKPEKAPKLLIYDASNLESGVPSRFSGSGSGTEFTLTINSLQPDDFATYYCQQYNSYPWTFGQGTKVDIK

SEQ ID NO: 355 - protein, 8 aa, artificial sequence
  synthetic polypeptide
GDTFTSYT

SEQ ID NO: 356 - protein, 8 aa, artificial sequence
  synthetic polypeptide
INAGNGYT

SEQ ID NO: 357 - protein, 12 aa, artificial sequence
  synthetic polypeptide
AKCTMIVDYFDY

SEQ ID NO: 358 - protein, 6 aa, artificial sequence
  synthetic polypeptide
QSISGW

SEQ ID NO: 359 - protein, 3 aa, artificial sequence
  synthetic polypeptide
DAS

SEQ ID NO: 360 - protein, 9 aa, artificial sequence
  synthetic polypeptide
QQYNSYPWT

SEQ ID NO: 361 - DNA, 357 bp, artificial sequence
  synthetic polynucleotide
gaggtgcagctggtgcagtctggggctgaggtgaagaagcctggggcctcagtgaaggtctcctgcaaggcttctgggtacaccttcaccagttatgatatcaactgggtgcgacaggccactggacaagggcttgagtggatgggatggatgaaccctcacagtgataccacaggctatgcacagaagttccagggcagagtcaccatgaccaggaacacctccataaccacagcctacatggagctgagcagcctgagatctgaggacacggccgtgtattactgtgctcagggacccatagcagtgaactacatggacgtctggggcaaagggaccacggtcaccgtctcctca

SEQ ID NO: 362 - protein, 119 aa, artificial sequence
  synthetic polypeptide
EVQLVQSGAEVKKPGASVKVSCKASGYTFTSYDINWVRQATGQGLEWMGWMNPHSDTTGYAQKFQGRVTMTRNTSITTAYMELSSLRSEDTAVYYCAQGPIAVNYMDVWGKGTTVTVSS

SEQ ID NO: 363 - DNA, 325 bp, artificial sequence
  synthetic polynucleotide
cagcctgtgctgactcagccaccctcagtgtcagtggccccaggaaagacggccaggattacctgtgggggaagcaacattggaagtaaaagtgtgcactggtaccagcagaagccaggccaggcccctgtgctgatcatctattatgatagcgaccggccctcagggatccctgagcgattctctggctccaactctgggaacacggccaccctgaccatcagcagggtcgaagccggggatgaggccgacttttactgtcaggtgtgggatagtagtactgatcatgtggtattcggcggggggaccaagctgaccgtcctag

SEQ ID NO: 364 - protein, 108 aa, artificial sequence
  synthetic polypeptide
QPVLTQPPSVSVAPGKTARITCGGSNIGSKSVHWYQQKPGQAPVLIIYYDSDRPSGIPERFSGSNSGNTATLTISRVEAGDEADFYCQVWDSSTDHVVFGGGTKLTVL

SEQ ID NO: 365 - protein, 8 aa, artificial sequence
  synthetic polypeptide
GYTFTSYD

SEQ ID NO: 366 - protein, 8 aa, artificial sequence
  synthetic polypeptide
MNPHSDTT

SEQ ID NO: 367 - protein, 12 aa, artificial sequence
  synthetic polypeptide
AQGPIAVNYMDV

SEQ ID NO: 368 - protein, 6 aa, artificial sequence
  synthetic polypeptide
NIGSKS

SEQ ID NO: 369 - protein, 3 aa, artificial sequence
  synthetic polypeptide
YDS

SEQ ID NO: 370 - protein, 11 aa, artificial sequence
  synthetic polypeptide
QVWDSSTDHVV

SEQ ID NO: 371 - DNA, 376 bp, artificial sequence
  synthetic polynucleotide
gaggtgcagctggtggagtctgggggaggcttggtcaagcctggagagtccctgagactctcctgtgcagcctctggattcaccttcagtgactactacatgacctggatccgccaggctccagggaaggggctggagtgggtttcatacattaggagtagtggtcatactatatactacgcagactctgtgaagggccgattcaccatctccagggacaacgccaagaactcactgtatctacaaatgaacagcctgagagtcgaggacacggccgtgtattactgtgcgagaggaggggttttacgatttttggagtggcctctcaatgcttttgatatctggggccaagggacaatggtcaccgtctcttcag

SEQ ID NO: 372 - protein, 125 aa, artificial sequence
  synthetic polypeptide
EVQLVESGGGLVKPGESLRLSCAASGFTFSDYYMTWIRQAPGKGLEWVSYIRSSGHTIYYADSVKGRFTISRDNAKNSLYLQMNSLRVEDTAVYYCARGGVLRFLEWPLNAFDIWGQGTMVTVSS

SEQ ID NO: 373 - DNA, 322 bp, artificial sequence
  synthetic polynucleotide
gacatccagttgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacaatgccctcgggacgttcggccaagggaccaaggtggagatcaaac

SEQ ID NO: 374 - protein, 107 aa, artificial sequence
  synthetic polypeptide
DIQLTQSPSSLSASVGDRVTITCRASQGISNYLAWYQQKPGKVPKLLIYAASTLQSGVPSRFSGSGSGTDFTLTISSLQPEDVATYYCQKYNNALGTFGQGTKVEIK

SEQ ID NO: 375 - protein, 8 aa, artificial sequence
  synthetic polypeptide
GFTFSDYY

SEQ ID NO: 376 - protein, 8 aa, artificial sequence
  synthetic polypeptide
IRSSGHTI

SEQ ID NO: 377 - protein, 18 aa, artificial sequence
  synthetic polypeptide
ARGGVLRFLEWPLNAFDI

SEQ ID NO: 378 - protein, 6 aa, artificial sequence
  synthetic polypeptide
QGISNY

SEQ ID NO: 379 - protein, 3 aa, artificial sequence
  synthetic polypeptide
AAS

SEQ ID NO: 380 - protein, 9 aa, artificial sequence
  synthetic polypeptide
QKYNNALGT

SEQ ID NO: 381 - DNA, 364 bp, artificial sequence
  synthetic polynucleotide
gaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcagccctaacagtggtggcacaaactatgcacagaagtttcagggcagggtcaccatgaccagggacacgtccatcaccacagcctacatggacctgagcaggctgagatctgacgacacggccgtgtattactgtgcgagaggttattactatgaagccctcgatgcttttgatatctggggccaagggacaatggtcaccgtctcttcag

SEQ ID NO: 382 - protein, 121 aa, artificial sequence
  synthetic polypeptide
EVQLVQSGAEVKKPGASVKVSCKASGYTFTGYYMHWVRQAPGQGLEWMGWISPNSGGTNYAQKFQGRVTMTRDTSITTAYMDLSRLRSDDTAVYYCARGYYYEALDAFDIWGQGTMVTVSS

SEQ ID NO: 383 - DNA, 331 bp, artificial sequence
  synthetic polynucleotide
cagtctgtcgtgacgcagcctgcctccgtgtctgggtctcctggacagtcgatcaccatctcctgcactggaaccagcagtgacgttggtggttataactttgtctcctggtaccaacagcacccaggcaaagcccccaaactcatgatttatgaggtcagtaatcggccctcaggggtttctaatcgcttctctggctccaagtctggcatcacggcctccctgaccatctctgggctccaggctgaggacgaggctgattattactgcaactcatatacaagcaacagtactcgggtattcggcggagggaccaagctgaccgtcctag

SEQ ID NO: 384 - protein, 110 aa, artificial sequence
  synthetic polypeptide
QSVVTQPASVSGSPGQSITISCTGTSSDVGGYNFVSWYQQHPGKAPKLMIYEVSNRPSGVSNRFSGSKSGITASLTISGLQAEDEADYYCNSYTSNSTRVFGGGTKLTVL

SEQ ID NO: 385 - protein, 8 aa, artificial sequence
  synthetic polypeptide
GYTFTGYY

SEQ ID NO: 386 - protein, 8 aa, artificial sequence
  synthetic polypeptide
ISPNSGGT

SEQ ID NO: 387 - protein, 14 aa, artificial sequence
  synthetic polypeptide
ARGYYYEALDAFDI

SEQ ID NO: 388 - protein, 9 aa, artificial sequence
  synthetic polypeptide
SSDVGGYNF

SEQ ID NO: 389 - protein, 3 aa, artificial sequence
  synthetic polypeptide
EVS

SEQ ID NO: 390 - protein, 10 aa, artificial sequence
  synthetic polypeptide
NSYTSNSTRV

SEQ ID NO: 391 - DNA, 382 bp, artificial sequence
  synthetic polynucleotide
caggtgcagctggtggagtctgggggaggcttggtccagcctggggggtccctgagactctcctgtgcagcctctggattcaccgtcagtagcaactacatgacctgggtccgccaggctccagggaaggggctggagtgggtctcagttatttatagcggtggtagcacatactacgcagactccgtgaagggcagattcaccatctccagagacaattccaagaacacgctatatcttcaaatgaacagcctgagagccgacgacacggctgtatattactgtgcgagagactctacagccgattacgatttttggagtggttattatgtaggtgcttttcatatctggggccaagggacaatggtcaccgtctcttcag

SEQ ID NO: 392 - protein, 127 aa, artificial sequence
  synthetic polypeptide
QVQLVESGGGLVQPGGSLRLSCAASGFTVSSNYMTWVRQAPGKGLEWVSVIYSGGSTYYADSVKGRFTISRDNSKNTLYLQMNSLRADDTAVYYCARDSTADYDFWSGYYVGAFHIWGQGTMVTVSS

SEQ ID NO: 393 - DNA, 331 bp, artificial sequence
  synthetic polynucleotide
cagactgtgctgactcagcctgcctccgtgtctgggtctcctggacagtcgatcaccatctcctgcactggaaccagcagtgacgttggtggttacaactatgtctcctggtaccaacagcacccaggcaaagcccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcaacaattgggtgttcggcggagggaccaagctgaccgtcctag

SEQ ID NO: 394 - protein, 110 aa, artificial sequence
  synthetic polypeptide
QTVLTQPASVSGSPGQSITISCTGTSSDVGGYNYVSWYQQHPGKAPKLMIYEVTKRPSGVPDRFSGSKSGNTASLTVSGLQAEDEADYYCSSYAGSNNWVFGGGTKLTVL

SEQ ID NO: 395 - protein, 8 aa, artificial sequence
  synthetic polypeptide
GFTVSSNY

SEQ ID NO: 396 - protein, 7 aa, artificial sequence
  synthetic polypeptide
IYSGGST

SEQ ID NO: 397 - protein, 21 aa, artificial sequence
  synthetic polypeptide
ARDSTADYDFWSGYYVGAFHI

SEQ ID NO: 398 - protein, 9 aa, artificial sequence
  synthetic polypeptide
SSDVGGYNY

SEQ ID NO: 399 - protein, 3 aa, artificial sequence
  synthetic polypeptide
EVT

SEQ ID NO: 400 - protein, 10 aa, artificial sequence
  synthetic polypeptide
SSYAGSNNWV

SEQ ID NO: 401 - protein, 8 aa, Artificial Sequence
  synthetic polypeptide
  misc_feature (6)..(6): Xaa can be any naturally occurring amino acid
GFTFTXSA

SEQ ID NO: 402 - protein, 8 aa, Artificial Sequence
  synthetic polypeptide
IVVGSGNT

SEQ ID NO: 403 - protein, 15 aa, Artificial Sequence
  synthetic polypeptide
  misc_feature (4)..(4): Xaa can be any naturally occurring amino acid
  misc_feature (6)..(7): Xaa can be any naturally occurring amino acid
  MOD_RES (8)..(8): S or T
  misc_feature (10)..(10): Xaa can be any naturally occurring amino acid
  misc_feature (12)..(12): Xaa can be any naturally occurring amino acid
AAPXCXXXCXDXFDI

SEQ ID NO: 404 - protein, 7 aa, Artificial Sequence
  synthetic polypeptide
  misc_feature (4)..(4): Xaa can be any naturally occurring amino acid
QSVXSSY

SEQ ID NO: 405 - skipped

SEQ ID NO: 406 - protein, 9 aa, Artificial Sequence
  synthetic polypeptide
  misc_feature (8)..(8): Xaa can be any naturally occurring amino acid
QQYGSSPXT

SEQ ID NO: 407 - protein, 8 aa, Artificial Sequence
  synthetic polypeptide
  misc_feature (2)..(2): Xaa can be any naturally occurring amino acid
  misc_feature (5)..(6): Xaa can be any naturally occurring amino acid
GXTCXXNY

SEQ ID NO: 408 - protein, 7 aa, Artificial Sequence
  synthetic polypeptide
  misc_feature (3)..(3): Xaa can be any naturally occurring amino acid
  misc_feature (6)..(6): Xaa can be any naturally occurring amino acid
IYXGGXT

SEQ ID NO: 409 - protein, 4 aa, Artificial Sequence
  synthetic polypeptide
RRAR

SEQ ID NO: 410 - protein, 4 aa, Artificial Sequence
  synthetic polypeptide
GSAS

SEQ ID NO: 411 - protein, 8 aa, Artificial Sequence
  synthetic polypeptide
HHHHHHHH

SEQ ID NO: 412 - protein, 4 aa, Artificial Sequence
  synthetic polypeptide
RRKR

SEQ ID NO: 413 - protein, 4 aa, Artificial Sequence
  synthetic polypeptide
RRSR

SEQ ID NO: 414 - protein, 4 aa, Artificial Sequence
  synthetic polypeptide
RSVG

SEQ ID NO: 415 - protein, 4 aa, Artificial Sequence
  synthetic polypeptide
ASVG

SEQ ID NO: 416 - protein, 4 aa, Artificial Sequence
  synthetic polypeptide
RSAR

SEQ ID NO: 417 - DNA, 20 bp, Artificial Sequence
  synthetic polynucleotide
atgctgcaatcgtgctacaa

SEQ ID NO: 418 - DNA, 17 bp, Artificial Sequence
  synthetic polynucleotide
gactgccgcctctgctc

SEQ ID NO: 419 - DNA, 20 bp, Artificial Sequence
  synthetic polynucleotide
  misc_feature (1)..(1): 56-FAM
  misc_feature (9)..(10): ZEN
  misc_feature (20)..(20): 3IABkFQ
tcaaggaacaacattgccaa

SEQ ID NO: 420 - DNA, 41 bp, Artificial Sequence
  synthetic polynucleotide
ctacggctttcagcccacatacggtgtgggctaccagcctt

SEQ ID NO: 421 - DNA, 41 bp, Artificial Sequence
  synthetic polynucleotide
aaggctggtagcccacaccgtatgtgggctgaaagccgtag

SEQ ID NO: 422 - DNA, 20 bp, Artificial Sequence
  synthetic polynucleotide
cagctcctgggcaacgtgct

SEQ ID NO: 423 - DNA, 19 bp, Artificial Sequence
  synthetic polynucleotide
cgtaaaaggagcaacatag

SEQ ID NO: 424 - DNA, 27 bp, Artificial Sequence
  synthetic polynucleotide
gggcagaccggcacgatcgccgactac

SEQ ID NO: 425 - DNA, 27 bp, Artificial Sequence
  synthetic polynucleotide
gtagtcggcgatcgtgccggtctgccc

SEQ ID NO: 426 - DNA, 35 bp, Artificial Sequence
  synthetic polynucleotide
cagggcagaccggcaatatcgccgactacaattac

SEQ ID NO: 427 - DNA, 35 bp, Artificial Sequence
  synthetic polynucleotide
gtaattgtagtcggcgatattgccggtctgccctg

SEQ ID NO: 428 - protein, 18 aa, Artificial Sequence
  synthetic polypeptide
CAAPHCNSTSCYDAFDIW

SEQ ID NO: 429 - protein, 17 aa, Artificial Sequence
  synthetic polypeptide
CAAPACGTSCSDAFDIW

SEQ ID NO: 430 - protein, 18 aa, Artificial Sequence
  synthetic polypeptide
CAAPHCIGGSCHDAFDIW

SEQ ID NO: 431 - protein, 11 aa, Artificial Sequence [sequence omitted]

SEQ ID NO: 432 - protein, 11 aa, Artificial Sequence [sequence omitted]

SEQ ID NO: 433 - protein, 58 aa, Artificial Sequence
  synthetic polypeptide
YADSVKGRFTISRDSSMNTLYLQMNSLRAEDTAVYYCARDLMVYGIDVWGQGTTVTVS

SEQ ID NO: 434 - protein, 59 aa, Artificial Sequence
  synthetic polypeptide
YADSVKGRFTISRDNSKNTLYLQMNSLRAEDTAVYYCARDLDVSGGMDVWGQGTTVTVS

SEQ ID NO: 435 - protein, 56 aa, Artificial Sequence
  synthetic polypeptide
YADSVKGRFTISRHNSKNTLYLQMNSLEAEDTAVYYCAREAYGMDVWGQGTTVTVS

SEQ ID NO: 436 - protein, 58 aa, Artificial Sequence
  synthetic polypeptide
YADSVKGRFTISRDNSKNTLYLQMNSLRAEDTAVYHCARDLVVYGMDVWGQGTTVTVS

SEQ ID NO: 437 - protein, 58 aa, Artificial Sequence
  synthetic polypeptide
YADSVKGRFTISRDNSKNTLYLQMSSLRAEDTAVYYCARDLGPYGMDVWGQGTTVTVS

SEQ ID NO: 438 - protein, 59 aa, Artificial Sequence
  synthetic polypeptide
YADSVKGRFTISRDKSKNTLYLQMNSLRAEDTAVYYCARDLGEAGGMDVWGQGTTVTVS

SEQ ID NO: 439 - protein, 58 aa, Artificial Sequence
  synthetic polypeptide
YADSVKGRFTISRDNSKNTLYLQMHSLRAEDTAVYYCARDLGSGDMDVWGKGTTVTVS

SEQ ID NO: 440 - protein, 59 aa, Artificial Sequence
  synthetic polypeptide
YADSVKGRFTISRDNSKNTLYLQMNSLRAEDTAVYYCARDLERAGGMDVWGQGTTVTVS

SEQ ID NO: 441 - protein, 58 aa, Artificial Sequence
  synthetic polypeptide
YADSVKGRFTISRDNSKNTLYLQMNSLRAEDTAVYYCARDLDVYGLDVWGQGTTVTVS

SEQ ID NO: 442 - protein, 55 aa, Artificial Sequence
  synthetic polypeptide
YADSVKGRFTISRDNSKNTLYLQMNSLRAEDTAVYYCARDLRGEVWGQGTMVTVS

SEQ ID NO: 443 - protein, 58 aa, Artificial Sequence
  synthetic polypeptide
YADSVKGRFTISRDNSKSTLYLQMNSLRVEDTAVYYCARDFGDFYFDYWGQGTLVTVS

SEQ ID NO: 444 - protein, 58 aa, Artificial Sequence
  synthetic polypeptide
YADSVKGRFTISRDNSKNTLNLQMNSLRAEDTAVYYCARDYGDYYFDYWGQGTLVTVS

SEQ ID NO: 445 - protein, 60 aa, Artificial Sequence
  synthetic polypeptide
YADSVKGRFTISRDNSMNTLFLQMNSLRAEDTAVYYCARVLPMYGDYLDYWGQGTLVTVS

SEQ ID NO: 446 - protein, 59 aa, Artificial Sequence
  synthetic polypeptide
YADSVKGRFTISRDNSKNTLYLQMNSLRVEDTAVYYCARDLQELGSLDYWGQGTLVTVS

SEQ ID NO: 447 - protein, 60 aa, Artificial Sequence
  synthetic polypeptide
YADSVKGRFTISRDNSKNTLYLQMNSLGAEDTAVYYCARGEGSPGNWFDPWGQGTLVTVS

SEQ ID NO: 448 - protein, 58 aa, Artificial Sequence
  synthetic polypeptide
YADSVKGRFTISRDNSKNTLSLQMNSLRAEDTAIYYCARDFYEGSFDIWGQGTMVTVS

SEQ ID NO: 449 - protein, 15 aa, Artificial Sequence
  synthetic polypeptide
CAASGVTVSSNYMSW

SEQ ID NO: 450 - protein, 14 aa, Artificial Sequence
  synthetic polypeptide
WVSIIYSGGTTYYA

SEQ ID NO: 451 - protein, 13 aa, Artificial Sequence
  synthetic polypeptide
CARDLMVYGIDVW

SEQ ID NO: 452 - protein, 15 aa, Artificial Sequence
  synthetic polypeptide
CAASGVTVSSNYMSW

SEQ ID NO: 453 - protein, 14 aa, Artificial Sequence
  synthetic polypeptide
WVSIIYPGGSTFYA

SEQ ID NO: 454 - protein, 13 aa, Artificial Sequence
  synthetic polypeptide
CARDLGSGDMDVW

SEQ ID NO: 455 - protein, 15 aa, Artificial Sequence
  synthetic polypeptide
CAASGLTVSSNYMSW

SEQ ID NO: 456 - protein, 14 aa, Artificial Sequence
  synthetic polypeptide
WVSVIYSGGSTFYA

SEQ ID NO: 457 - protein, 15 aa, Artificial Sequence
  synthetic polypeptide
CARGEGSPGNWFDPW

SEQ ID NO: 458 - protein, 15 aa, Artificial Sequence
  synthetic polypeptide
CAASGLTVNRNYMSW

SEQ ID NO: 459 - protein, 14 aa, Artificial Sequence
  synthetic polypeptide
WVSVIYSGGSTFYA

SEQ ID NO: 460 - protein, 13 aa, Artificial Sequence
  synthetic polypeptide
CARDFYEGSFDIW

SEQ ID NO: 461 - protein, 15 aa, Artificial Sequence
  synthetic polypeptide
CAASGLTVSRNYMSW

SEQ ID NO: 462 - protein, 14 aa, Artificial Sequence
  synthetic polypeptide
WVSLIYSGGSTYYA

SEQ ID NO: 463 - protein, 10 aa, Artificial Sequence
  synthetic polypeptide
CARDLRGEVW

SEQ ID NO: 464 - protein, 13 aa, Artificial Sequence
  synthetic polypeptide
CRASQGISSYLAW

SEQ ID NO: 465 - protein, 13 aa, Artificial Sequence
  synthetic polypeptide
CQQLDSYPPGYTF

SEQ ID NO: 466 - protein, 13 aa, Artificial Sequence
  synthetic polypeptide
CRASQGISSYLAW

SEQ ID NO: 467 - protein, 11 aa, Artificial Sequence [sequence omitted]

SEQ ID NO: 468 - protein, 14 aa, Artificial Sequence
  synthetic polypeptide
CRASQSVPSSYLAW

SEQ ID NO: 469 - protein, 10 aa, Artificial Sequence
  synthetic polypeptide
CQHYDTSPRF

SEQ ID NO: 470 - protein, 13 aa, Artificial Sequence
  synthetic polypeptide
CRASQGISSYLAW

SEQ ID NO: 471 - protein, 12 aa, Artificial Sequence
  synthetic polypeptide
CQQLNSYPAPVF

SEQ ID NO: 472 - protein, 13 aa, Artificial Sequence
  synthetic polypeptide
CQASQDISNFLNW

SEQ ID NO: 473 - protein, 11 aa, Artificial Sequence [sequence omitted]

(19) United States
(12) Patent Application Publication
(10) Pub. No.: US 2024/0043507 A1
(43) Pub. Date: Feb. 8, 2024

(54) ANTIBODIES

(71) Applicant: RQ Biotechnology Limited, London (GB)

(72) Inventors: Gavin SCREATON, Bicester (GB); Juthathip MONGKOLSAPAYA, Oxford (GB)

(21) Appl. No.: 18/264,226

(22) PCT Filed: Feb. 4, 2022
(86) PCT No.: PCT/GB2022/050306

§ 371 Date: Aug. 3, 2023

(30) Foreign Application Priority Data
Feb. 4, 2021 (GB) 2101578.9
Feb. 4, 2021 (GB) 2101580.5
Feb. 19, 2021 (GB) 2102401.3
Mar. 11, 2021 (GB) 2103388.1
Aug. 27, 2021 (GB) 2112297.3
Nov. 3, 2021 (GB) 2115824.1
Dec. 17, 2021 (GB) 2118423.9
Dec. 17, 2021 (GB) 2118426.2

(51) Int. Cl. C07K 16/10; A61P 31/14; G01N 33/569

(52) U.S. Cl. CPC C07K 16/1003; A61P 31/14; G01N 33/56983; C07K 2317/565; C07K 2317/14; G01N 2800/26

(57) ABSTRACT

The present invention relates to antibodies capable of binding to the spike protein of coronavirus SARS-CoV-2, and methods and uses thereof in the prevention, treatment and/or diagnosis of coronavirus infections, and diseases and/or complications associated with coronavirus infections, including COVID-19.

### FIELD OF INVENTION

The invention relates to antibodies useful for the prevention, treatment and/or diagnosis of coronavirus infections, and diseases and/or complications associated with coronavirus infections, including COVID-19.

### BACKGROUND OF THE INVENTION

A severe viral acute respiratory syndrome named COVID-19 was first reported in Wuhan, China in December 2019. The virus rapidly disseminated globally leading to the pandemic with >70M confirmed infections and over 1.6M deaths in 12 months. The causative agent, SARS-CoV-2, is a beta coronavirus, related to SARS-CoV-1 and MERS coronaviruses, which both cause severe respiratory syndromes.

Coronaviruses have 4 structural proteins, nucleocapsid, envelope, membrane and spike (S) proteins. The spike protein is the most prominent surface protein. It has an elongated trimeric structure and is responsible for engagement of target cells and triggering fusion of viral and host membranes. The spike protein from SARS-CoV-2 and SARS-CoV-1 both use angiotensin-converting enzyme 2 (ACE2) as a cell surface receptor. ACE2 is expressed in a number of tissues, including epithelial cells of the upper and lower respiratory tracts.

The S protein consists of two subunits, S1, which mediates receptor binding, and S2, responsible for viral and host cell membrane fusion. It is a dynamic structure capable of transitioning to a post-fusion state (Cai et al., 2020) by cleavage between S1 and S2 following receptor binding or trypsin treatment. In some SARS-CoV-2 sequences a furin protease cleavage site is inserted between the S1 and S2 subunits and, a mutation of the cleavage site attenuates disease in animal models (Johnson et al., 2020). The S1 fragment occupies the membrane distal tip of S and can be subdivided into an N-terminal domain (NTD) and receptor binding domain (RBD). While both regions are immunogenic, the RBD contains the interacting surface for ACE2 binding (Lan et al., 2020). Although usually packed down against the top of S2, RBDs can swing upwards to engage ACE2 (Roy et al., 2020). Monoclonal antibodies (mAbs) recognize one or both of ‘up’ and ‘down’ conformations (Zhou et al., 2020; Liu et al., 2020). The S protein is relatively conserved between SARS-CoV-2 and SARS-CoV-1 (76%), but the RBD and NTD are less conserved (74% and 50% respectively) than the S2 domain (90%) (Jaimes et al., 2020). Conservation with MERS-CoV and the seasonal human coronaviruses is much lower (19-21%). Overall SARS-CoV-2 antibodies show limited cross-reactivity even with SARS-CoV-1 (Tian et al., 2020).

The S protein has been studied intensively as a target for therapeutic antibodies. Previous studies on SARS-CoV-2 indicated that most potent antibodies bind close to the ACE2 interacting surface on the receptor binding domain (RBD) to block the interaction with ACE2 (Zost et al., 2020; Liu et al., 2020) expressed on target cells or disrupt the pre-fusion conformation (Huo et al., 2020; Yuan et al., 2020a; Zhou et al., 2020). However, SARS-CoV-2 therapeutic antibodies are not yet available for use in clinic.

Variant B.1.1.7 is now dominant in the UK, with increased transmission. B.1.1.7 harbours 9 amino-acid changes in the spike, including N501Y in the ACE2 interacting surface. Unrelated variants have been detected in South Africa (501Y.V2 also known as B.1.351) and Brazil (P.1, 501Y.V2), which have 10 and 12 amino-acid changes in the spike protein, respectively. All of these contain mutations in the ACE2 receptor binding footprint of the RBD, N501Y in B.1.1.7, K417N, E484K and N501Y in B.1.351 and K417T, E484K and N501Y in P.1, with the N501Y mutation being common to all. It is believed that these mutations in the ACE2 receptor binding domain increase the affinity for ACE2 (Zahradnik et al., 2021). These mutations also fall within the footprint of a number of potent neutralizing antibodies likely to afford vaccine induced protection and of several potential therapeutic monoclonal antibodies (Cheng et al., 2021; Nelson et al., 2021), thus affording mutant viruses greater fitness to infect new hosts but also to escape from pre-existing antibody response.

SARS-CoV-2 detection kits using monoclonal antibodies have also been developed. Examples include lateral flow tests by, e.g. Innova (SARS-CoV-2 Antigen Rapid Qualitative Test) and Quidel (Sofia 2 SARS Antigen FIA). However, these tests are reported to be highly inaccurate.

It is an object of the invention to identify further and improved antibodies useful for preventing, treating and/or diagnosing coronavirus infections, and diseases and/or complications associated with coronavirus infections, including COVID-19.

### SUMMARY OF THE INVENTION

The inventors initially identified 42 human monoclonal antibodies (mAbs) recognizing the spike protein of SARS-CoV-2 (see Table 1). These antibodies showed potent neutralisation activity against SARS-CoV-2, effective blocking of the interaction between the spike protein and ACE2 blocking and/or high affinity binding to the spike protein. It was found that nearly all highly potent neutralizing mAbs (IC₅₀<0.1 μg/ml) block the interaction with the ACE2, although one binds a unique epitope in the N-terminal domain. Some of the Table 1 antibodies demonstrated potent neutralization effects that were broadly effective against the hCoV-19/Wuhan/WIV04/2019 strain, as well as SARS-CoV-2 strains from various lineages, such as members of the B.1.1.7 (Alpha), B.1.351 (Beta), P.1 (Gamma), B.1.617 (Delta) and B.1.1.529 (Omicron) lineages.

Many of the Table 1 mAbs used public V-genes (V-genes shared by the majority of the population) and have few mutations relative to the germline. It was also found that several of the most potently inhibitory antibodies in Table 1 bind to unique epitopes compared to the antibodies previously described. Furthermore, N-glycosylation appears to improve antibody neutralisation activity. The most potent mAbs neutralized the virus in the low picomolar range, and showed beneficial effects when administered prior to or post infection in a murine model of COVID-19, hence demonstrating prophylactic and therapeutic effects.

The inventors generated further antibodies by swapping the light and heavy chains of the Table 1 antibodies. It was found that antibodies derived from the same public V-genes provided particularly useful mixed chain antibodies. For example, some of the resulting mixed chain antibodies exhibited potent neutralization effects that were broadly effective against the hCoV-19/Wuhan/WIV04/2019 strain, as well as SARS-CoV-2 strains from various lineages, such as members of the B.1.1.7, B.1.351 and/or P.1 linages.

Accordingly, an aspect of the invention provides an antibody capable of binding to the spike protein of coronavirus SARS-CoV-2, wherein the antibody: (a) comprises at least three CDRs of any one of the 42 antibodies in Table 1; and/or (b) binds to the same epitope as or competes with antibody 159, 45 or 384.

The invention also provides a combination of antibodies comprising two or more antibodies according to the invention.

The invention also provides a polynucleotide encoding the antibody according to the invention, a vector comprising said polynucleotide, or a host cell comprising said vector.

The invention also provides a method for producing an antibody that is capable of binding to the spike protein of coronavirus SARS-CoV-2, comprising culturing the host cell of the invention and isolating the antibody from said culture.

The invention also provides a pharmaceutical composition comprising: (a) the antibody or the combination of antibodies according to the invention, and (b) at least one pharmaceutically acceptable diluent or carrier.

The invention also provides the antibody, the combination of antibodies or the pharmaceutical composition according to the invention for use in a method for treatment of the human or animal body by therapy.

The invention also provides the antibody, the combination of antibodies or the pharmaceutical composition according to the invention, for use in a method of treating or preventing a disease or a complication associated with coronavirus infection.

The invention also provides a method of treating a subject comprising administering a therapeutically effective amount of the antibody, the combination of antibodies or the pharmaceutical composition according to the invention to said subject.

The invention also provides the use of the antibody, the combination of antibodies or the pharmaceutical composition according to the invention in the manufacture of a medicament for treating a subject.

The invention also provides a method of identifying the presence of coronavirus, or a protein or a protein fragment thereof, in a sample, comprising: (i) contacting the sample with the antibody or the combination of antibodies according to the invention, and (ii) detecting the presence or absence of an antibody-antigen complex, wherein the presence of the antibody-antigen complex indicates the presence of coronavirus, or a protein or a protein fragment thereof, in the sample.

The invention also provides a method of treating or preventing coronavirus infection, or a disease or complication associated therewith, in a subject, comprising identifying the presence of coronavirus according to the method of the invention, and treating the subject with an anti-viral or an anti-inflammatory agent.

The invention also provides an anti-viral or an anti-inflammatory agent for use in a method of treating or preventing coronavirus infection, or a disease or complication associated therewith, in a subject, wherein the method comprises identifying the presence of coronavirus according to the method of the invention, and treating the subject with a therapeutically effective amount of the anti-viral or the anti-inflammatory agent.

The invention also provides the use of the antibody, the combination of antibodies, or the pharmaceutical composition according to the invention for preventing, treating and/or diagnosing coronavirus infection, or a disease or complication associated therewith.

The invention also provides the use of the antibody, the combination of antibodies, or the pharmaceutical composition according to the invention for identifying the presence of coronavirus, or a protein or a protein fragment thereof, in a sample.

The invention also provides the antibody, the combination, or the pharmaceutical composition of the invention for use in a method of preventing, treating or diagnosing coronavirus infections caused by a SARS-CoV-2 strain comprising substitution at positions 417, 484 and/or 501 in the spike protein relative to the spike protein of the hCoV-19/Wuhan/WIV04/2019 strain, e.g. it is a member of lineage B.1.1.7, B.1.351 or P.1, or it is a member of lineage B.1.1.7, B.1.351, P.1, or B.1.1.529.

The invention also provides a method of preventing, treating or diagnosing coronavirus infections caused by a SARS-CoV-2 strain in a subject, wherein the method comprises administering the antibody, the combination, or the pharmaceutical composition of the invention to the subject, wherein the SARS-CoV-2 strain comprises substitution at positions 417, 484 and/or 501 in the spike protein relative to the spike protein of the hCoV-19/Wuhan/WIV04/2019 strain, e.g. it is a member of lineage B.1.1.7, B.1.351 or P.1, or it is a member of lineage B.1.1.7, B.1.351, P.1, or B.1.1.529.

The invention also provides the use of the antibody, the combination, or the pharmaceutical composition of the invention for the manufacture of a medicament for preventing, treating or diagnosing coronavirus infections caused by a SARS-CoV-2 strain comprising substitution at positions 417, 484 and/or 501 in the spike protein relative to the spike protein of the hCoV-19/Wuhan/WIV04/2019 strain, e.g. it is a member of lineage B.1.1.7, B.1.351 or P.1, or it is a member of lineage B.1.1.7, B.1.351, P.1, or B.1.1.529.

### BRIEF DESCRIPTION OF THE FIGURES

FIG. 1 shows the characterization of SARS-CoV-2-specific monoclonal antibodies (mAbs). (A) Cross-reactivity of 299 anti-spike (non-RBD) and 78 anti-RBD antibodies to trimeric spike of human alpha and beta-coronaviruses by capture ELISA. (B) Comparison of neutralisation potencies (IC₅₀) between anti-spike (non-RBD) and anti-RBD antibodies against authentic SARS-CoV-2 using focus reduction neutralisation test (FRNT). The Mann-Whitney U test was used for the analysis and two-tailed P values were calculated. (C) Correlation between SARS-CoV-2 neutralisation and RBD:ACE2 blocking by anti-RBD antibodies. Antibodies with IC₅₀<0.1 μg/ml, 0.1-1 μg/ml and 1-10 μg/ml are highlighted in red, blue and orange, respectively. (D) Plasma was depleted of RBD-specific antibodies using Ni-NTA beads coated with or without RBD, then evaluated for SARS-CoV-2 neutralizing activity by FRNT assay (n=8). Results are expressed as percent neutralisation of control without plasma. The percentage of depletion neutralizing antibodies for each sample tested is indicated at the top of each panel.

FIG. 2 shows the RBD anatomy and epitope definition based on mapping results. (A) Pale grey RBD surface with cartoon depiction of one monomer rainbow coloured from blue (N-terminus) to red (C-terminus) alongside grey surface depiction of RBD labelled to correspond to the adjacent torso (Torso Gaddi, Wikipedia, CC BY-SA 3.0, modified in Adobe Photoshop) used by analogy to enable definition of epitopes. (B) Cluster maps showing the output of the mapping algorithm with each spot corresponding to a ‘located’ antibody and colour-coded according to epitope. (C) BLI antibody data competition matrix (calculated values) output from cluster analysis showing the clustering into 5 epitopes. (D) The site of attachment of ACE2 (shown in purple), RBD residues contacting ACE2 are shown in green. (E) Located antibodies mapped onto the RBD shown as a grey surface with the ACE2-binding site in green. The individual antibodies are depicted as spheres and colour coded as in (B), those central to this paper are labelled. (F) as for (E) but antibodies are colour coded according to their ability to neutralize see inset scale, red strongest neutralizers, blue weakest neutralizers.

FIG. 3. RBD complexes. The Fab-RBD complexes reported in this paper as determined by a combination of X-ray crystallography and cryo-EM with the depictions here based on the crystallographically determined structures apart from the complex with Fab 40. Panel (A) shows the front view and panel (B) the back view with the RBD surface shown in grey and Fabs drawn as cartoons with the heavy chain in red and the light chain in blue. The ACE2 footprint on the RBD is coloured in green.

FIG. 4. spike morphology and Fab binding. (A) Orthogonal views of the trimeric spike as a pale grey surface with one monomer depicted as a cartoon and rainbow coloured from the N- to the C-terminus (blue to red). (B) Surface depiction of the electron potential map for the spike-mAb 159 complex determined by cryo-EM to 4.6 Å resolution. The spike is shown tilted forwards and coloured in teal apart from the RBDs (grey) and the fragment of mAb 159 that can be visualized is shown in orange. (C) Grey surface depiction of the RBD with a blue sphere denoting the location of Fab 45 as predicted using the mapping algorithm reported here. (D) Grey surface depiction of the X-ray crystallographic structure of the observed RBD-Fab 45 complex. Fab 45 binds close to the predicted position but is slightly translated. The S309 Fab (the closest structure in the competition matrix on which the mapping algorithm was based) is shown superimposed. Both Fabs are depicted as a cartoon with the heavy chain in magenta and light chain in blue. (E) Orthogonal grey surface depictions of the RBD with Fab 384 bound and Fab CV07-270 superimposed onto the complex. These Fabs use the same heavy chain V-gene but bind differently. They are drawn as cartoons with the heavy and light chains for Fab 384 in magenta and blue and those for CV07-270 in pale pink and light blue respectively.

FIG. 5. Determinants of binding, CDR length (A). Fab 384 interaction: left panel overview of the interacting CDRs from the heavy chain (magenta) and light chain (cyan) with the RBD (grey surface). The interactions of the H3, H2 and L1 and L3 loops are shown in the adjacent panels. (B) The distribution of IGHV, IGKV and IGLV gene usage of anti-RBD antibodies. Antibodies are grouped and coloured according to their neutralisation IC₅₀ values. (C) Left panel overview of the CDR interactions for Fabs 150 (magenta), 158 (cyan) and 269 (orange). Adjacent panels (top) show a close up of the H3 loop interactions for each of these antibodies retaining the same colour coding and the bottom panel shows the interactions of the L3 loop and also the sequence alignment for the loops (150 H3 loop (SEQ ID NO: 157), 158 H3 loop (SEQ ID NO: 167), 269 H3 loop (SEQ ID NO: 277), 150 L3 loop (SEQ ID NO: 160), 158 L3 loop (SEQ ID NO: 170) and 269 L3 loop (SEQ ID NO: 280)). (D) Back and side views of the complex of Fab 40 and RBD (grey surface) with the Fab drawn as a cartoon with the heavy chain in magenta and the light chain in blue. Fab 158 (grey cartoon) is superimposed. Note despite Fab 40 using the IGVH3-66 public V-gene whilst 158 uses IGVH3-53 they bind almost identically. (E) Fab 75-RBD complex with the RBD drawn as a cartoon in magenta and the Fab similarly depicted with the heavy chain in orange and the light chain in grey. This antibody uses IGHV3-30 and is not a potent neutralizer. It can be seen that the only heavy chain contact is via the extended H3 loop.

FIG. 6. Determinants of binding, light chain swapping and glycosylation. (A) Table of sequences of MAbs 253 (heavy chain AA junction: SEQ ID NO: 428; light chain AA junction SEQ ID NO: 431), 55 (heavy chain AA junction: SEQ ID NO: 429; light chain AA junction: SEQ ID NO: 432) and 165 (heavy chain AA junction: SEQ ID NO: 430; light chain AA junction: SEQ ID NO: 432). (B) Neutralisation activity of authentic SARS-CoV-2 by the original mAb253, chimeric mAb253H55L and chimeric 253H165L (presented as IC₅₀ values). Immunoglobulin heavy and light-chain gene alleles are presented in the table. Data are from 3 independent experiments, each with duplicate wells and the data are shown as mean±s.e.m. (C) The chimeric Fab 253H55L ((mAb 253 (IGVH1-58, IGVK3-20) heavy chain combined with the light chain of mAb 55 also (IGVH1-58, IGVK3-20) but containing the IGKJ1 region in contrast of IGKJ2 in mAb 253 in complex with the RBD here shown as a hydrophobic surface. The Fab is drawn as a ribbon with the heavy chain in magenta and the light chain in blue. This 10-fold increase in neutralisation titre of this Fab compared to 253 appears to come from the single substitution of a tryptophan for a tyrosine making a stabilizing hydrophobic interaction. (D) CDRs with sugar bound in the RBD complexes with Fabs 88 (top panel) sugar bound to N35 in the H1 loop, 316 (middle panel) sugar bound to N59 in the H2 loop and 253 (bottom panel) sugar bound to N102 in the H3 loop. Note Phe 486, is marked by a diamond to enable the various orientations to be related.

FIG. 7. Determinants of binding, RBD conformation, valency of interaction. (A) Cryo-EM spike-Fab complexes showing different RBD conformations. The density for the spike is shown in teal, the RBD in grey and Fab in orange. Left ‘all RBDs down’ conformation with Fab 316 bound, middle ‘one RBD up’ conformation with one Fab 158 bound, right ‘all RBDs up’ conformation with 3 Fab 88s bound. (B) Potently neutralizing Fab 159 (cartoon representation with red heavy chain and blue light chain) in complex with the NTD (grey transparent surface) and adjacent depicted with another NTD binding Fab (4A8) superimposed as a grey ribbon, the binding sites are separated by ˜15 Å. (C) Fab 159 (HC magenta, LC blue) is drawn as a cartoon in its binding location on top of the NTD of the spike which is drawn as a grey surface and viewed from the top (a full IgG is modelled onto one monomer showing that it cannot reach across to bind bivalently). (D) ELISA binding (blue) and FRNT neutralisation (red) curves of ten full-length antibodies (solid lines) and corresponding Fab molecule (dash lines) against SARS-CoV-2. Data are from 2 independent experiments (mean±s.e.m.)

FIG. 8. In vivo studies. Neutralizing antibodies protect against SARS-CoV-2 in K18-hACE2 transgenic mice. A-G. Seven to eight-week-old male and female K18-hACE2 transgenic mice were inoculated by an intranasal route with 103 PFU of SARS-CoV-2. At 1 day post infection (dpi), mice were given a single 250 μg (10 mg/kg) dose of the indicated mAb by intraperitoneal injection. A, Weight change (mean±SEM; n=5-10, two independent experiments: two-way ANOVA with Sidak's post-test: ns, not significant, * P<0.05, ** P<0.01, **** P<0.0001; comparison to the isotype control mAb treated group). B-G. At 7 days post infection (dpi) tissues were harvested and viral burden was determined in the lung (B-C), heart (D), spleen (E), nasal washes (F), and brain (G) by plaque (B) or RT-qPCR (C-G) assay (n=7-11 mice per group; Kruskal-Wallis test with Dunn's post-test: ns, not significant, * P<0.05, ** P<0.01, *** P<0.001, **** P<Dotted lines indicate the limit of detection.

FIG. 9. SARS-CoV-2 elicits binding and neutralizing antibodies against trimeric spike, RBD and NP proteins. (A) Plasma from donors with confirmed SARS-CoV-2 infection were collected at 1-2 months after onset of symptoms and tested for binding to SARS-CoV-2 spike, RBD and N proteins by capture ELISA. (B) neutralizing titres to authentic live virus. Data are representative of one experiment with 42 samples and presented as means±s.e.m. (C) Comparison of the frequency of spike-reactive IgG expressing B cells in mild cases and severe cases measured by FACS. Small horizontal lines indicate the median. Data are representative of one experiment with 16 samples. The Mann-Whitney U test was used for the analysis and two-tailed P values were calculated (in B and C).

FIG. 10. SARS-CoV-2 antibody isolation strategies. Human monoclonal antibodies from memory B cells were generated using two different strategies. (A) IgG expressing B cells were isolated and cultured with IL-2, IL-21 and 3T3-msCD40L cells for 13-14 days. Supernatants were harvested and tested for reactivity to spike protein by ELISA. (B) Antigen-specific single B cells were isolated using labelled recombinant spike or RBD proteins as baits. The IgG heavy and light chain variable genes from both strategies were amplified by nested PCR and cloned into expression vectors to produce full-length IgG1 antibodies.

FIG. 11. Specificity and sequence analysis of 377 human antibodies. (A) Epitope mapping of SARS-CoV-2-specific antibodies against the RBD, 51 subunit (aa 16-685) and S2 subunit (aa 686-1213) were evaluated by ELISA, and the NTD-binders were identified by cell-based fluorescent immunoassay. Antibodies interacting with none of the subdomains were defined as trimeric spike. The number in the centres indicate the total number of tested antibodies. (B) Frequency of amino acid substitutions from germline in SARS-CoV2-specific heavy and light chains (n=377). (C) Repertoire analysis of antibody heavy and light chains of anti-S(Non-RBD) and anti-RBD antibodies. At the centre is the number of antibodies. Each slice represents a distinct clone and is proportional to the clone size. (D) Frequency of amino acid substitutions from germline in heavy and light chains of antibodies cross-reacting between SARS-CoV-2 and the 4 seasonal coronaviruses (n=20).

FIG. 12. Crystal structures of the ternary complexes. (A) RBD-88-45, (B) RBD-253-75, (C) RBD-253H55L-75 and (D) RBD-384-S309 complexes.

FIG. 13. Cryo-EM Data Resolution and map quality at the RBD-Fab/IgG interface. (A-K) [left] Gold-standard FSC curve (FSC=0.143 marked) generated by cryoSPARC for fab (or IgG in the case of 159)-spike structures [right] showing map quality at the antigen/antibody interface with 40, 88, 150, 158, 316, 384, 253H55L RBD up, 253H55L RBD down, 253H165L, 159 RBD down, 159 RBD up, respectively.

FIG. 14. Overrepresentation of binding modes. (A) Sequence alignment for HC CDR3s using public V-region 3-53, antibodies are represented by a number (from this study) or by PBD code and a name (antibody 150: SEQ ID NO: 433, CV30(6XE1): SEQ ID NO: 434, B38(7bZ5): SEQ ID NO: 435, p2c-lf11(7CDI): SEQ ID NO: 436, BD604(7CH4): SEQ ID NO: 437, BD236(7CHB): SEQ ID NO: 438, antibody 158: SEQ ID NO: 439, COVA2-04(7JMO): SEQ ID NO: 440, CC12.1(6XC3): SEQ ID NO: 441, antibody 175: SEQ ID NO: 442, CC12.3(6XC4): SEQ ID NO: 443, BD629(7CHC): SEQ ID NO: 444, CB6(7C01): SEQ ID NO: 445, 7CJF: SEQ ID NO: 446, antibody 222: SEQ ID NO: 447, antibody 269: SEQ ID NO: 448). (B) Comparison of binding modes of 150 (orange), 158 (cyan), 269 (magenta). (C) Superimposition of RBD-Fab complexes available in PDB (up to 21st Oct. 2020). RBD is shown as grey surface, Fabs as Ca traces with heavy chains in warm colour and light chains in cool colour. (D) The bound Fabs can be divided into four major clusters, neck (B38(7bZ5), CB6(7C01), CV30(6XE1), CC12.3(6XC4), CC12.1(6XC3), COV2-04(7JMO), BD629(7CHC), BD604(7CH4), BD236(7CHB)), left shoulder (p2b-2f6(7BWJ), BD368(7CHC), C07-270(6XKP)), left flank (EY6A(6ZCZ), CR3022(6YLA), 5304(7JX3), COVA1-16(7JMW)) and right flank (S309 (7JX3)), according to their binding modes on RBD. (E) Outliers that include right shoulder binders (REGN10987 (6XDG), COVA2-39 (7JMP), CV07-250 (6XKQ), S2H14 (7JX3)). One Fab in the neck cluster is drawn as red and blue surface to show the relative position of the outliers.

FIG. 15. Importance of antibody glycosylation. (A-C) Effect of mutation of the Asn residue glycosylated in the heavy chains of antibodies 88, 253 and 316 respectively. (D-F)|2Fo-Fc| electron density maps contoured at 1.2 σ showing the glycans at glycosylation sites at N35 of 88 (D) N59 of 316 (E) and N102 of 253 (F). (G) Relative binding position and orientation of CDR-H3 and glycans between 316 (green) and 88 (orange), and (H) between 316 and 253 (cyan). RBD is shown as a grey surface.

FIG. 16. Classification of Cryo-EM datasets show spike heterogeneity for 384 and 158. (A) Gaussian filtered reconstructed volume (transparent grey) with refined spike (from two clusters of 384 following local variability analysis using cryoSPARC). At very low contour levels, and with Gaussian filtering, there is slight evidence of one (right), or two (left) additional bound fabs. (B) Reconstructed volume for 159 in the RBD up (left) and down (right) positions, coloured by spike chain (blue, green, purple) and IgG (orange). The RBD in the up position is indicated by a red arrow.

FIG. 17. Prophylaxis with mAbs 40 and 88 protects against weight loss and decreases viral burden. A-G. Seven to eight-week-old male and female K18-hACE2 transgenic mice were given a single 250 g dose of the indicated mAbs by intraperitoneal injection. One day later, mice were inoculated by intranasal route with 103 PFU of SARS-CoV-2. (A) Weight change (mean±SEM; n=6, two independent experiments: two-way ANOVA with Sidak's post-test: ns, not significant, * P<0.05, **** P<0.0001; comparison is to the isotype control mAb treated group). B-G. At 7 days post infection (dpi) tissues were harvested and viral burden was determined in the lung (B-C), heart (D), spleen (E), nasal washes (F), and brain (G) by plaque assay (B) or RT-qPCR (C-G) assay (n=6 mice per group; Kruskal-Wallis test with Dunn's post-test: ns, not significant, * P<0.05, ** P<0.01 ***P<0.001). Dotted lines indicate the limit of detection.

FIG. 18. The B.1.1.7 (Kent) Variant Spike protein. The SARS-CoV-2 spike trimer is depicted as a grey surface with mutations highlighted in yellow-green or with symbols. The RBD N501Y and the NTD 144 and 69-70 deletions are highlighted with green stars and red triangles respectively. On the left a protomer is highlighted as a coloured ribbon within the transparent grey spike surface, illustrating its topology and marking key domains.

FIG. 19. ACE2 binding comparison and effect on ACE binding of the N501Y mutation. (A) The RBD ‘torso’ analogy. The RBD is represented as a grey surface with the ACE2 receptor binding site in dark green. Binding sites for the panel of antibodies on which this study draws are represented by spheres coloured according to their neutralisation, from red (potent) to blue (non-neutralising). The position of the B.1.1.7 N501Y mutation in the RBD is highlighted in light green towards the right shoulder. (B) Proximity of ACE2 to N501Y. The RBD is depicted as in (A) with ACE2 bound (in yellow cartoon format) with glycosylation drawn as sticks. (C) Left panel: interactions of N501 of WT RBD with residues Y41 and K353 (Lan et al., 2020). When the 501 is mutated to a tyrosine with the conformation seen in the N501Y RBD-269 Fab complex (right panel), Y501 makes T-shaped ring stacking interactions with Y41 and more hydrophobic contacts with K353 of ACE2 (note there are minor clashes of the side chain of Y501 to the end of the K353 side chain, which has ample room to adjust to optimise interactions). (D) BLI plots for WT (left) and N501Y (right) RBDs binding to ACE2. A titration series is shown for each (see Methods). Note the much slower off-rate for the mutant.

FIG. 20. mAb binding to WT and N501Y RBD. (A) Structural overlay of RBD-Fab complexes in which Fabs have direct contact with N501. The overlay was done by superimposing the RBD. Structures of 38 antibody Fabs in complex with RBD were analysed. 18 have direct contact with N501 (left), which includes 14 IGHV3-53, 2 IGHV3-66 and two others. 20 Fabs don't have direct contact with N501 of the RBD (right), these include 3 IGHV3-53 or IGHV3-66 Fabs (Table 13). The RBD is shown as grey surface with residue N501 highlighted in magenta. The Ca backbones of Fabs are drawn as thin sticks. (B) Examples of optimised binding to the asparagine 501 side chain for antibodies B38 and 158. (C) BLI results for potent binders selected from a panel of antibodies comparing 501Y RBD with 501N RBD. (D) Left pair: BLI data mapped onto the RBD using the method described herein. Front and back views of the RBD are depicted as in (A) but with the spheres representing the antibody binding sites coloured according to the ratio (KD501Y/KD501N). For white the ratio is 1, for red it is <0.1 (i.e. at least 10-fold reduction). Right pair: As for the left pair but coloured according to the ratio of neutralisation titres (IC50501Y/IC50501N). For white the ratio is 1, for red it is <0.01 (i.e. at least 100-fold reduction). Note the strong concordance between the two effects, with 269 being the most strongly affected. The nearby pink antibodies are mainly the IGHV3053 and IGHV3-66 antibodies.

FIG. 21. Molecular Mechanisms of Escape and comparison of N501Y RBD/269 Fab and RBD/scFv269 complexes. (A) CDR-L1 (thin sticks) positions of a panel of V3-53 Fabs relative to N501 of RBD (surface, with N501 highlighted in green). (B) The side chain of N501 makes extensive contacts with residues from CDR-L1 in the RBD-158 Fab complex (left). In the right panel, N501 does not make any contact with p2c-2f11 Fab whose LC is most similar in sequence and has the same CDR-L1, L2 and L3 lengths to mAb 222 shown by a blast of 222 LC against the PDB. The orientation and position of Y501 in the N501Y RBD-269 Fab complex is shown by overlapping the RBDs in both panels. (C) Crystallographic structure of N501Y RBD/Fab 269. Overlay of Cαs of N501Y RBD/Fab 269 (blue) with RBD/scFv269 (salmon) by superimposing the RBDs of the two complexes. (D) Structure changes in the 496-501 loop of the RBD and the CDR-L1 loop that contacts the mutation site. (E) Structural difference of the CDR-L3 loops between the two complexes.

FIG. 22. Neutralization of SARS-CoV-2 strains Victoria and B.1.1.7 by mAb. (A) Neutralization curves of potent (FRNT50<100 ng/ml) anti-RBD antibodies including those expressing the public heavy chain VH3-53. (B) Regeneron antibodies REGN10933; REGN10987 and AstraZeneca antibodies AZD8895 and AZD7442 (AZD1061 plus AZD8895) are included for comparison. Neutralization of SARS-CoV-2 was measured using a focus reduction neutralization test (FRNT).

FIG. 23. Neutralization activity of convalescent plasma and vaccine sera. (A) Neutralization titres of 34 convalescent plasma collected 4-9 weeks following infection are shown with the WHONIBSC 20/130 reference serum (B) Neutralization titres of serum from volunteers vaccinated with the AstraZeneca vaccine ADZ1222, samples were taken at (i) 14 days following the second dose (n=10) and (ii) 28 days following the second dose (n=15). (C) Neutralization titres of serum taken from volunteer healthcare workers recruited following vaccination with Pfizer-BioNTech BNT162b2 (n=25). Neutralization was measured by FRNT, the Mann-Whitney U test was used for the analysis and two-tailed P values were calculated, mean values are indicated above each column.

FIG. 24. Neutralization activity of serum taken from patients suffering infection with B.1.1.7. (A) Neutralization titres of plasma from 13 patients infected with B.1.1.7 at various time points following infection. The days since infection are indicated in each panel Neutralization was measured by FRNT. (B) Comparison of FRNT50 titres of individual sera against Victoria and B.1.1.7 strains, the number above each column is the mean, the Mann-Whitney U test was used for the analysis and two-tailed P values were calculated.

FIG. 25. N5-1Y containing sequences in the UK. (A) proportion of three subgroups of B.1.1.7 expressed as percentage of total 501Y-containing identifiable sequences. Black line shows dominant form with 501Y and Δ69-70. Blue, orange lines both lack 69-70 and have either wild-type or S982A mutation respectively. (B) associated mutations for blue (left), orange (middle) and black (right) plotted on Spike protein structure where modelled, with extended modelled N-terminus (PDB code 6ZWV).

FIG. 26. Electron density maps for residue 501. Electron density maps for residue 501 refined as a tyrosine in (A) and as an asparagine in (B). 2Fo-Fc maps are contoured at 1.2 σ and coloured in blue in both panels. The negative density (red) in (A) is contoured at −3 σ, and the positive density (green) in (B) at 3 σ.

FIG. 27. Evolution of B.1.351 Variant: (A-B) Sliding 7-day window depicting proportion of sequences with wild-type (grey), 501Y mutation only (green), NTD deletion only (purple) and double mutation variant (black) for (A) sequences selected containing UK, NTD deletion 69-70 and (B) South Africa, NTD deletion 241-243. (C) structure plot showing distribution of mutations of South African variant sequences as defined by 501Y and deletion 241-243. Structure plots use Spike protein structure (original frame from PDB code 6ZWV) where modelled, and models were extended in Coot for missing loops. (D) Positions of major changes in the spike protein are highlighted in the NTD and RBD (E) positions of the K417N, E484K and N501Y (yellow) mutations within the ACE2 interacting surface (dark green) of RBD.

FIG. 28. Neutralization of Victoria and B.1.351 viruses by Convalescent plasma. Plasma was collected in the UK before June 2020, during the first wave of SARS-CoV-2, in the early convalescent phase 4-9 weeks following admission to hospital. (A) FRNT assays comparing neutralization of Victoria (orange) and B.1.351 (green) (n=34). (B) Neutralization assays of Victoria and B.1.351 with plasma obtained from patients suffering B.1.1.7 infection at the indicated times following infection. (C-D) Comparison of FRNT₅₀ titres between B.1.351 and Victoria strains for convalescent and B.1.1.7 plasma respectively, the Wilcoxon matched-pairs signed rank test was used for the analysis and two-tailed P values were calculated, geometric mean values are indicated above each column. Individual FRNT₅₀ values are shown in Table 14.

FIG. 29. Neutralization of B.1.351 by Vaccine serum. Neutralization FRNT curves for Victoria and B.1.351 strains by (A) 25 sera taken 7-17 days following the second dose of the Pfizer BioNTech vaccine. (B) 25 sera taken 14 or 28 days following the second dose of the Oxford-AstraZeneca vaccine. (C-D) Comparison of FRNT₅₀ titres between B.1.351 and Victoria strains for the Pfizer-BioNTech and Oxford-AstraZeneca vaccines respectively, the Wilcoxon matched-pairs signed rank test was used for the analysis and two-tailed P values were calculated, geometric mean values are indicated above each column. Individual FRNT₅₀ values are shown in Table 15.

FIG. 30. Neutralization by potent monoclonal antibodies. (A) Neutralization curves for Victoria and B.1.351 using 22 human monoclonal antibodies. (B) Neutralization curves of Victoria and B.1.351 strains using monoclonal antibody pairs from Regeneron and AstraZeneca. Individual FRNT₅₀ values are shown in Table 16.

FIG. 31. Interactions of mutation site residues with a selection of RBD binding mAbs. (A) Interactions of Fab 88 with K417 and E484 of the RBD (PDB ID 7BEL), (B) 150 with N501 and K417 (PDB ID 7BEI), (C) 253 has no contact with any of the three mutation sites (PDB ID 7BEN) and (D) Fab 384 with only E484 (PDB ID 7BEP). (E) Structures of IGHV3-51 and IGHV3-66 Fabs by overlapping the Ca backbones of the RBD. (F) Interactions of K417 with CB6 Fab (PDB ID 7C01 (Wajnberg et al., 2020)). (G) The K417N mutation is modelled in the RBD/CB6 complex. In (A) to (G), the Fab light chain, heavy chain and RBD are in blue, salmon and grey respectively. Ca backbones are drawn in thinner sticks and side chains in thicker sticks. Contacts (≤4 Å) are shown as yellow dashed lines, hydrogen bonds and salt bridges as blue dashed lines. (H) Positions of mutations and the deletion in the Spike NTD of the B.1.351 variant relative to the bound antibodies 159 (PDB ID 7NDC), and (I) to 4A8 (PDB ID 7C2L), the 242-244 deletion would be predicted to disrupt the interaction of 159 and 4A4. The VH and VL domains of the Fabs are shown as salmon and blue surfaces respectively, NTD as grey sticks. The mutation sites are drawn as green spheres and deletions as magenta spheres.

FIG. 32. Antibody RBD interaction and structural modelling. BLI plots showing a titration series of binding to ACE2 (see Methods) for (A) Wuhan RBD and (B) K417N, E484K, N501Y B.1.351 RBD. Note the much slower off-rate for B.1.351. (C and D) KD of RBD/mAb interaction measured by BLI for WT Wuhan RBD (left dots) and K417N, E484K, N501Y B.1.351 RBD (right dots). (E) Epitopes as defined by the clustering of mAbs on the RBD (grey). (F) BLI data mapped onto the RBD using the method described herein. Front and back views of the RBD are depicted with the spheres representing the antibody binding sites coloured according to the ratio (KDB.1.351/KDWuhan). For white the ratio is 1, for red it is <0.1 (i.e. at least 10-fold reduction) black dots refer to mapped antibodies not included in this analysis, dark green RBD ACE2 binding surface, yellow mutated K417N, E484K, N501Y. (G) As for the left pair but coloured according to the ratio of neutralisation titres (IC50B.1.351/IC50Victoria), for white the ratio is 1, for red it is <0.01 (i.e. at least 100-fold reduction). Note the strong concordance between the two effects, with 269 being the most strongly affected. The nearby pink antibodies are mainly the IGHV3-53 and IGHV3-66 antibodies.

FIG. 33. Mutational landscape of P.1. Schematic showing the locations of amino acid substitutions in (A) P.1, (B) B.1.351 and (C) B.1.1.7 relative to the Wuhan SARS-CoV-2 sequence. Under the structural cartoon is a linear representation of S with changes marked on. Where there is a charge change introduced by mutations the change is coloured (red if the change makes the mutant more acidic/less basic, blue more basic/less acidic). (D) Depiction of the RBD as a grey surface with the location of the three mutations K417T, E484K and N501Y (magenta) the ACE2 binding surface of RBD is coloured green. (E) locations of N-linked glycan (red spheres) on the spike trimer shown in a pale blue surface representation, the two new sequins found in P.1 are marked blue.

FIG. 34. Comparison of WT RBD/ACE2 and P.1 RBD/ACE2 complexes. (A) Comparison of P.1 RBD/ACE2 (grey and salmon) with WT RBD/ACE2 (blue and cyan) (PDB ID 6LZG) by overlapping the RBDs. The mutations in the P.1 RBD are shown as sticks. (B), (C) Open book view of electrostatic surface of the WT RBD/ACE2 complex, and (C), (D) of the P.1 RBD/ACE2 complex. Note the charge difference between the WT and the mutant RBDs. The charge range displayed is ±5 kJ/mol. (E) The K417 of the WT RBD forms a salt bridge with D30 of ACE2. (F) and (G) Effect of E484K mutation on the electrostatic surface (H) Y501 of the P.1 RBD makes a stacking interaction with Y41 of ACE2. (I) KD of RBD/mAb interaction measured by BLI for RBDs of Victoria, B.1.1.7, P.1 and B.1.351 (left to right) (J) BLI data mapped onto the RBD using the method described. Front and back views of the RBD are shown. In the left pair the spheres represent the antibody binding sites coloured according to the ratio (KDP.1/KDWuhan). For white the ratio is 1, for red it is <0.1 (i.e. at least 10-fold reduction) black dots refer to mapped antibodies not included in this analysis, dark green RBD ACE2 binding surface, yellow mutated K417T, E484K, N501Y. For the right pair atoms are coloured according to the ratio of neutralisation titres (IC50B.1.351/IC50Victoria), for white the ratio is 1, for red it is <0.01 (i.e. at least 100-fold reduction). Note the strong agreement between KD and IC50. 269 is very strongly affected and is close to the IGHV3-53 and IGHV3-66 antibodies (e.g. 222).

FIG. 35. Neutralization of P.1 by monoclonal antibodies. (A) Neutralization of P.1 by a panel of 20 potent human monoclonal antibodies. Neutralization was measured by FRNT, curves for P.1 are superimposed onto curves for Victoria, B.1.1.7 and B.1.351. FRNT₅₀ titres are reported in Table 18 Neutralization curves for monoclonal antibodies in different stages of development for commercial use. (B) Shows equivalent plots for the Vir, Regeneron, AstraZeneca, Lilly and Adagio antibodies therapeutic antibodies.

FIG. 36. Structures of Fab 222 in complex with P.1 RBD. (A) Ribbon depiction of Fab 159/NTD complex with P1 mutations in the NTD highlighted as cyan spheres. (B) Front and back surfaces of the RBD bound to a typical VH3-53. P1 mutations in the RBD are highlighted in dark green and labelled. In this group, monoclonal antibody 222 has a slightly longer CDR3. Sequences of VH3-53 CDR1-3 heavy and light chains are also shown (150 CDR-H1 (SEQ ID NO: 449), 150 CDR-H2 (SEQ ID NO: 450), 150 CDR-H3 (SEQ ID NO: 451), 150 CDR-L1 (SEQ ID NO: 464), 150 CDR-L3 (SEQ ID NO: 465); 158 CDR-H1 (SEQ ID NO: 452), 158 CDR-H2 (SEQ ID NO: 453), 158 CDR-H3 (SEQ ID NO: 454), 158 CDR-L1 (SEQ ID NO: 466), 158 CDR-L3 (SEQ ID NO: 467); 222 CDR-H1 (SEQ ID NO: 455), 222 CDR-H2 (SEQ ID NO: 456), 222 CDR-H3 (SEQ ID NO: 457), 222 CDR-L1 (SEQ ID NO: 468), 222 CDR-L3 (SEQ ID NO: 469); 269 CDR-H1 (SEQ ID NO: 458), 269 CDR-H2 (SEQ ID NO: 459), 269 CDR-H3 (SEQ ID NO: 460), 269 CDR-L1 (SEQ ID NO: 470), 269 CDR-L3 (SEQ ID NO: 471); 175 CDR-H1 (SEQ ID NO: 461), 175 CDR-H2 (SEQ ID NO: 462), 175 CDR-H3 (SEQ ID NO: 463), 175 CDR-L1 (SEQ ID NO: 472), 175 CDR-L3 (SEQ ID NO: 473)). (C) Crystal structure of P1 RBD, 222 Fab and EY6A Fab (Zhou et al., 2020). (D) Close up of 222 CDRs interacting with the RBD (grey) mutations are highlighted in yellow on the green ACE2 interface. (E) K417 interactions with Fab 222 (F) N501 interactions with Fab 222. (G), (H) Fab 222 chimera models.

FIG. 37. Neutralization curves of VH3-53 chimeric antibodies. Neutralization curves of Victoria, B.1.1.7, B.1.351 and P.1. Left hand column; neutralization curves using the native antibodies 222, 150, 158, 175 and 269. Right hand column; neutralization curves for chimeric antibodies, the heavy chains of 150, 158, 175 and 269 are combined with the light chain of 222, native 222 is used as the control. FRNT₅₀ titres are given in Table 18.

FIG. 38. Neutralization of P.1 by convalescent plasma. Plasma (n=34) was collected from volunteers 4-9 weeks following SARS-CoV-2 infection, all samples were collected before June 2020 and therefore represent infection before the emergence of B.1.1.7 in the UK. (A) Neutralization of P.1 was measured by FRNT, comparison is made with neutralization curves for Victoria, B.1.1.7 and B.1.351 that we have previously generated. (B) Neutralization of P.1 by plasma taken from volunteers who had suffered infection with B.1.1.7 as evidenced by sequencing or S-gene drop out by diagnostic PCR. Samples were taken at varying times following infection. (C-D) Comparison of FRNT₅₀ titres between Victoria and P.1, data for B.1.1.7 and B.1.351 are included for comparison and, the Wilcoxon matched-pairs signed rank test was used for the analysis and two-tailed P values were calculated, geometric mean values are indicated above each column.

FIG. 39. Neutralization of P.1 by vaccine serum. (A) Pfizer vaccine, serum (n=25) was taken 7-17 days following the second dose of the Pfizer-BioNTech vaccine. FRNT titration curves are shown with Victoria, B.1.1.7 and B.1.351 as comparison. (B) AstraZeneca vaccine, serum was taken 14 or 28 days following the second dose of the Oxford-AstraZeneca vaccine (n=25). (C-D) Comparison of FRNT₅₀ titres for individual samples for the Pfizer and AstraZeneca vaccine between Victoria, B.1.1.7, B.1.351 and P.1, the Wilcoxon matched-pairs signed rank test was used for the analysis and two-tailed P values were calculated, geometric mean values are indicated above each column.

FIG. 40. Sliding 7-day window depicting proportion of sequences containing K417T.

FIG. 41. BLI titration for the attachment and dissociation of ACE2 from P.1 RBD attached to the tip.

FIG. 42. Cross reactivity of panel of mAbs identified from recovered COVID-19 patients. Neutralization assays performed against Victoria, Alpha (N501Y), Beta (K417N, E484K, N501Y), Gamma (K417T, E484K, N501Y), Delta (L452R, T478K), and Omicron (G339D, 5371L, S373P, S375F, K417N, N440K, G446S, S477N, T478K, E484A, Q493R, G496S, Q498R, N501Y, Y505H) live viral isolates with the selected mAbs. Titration curves are shown FRNT₅₀ values are reported in Table 26.

FIG. 43. (A) Table defining the mutations in the spike protein from different strains when compared with the Wuhan SARS-CoV-2 spike protein sequence. (B) Graphs showing IC₅₀ curves of selected antibodies against a panel of psuedoviral constructs with the mutations when compared with the Wuhan SARS-CoV-2 spike protein sequence in Table 28.

### DETAILED DESCRIPTION OF THE INVENTION

### Antibodies of the Invention

An antibody of the invention specifically binds to the spike protein of SAR-CoV-2. In particular, it specifically binds to the 51 subunit of the spike protein, such as the receptor binding domain (RBD) or N-terminal domain (NTD).

An antibody of the invention may comprise at least three CDRs of an antibody in Table 1. Table 1 lists 42 individual antibodies that were identified from recovered COVID-19 patients. Table 1 also lists the SEQ ID NOs for the heavy chain variable region and light chain variable region nucleotide and amino acid sequences, and the complementarity determining regions (CDRs) of the variable chains, of each of the antibodies. The CDRs of the heavy chain (CDRH) and light chain variable domain (CDRL) are located at residues 27-38 (CDR1), residues 56-65 (CDR2) and residues 105-117 (CDR3) of each chain according to the IMGT numbering system (http://www.imgt.org; Lefranc M P, 1997, J, Immunol. Today, 18, 509). This numbering system is used in the present specification except where otherwise indicated.

The antibody in Table 1 may be any antibody selected from the group consisting of: 253H55L, 253H165L, 253, 222, 318, 55, 165, 384, 159, 88, 40 and 316. The antibody in Table 1 may be any antibody selected from the group consisting of: 253H55L, 253H165L, 253, 222, 318, 55 and 165. The antibody in Table 1 may be any antibody selected from the group consisting of: 384, 159, 253H55L, 253H165L, 253, 88, 40 and 316.

Antibodies 253H/55L, 253H/165L, 253, 222, 318, 55 and 165 are all highly potent neutralising mAbs that have been shown to neutralize the Victoria, Kent (B.1.1.7), South Africa (B.1.351) and Brazilian (P.1) strains, without a loss in potency.

The antibody in Table 1 may be any antibody selected from the group consisting of 88, 159, 222, 281, 316, 384 and 398. These antibodies were found to have potent cross-lineage neutralisation effects, e.g. they are effective against the Victoria and B.1.1.7 strains (e.g. a B.1.1.7:Victoria ratio of less than 2 and/or an IC50 as shown in Table 11 of less than 0.1 μg/ml).

The antibody in Table 1 may be any antibody selected from the group consisting of: 55, 58, 150, 165, 222, 253, 278, 318, 253H55L and 253H165L. These antibodies were found to have potent cross-lineage neutralisation effects, e.g. they are effective against the Victoria and B.1.351 strains (e.g. a B.1.1.7:Victoria ration of less than 3 and/or an IC50 of less than 0.1 μg/ml, see Tables 11 and 16A).

The antibody in Table 1 may be any antibody selected from the group consisting of: 222, 318, 253H55L and 253H165. These antibodies were found to have potent cross-lineage neutralisation effects, e.g. they are effective against the Victoria and B.1.351 strains (e.g. a B.1.1.7:Victoria ration of less than 3 and/or an IC50 of less than 0.1 μg/ml, see Tables 11 and 16A) and bind to the spike protein with high affinity, e.g. having KD≤4 nM (see Table 16A).

The antibody in Table 1 may be 222 or 253H165L. These antibodies were found to have potent cross-lineage neutralisation effects, e.g. they have an IC50 of ≤0.02 μg/ml against the Victoria strain, B.1.1.7 strain, B.1.351 strain and P.1 strain, and bind to the spike protein with high affinity (see Table 18).

The antibody in Table 1 may be any antibody selected from the group consisting of: 58, 222, 253 and 253H/55L. These antibodies were found to neutralize the omicron (B.1.1.529) strain with an IC50 of ≤5 μg/ml. The antibody in Table 1 may be 58 or 222. These antibodies were found to strongly neutralize the omicron strain with an IC50 of ≤0.25 μg/ml.

The 253H55L antibody is generated from the combination of antibody 253 and antibody 55. These antibodies individually were not the most potent antibodies identified. However, once the heavy chain from antibody 253, and the light chain from antibody 55 were combined, the resultant antibody unexpectedly had improved neutralisation and antigen-binding. For example, 253H55L conferred one of the greatest reductions in viral RNA levels in in vivo models of SARS-CoV-2 infection. Therefore, in a preferred embodiment, the antibody in Table 1 is 253H55L.

Hence, an antibody of the invention may comprise at least three CDRs of antibody 253H55L. In one embodiment, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 68, 69 and 70, respectively.

Antibody 253H/165L was identified in a similar manner to 253H165L. It was surprisingly found that 253H/165L bound more strongly to SARS-CoV-2 than antibody 253 or antibody 165 alone (Table 3).

Accordingly, an antibody of the invention may comprise at least three CDRs of antibody 253H165L. In one embodiment, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 188, 189 and 190, respectively.

Furthermore, it was identified that antibodies 253, 165 and 55 unexpectedly retained potent neutralisation of SARS-CoV-2 variants B.1.1.7 and B.1.351.

Accordingly, an antibody of the invention may comprise at least three CDRs of antibody 253, 165 or 55. In one embodiment, an antibody may comprise the heavy chain CDRs of antibody 253, 165 or 55, and the light chain CDRs of antibody 253, 165 or 55. In one embodiment, an antibody may comprise the light chain CDRs of a first antibody and the heavy chain CDRs of a second antibody, wherein the two antibodies were derived from the same public v-regions. In one embodiment, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 268, 269 and 270, respectively. In one embodiment, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 188, 189 and 190, respectively. In one embodiment, an antibody of the invention may comprise a CDRH1, CDRH2 and CDRH3 having the amino acid sequences specified in SEQ ID NOs: 65, 66 and 67, respectively, and a CDRL1, CDRL2 and CDRL3 having the amino acid sequences specified in SEQ ID NOs: 68, 69 and 70, respectively.

Antibody 222 was surprisingly found to retain strong neutralisation of the SARS-CoV-2 variants, Victoria, B.1.1.7, B.1.351 and P.1 strains, e.g. an IC50 of ≤0.02 μg/ml against the Victoria, B.1.1.7, B.1.351 and P.1 strains (see Table 18). Accordingly, in one embodiment, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 258, 259 and 260, respectively.

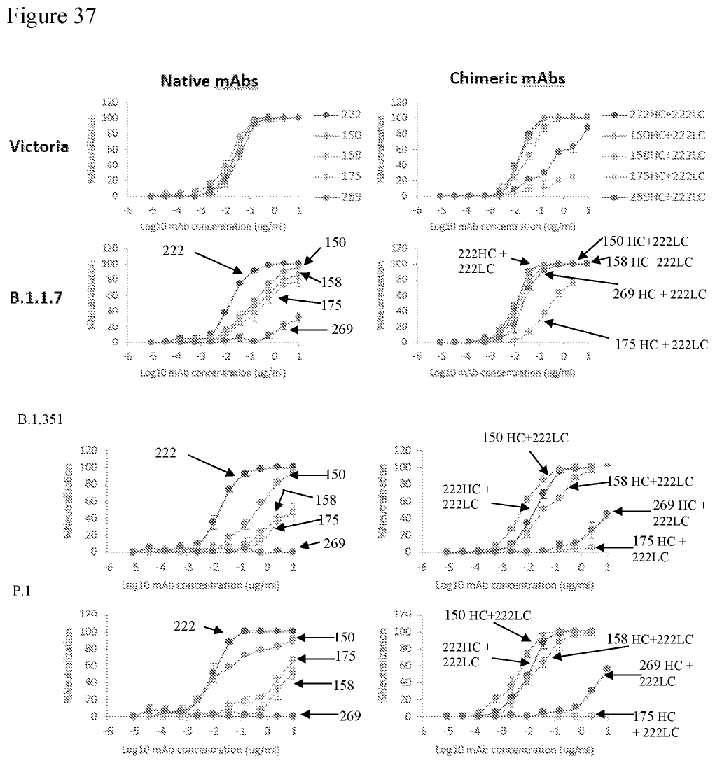

Interestingly, antibodies comprising the light chain of antibody 222 exhibited potent cross-lineage neutralisation effects, e.g. they are effective against all tested SARS-CoV-2 strains in the Examples (as shown in Table 18 and FIG. 37). Such mixed chain antibodies are discussed further below. Accordingly, an antibody of the invention may comprise CDRL1, CDRL2 and CDRL3 having the amino acid sequences specified in SEQ ID NOs: 258, 259 and 260, respectively. The antibody may further comprise a CDRH1, CDRH2 and CDRH3 from an antibody derived from IGHV3-53 or IGHV3-66, such as IGHV3-53.

Antibody 318 was surprisingly found to retain strong neutralisation of the SARS-CoV-2 variants, Victoria, B.1.1.7 and B.1.351. Accordingly, in one embodiment, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 338, 339 and 340, respectively.

Antibody 316 was one of the most potent neutralising antibodies identified and was surprisingly found to retain strong neutralisation of the B.1.1.7 strain. Accordingly, in one embodiment, an antibody of the invention may comprise a CDRH1, CDRH2 and CDRH3 having the amino acid sequences specified in SEQ ID NOs: 325, 326 and 32, respectively, and a CDRL1, CDRL2 and CDRL3 having the amino acid sequences specified in SEQ ID NOs: 328, 329 and 330, respectively.

Antibodies 159, 384, 88, 40 and 253H55L are all highly potent neutralising mAbs, which have been shown to protect, prophylactically or therapeutically, in animal models.

Antibody 159 binds to the NTD of the spike protein and did not block ACE2 binding, but was unexpectedly one of the most potent neutralising antibodies observed. Prior art antibodies that co-localised to the NTD, in comparison, did not show appreciable neutralisation in the assays used herein. Antibody 159 has also been shown to have beneficial properties in animal models. Therefore, in a preferred embodiment, the antibody in Table 1 is 159.

Hence, an antibody of the invention may comprise at least three CDRs of antibody 159. For example, an antibody of the invention may comprise the CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 178, 179 and 180, respectively. In one embodiment, an antibody of the invention may comprise a CDRH1, CDRH2 and CDRH3 having the amino acid sequences specified in SEQ ID NOs: 175, 176 and 177, respectively.

Antibody 384 binds to a unique epitope of the RBD and is distinct from all previously reported binding modes, and was the most potently neutralising mAb described herein. The increased potency of antibody 384, when compared to other antibodies derived from the same v-region, is suggested to be due to the 18-residue long CDRH3 which forms an extended interaction across the ACE2 binding site of the RBD. Antibody 384 has also been shown to have beneficial properties in animal models. Therefore, in a preferred embodiment, the antibody in Table 1 is 384.

Hence, an antibody of the invention may comprise at least three CDRs of antibody 384. For example, an antibody of the invention may comprise a CDRH1, CDRH2 and CDRH3 having the amino acid sequences specified in SEQ ID NOs: 375, 376 and 377, respectively, and a CDRL1, CDRL2 and CDRL3 having the amino acid sequences specified in SEQ ID NOs: 378, 379 and 380, respectively.

In one embodiment, an antibody of the invention may comprise a CDRH2 and CDRH3 having the amino acid sequences specified in SEQ ID NOs: 376 and 377, respectively, and a CDRL1 and CDRL3 set forth in SEQ ID NOs: 378 and 380, respectively.

Antibody 88 was one of the most potent neutralising antibodies discovered herein. Antibody 99 comprises an N-glycosylation site in CDRH1 that is not essential for RBD binding, but is essential for neutralisation. Antibody 88 has also been shown to have beneficial properties in animal models. Therefore, in a preferred embodiment, the antibody in Table 1 is 88.

Hence, an antibody of the invention may comprise at least three CDRs of antibody 88. For example, an antibody of the invention may comprise a CDRH1, CDRH2 and CDRH3 having the amino acid sequences specified in SEQ ID NOs: 105, 106 and 107, respectively, and a CDRL1, CDRL2 and CDRL3 having the amino acid sequences specified in SEQ ID NOs: 108, 109 and 110, respectively.

Antibody 40 comprises a heavy chain derived from the IGHV3-66 v-region and was one of the potent neutralizers identified herein. Antibody 40 has also been shown to have beneficial properties in animal models. Therefore, in a preferred embodiment, the antibody in Table 1 is 40.

Hence, an antibody of the invention may comprise at least three CDRs of antibody 40. For example, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 28, 29 and 30, respectively.

Antibodies 58, 222, 253 and 253H/55L are mAbs that have been shown to neutralize the omicron strain.

Antibody 58 was unexpectedly one of the most potent neutralising antibodies observed against the omicron strain. Therefore, in a preferred embodiment, the antibody in Table 1 is 58.

Hence, an antibody of the invention may comprise at least three CDRs of antibody 58. For example, an antibody of the invention may comprise the CDRH1, CDRH2 and CDRH3 having the amino acid sequences specified in SEQ ID NOs: 75, 76 and 77, respectively, and a CDRL1, CDRL2 and CDRL3 having the amino acid sequences specified in SEQ ID NOs: 78, 79 and 80, respectively.

Antibody 222 was one of the most potent neutralising antibodies observed against the omicron strain. Antibody 222 also potently neutralizes all other strains tested in Table 26. Therefore, in a preferred embodiment, the antibody in Table 1 is 222.

Hence, an antibody of the invention may comprise at least three CDRs of antibody 222. For example, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 258, 259 and 260, respectively.

Antibody 253 was found to neutralize the omicron strain. Antibody 253 also potently neutralizes all other strains tested in Table 26. Therefore, in a preferred embodiment, the antibody in Table 1 is 253.

Hence, an antibody of the invention may comprise at least three CDRs of antibody 253. For example, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 268, 269 and 270, respectively.

Antibody 253H/55L was found to neutralize the omicron strain. Antibody 253H/55L also potently neutralizes all other strains tested in Table 26. Therefore, in a preferred embodiment, the antibody in Table 1 is 253H/55L.

Hence, an antibody of the invention may comprise at least three CDRs of antibody 253h/55L. For example, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 68, 69 and 70, respectively.

The antibody of the invention may comprise at least four, five, or all six CDRs of an antibody in Table 1. The antibody may comprise at least one, at least two or all three heavy chain CDRs (CDRHs). The antibody may comprise at least one, at least two or all three light chain CDRs (CDRLs). The antibody typically comprises all six (i.e. three heavy and three light chain) CDRs.

The antibody of the invention may comprise a heavy chain variable domain having ≥80%, ≥90%, ≥95%, ≥96%, ≥97%, ≥98%, ≥99% or 100% sequence identity to the heavy chain variable domain amino acid sequence of an antibody in Table 1 (e.g. 253H/55L, 253H/165L, 222, 318, 165, 55, 159, 384, 88, 318 or 40).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 150 (i.e. SEQ ID NO: 152).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 158 (i.e. SEQ ID NO: 162).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 253H55L (i.e. SEQ ID NO: 262).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 222 (i.e. SEQ ID NO: 252).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 58 (i.e. SEQ ID NO: 72). In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 318 (i.e. SEQ ID NO: 332).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 165 (i.e. SEQ ID NO: 182).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 55 (i.e. SEQ ID NO: 62).

In the embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 159 (i.e. SEQ ID NO: 172).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 384 (i.e. SEQ ID NO: 372).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 88 (i.e. SEQ ID NO: 102).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 40 (i.e. SEQ ID NO: 22).

In one embodiment, an antibody of the invention may comprise a heavy chain variable domain having ≥80%, ≥90%, ≥95%, ≥96%, ≥97%, ≥98%, ≥99% or 100% sequence identity to the heavy chain variable domain of antibody 316 (i.e. SEQ ID NO: 322).

The antibody of the invention may comprise a light chain variable domain having ≥80%, ≥90%, ≥95%, ≥96%, ≥97%, ≥98%, ≥99% or 100% sequence identity to the light chain variable domain amino acid sequence of an antibody in Table 1 (e.g. 253H/55L, 253H/165L, 222, 318, 253, 165, 55, 159, 384, 88, 40 or 316).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 150 (i.e. SEQ ID NO: 154).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 158 (i.e. SEQ ID NO: 164).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 253H55L (i.e. SEQ ID NO: 64).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 253H165L (i.e. SEQ ID NO: 184).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 222 (i.e. SEQ ID NO: 254).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 253 (i.e. SEQ ID NO: 264).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 58 (i.e. SEQ ID NO: 74).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 318 (i.e. SEQ ID NO: 334).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 253 (i.e. SEQ ID NO: 264).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 159 (i.e. SEQ ID NO: 174).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 384 (i.e. SEQ ID NO: 374).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 88 (i.e. SEQ ID NO: 104).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 40 (i.e. SEQ ID NO: 24).

In one embodiment, an antibody of the invention may comprise a light chain variable domain having ≥80%, ≥90%, ≥95%, ≥96%, ≥97%, ≥98%, ≥99% or 100% sequence identity to the light chain variable domain of antibody 316 (i.e. SEQ ID NO: 324).

The antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain amino acid sequence and light chain variable domain amino acid sequence, respectively, of an antibody in Table 1 (e.g. 253H/55L, 253H/165L, 222, 318, 253, 165, 55, 159, 384, 88, 40 or 316).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 253H55L (SEQ ID NOs: 262 and 64, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 253H165L (SEQ ID NOs: 262 and 184, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 222 (SEQ ID NOs: 252 and 254, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 58 (SEQ ID NOs: 72 and 74, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 253 (SEQ ID NOs: 262 and 264, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 318 (SEQ ID NOs: 332 and 334, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 253 (SEQ ID NOs: 262 and 264, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 165 (SEQ ID NOs: 182 and 184, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 55 (SEQ ID NOs: 62 and 64, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 159 (SEQ ID NOs: 172 and 174, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 384 (SEQ ID NOs: 372 and 374, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 88 (SEQ ID NOs: 102 and 104, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 40 (SEQ ID NOs: 22 and 24, respectively).

In one embodiment, an antibody of the invention may comprise a heavy chain variable domain and a light chain variable domain having ≥80%, ≥90%, ≥95%, ≥96%, ≥97%, ≥98%, ≥99%, 100% sequence identity to the heavy chain variable domain and light chain variable domain, respectively, of antibody 316 (SEQ ID NOs: 322 and 324, respectively).

Alternatively, an antibody of the invention may comprise the light chain variable domain amino acid sequence from one antibody in Table 1 and the heavy chain variable domain amino acid sequence from another antibody in Table 1. Hence, an antibody of the invention may comprise: (a) a heavy chain variable domain having ≥80%, ≥90%, ≥95%, ≥96%, ≥97%, ≥98%, ≥99%, 100% sequence identity to the heavy chain variable domain of a first antibody in Table 1; and (b) a light chain variable domain having ≥80%, ≥90%, ≥95%, ≥96%, ≥97%, ≥98%, ≥99%, 100% of the light chain variable domain of a second antibody in Table 1.

In one embodiment, the first antibody in Table 1 is 253 and the second antibody in Table 1 is 55, resulting in the antibody 253H55L. In another embodiment, the first antibody in Table 1 is 253 and the second antibody in Table 1 is 165, resulting in the antibody 253H165L.

The first and/or second antibody in Table 1 may be derived from a major public v-region. The first and second antibodies in Table 1 may be derived from the same germline heavy chain v-region. The heavy chain v-region may be IGHV3-53, IGHV1-58 or IGHV3-66 (described further below).

For example, an antibody of the invention may comprise a heavy chain variable domain amino acid sequence having ≥80%, ≥90%, ≥95%, ≥96%, ≥97%, ≥98%, ≥99%, 100% sequence identity to the heavy chain variable domain from a first antibody in Table 1, and a light chain variable domain amino acid sequence having ≥80%, ≥90%, ≥95%, ≥96%, ≥97%, ≥98%, ≥99%, 100% sequence identity to the light chain variable domain from a second antibody in Table 1, wherein the first and second antibodies derive from the same germline heavy chain v-region, optionally wherein the heavy chain v-region is IGHV3-53, IGHV1-58 or IGHV3-66.

In one embodiment, the invention provides any one of the antibodies listed in Table 1, 21, 22, 23, 24 or 25.

An antibody of the invention may be or may comprise a modification from the amino acid sequence of an antibody in Table 1, whilst maintaining the activity and/or function of the antibody. The modification may a substitution, deletion and/or addition. For example, the modification may comprise 1, 2, 3, 4, 5, up to 10, up to 20, up to 30 or more amino acid substitutions and/or deletions from the amino acid sequence of an antibody in Table 1. For example, the modification may comprise an amino acid substituted with an alternative amino acid having similar properties. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

The modification may comprise a derivatised amino acid, e.g. a labelled or non-natural amino acid, providing the function of the antibody is not significantly adversely affected.

Modification of antibodies of the invention as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Antibodies of the invention may be modified (e.g. as described above) to improve the potency of said antibodies or to adapt said antibodies to new SARS-CoV-2 variants. The modifications may be amino acid substitutions to adapt the antibody to substitutions in a virus variant. For example, the known mode of binding of an antibody to the spike protein (e.g. by crystal structure determination, or modelling) may be used to identify the amino acids of the antibody that interact with the substitution in the virus variant. This information can then be used to identify possible substitutions of the antibody that will compensate for the change in the epitope characteristics. For example, a substitution of a hydrophobic amino acid in the spike protein to a negatively changes amino acid may be compensated by substituting the amino acid from the antibody that interacts with said amino acid in the spike protein to a positively charged amino acid. Methods for identifying residues of an antibody that may be substituted are encompassed by the present disclosure, for example, by determining the structure of antibody-antigen complexes as described herein.

The antibodies of the invention may contain one or more modifications to increase their cross-lineage neutralisation property. For example, E484 of the spike protein, which is a key residue that mediates the interaction with ACE2, is mutated in some SARS-CoV-2 strains (e.g. Victoria strain which contains E484, but P.1 and B.1.351 strains contain E484K) resulting in differing neutralisation effects of the antibodies (see Example 24). Thus, antibodies that bind to E484 can be modified to compensate for the changes in E484 of the spike protein. For example, E484 is mutated from a positively charge to negatively charged amino acid in SAR-CoV-2 strains of B.1.351 or P.1 lineage. The amino acid residues of antibodies that bind to or near E484 may be mutated to compensate for the change in charge. Examples of such amino acid residues may be G104 and/or K108 in SEQ ID NO: 102 of antibody 88, or R52 in SEQ ID NO: 372 of antibody 384 (see Example 24).

Antibodies of the invention may be isolated antibodies. An isolated antibody is an antibody which is substantially free of other antibodies having different antigenic specificities.

The term ‘antibody’ as used herein may relate to whole antibodies (i.e. comprising the elements of two heavy chains and two light chains inter-connected by disulphide bonds) as well as antigen-binding fragments thereof. Antibodies typically comprise immunologically active portions of immunoglobulin (Ig) molecules, i.e., molecules that contain an antigen binding site that specifically binds (immunoreacts with) an antigen. By “specifically binds” or “immunoreacts with” is meant that the antibody reacts with one or more antigenic determinants of the desired antigen and does not react with other polypeptides. Each heavy chain is comprised of a heavy chain variable region (abbreviated herein as HCVR or VH) and at least one heavy chain constant region. Each light chain is comprised of a light chain variable region (abbreviated herein as LCVR or VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). Antibodies may include, but are not limited to, polyclonal, monoclonal, chimeric, dAb (domain antibody), single chain, Fab, Fab′ and F(ab′)2 fragments, scFvs, and Fab expression libraries

An antibody of the invention may be a monoclonal antibody. Monoclonal antibodies (mAbs) of the invention may be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press).

An antibody of the invention may be multispecific, such as bispecific, i.e. one ‘arm’ of the body binds the spike protein of SARS-CoV-2, and the other ‘arm’ binds a different antigen. In one embodiment, a bispecific antibody of the invention may bind to two separate epitopes on the spike protein. In one embodiment, a bispecific antibody of the invention binds to the NTD of the spike protein with one ‘arm’ and to the RBD of the spike protein with another ‘arm’. In one embodiment, a bispecific antibody of the invention binds to two different antibodies on the RBD of the spike protein. In one embodiment, a bispecific antibody of the invention binds to different proteins with each ‘arm’. For example, one or more (e.g. two) antibodies of the invention can be coupled to form a multispecific (e.g. bispecific) antibody.

An antibody may be selected from the group consisting of single chain antibodies, single chain variable fragments (scFvs), variable fragments (Fvs), fragment antigen-binding regions (Fabs), recombinant antibodies, monoclonal antibodies, fusion proteins comprising the antigen-binding domain of a native antibody or an aptamer, single-domain antibodies (sdAbs), also known as VHH antibodies, nanobodies (Camelid-derived single-domain antibodies), shark IgNAR-derived single-domain antibody fragments called VNAR, diabodies, triabodies, Anticalins, aptamers (DNA or RNA) and active components or fragments thereof.

The constant region domains of an antibody molecule of the invention, if present, may be selected having regard to the proposed function of the antibody molecule, and in particular the effector functions which may be required. For example, the constant region domains may be human IgA, IgD, IgE, IgG or IgM domains. Typically, the constant regions are of human origin. In particular, human IgG (i.e. IgG1, IgG2, IgG3 or IgG4) constant region domains may be used. Typically, a human IgG1 constant region.

The light chain constant region may be either lambda or kappa.

Antibodies of the invention may be mono-specific or multi-specific (e.g. bispecific). A multi-specific antibody comprises at least two different variable domains, wherein each variable domain is capable of binding to a separate antigen or to a different epitope on the same antigen.

An antibody of the invention may be a chimeric antibody, a CDR-grafted antibody, a nanobody, a human or humanised antibody. Typically, the antibody is a human antibody. Fully human antibodies are those antibodies in which the variable regions and the constant regions (where present) of both the heavy and the light chains are all of human origin, or substantially identical to sequences of human origin, but not necessarily from the same antibody.

The antibody of the invention may be a full-length antibody. Hence, the invention also provides an antibody which is a full length antibody of any one of the antibodies in Tables 1 and 21 to 25. In other words, an antibody of the invention comprises a heavy chain variable domain and a light chain variable domain consisting of the heavy chain variable domain and light chain variable domain, respectively, of any one of the antibodies in Tables 1 and 21 to 25, and a IgG (e.g. IgG1) constant region. For example, the full-length antibody may be 222, 253H55L, 253H165L, 318, 253, 55, 165, 384, 159, 88, 40, 316, or 58.

The antibody of the invention may be an antigen-binding fragment. An antigen-binding fragment of the invention binds to the same epitope of the parent antibody, i.e. the antibody from which the antigen-binding fragment is derived. An antigen-binding fragment of the invention typically retains the parts of the parent antibody that interact with the epitope. The antigen-binding fragment typically comprise the complementarity-determining regions (CDRs) that interact with the antigen, such as one, two, three, four, five or six CDRs. In some embodiments, the antigen-binding fragment further comprises the structural scaffold surrounding the CDRs of the parent antibody, such as the variable region domains of the heavy and/or light chains. Typically, the antigen-binding fragment retains the same or similar binding affinity to the antigen as the parent antibody.

An antigen-binding fragment does not necessarily have an identical sequence to the parent antibody. In one embodiment, the antigen-binding fragment may have ≥70%, ≥80%, ≥90%, ≥95%, ≥96%, ≥97%, ≥98%, ≥99%, 100% sequence identity with the respective CDRs of the parent antibody. In one embodiment, the antigen-binding fragment may have ≥70%, ≥80%, ≥90%, ≥95%, ≥96%, ≥97%, ≥98%, ≥99%, 100% sequence identity with the respective variable region domains of the parent antibody. Typically, the non-identical amino acids of a variable region are not in the CDRs.

The antigen-binding fragments of antibodies of the invention retain the ability to selectively bind to an antigen. Antigen-binding fragments of antibodies include single chain antibodies (i.e. a full-length heavy chain and light chain); Fab, modified Fab, Fab′, modified Fab′, F(ab′)2, Fv, Fab-Fv, Fab-dsFv, single domain antibodies (e.g. VH or VL or VHH), scFv.

An antigen-binding function of an antibody can be performed by fragments of a full-length antibody. The methods for creating and manufacturing these antibody fragments are well known in the art (see for example Verma R et al., 1998, J. Immunol. Methods, 216, 165-181).

Methods for screening antibodies of the invention that do not share 100% amino acid sequence identity with one of the antibodies disclosed herein, that possess the desired specificity, affinity and functional activity include the methods described herein, e.g. enzyme linked immunosorbent assays, biacore, focus reduction neutralisation assay (FRNT), and other techniques known within the art.

With regards to function, an antibody of the invention may be able to neutralize at least one biological activity of SAR-CoV-2 (a neutralising antibody), particularly to neutralize virus infectivity.

Neutralisation may also be determined using IC₅₀ or IC₉₀ values. For example, the antibody may have an IC₅₀ value of ≤0.1 μg/ml, ≤0.05 μg/ml, ≤0.01 μg/ml or ≤0.005 μg/ml. In some instances an antibody of the invention may have an IC₅₀ value of between 0.005 μg/ml and 0.1 μg/ml, sometimes between 0.005 μg/ml and 0.05 μg/ml or even between μg/ml and 0.05 μg/ml.

For example, the IC₅₀ values of some of the antibodies of Table 1 are provided in Tables 3 and 9.

The ability of an antibody to neutralize virus infectivity may be measured using an appropriate assay, particularly using a cell-based neutralisation assay, as shown in the Examples. For example, the neutralisation ability may be measured in a focus reduction neutralisation assay (FRNT) where the reduction in the number of cells (e.g. human cells) infected with the virus (e.g. for 2 hours at 37° C.) in the presence of the antibody is compared to a negative control in which no antibodies were added.

The Examples show that the neutralisation activity may be influenced by N-glycosylation sequins (having a consensus sequence of N-X-S/T; X is any amino acid except proline) in the heavy chain variable region. In particular, it was shown that some of the Table 1 antibodies are potently inhibitory antibodies having a neutralisation IC50 of less than 0.1 μg/ml, and mutations of these antibodies to remove the N glycan has a negative effect on neutralisation even though they could be de-glycosylated without denaturation or loss of RBD affinity.

In one embodiment, an antibody of the invention comprises an N-glycosylation sequon starting at position 35 (KABAT numbering, using EU Index) of the heavy chain variable region (having a consensus sequence of N-X-S/T). In one embodiment, an antibody of the invention comprises CDRH1 of antibody 88 as specified in SEQ ID NO: 105. In one embodiment, an antibody of the invention comprises CDRH1, CDRH2 and CDRH3 of antibody 88 as specified in SEQ ID NOs: 105, 106 and 107, respectively. In one embodiment, an antibody of the invention comprises the VH domain of antibody 88 as specified in SEQ ID NOs: 102.

In one embodiment, an antibody of the invention comprises an N-glycosylation sequon starting at positions 59 (KABAT numbering, using EU Index) of the heavy chain variable region (having a consensus sequence of N-X-S/T). In one embodiment, an antibody of the invention comprises CDRH1 of antibody 316 as specified in SEQ ID NO: 326 and extended so as to include the N-glycosylation sequon. In one embodiment, an antibody of the invention comprises CDRH1, CDRH2 and CDRH3 of antibody 316 as specified in SEQ ID NOs: 325, 326 and 327, respectively. In one embodiment, an antibody of the invention comprises the VH domain of antibody 316 as specified in SEQ ID NOs: 322.

In one embodiment, an antibody of the invention comprises an N-glycosylation sequon starting at positions 102 of the heavy chain variable region (having a consensus sequence of N-X-S/T). In one embodiment, an antibody of the invention comprises CDRH1 of antibody 253 as specified in SEQ ID NO: 267. In one embodiment, an antibody of the invention comprises CDRH1, CDRH2 and CDRH3 of antibody 253 as specified in SEQ ID NOs: 265, 266 and 267, respectively. In one embodiment, an antibody of the invention comprises the VH domain of antibody 253 as specified in SEQ ID NOs: 262.

An antibody of the invention may block the interaction between the spike protein of SAR-CoV-2 with the cell surface receptor, angiotensin-converting enzyme 2 (ACE2), of the target cell, e.g. by direct blocking or by disrupting the pre-fusion conformation of the spike protein.

Blocking of the interaction between spike and ACE2 can be total or partial. For example, an antibody of the invention may reduce spike-ACE2 formation by ≥50%, ≥60%, ≥70%, ≥80%, ≥90%, ≥95%, ≥99% or 100%. Blocking of spike-ACE2 formation can be measured by any suitable means known in the art, for example, by ELISA.

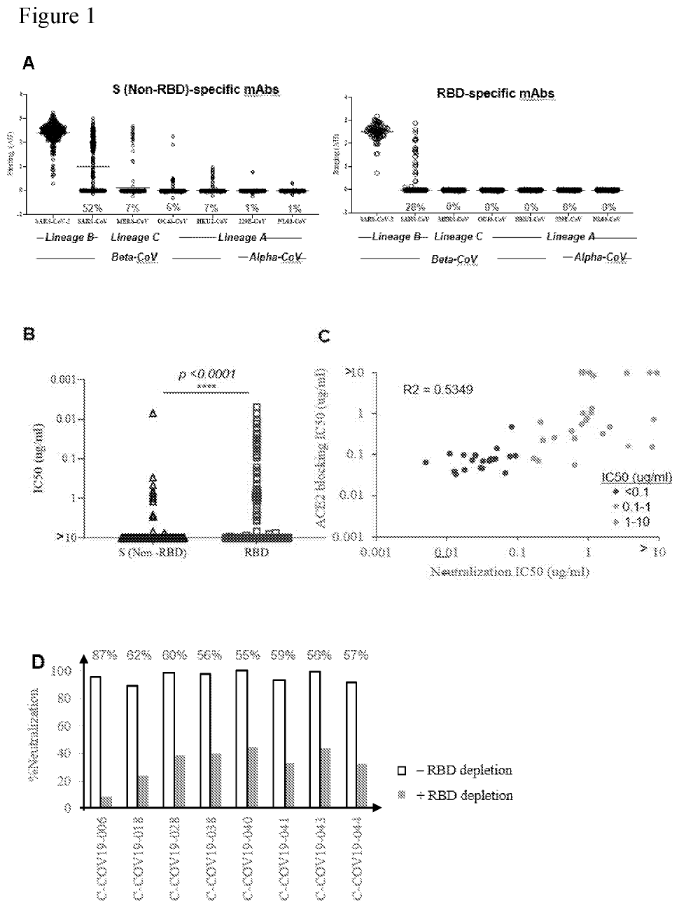

Most antibodies showing neutralisation also showed blocking of the interaction between the spike protein and ACE2. (see FIG. 1C). Furthermore, a number of non-neutralising antibodies were good ACE2 blockers.

In terms of binding kinetics, an antibody of the invention may have an affinity constant (K_{D}) value for the spike protein of SARS-CoV-2 of ≤5 nM, ≤4 nM, ≤3 nM, ≤2 nM, ≤1 nM, ≤0.5 nM, ≤0.4 nM, ≤0.3 nM, ≤0.2 nM or ≤0.1 nM. The K D values of some of the antibodies of Table 1 are provided in Tables 3 and 9.

The KD value can be measured by any suitable means known in the art, for example, by ELISA or Surface Plasmon Resonance (Biacore) at 25° C.

Binding affinity (K_{D}) may be quantified by determining the dissociation constant (K_{d}) and association constant (Kₐ) for an antibody and its target. For example, the antibody may have an association constant (Kₐ) of ≥10000 M⁻¹s⁻¹, ≥50000 M⁻¹s⁻¹, ≥100000 M⁻¹s⁻¹, ≥200000 M⁻¹s⁻¹ or ≥500000 M⁻¹s⁻¹ and/or a dissociation constant (Kₐ) of ≤0.001 s⁻¹, ≤0.0005 s⁻¹, ≤0.004 s⁻¹, ≤0.003 s⁻¹, ≤0.002 s⁻¹ or ≤0.0001 s⁻¹. For example, see Table 3.

An antibody of the invention is preferably able to provide in vivo protection in coronavirus (e.g. SARS-CoV-2) infected animals. For example, administration of an antibody of the invention to coronavirus (e.g. SARS-CoV-2) infected animals may result in a survival rate of ≥30%, ≥40%, ≥50%, ≥60%, ≥70%, ≥80%, ≥90%, ≥95% or 100%. Survival rates may be determined using routine methods.

Antibodies of the invention may have any combination of one or more of the above properties.

Antibodies of the invention may bind to the same epitope as, or compete for binding to SARS-CoV-2 spike protein with, any one of the antibodies described herein (i.e. in particular with antibodies with the heavy and light chain variable regions described above). Methods for identifying antibodies binding to the same epitope, or cross-competing with one another, are used in the Examples and discussed further below.

An antibody may bind to the same epitope as or competes with antibody 159. Antibody 159 binds to the NTD of the spike protein. In one embodiment, the antibody of the invention binds the NTD such that it does not block ACE2 binding. In one embodiment, the antibody of the invention binds to an epitope comprising residues 144-147, 155-158 and 250-253 of the NTD (numbering of the NTD and RBD is based on the spike protein as a whole, as used herein, unless stated otherwise). All 3 CDRs of antibody 159 contribute to the binding footprint, whereas the light chain has little contact. Accordingly, in one embodiment, the antibody of the invention comprises CDRH1, CDRH2 and CDRH3 of antibody 159, as set forth in SEQ ID NOs: 175 to 177, respectively. In one embodiment, an antibody of the invention comprises the heavy chain variable region of antibody 159, as set forth in SEQ ID NO: 172.

An antibody of the invention may bind to the same epitope as or competes with antibody 45. In one embodiment, an antibody does not compete for binding with the potent neutralizer S309 0 Piccoli et al., 2020). In one embodiment, an antibody of the invention competes for binding to SARS-CoV-2 spike protein with antibody 45.

In one aspect, an antibody binds to the same epitope as or competes with antibody 384. The binding epitope of antibody 384 is unique among SARS-CoV-2 antibodies reported to date. This epitope comprises residues F104, L105, L455, F456 and G482 to F486 of the RBD domain, which are bound by the CDRH3 of antibody 384. In one embodiment, an antibody of the invention binds to this epitope using interactions from CDRH3 alone. In one embodiment, an antibody of the invention comprises CDRH3 of antibody 384, as set forth in SEQ ID NO: 377. In another embodiment, an antibody of the invention comprises CDRH2 and CDRH3 of antibody 384, as set forth in SEQ ID NOs: 376 and 377. Antibody 384 interacts with the spike protein through CDRH2 and CDRH3 of the heavy chain alone. In one embodiment, an antibody of the invention comprises CDRL1 of antibody 384, as set forth in SEQ ID NO: 378 and CDRL3 of antibody 384, as set forth in SEQ ID NOs: 380. In one embodiment, an antibody of the invention comprises CDRH2, CDRH3, CDRL1 and CDRL3 of antibody 384, as set forth in SEQ ID NOs: 376-378 and 380, respectively. Antibody 384 interacts with the spike protein through CDRH2, CDRH3, CDRL1 and CDRL3 of the antibody alone. In another aspect, an antibody binds to the same epitope as CDRH2 and CDRH3 of antibody 384. In a further embodiment, the an antibody of the invention does not contact the right chest of the RBD domain of the spike protein. In an embodiment, an antibody of the invention comprises the heavy chain CDRs set forth in SEQ ID NOs: 375, 376 and 377, and optionally, the light chain CDRs set forth in SEQ ID NOs: 378, 379 and 380. In another aspect, an antibody of the invention, W107 of CDRH3 makes strong π-interactions with G485 of the RBD, Y59 of CDRH2 contacts V483 and makes bifurcated H-bonds to the carbonyl oxygen of G482 and amino nitrogen of E484 of RBD, which in turn salt-bridges with R52 and H-bonds to the side-chains of T57 and Y59. E484-F486 of RBD also form a two-stranded antiparallel (3-sheet with residues A92-A94 of CDRL3 and make stacking interactions from F486 to Y32 of CDRL1. The preponderance of main-chain RBD interactions may confer resilience to mutational escape.

The skilled person is readily able to determine the binding site (epitope) of an antibody using standard techniques, such as those described in the Examples of the application. The skilled person could also readily determine whether an antibody binds to the same epitope as, or competes for binding with, an antibody described herein by using routine methods known in the art.

For example, to determine if a test antibody (i.e. where it is not known whether the test antibody competes with other antibodies for binding to an antigen) binds to the same epitope as an antibody described herein (referred to a “reference antibody” in the following paragraphs), the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention.

To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

As well as sequences defined by percentage identity or number of sequence changes, the invention further provides an antibody defined by its ability to cross-compete with one of the specific antibodies set out herein. It may be that the antibody also has one of the recited levels of sequence identity or number of sequence changes as well.

Cross-competing antibodies can be identified using any suitable method in the art, for example by using competition ELISA or BIAcore assays where binding of the cross competing antibody to a particular epitope on the spike protein prevents the binding of an antibody of the invention or vice versa. In one embodiment, the antibody produces ≥50%, ≥60%, ≥70%, ≥80%, ≥90% or 100% reduction of binding of the specific antibody disclosed herein.

The antibodies described below in the Examples may be used as reference antibodies.

Other techniques that may be used to determine antibody epitopes include hydrogen/deuterium exchange, X-ray crystallography and peptide display libraries (as described in the Examples). A combination of these techniques may be used to determine the epitope of the test antibody.

The approaches used herein could be applied equally to other data, e.g. surface plasmon resonance or ELISA, and provides a general way of rapidly determining locations from highly redundant competition experiments.

### Fc Regions

An antibody of the invention may or may not comprise an Fc domain.

The antibodies of the invention may be modified in the Fc region in order to improve their stability. Such modifications are known in the art. Modifications may improve the stability of the antibody during storage of the antibody. The in vivo half-life of the antibody may be improved by modifications of the Fc-region.

For example, cysteine residue(s) can be introduced into the Fc region, thereby allowing interchain disulphide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)).

For example, an antibody of the invention may be modified to promote the interaction of the Fc domain with FcRn. The Fc domain may be modified to improve the stability of the antibody by affecting Fc and FcRn interaction at low pH, such as in the endosome. The M252Y/S254T/T256E (YTE) mutation may be used to improve the half-life of an IgG1 antibody.

The antibody may be modified to affect the interaction of the antibody with other receptors, such as FcγRI, FcγRIIA, FcγRIIB, FcγRIII, and FcαR. Such modifications may be used to affect the effector functions of the antibody.

In one embodiment, an antibody of the invention comprises an altered Fc domain as described herein below. In another preferred embodiment an antibody of the invention comprises an Fc domain, but the sequence of the Fc domain has been altered to modify one or more Fc effector functions.

In one embodiment, an antibody of the invention comprises a “silenced” Fc region. For example, in one embodiment an antibody of the invention does not display the effector function or functions associated with a normal Fc region. An Fc region of an antibody of the invention does not bind to one or more Fc receptors.

In one embodiment, an antibody of the invention does not comprise a CH₂ domain. In one embodiment, an antibody of the invention does not comprise a CH₃ domain. In one embodiment, an antibody of the invention comprises additional CH₂ and/or CH₃ domains.

In one embodiment, an antibody of the invention does not bind Fc receptors. In one embodiment, an antibody of the invention does not bind complement. In an alternative embodiment, an antibody of the invention does not bind FcγR, but does bind complement.

In one embodiment, an antibody of the invention in general may comprise modifications that alter serum half-life of the antibody. Hence, in another embodiment, an antibody of the invention has Fc region modification(s) that alter the half-life of the antibody. Such modifications may be present as well as those that alter Fc functions. In one preferred embodiment, an antibody of the invention has modification(s) that alter the serum half-life of the antibody.

In one embodiment, an antibody of the invention may comprise a human constant region, for instance IgA, IgD, IgE, IgG or IgM domains. In particular, human IgG constant region domains may be used, especially of the IgG1 and IgG3 isotypes when the antibody molecule is intended for therapeutic uses where antibody effector functions are required. Alternatively, IgG2 and IgG4 isotypes may be used when the antibody molecule is intended for therapeutic purposes and antibody effector functions are not required.

In one embodiment, the antibody heavy chain comprises a CH₁ domain and the antibody light chain comprises a CL domain, either kappa or lambda. In one embodiment, the antibody heavy chain comprises a CH₁ domain, a CH₂ domain and a CH₃ domain and the antibody light chain comprises a CL domain, either kappa or lambda.

The four human IgG isotypes bind the activating Fcγ receptors (FcγRI, FcγRIIa, FcγRIIc, FcγRIIIa), the inhibitory FcγRIIb receptor, and the first component of complement (C1q) with different affinities, yielding very different effector functions (Bruhns P. et al., 2009. Specificity and affinity of human Fcγ receptors and their polymorphic variants for human IgG subclasses. Blood. 113(16):3716-25), see also Jeffrey B. Stavenhagen, et al. Cancer Research 2007 Sep. 15; 67(18):8882-90. In one embodiment, an antibody of the invention does not bind to Fc receptors. In another embodiment of the invention, the antibody does bind to one or more type of Fc receptors.

In one embodiment the Fc region employed is mutated, in particular a mutation described herein. In one embodiment the Fc mutation is selected from the group comprising a mutation to remove or enhance binding of the Fc region to an Fc receptor, a mutation to increase or remove an effector function, a mutation to increase or decrease half-life of the antibody and a combination of the same. In one embodiment, where reference is made to the impact of a modification it may be demonstrated by comparison to the equivalent antibody but lacking the modification.

Some antibodies that selectively bind FcRn at pH 6.0, but not pH 7.4, exhibit a higher half-life in a variety of animal models. Several mutations located at the interface between the CH₂ and CH₃ domains, such as T250Q/M428L (Hinton P R. et al., 2004. Engineered human IgG antibodies with longer serum half-lives in primates. J Biol Chem. 279(8):6213-6) and M252Y/S254T/T256E+H433K/N434F (Vaccaro C. et al., 2005. Engineering the Fc region of immunoglobulin G to modulate in vivo antibody levels. Nat Biotechnol. 23(10):1283-8), have been shown to increase the binding affinity to FcRn and the half-life of IgG1 in vivo. Hence, modifications may be present at M252/5254/T256+H44/N434 that alter serum half-life and in particular M252Y/S254T/T256E+H433K/N434F may be present. In one embodiment, it is desired to increase half-life. In another embodiment, it may be actually desired to decrease serum half-life of the antibody and so modifications may be present that decrease serum half-life.

Numerous mutations have been made in the CH₂ domain of human IgG1 and their effect on ADCC and CDC tested in vitro (Idusogie E E. et al., 2001. Engineered antibodies with increased activity to recruit complement. J Immunol. 166(4):2571-5). Notably, alanine substitution at position 333 was reported to increase both ADCC and CDC. Hence, in one embodiment a modification at position 333 may be present, and in particular one that alters ability to recruit complement. Lazar et al. described a triple mutant (S239D/I332E/A330L) with a higher affinity for FcγRIIIa and a lower affinity for FcγRIIb resulting in enhanced ADCC (Lazar G A. et al., 2006). Hence, modifications at S239/I332/A330 may be present, particularly those that alter affinity for Fc receptors and in particular S239D/I332E/A330L. Engineered antibody Fc variants with enhanced effector function. PNAS 103(11): 4005-4010). The same mutations were used to generate an antibody with increased ADCC (Ryan M C. et al., 2007. Antibody targeting of B-cell maturation antigen on malignant plasma cells. Mol. Cancer Ther., 6: 3009-3018). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards J O et al 2008. Optimization of antibody binding to Fcgamma RIIa enhances macrophage phagocytosis of tumor cells. Mol Cancer Ther. 7(8):2517-27). In one embodiment, S239D/I332E/G236A modifications may be therefore present.

In another embodiment, an antibody of the invention may have a modified hinge region and/or CH₁ region. Alternatively, the isotype employed may be chosen as it has a particular hinge regions.

### SARS-CoV-2 Variants

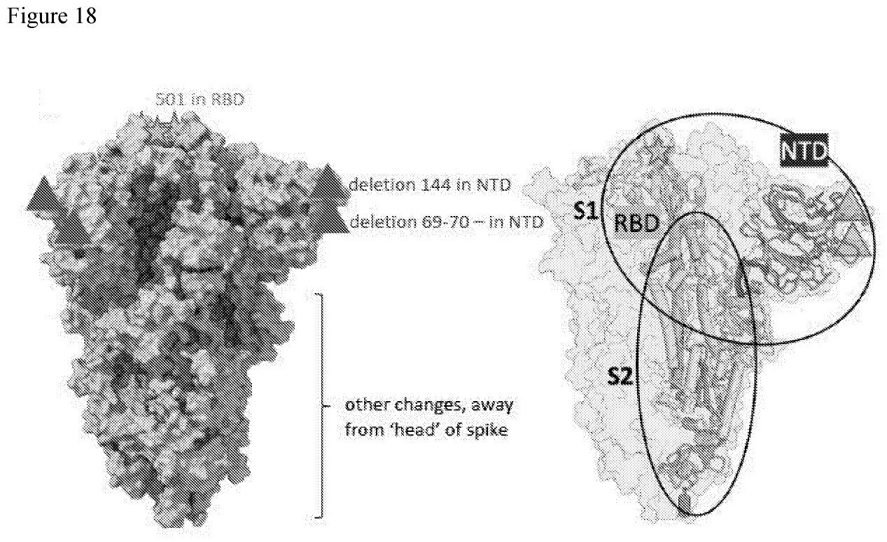

The B.1.1.7 variant was first identified in a sequence taken from a patient at the end of September 2020 (Rambaut et al., 2020). The variant has rapidly become dominant in many areas of the UK which has coincided with a rapid increase of infections during the second wave of the pandemic, with cases and hospitalizations in excess of those seen during the first phase. The B.1.1.7 variant is estimated to be 30-60% more infectious than strains encountered in the first wave (Walker et al., 2021) and able to overcome public health efforts to contain infection.B.1.1.7 contains a total of 9 changes in the spike protein: residues 69-70 are deleted, 144 is deleted, N501Y, A570D, D614G, P681H, T716I, S982A and D1118H of which the N501Y is potentially of the greatest concern as it has the potential to increase RBD/ACE2 affinity whilst also disrupting the binding of potent neutralizing antibodies (FIG. 18).

The B.1.351 variant has acquired mutations in the ACE2-interactive surface of the RBD at positions K417N, E484K and N501Y. B.1.351 has 10 changes relative to the Wuhan sequence: L18F, D80A, D215G, L242-244 deleted, R246I, K417N, E484K, N501Y, D614G, A701V. The 501Y.V2 variant has acquired mutations in the ACE2-interactive surface of the RBD at positions K417T, E484K and N501Y.

The B.1.617.2 (delta) variant has acquired the mutations L452R, T478K in the RBDrelative to the Wuhan sequence. The B.1.1.529 (omicron) variant has acquired the mutations G339D, S371L, S373P, S375F, K417N, N440K, G446S, S477N, T478K, E484A, Q493R, G496S, Q498R, N501Y, Y505H relative to the Wuhan sequence.

Where not otherwise specified herein, the strain referred to is SARS-CoV-2/human/AUS/VIC01/2020 (see Example 13, FRNT assay). This strain is an early strain related to the original Wuhan strain hCoV-19/Wuhan/WIV04/2019 (WIV04) (GISAID accession no. EPI_ISL_402124) and differs in a single amino acid.

It has been discovered that serum recovered from convalescent samples from patients in the first wave of COVID19 is less effective at neutralising variant strains. For example, convalescent serum was found to be 3-fold less effective against the B.1.1.7 variant when compared to the Victoria strain used herein and 13.3-fold less effective against the B.1.351 variant when compared to the Victoria strain. Serum from subjects vaccinated with the Pfizer and AstraZeneca vaccines was found to be 3-fold less-effective against the B.1.1.7 variant and 7.6-fold and 9-fold less effective against the B.1.351 variant, when compared to the Victoria strain. Accordingly, it is expected that antibodies are less effective than these variants.

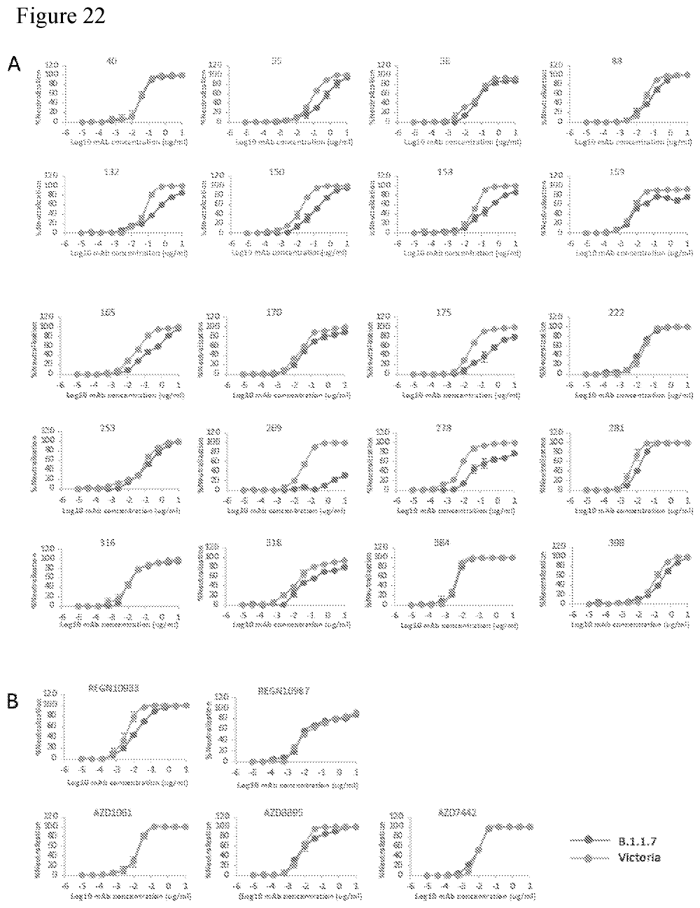

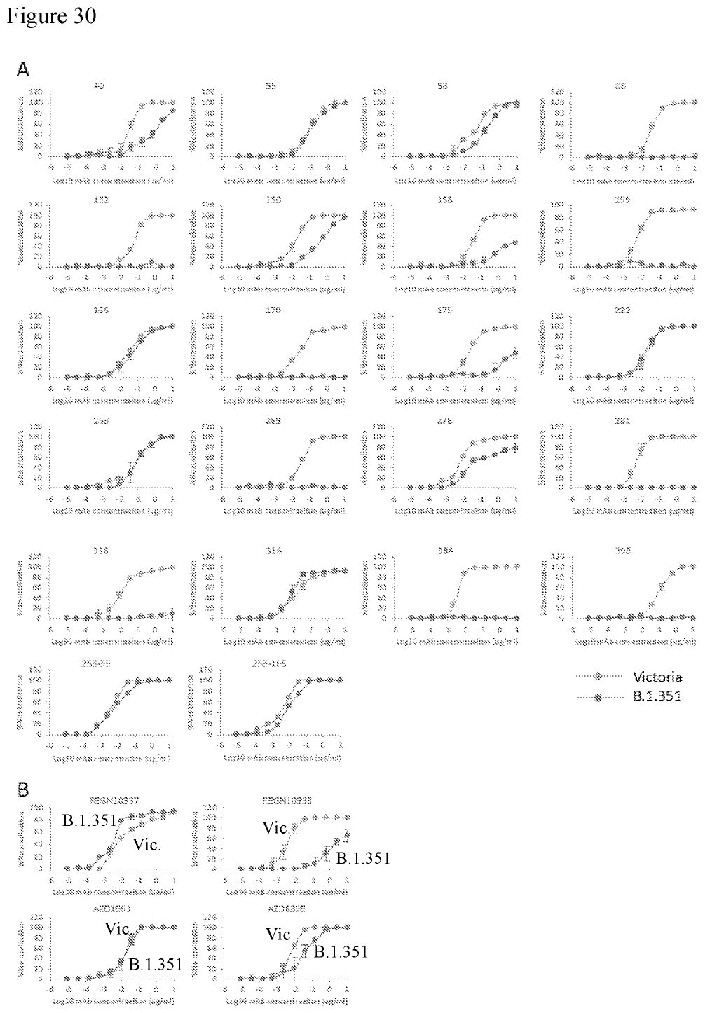

However, the inventors have surprisingly discovered that a number of antibodies described herein retain their neutralisation potency against the UK Kent (B.1.1.7) variant and the South Africa (B.1.351) variant. The neutralisation IC50s of the highly potent mAbs identified herein, against the variant strains, are shown in FIGS. 22 and 30, and Tables 11, 12 and 16.

Accordingly, in one embodiment, an antibody of the invention comprises at least 3, 4, 5 or all 6 of the CDRs of an antibody shown in Tables 12 or 16A. In one embodiment, an antibody of the invention retains strong neutralisation against the B.1.1.7 and/or the B.1.351 strain (such as less than a 10 fold drop in the IC50). In one embodiment, an antibody of the invention retains strong neutralisation against the B.1.1.7, the B.1.351 and/or the P.1 strain (such as less than a 10 fold drop in the IC50). A fold drop in the IC50 can be calculated by comparison to the IC50 of a reference strain, such as the Victoria strain tested used herein.

### Major Public V Regions

Public V-regions, also described as public V-genes herein, are the V regions of the germline heavy chain and light chain regions that are found in a large proportion of the population. That is to say, many individuals share the same public v-regions in their germline v-region repertoire.

As used herein, an antibody “derived” from a specific v-region refers to antibodies that were generated by V(D)J recombination using that germline v-region sequence. For example, the germline IGHV3-53 v-region sequence may undergo somatic recombination and somatic mutation to arrive at an antibody that specifically binds to the spike protein of SARS-CoV-2. The nucleotide sequence encoding the antibody may no longer comprise a sequence identical to the IGHV3-53 germline sequence, nevertheless, the antibody is still derived from this v-region. An antibody of the invention typically comprises no more than 20 non-silent mutations in the v-region, when compared to the germline sequence, such as no more than 15, 10, 9, 8, 7, 6, 5, 4, 3, 2 or 1 non-silent mutations. Germline v-region sequences are well known in the art, and methods of identifying whether a certain region of an antibody is derived from a particular germline v-region sequence are also well known in the art.

In one embodiment, an antibody of the invention derives from a v-region selected from IGHV3-53, IGHV1-58 and IGHV3-66. The inventors found that the potent neutralising antibodies identified herein comprised relatively few mutations in the CDRs of these v-regions. Thus, in one embodiment, an antibody of the invention encoded by a v-region selected from IGHV3-53, IGHV1-58 and IGHV3-66 and having 3-10 non-silent amino acid mutations, or 2-5 non-silent mutations, such as 10 or less, 9 or less, 8 or less, 7 or less, 6 or less, 5 or less, 4 or less 3 or less or 2 non-silent mutations when compared to the naturally occurring germline sequence.

In one embodiment, an antibody of the invention comprises the CDRs of an heavy chain variable domain of an antibody derived from a major public v-region selected from IGHV3-53, IGHV1-58 and IGHV3-66, such as antibodies 150, 158, 175, 222 and 269 for IGHV3-53, antibodies 55, 165, 253 and 318 for IGHV1-58, and antibodies 40 and 282 for IGHV3-66. The SEQ ID NOs corresponding to the CDRs of each of these antibodies are shown in Table 1.

In one embodiment, an antibody of the invention comprises the heavy chain variable domain of an antibody derived from a major public v-region selected from IGHV3-53, IGHV1-58 and IGHV3-66, such as antibodies 150, 158, 175, 222 and 269 for IGHV3-53, antibodies 55, 165, 253 and 318 for IGHV1-58, and antibodies 40 and 282 for IGHV3-66. The SEQ ID NOs corresponding to the heavy chain variable domains of each of these antibodies are shown in Table 1.

In a preferred embodiment, an antibody of the invention comprises the heavy chain CDRs 1-3 set forth in SEQ ID NOs: 265 to 267, respectively. In another embodiment, an antibody comprises the heavy chain variable domain of antibody 253 set forth in SEQ ID NO: 262.

There is a close association between potent neutralizers and public V-genes suggesting that vaccination responses should be strong (Yuan et al., 2020b). Three public V-region genes are represented at least twice in the set of 21, i) IGHV3-53: mAbs 150, 158, 175, 222 and 269, ii) IGHV1-58: 55, 165, 253 and 318 and iii) IGHV3-66: 282 and 40. In all cases the potent binders focus around the neck cluster, often with binding pose determined by the H1 and H2 loops. By switching light chains within these sets, antibody 253 could improve functionally by an order of magnitude by using an alternate light chain to achieve better hydrophobic interactions with the key bridging region identified, E484-F486. The most highly potent mAb, 384, adopts a unique pose, with a footprint extending from the left shoulder epitope across to the neck epitope via an extended H3.

Five of the potent monoclonal antibodies used herein (150, 158, 175, 222 and 269), belong to the VH3-53 family and a further 2 (282 and 40) belong to the almost identical VH3-66. Accordingly, embodiments related to the VH3-53 family may equally apply to the VH3-66 family.

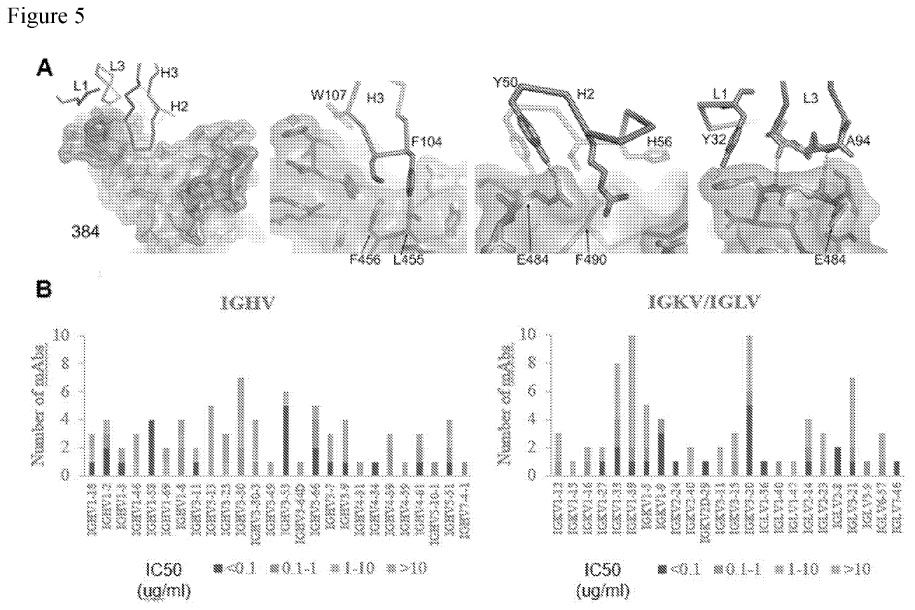

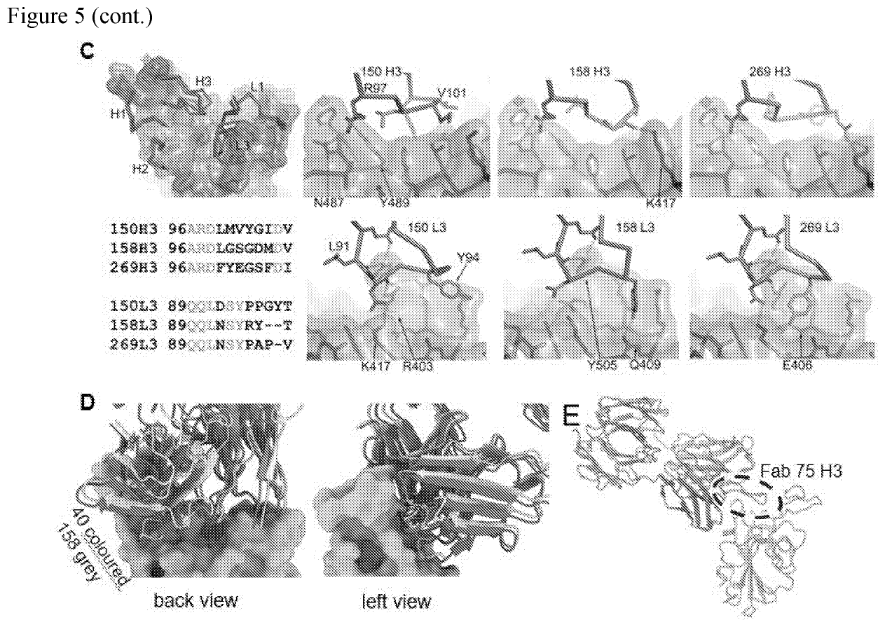

As shown in FIG. 5B, other public v-regions were overrepresented in the highly potent antibodies identified herein. Accordingly, in one aspect, an antibody comprises a variable domain sequence derived from a V-region selected from the following list: IGHV1-2, IGHV1-58, IGHV3-66, IGHV7-4-1, IGKV1-33, IGKV1-9, IGKV3-20, IGLV2-14, IGLV2-8 and/or IGLV3-21.

In on embodiment, an antibody comprises a heavy chain variable domain sequence derived from a V-region selected from: IGHV1-58, IGHV1-18 or IGHV3-9; and/or a light chain variable domain sequence derived from a V-region selected from: IGκV3-20, IG λ 3-21, or IGκ1-39 or κ1D-39. Antibodies derived from these regions (e.g. antibody 55, 58, 165, 253, 278 and 318) have shown to be particularly effective in cross-lineage neutralisation effects (e.g. against both Victoria and B.1.351 strains) and have good binding affinity to spike protein (see Table 16A).

Furthermore, and as described in the examples, it has been surprisingly shown that antibodies derived from particular public V-regions are able to maintain or improve neutralisation against the B.1.1.7 and/or B.1.351 strains when compared to the Victoria strain. In particular, an antibody of the invention is derived from a IGHV1-58 v-region (antibodies 55, 165, 253 and 318). In one embodiment, the light chains of an antibody with a heavy chain derived from IGHV1-58 may be exchanged with the light chain of a second antibody also derived from the same heavy chain V-region. When exchanging the chains of antibodies, the light chain and heavy chain of each antibody are preferably derived from the same V-regions. For example, antibodies 55, 165 and 253 all have heavy chains derived from the IGHV1-58 v-region, and light chains derived from Kappa 3-20. It is shown herein that combining the light chains of 55 or 165 with the heavy chain of 253 leads to a >1 log increase in neutralization titres. Other combinations may be envisaged as the structures of 253 and of 253/55 and 253/165 with either RBD or Spike show that they bind almost identically to the same epitope and don't contact any of the three mutation site residues in the B.1.351 variant.

Accordingly, in one embodiment, the invention provides a method of generating an antibody that binds specifically to the spike protein of SARS-CoV-2 (e.g. a SARS-CoV-2 strain of the B.1.351 lineage), the method comprising identifying two or more antibodies derived from the same light chain and/or heavy chain v-regions, replacing the light chain of a first antibody with the light chain of a second antibody, to thereby generate a mixed-chain antibody comprising the heavy chain of the first antibody and the light chain of the second antibody. In one embodiment, the method further comprises determining the affinity for and/or neutralisation of SARS-CoV-2 of the mixed-chain antibody. The method may further comprise comparing the affinity of the mixed-chain antibody with that of the first and/or second antibodies. The method may further comprise selecting a mixed chain antibody that has the same or greater affinity than the first and/or second antibodies. In some embodiments, the heavy chain v-region is IGHV 1-58 and/or the light chain v-region is IGLV Kappa 3-20.

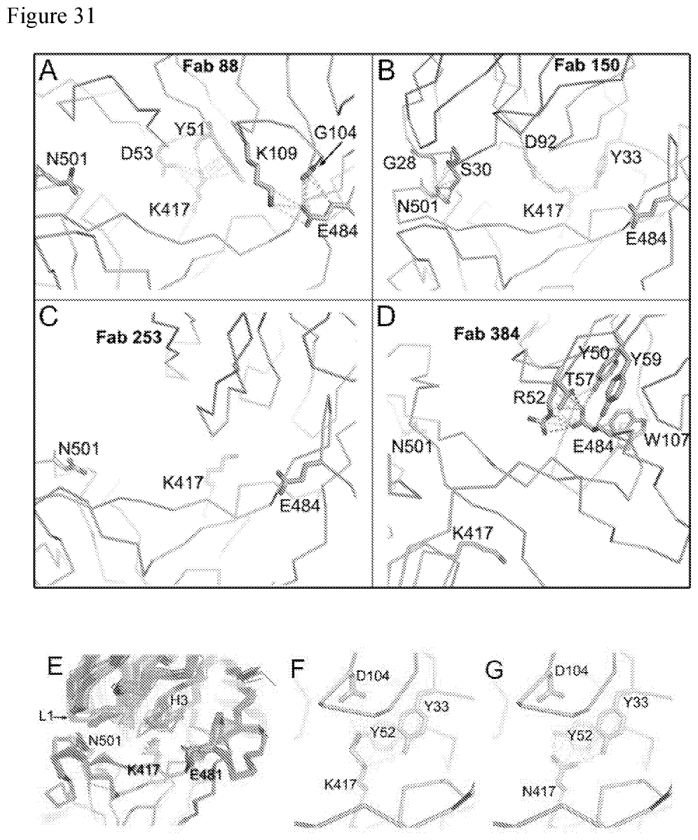

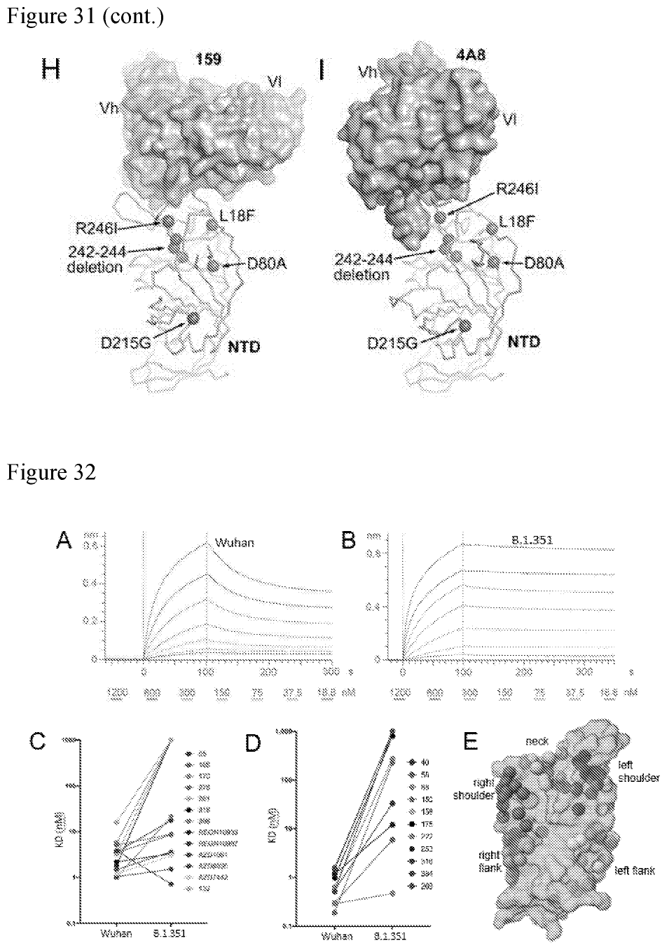

In one embodiment, the antibody of the invention comprises at least three CDRs of antibody 222. A number of the antibodies identified herein use the public HC V-region IGHV3-53. Four of these, 150, 158, 175 and 269, have their neutralization and binding abilities against the B.1.351 variant severely compromised or abolished. However, antibody 222 is an exception, since its binding is unaffected by the B.1.351 variant. The family of IGHV3-53 antibodies bind at the same epitope at the back of the neck of the RBD with very similar approach orientations also shared by the IGHV3-66 Fabs. The majority of these make direct contacts to K417 and N501, but none of them contact E484. The rather short HC CDR3s of these Fabs are usually positioned directly above K417, making hydrogen bonds or salt bridges as well as hydrophobic interactions, while N501 interacts with the LC CDR-1 loop. MAb 150 is a little different, forming both a salt-bridge between K417 and the LC CDR3 D92 and a H-bond between N501 and S30 in the LC CDR1 (FIG. 31B), whereas 158 is more typical, making a hydrogen bond from the carbonyl oxygen of G100 of the HC CDR3 and K417 and hydrophobic contacts from S30 of the LC CDR1 to N501. It would therefore be expected that the combined effects of the K417N and N501Y mutations would severely compromise the binding of most IGHV3-53 and IGHV3-66 class mAbs. However antibody 222 is unaffected by either the B.1.1.7 or B.1.351 variant. Furthermore, antibody 222 is amongst the most potent neutralising antibodies against the B.1.1.529 (omicron) variant tested.

Surprisingly, the neutralisation of antibodies 150, 158, 175 and 269 against the B.1.1.7, B.1.351 and/or P.1 variants can be restored by switching the original light chain of antibodies 150, 158, 175 and 269 with the light chain of 222. As described in the Examples, the CDRH3 of the IGHV3-53-derived antibodies makes a relatively weak contact with the RBD.

Accordingly, an antibody of the invention comprises: (i) the CDRL1, CDRL2 and CDRL3 of antibody 222 having the amino acid sequences specified in SEQ ID NOs: 258, 259 and 260, respectively; and (ii) the CDRH1 and CDRH2 independently selected from any one of the antibodies consisting of: 150, 158, 175, 269, 40 and 398. The antibody may optionally further comprise the CDRH3 of any one of the antibodies selected from: 150, 158, 175, 269, 40 and 398. In one embodiment, the CDRH1 and CDRH2 are independently selected from: 150, 158, 175 and 269, most preferably 150, 158.

Based on sequence similarity between antibodies 150, 158, 175, 269, 40 and 398, a consensus sequence for CDRH1 and CDRH2 of the antibody may be obtained as follows:

CDRH1:
(SEQ ID NO: 407)
G-X¹-T-C-X²-X³-N-Y
CDRH2:
(SEQ ID NO: 408)
I-Y-X⁴-G-G-X⁵-T

Xⁿ may be any amino acid. X¹ is preferably non-polar, more preferably L, V or F, most preferably L or V. X² is preferably a polar side chain, more preferably S or N, most preferably S. X³ is preferably a polar or charged side chain, more preferably, S or R, most preferably S. X⁴ is preferably a polar or non-polar side chain, more preferably S or P. X⁵ is preferably a non-polar side chain, more preferably S or T. Accordingly, an antibody of the invention may comprise a CDRH1 and CDRH2 according to the consensus sequence, for example, in combination with the CDRL1, CDRL2 and CDRL3 of antibody 222.

Based on the known CDR sequences of antibodies derived from the same public v-regions, together with structural data showing the interactions between said antibodies and the viral spike protein, a consensus sequence for the CDRs from antibodies 55, 165 and 253 may be obtained, as follows:

CDRH1:
(SEQ ID NO: 401)
G-F-T-F-T-X1-S-A
CDRH2:
(SEQ ID NO: 402)
I-V-V-G-S-G-N-T
CDRH3:
(SEQ ID NO: 403)
A-A-P-X2-C-X3-X4-S/T-C-X5-D-X6-F-D-I
CDRL1:
(SEQ ID NO: 404)
Q-S-V-X7-S-S-Y
CDRL2:
(SEQ ID NO:405)
G-A-S
CDRL3:
(SEQ ID NO: 406)
Q-Q-Y-G-S-S-P-X8-T

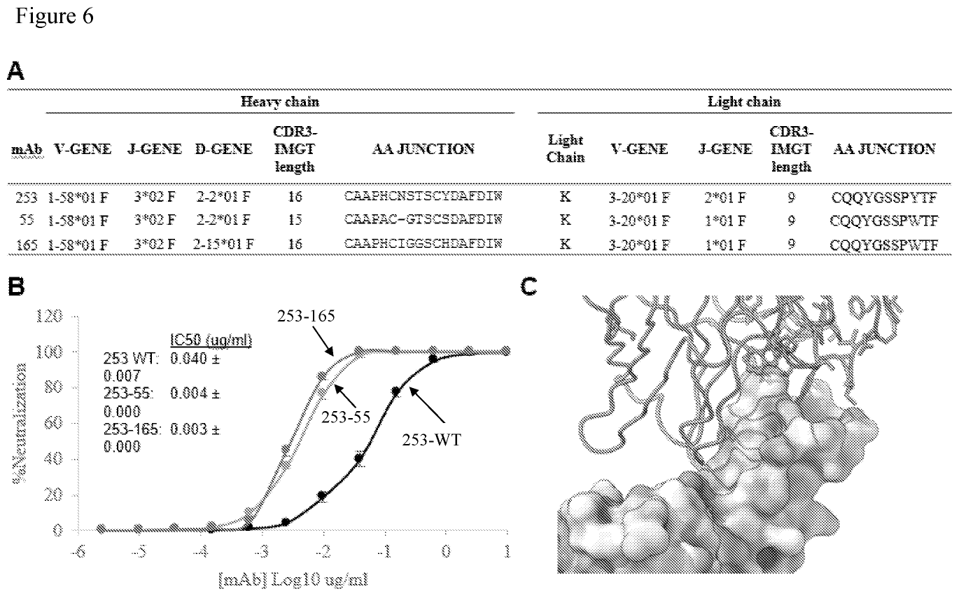

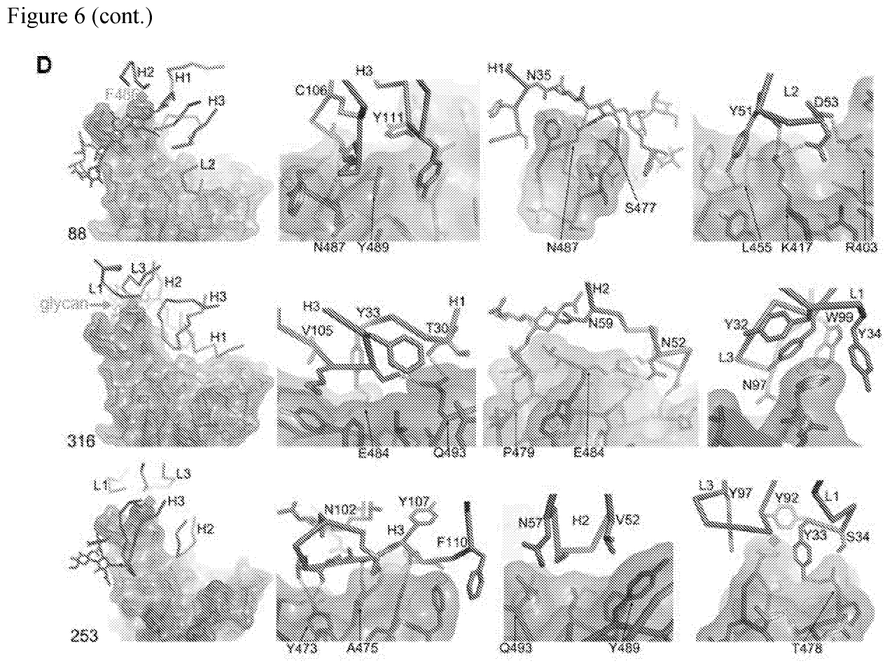

X1-X8 may be any amino acid. X1 is preferably a polar amino acid or is S or T. The structural data provided in FIG. 6 B indicates that the invariant side chains of S105, D108 and F110 interact with the spike protein. A disulphide bond is also formed between the two cysteines in the CDRH3. Based on the variation in the CDRH3 sequences in combination with the structural data, is it plausible that the variable amino acids may be any amino acid. X2 is preferably A or H. Furthermore, it has been shown by structural analysis and by biochemical characterisation that the glycan of antibody 253 does not directly interact with the spike protein. Accordingly, X3 may be any amino acid and X4 may be any one or any two amino acids. If X4 is a single amino acid, then one of X3 and X4 is preferably a glycine, so that the disulphide bond between the cysteines residues of the CDRH3 may be formed. X3 is preferably any non-polar or polar amino acid, or G/I/N. X4 is preferably T/GG/ST/S. X5 is preferably any polar or charged amino acid, or S/H/Y. X6 is preferably A. X7 is preferably any polar/charged amino acid, or more preferably R/S. X8 is preferably a hydrophobic amino acid, such as an aromatic amino acid, W/Y/F or W/Y.

Hence, an antibody of the invention may comprise at least three CDRs of the consensus sequence as defined in the previous paragraph, i.e. as selected from SEQ ID NOs: 401 to 406. For example, the antibody may comprise a CDRH1, CDRH2 and CDRH3 having the amino acid sequences specified in SEQ ID NOs: 401, 402 and 403, respectively, and a CDRL1, CDRL2 and CDRL3 having the amino acid sequences specified in SEQ ID NOs: 404, 405 and 406, respectively. Such antibodies have effective cross-lineage neutralisation effects, e.g. against the Victoria, B.1.1.7 and B.1.351 strains described herein.

Furthermore, it is envisaged that although it is plausible that any antibody comprising the consensus CDRs are effective against SARS-CoV-2, the skilled person can readily screen for antibodies having the desired effect. Thus, the invention also comprises methods for screening said antibodies using any method known to the skilled person, such as those described herein.

### Mixed Chain Antibodies

An antibody of the invention may comprise a light chain variable domain comprising CDRL1, CDRL2 and CDRL3 from a first antibody in Table 1 and a heavy chain variable domain comprising CDRH1, CDRH2 and CDRH3 from a second antibody in Table 1. Such antibodies are referred to as mixed chain antibodies or chimeric antibodies herein. Examples of the mixed chain antibodies are provided in Tables 21 to 25. The mixed chain antibodies have particularly potent cross-lineage neutralisation effects, as demonstrated in the Examples.

Hence, in one embodiment, an antibody of the invention comprises a heavy chain variable domain comprising CDRH1, CDRH2 and CDRH3 from a first antibody in Table 1 and a light chain variable domain comprising CDRL1, CDRL2 and CDRL3 from a second antibody in Table 1. The antibody may comprise a heavy chain variable domain amino acid sequence having at least 80% sequence identity to the heavy chain variable domain from a first antibody in Table 1, and a light chain variable domain amino acid sequence having at least 80% sequence identity to the light chain variable domain from a second antibody in Table 1.

The first and second antibodies in Table 1 may be derived from the same germline heavy chain or light chain v-region. For example, the heavy chain v-region may be IGHV3-53, IGHV1-58 or IGHV3-66. The light chain v-region may be IGκV3-20 or IGκV1-9. In one embodiment, the first antibody is 150 and the second antibody is 222. In another embodiment, the first antibody is 253 and the second antibody is 55. In another embodiment, the first antibody is 253 and the second antibody is 165.

In one embodiment, the second antibody is 222. Hence, in one embodiment, the CDRL1, CDRL2 and CDRL3 have the amino acid sequences specified in SEQ ID NOs: 258, 259 and 260, respectively.

It was unexpectedly found that the light chain of antibody 222 could act as a “universal” light chain when combined with the heavy chain of another antibody in Table 1, such as an antibody derived from IGHV3-53 or IGHV3-66. The 222 light chain was able to cause the resultant mixed chain antibody to bind to and neutralize SARS-CoV-2 strains that would otherwise not have been bound or neutralised by the parent antibody of the heavy chain.

In particular, it was found that by combing the light chain of antibody 222 with the heavy chain of another antibody derived from IGHV3-53 (e.g. antibodies 150, 158, 175 and 269), the resultant mixed chain antibodies showed increased neutralisation when compared to a parent antibody. For example, by combining the 222 light chain with the 175 or 269 heavy chain, the resultant mixed chain antibodies had increased neutralisation effects against the B.1.1.7 variant (see Table 18 and FIG. 37). Furthermore, by combining the 222 light chain with the 150 or 158 heavy chain, the resultant mixed chain antibodies had increased neutralisation effects against the B.1.1.7, B.1.351 and P.1 variants (see Table 18 and FIG. 37). Hence, antibodies 222, 150H222L, 158H222L, 175H222L and 260H222L are particularly useful with the invention, particularly antibodies 222, 150H222L, and 158H222L as they have potent cross-lineage neutralisation effects.

Due to the similarity between IGHV3-53 and IGHV3-66, it is expected that similar results would be achieved by combining the light chain of antibody 222 with the heavy chains from antibodies derived from IGHV3-53 or IGHV3-66.

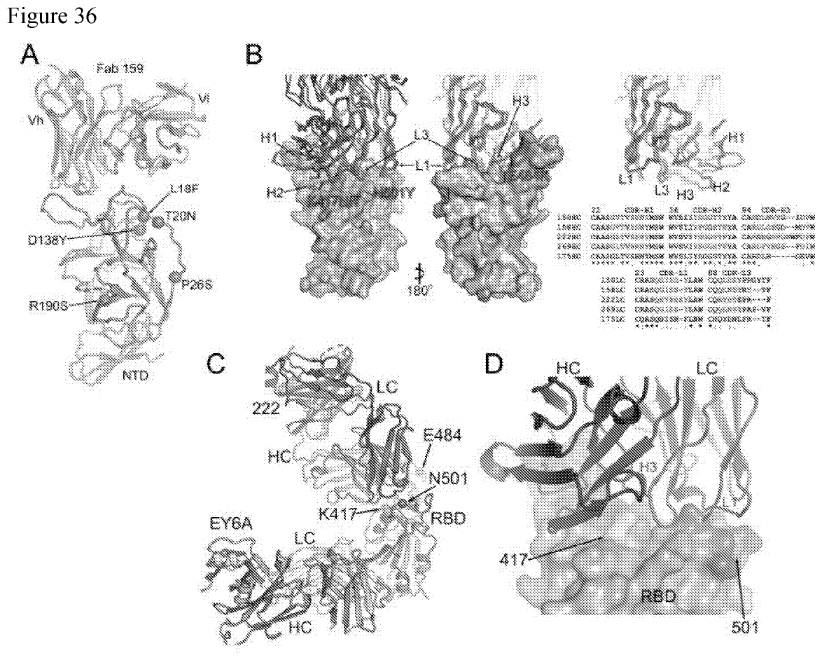

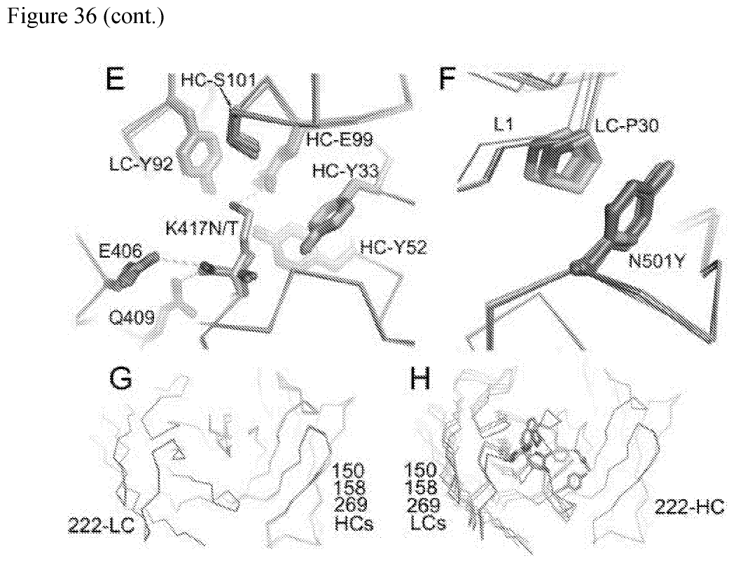

It appears however that the heavy chain of 222 may not be useful as a universal heavy chain for the IGH3-53 antibodies because the modelling studies in Example 33 show that when the light chains of the VH3-53 mAbs (e.g. 348 150, 158, 175 and 269) were docked onto the heavy chain of antibody 222, there may be some steric clashes (see FIG. 36H).

The antibody may comprise a heavy chain variable domain comprising CDRH1, CDRH2 and CDRH3 from a first antibody in Table 1 and a light chain variable domain comprising CDRL1, CDRL2 and CDRL3 from a second antibody in Table 1, wherein the first and second antibodies are derived from the same germline heavy chain IGHV3-53. Hence, the first or second Table 1 antibody may be selected from 150, 158, 175, 222 and 269.

In one embodiment, the first antibody may be 150 from Table 1 and the second antibody may be 222 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 152 and a light chain variable domain consisting of SEQ ID NO: 254, i.e. the antibody is 150H222L. This antibody has potent cross-lineage neutralisation effects, e.g. it is effective against all tested SARS-CoV-2 strains in the Examples (as shown in Table 18 and FIG. 37).

In one embodiment, the first antibody may be 158 from Table 1 and the second antibody may be 222 from Table 1. Hence, an antibody of the invention may comprise a CDRH1, CDRH2 and CDRH3 having the amino acid sequences specified in SEQ ID NOs: 165, 166 and 167, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 162 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 162 and a light chain variable domain consisting of SEQ ID NO: 254, i.e. the antibody is 158H222L. This antibody has potent cross-lineage neutralisation effects, e.g. it is effective against all tested SARS-CoV-2 strains in the Examples (as shown in Table 18 and FIG. 37).

In one embodiment, the first antibody may be 175 from Table 1 and the second antibody may be 222 from Table 1. Hence, an antibody of the invention may comprise a CDRH1, CDRH2 and CDRH3 having the amino acid sequences specified in SEQ ID NOs: 205, 206 and 207, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 202 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 202 and a light chain variable domain consisting of SEQ ID NO: 254, i.e. the antibody is 175H222L. This antibody is capable of exhibiting potent cross-lineage neutralisation effects, e.g. it is effective against the Victoria strain and B.1.1.7 strain (see FIG. 37).

In one embodiment, the first antibody may be 269 from Table 1 and the second antibody may be 222 from Table 1. Hence, an antibody of the invention may comprise a CDRH1, CDRH2 and CDRH3 having the amino acid sequences specified in SEQ ID NOs: 275, 276 and 277, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 272 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 272 and a light chain variable domain consisting of SEQ ID NO: 254, i.e. the antibody is 269H222L. This antibody is capable of exhibiting potent cross-lineage neutralisation effects, e.g. it is effective against the Victoria strain and B.1.1.7 strain (see FIG. 37).

In one embodiment, the first antibody may be 150 from Table 1 and the second antibody may be 158 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 152 and a light chain variable domain consisting of SEQ ID NO: 164, i.e. the antibody is 150H158L.

In one embodiment, the first antibody may be 150 from Table 1 and the second antibody may be 175 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 152 and a light chain variable domain consisting of SEQ ID NO: 204, i.e. the antibody is 150H175L.

In one embodiment, the first antibody may be 150 from Table 1 and the second antibody may be 269 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 152 and a light chain variable domain consisting of SEQ ID NO: 274, i.e. the antibody is 150H269L.

In one embodiment, the first antibody may be 158 from Table 1 and the second antibody may be 150 from Table 1. Hence, an antibody of the invention may comprise a CDRH1, CDRH2 and CDRH3 having the amino acid sequences specified in SEQ ID NOs: 165, 166 and 167, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 162 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 162 and a light chain variable domain consisting of SEQ ID NO: 154, i.e. the antibody is 158H150L.

In one embodiment, the first antibody may be 158 from Table 1 and the second antibody may be 175 from Table 1. Hence, an antibody of the invention may comprise a CDRH1, CDRH2 and CDRH3 having the amino acid sequences specified in SEQ ID NOs: 165, 166 and 167, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 162 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 162 and a light chain variable domain consisting of SEQ ID NO: 204, i.e. the antibody is 158H175L.

In one embodiment, the first antibody may be 158 from Table 1 and the second antibody may be 269 from Table 1. Hence, an antibody of the invention may comprise a CDRH1, CDRH2 and CDRH3 having the amino acid sequences specified in SEQ ID NOs: 165, 166 and 167, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 162 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 162 and a light chain variable domain consisting of SEQ ID NO: 274, i.e. the antibody is 158H269L.

In one embodiment, the first antibody may be 175 from Table 1 and the second antibody may be 150 from Table 1. Hence, an antibody of the invention may comprise a CDRH1, CDRH2 and CDRH3 having the amino acid sequences specified in SEQ ID NOs: 205, 206 and 207, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 202 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 202 and a light chain variable domain consisting of SEQ ID NO: 154, i.e. the antibody is 175H150L.

In one embodiment, the first antibody may be 175 from Table 1 and the second antibody may be 158 from Table 1. Hence, an antibody of the invention may comprise a CDRH1, CDRH2 and CDRH3 having the amino acid sequences specified in SEQ ID NOs: 205, 206 and 207, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 202 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 202 and a light chain variable domain consisting of SEQ ID NO: 164, i.e. the antibody is 175H158L.

In one embodiment, the first antibody may be 175 from Table 1 and the second antibody may be 269 from Table 1. Hence, an antibody of the invention may comprise a CDRH1, CDRH2 and CDRH3 having the amino acid sequences specified in SEQ ID NOs: 205, 206 and 207, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 202 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 202 and a light chain variable domain consisting of SEQ ID NO: 274, i.e. the antibody is 175H269L.

In one embodiment, the first antibody may be 222 from Table 1 and the second antibody may be 150 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 252 and a light chain variable domain consisting of SEQ ID NO: 154, i.e. the antibody is 222H150L.

In one embodiment, the first antibody may be 222 from Table 1 and the second antibody may be 158 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 252 and a light chain variable domain consisting of SEQ ID NO: 164, i.e. the antibody is 222H158L.

In one embodiment, the first antibody may be 222 from Table 1 and the second antibody may be 175 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 252 and a light chain variable domain consisting of SEQ ID NO: 204, i.e. the antibody is 222H175L.

In one embodiment, the first antibody may be 222 from Table 1 and the second antibody may be 269 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 252 and a light chain variable domain consisting of SEQ ID NO: 274, i.e. the antibody is 222H269L.

In one embodiment, the first antibody may be 269 from Table 1 and the second antibody may be 150 from Table 1. Hence, an antibody of the invention may comprise a CDRH1, CDRH2 and CDRH3 having the amino acid sequences specified in SEQ ID NOs: 275, 276 and 277, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 272 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 272 and a light chain variable domain consisting of SEQ ID NO: 154, i.e. the antibody is 269H150L.

In one embodiment, the first antibody may be 269 from Table 1 and the second antibody may be 158 from Table 1. Hence, an antibody of the invention may comprise a CDRH1, CDRH2 and CDRH3 having the amino acid sequences specified in SEQ ID NOs: 275, 276 and 277, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 272 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 272 and a light chain variable domain consisting of SEQ ID NO: 164, i.e. the antibody is 269H158L.

In one embodiment, the first antibody may be 269 from Table 1 and the second antibody may be 175 from Table 1. Hence, an antibody of the invention may comprise a CDRH1, CDRH2 and CDRH3 having the amino acid sequences specified in SEQ ID NOs: 275, 276 and 277, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 272 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 272 and a light chain variable domain consisting of SEQ ID NO: 204, i.e. the antibody is 269H175L.

Furthermore, due to the similarity between IGHV3-53 and IGHV3-66, swapping the light chain and heavy chain of these antibodies may generate antibodies useful for the invention. Hence, an antibody of the invention may comprise a heavy chain variable domain comprising CDRH1, CDRH2 and CDRH3 from a first antibody in Table 1 and a light chain variable domain comprising CDRL1, CDRL2 and CDRL3 from a second antibody in Table 1, wherein the first and second antibodies are derived from either the germline heavy chain IGHV3-53 or IGHV3-66. Hence, the first or second Table 1 antibody may be selected from 150, 158, 175, 222, 269, 40, 398.

In one embodiment, the first antibody may be 150 from Table 1 and the second antibody may be 40 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 28, 29 and 30, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 24. The antibody may comprise a heavy chain variable domain consisting of SEQ ID NO: 152 and a light chain variable domain consisting of SEQ ID NO: 24, i.e. the antibody is 150H40L.

In one embodiment, the first antibody may be 150 from Table 1 and the second antibody may be 398 from Table 1. Hence, an antibody of the invention may comprise a CDRH1, CDRH2 and CDRH3 having the amino acid sequences specified in SEQ ID NOs: 155, 156 and 157, respectively, and a CDRL1, CDRL2 and CDRL3 having the amino acid sequences specified in SEQ ID NOs: 398, 399 and 400, respectively. The antibody may comprise a heavy chain variable domain having ≥80%, ≥90%, ≥95%, ≥96%, ≥97%, ≥98%, ≥99% or 100% sequence identity to SEQ ID NO: 152 and a light chain variable domain having ≥80%, ≥90%, ≥95%, ≥96%, ≥97%, ≥98%, ≥99% or 100% sequence identity to SEQ ID NO: 394. The antibody may comprise a heavy chain variable domain consisting of SEQ ID NO: 152 and a light chain variable domain consisting of SEQ ID NO: 394, i.e. the antibody is 150H398L.

In one embodiment, the first antibody may be 40 from Table 1 and the second antibody may be 150 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 22 and a light chain variable domain consisting of SEQ ID NO: 154, i.e. the antibody is 40H150L.

In one embodiment, the first antibody may be 40 from Table 1 and the second antibody may be 158 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 22 and a light chain variable domain consisting of SEQ ID NO: 164, i.e. the antibody is 40H158L.

In one embodiment, the first antibody may be 40 from Table 1 and the second antibody may be 175 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 22 and a light chain variable domain consisting of SEQ ID NO: 204, i.e. the antibody is 40H175L.

In one embodiment, the first antibody may be 40 from Table 1 and the second antibody may be 222 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 22 and a light chain variable domain consisting of SEQ ID NO: 254, i.e. the antibody is 40H222L.

In one embodiment, the first antibody may be 40 from Table 1 and the second antibody may be 269 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 22 and a light chain variable domain consisting of SEQ ID NO: 274, i.e. the antibody is 40H269L.

In one embodiment, the first antibody may be 40 from Table 1 and the second antibody may be 398 from Table 1. Hence, an antibody of the invention may comprise a CDRH1, CDRH2 and CDRH3 having the amino acid sequences specified in SEQ ID NOs: 25, 26 and 27, respectively, and a CDRL1, CDRL2 and CDRL3 having the amino acid sequences specified in SEQ ID NOs: 398, 399 and 400, respectively. The antibody may comprise a heavy chain variable domain having ≥80%, ≥90%, ≥95%, ≥96%, ≥97%, ≥98%, ≥99% or 100% sequence identity to SEQ ID NO: 22 and a light chain variable domain having ≥80%, ≥90%, ≥95%, ≥96%, ≥97%, ≥98%, ≥99% or 100% sequence identity to SEQ ID NO: 394. The antibody may comprise a heavy chain variable domain consisting of SEQ ID NO: 22 and a light chain variable domain consisting of SEQ ID NO: 394, i.e. the antibody is 40H398L.

In one embodiment, the first antibody may be 398 from Table 1 and the second antibody may be 150 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 158, 159 and 160, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 154. The antibody may comprise a heavy chain variable domain consisting of SEQ ID NO: 392 and a light chain variable domain consisting of SEQ ID NO: 154, i.e. the antibody is 398H150L.

In one embodiment, the first antibody may be 398 from Table 1 and the second antibody may be 158 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 168, 169 and 170, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 164. The antibody may comprise a heavy chain variable domain consisting of SEQ ID NO: 392 and a light chain variable domain consisting of SEQ ID NO: 164, i.e. the antibody is 398H158L.

In one embodiment, the first antibody may be 398 from Table 1 and the second antibody may be 175 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 208, 209 and 210, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 204. The antibody may comprise a heavy chain variable domain consisting of SEQ ID NO: 392 and a light chain variable domain consisting of SEQ ID NO: 204, i.e. the antibody is 398H175L.

In one embodiment, the first antibody may be 398 from Table 1 and the second antibody may be 222 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 392 and a light chain variable domain consisting of SEQ ID NO: 254, i.e. the antibody is 398H222L.

In one embodiment, the first antibody may be 398 from Table 1 and the second antibody may be 269 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 278, 279 and 280, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 274. The antibody may comprise a heavy chain variable domain consisting of SEQ ID NO: 392 and a light chain variable domain consisting of SEQ ID NO: 274, i.e. the antibody is 398H269L.

In one embodiment, the first antibody may be 398 from Table 1 and the second antibody may be 40 from Table 1. Hence, an antibody of the invention may comprise a CDRH1, CDRH2 and CDRH3 having the amino acid sequences specified in SEQ ID NOs: 395, 396 and 397, respectively, and a CDRL1, CDRL2 and CDRL3 having the amino acid sequences specified in SEQ ID NOs: 28, 29 and 30, respectively. The antibody may comprise a heavy chain variable domain having ≥80%, ≥90%, ≥95%, ≥96%, ≥97%, ≥98%, ≥99% or 100% sequence identity to SEQ ID NO: 392 and a light chain variable domain having ≥80%, ≥90%, ≥95%, ≥96%, ≥97%, ≥98%, ≥99% or 100% sequence identity to SEQ ID NO: 24. The antibody may comprise a heavy chain variable domain consisting of SEQ ID NO: 392 and a light chain variable domain consisting of SEQ ID NO: 24, i.e. the antibody is 398H40L.

The antibody may comprise a heavy chain variable domain comprising CDRH1, CDRH2 and CDRH3 from a first antibody in Table 1 and a light chain variable domain comprising CDRL1, CDRL2 and CDRL3 from a second antibody in Table 1, wherein the first and second antibodies are derived from the same germline light chain IGκV3-20. Hence, the first or second Table 1 antibody may be selected from 55, 159, 165, 222, 253 and 318.

In one embodiment, the first antibody may be 55 from Table 1 and the second antibody may be 159 from Table 1. Hence, an antibody of the invention may comprise a CDRH1, CDRH2 and CDRH3 having the amino acid sequences specified in SEQ ID NOs: 65, 66 and 67, respectively, and a CDRL1, CDRL2 and CDRL3 having the amino acid sequences specified in SEQ ID NOs: 178, 179 and 180, respectively. The antibody may comprise a heavy chain variable domain having ≥80%, ≥90%, ≥95%, ≥96%, ≥97%, ≥98%, ≥99% or 100% sequence identity to SEQ ID NO: 62 and a light chain variable domain having ≥80%, ≥90%, ≥95%, ≥96%, ≥97%, ≥98%, ≥99% or 100% sequence identity to SEQ ID NO: 174. The antibody may comprise a heavy chain variable domain consisting of SEQ ID NO: 62 and a light chain variable domain consisting of SEQ ID NO: 174, i.e. the antibody is 55H159L.

In one embodiment, the first antibody may be 55 from Table 1 and the second antibody may be 222 from Table 1. Hence, an antibody of the invention may comprise a CDRH1, CDRH2 and CDRH3 having the amino acid sequences specified in SEQ ID NOs: 66 and 67, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 62 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 62 and a light chain variable domain consisting of SEQ ID NO: 254, i.e. the antibody is 55H222L.

In one embodiment, the first antibody may be 159 from Table 1 and the second antibody may be 55 from Table 1. Hence, an antibody of the invention may comprise a CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 68, 69 and 70, respectively. The antibody may comprise a heavy chain variable domain having ≥80%, ≥90%, ≥95%, ≥96%, ≥97%, ≥98%, ≥99% or 100% sequence identity to SEQ ID NO: 172 and a light chain variable domain having ≥80%, ≥90%, ≥95%, ≥96%, ≥97%, ≥98%, ≥99% or 100% sequence identity to SEQ ID NO: 64. The antibody may comprise a heavy chain variable domain consisting of SEQ ID NO: 172 and a light chain variable domain consisting of SEQ ID NO: 64, i.e. the antibody is 159H55L.

In one embodiment, the first antibody may be 159 from Table 1 and the second antibody may be 165 from Table 1. Hence, an antibody of the invention may comprise a CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 188, 189 and 190, respectively. The antibody may comprise a heavy chain variable domain having ≥80%, ≥90%, ≥95%, ≥96%, ≥97%, ≥98%, ≥99% or 100% sequence identity to SEQ ID NO: 172 and a light chain variable domain having ≥80%, ≥90%, ≥95%, ≥96%, ≥97%, ≥98%, ≥99% or 100% sequence identity to SEQ ID NO: 184. The antibody may comprise a heavy chain variable domain consisting of SEQ ID NO: 172 and a light chain variable domain consisting of SEQ ID NO: 184, i.e. the antibody is 159H165L.

In one embodiment, the first antibody may be 159 from Table 1 and the second antibody may be 222 from Table 1. Hence, an antibody of the invention may comprise a CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 172 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 172 and a light chain variable domain consisting of SEQ ID NO: 254, i.e. the antibody is 159H222L.

In one embodiment, the first antibody may be 159 from Table 1 and the second antibody may be 253 from Table 1. Hence, an antibody of the invention may comprise a CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 268, 269 and 270, respectively. The antibody may comprise a heavy chain variable domain having ≥80%, ≥90%, ≥95%, ≥96%, ≥97%, ≥98%, ≥99% or 100% sequence identity to SEQ ID NO: 172 and a light chain variable domain having ≥80%, ≥90%, ≥95%, ≥96%, ≥97%, ≥98%, ≥99% or 100% sequence identity to SEQ ID NO: 264. The antibody may comprise a heavy chain variable domain consisting of SEQ ID NO: 172 and a light chain variable domain consisting of SEQ ID NO: 264, i.e. the antibody is 159H253L.

In one embodiment, the first antibody may be 159 from Table 1 and the second antibody may be 318 from Table 1. Hence, an antibody of the invention may comprise a CDRH1, CDRH2 and CDRH3 having the amino acid sequences specified in SEQ ID NOs: 175, 176 and 177, respectively, and a CDRL1, CDRL2 and CDRL3 having the amino acid sequences specified in SEQ ID NOs: 338, 339 and 340, respectively. The antibody may comprise a heavy chain variable domain having ≥80%, ≥90%, ≥95%, ≥96%, ≥97%, ≥98%, ≥99% or 100% sequence identity to SEQ ID NO: 172 and a light chain variable domain having ≥80%, ≥90%, ≥95%, ≥96%, ≥97%, ≥98%, ≥99% or 100% sequence identity to SEQ ID NO: 334. The antibody may comprise a heavy chain variable domain consisting of SEQ ID NO: 172 and a light chain variable domain consisting of SEQ ID NO: 334, i.e. the antibody is 159H318L.

In one embodiment, the first antibody may be 165 from Table 1 and the second antibody may be 159 from Table 1. Hence, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 178, 179 and 180, respectively. The antibody may comprise a heavy chain variable domain having ≥80%, ≥90%, ≥95%, ≥96%, ≥97%, ≥98%, ≥99% or 100% sequence identity to SEQ ID NO: 182 and a light chain variable domain having ≥80%, ≥90%, ≥95%, ≥96%, ≥97%, ≥98%, ≥99% or 100% sequence identity to SEQ ID NO: 174. The antibody may comprise a heavy chain variable domain consisting of SEQ ID NO: 182 and a light chain variable domain consisting of SEQ ID NO: 174, i.e. the antibody is 165H159L.

In one embodiment, the first antibody may be 165 from Table 1 and the second antibody may be 222 from Table 1. Hence, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 182 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 182 and a light chain variable domain consisting of SEQ ID NO: 254, i.e. the antibody is 165H222L.

In one embodiment, the first antibody may be 222 from Table 1 and the second antibody may be 55 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 68, 69 and 70, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 64. The antibody may comprise a heavy chain variable domain consisting of SEQ ID NO: 252 and a light chain variable domain consisting of SEQ ID NO: 64, i.e. the antibody is 222H55L.

In one embodiment, the first antibody may be 222 from Table 1 and the second antibody may be 159 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 178, 179 and 180, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 174. The antibody may comprise a heavy chain variable domain consisting of SEQ ID NO: 252 and a light chain variable domain consisting of SEQ ID NO: 174, i.e. the antibody is 222H159L.

In one embodiment, the first antibody may be 222 from Table 1 and the second antibody may be 165 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 188, 189 and 190, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 184. The antibody may comprise a heavy chain variable domain consisting of SEQ ID NO: 252 and a light chain variable domain consisting of SEQ ID NO: 184, i.e. the antibody is 222H165L.

In one embodiment, the first antibody may be 222 from Table 1 and the second antibody may be 253 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 268, 269 and 270, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 264. The antibody may comprise a heavy chain variable domain consisting of SEQ ID NO: 252 and a light chain variable domain consisting of SEQ ID NO: 264, i.e. the antibody is 222H253L.

In one embodiment, the first antibody may be 222 from Table 1 and the second antibody may be 318 from Table 1. Hence, an antibody of the invention may comprise a CDRH1, CDRH2 and CDRH3 having the amino acid sequences specified in SEQ ID NOs: 255, 256 and 257, respectively, and a CDRL1, CDRL2 and CDRL3 having the amino acid sequences specified in SEQ ID NOs: 338, 339 and 340, respectively. The antibody may comprise a heavy chain variable domain having ≥80%, ≥90%, ≥95%, ≥96%, ≥97%, ≥98%, ≥99% or 100% sequence identity to SEQ ID NO: 252 and a light chain variable domain having ≥80%, ≥90%, ≥95%, ≥96%, ≥97%, ≥98%, ≥99% or 100% sequence identity to SEQ ID NO: 334. The antibody may comprise a heavy chain variable domain consisting of SEQ ID NO: 252 and a light chain variable domain consisting of SEQ ID NO: 334, i.e. the antibody is 222H318L.

In one embodiment, the first antibody may be 253 from Table 1 and the second antibody may be 159 from Table 1. Hence, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 178, 179 and 180, respectively. The antibody may comprise a heavy chain variable domain having ≥80%, ≥90%, ≥95%, ≥96%, ≥97%, ≥98%, ≥99% or 100% sequence identity to SEQ ID NO: 262 and a light chain variable domain having ≥80%, ≥90%, ≥95%, ≥96%, ≥97%, ≥98%, ≥99% or 100% sequence identity to SEQ ID NO: 174. The antibody may comprise a heavy chain variable domain consisting of SEQ ID NO: 262 and a light chain variable domain consisting of SEQ ID NO: 174, i.e. the antibody is 253H159L.

In one embodiment, the first antibody may be 253 from Table 1 and the second antibody may be 222 from Table 1. Hence, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 262 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 262 and a light chain variable domain consisting of SEQ ID NO: 254, i.e. the antibody is 253H222L.

In one embodiment, the first antibody may be 318 from Table 1 and the second antibody may be 159 from Table 1. Hence, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 178, 179 and 180, respectively. The antibody may comprise a heavy chain variable domain having ≥80%, ≥90%, ≥95%, ≥96%, ≥97%, ≥98%, ≥99% or 100% sequence identity to SEQ ID NO: 332 and a light chain variable domain having ≥80%, ≥90%, ≥95%, ≥96%, ≥97%, ≥98%, ≥99% or 100% sequence identity to SEQ ID NO: 174. The antibody may comprise a heavy chain variable domain consisting of SEQ ID NO: 332 and a light chain variable domain consisting of SEQ ID NO: 174, i.e. the antibody is 318H159L.

In one embodiment, the first antibody may be 318 from Table 1 and the second antibody may be 222 from Table 1. Hence, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 258, 259 and 260, respectively. The antibody may comprise a heavy chain variable domain having ≥80%, ≥90%, ≥95%, ≥96%, ≥97%, ≥98%, ≥99% or 100% sequence identity to SEQ ID NO: 332 and a light chain variable domain having ≥80%, ≥90%, ≥95%, ≥96%, ≥97%, ≥98%, ≥99% or 100% sequence identity to SEQ ID NO: 254. The antibody may comprise a heavy chain variable domain consisting of SEQ ID NO: 332 and a light chain variable domain consisting of SEQ ID NO: 254, i.e. the antibody is 318H222L.

The antibody may comprise a heavy chain variable domain comprising CDRH1, CDRH2 and CDRH3 from a first antibody in Table 1 and a light chain variable domain comprising CDRL1, CDRL2 and CDRL3 from a second antibody in Table 1, wherein the first and second antibodies are derived from the same germline light chain IGκV1-9. Hence, the first or second Table 1 antibody may be selected from 150, 158 and 269.

The antibody may comprise a heavy chain variable domain comprising CDRH1, CDRH2 and CDRH3 from a first antibody in Table 1 and a light chain variable domain comprising CDRL1, CDRL2 and CDRL3 from a second antibody in Table 1, wherein the first and second antibodies are derived from the same germline heavy chain IGHV1-58. Hence, the first or second Table 1 antibody may be selected from 55, 165, 253 and 318.

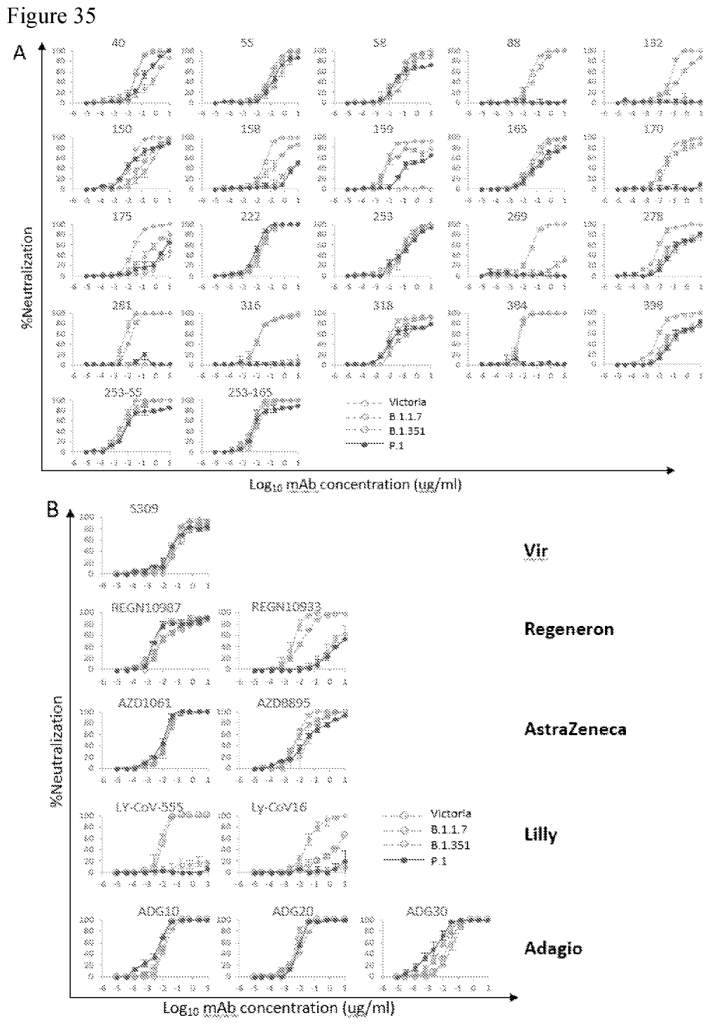

In one embodiment, the first antibody may be 253 from Table 1 and the second antibody may be 55 from Table 1. Hence, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 68, 69 and 70, respectively. The antibody may comprise a heavy chain variable domain having ≥80%, ≥90%, ≥95%, ≥96%, ≥97%, ≥98%, ≥99% or 100% sequence identity to SEQ ID NO: 262 and a light chain variable domain having ≥80%, ≥90%, ≥95%, ≥96%, ≥97%, ≥98%, ≥99% or 100% sequence identity to SEQ ID NO: 64. The antibody may comprise a heavy chain variable domain consisting of SEQ ID NO: 262 and a light chain variable domain consisting of SEQ ID NO: 64, i.e. the antibody is 253H55L. This antibody has potent cross-lineage neutralisation effects, e.g. it is effective against all tested SARS-CoV-2 strains in the Examples (as shown in Table 18 and FIG. 35).

In one embodiment, the first antibody may be 253 from Table 1 and the second antibody may be 165 from Table 1. Hence, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 188, 189 and 190, respectively. The antibody may comprise a heavy chain variable domain having ≥80%, ≥90%, ≥95%, ≥96%, ≥97%, ≥98%, ≥99% or 100% sequence identity to SEQ ID NO: 262 and a light chain variable domain having ≥80%, ≥90%, ≥95%, ≥96%, ≥97%, ≥98%, ≥99% or 100% sequence identity to SEQ ID NO: 186. The antibody may comprise a heavy chain variable domain consisting of SEQ ID NO: 262 and a light chain variable domain consisting of SEQ ID NO: 186, i.e. the antibody is 253H165L. This antibody has potent cross-lineage neutralisation effects, e.g. it is effective against all tested SARS-CoV-2 strains in the Examples (as shown in Table 18 and FIG. 35).

In one embodiment, the first antibody may be 55 from Table 1 and the second antibody may be 165 from Table 1. Hence, an antibody of the invention may comprise a CDRH1, CDRH2 and CDRH3 having the amino acid sequences specified in SEQ ID NOs: 66 and 67, respectively, and a CDRL1, CDRL2 and CDRL3 having the amino acid sequences specified in SEQ ID NOs: 188, 189 and 190, respectively. The antibody may comprise a heavy chain variable domain having ≥80%, ≥90%, ≥95%, ≥96%, ≥97%, ≥98%, ≥99% or 100% sequence identity to SEQ ID NO: 62 and a light chain variable domain having ≥80%, ≥90%, ≥95%, ≥96%, ≥97%, ≥98%, ≥99% or 100% sequence identity to SEQ ID NO: 184. The antibody may comprise a heavy chain variable domain consisting of SEQ ID NO: 62 and a light chain variable domain consisting of SEQ ID NO: 184, i.e. the antibody is 55H165L.

In one embodiment, the first antibody may be 55 from Table 1 and the second antibody may be 253 from Table 1. Hence, an antibody of the invention may comprise a CDRH1, CDRH2 and CDRH3 having the amino acid sequences specified in SEQ ID NOs: 66 and 67, respectively, and a CDRL1, CDRL2 and CDRL3 having the amino acid sequences specified in SEQ ID NOs: 268, 269 and 270, respectively. The antibody may comprise a heavy chain variable domain having ≥80%, ≥90%, ≥95%, ≥96%, ≥97%, ≥98%, ≥99% or 100% sequence identity to SEQ ID NO: 62 and a light chain variable domain having ≥80%, ≥90%, ≥95%, ≥96%, ≥97%, ≥98%, ≥99% or 100% sequence identity to SEQ ID NO: 264. The antibody may comprise a heavy chain variable domain consisting of SEQ ID NO: 62 and a light chain variable domain consisting of SEQ ID NO: 264, i.e. the antibody is 55H253L.

In one embodiment, the first antibody may be 55 from Table 1 and the second antibody may be 318 from Table 1. Hence, an antibody of the invention may comprise a CDRH1, CDRH2 and CDRH3 having the amino acid sequences specified in SEQ ID NOs: 65, 66 and 67, respectively, and a CDRL1, CDRL2 and CDRL3 having the amino acid sequences specified in SEQ ID NOs: 338, 339 and 340, respectively. The antibody may comprise a heavy chain variable domain having ≥80%, ≥90%, ≥95%, ≥96%, ≥97%, ≥98%, ≥99% or 100% sequence identity to SEQ ID NO: 62 and a light chain variable domain having ≥80%, ≥90%, ≥95%, ≥96%, ≥97%, ≥98%, ≥99% or 100% sequence identity to SEQ ID NO: 334. The antibody may comprise a heavy chain variable domain consisting of SEQ ID NO: 62 and a light chain variable domain consisting of SEQ ID NO: 334, i.e. the antibody is 55H318L.

In one embodiment, the first antibody may be 165 from Table 1 and the second antibody may be 55 from Table 1. Hence, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 68, 69 and 70, respectively. The antibody may comprise a heavy chain variable domain having ≥80%, ≥90%, ≥95%, ≥96%, ≥97%, ≥98%, ≥99% or 100% sequence identity to SEQ ID NO: 182 and a light chain variable domain having ≥80%, ≥90%, ≥95%, ≥96%, ≥97%, ≥98%, ≥99% or 100% sequence identity to SEQ ID NO: 64. The antibody may comprise a heavy chain variable domain consisting of SEQ ID NO: 182 and a light chain variable domain consisting of SEQ ID NO: 62, i.e. the antibody is 165H55L.

In one embodiment, the first antibody may be 165 from Table 1 and the second antibody may be 253 from Table 1. Hence, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 268, 269 and 270, respectively. The antibody may comprise a heavy chain variable domain having ≥80%, ≥90%, ≥95%, ≥96%, ≥97%, ≥98%, ≥99% or 100% sequence identity to SEQ ID NO: 182 and a light chain variable domain having ≥80%, ≥90%, ≥95%, ≥96%, ≥97%, ≥98%, ≥99% or 100% sequence identity to SEQ ID NO: 264. The antibody may comprise a heavy chain variable domain consisting of SEQ ID NO: 182 and a light chain variable domain consisting of SEQ ID NO: 264, i.e. the antibody is 165H253L.

In one embodiment, the first antibody may be 165 from Table 1 and the second antibody may be 318 from Table 1. Hence, an antibody of the invention may comprise a CDRH1, CDRH2 and CDRH3 having the amino acid sequences specified in SEQ ID NOs: 185, 186 and 187, respectively, and a CDRL1, CDRL2 and CDRL3 having the amino acid sequences specified in SEQ ID NOs: 338, 339 and 340, respectively. The antibody may comprise a heavy chain variable domain having ≥80%, ≥90%, ≥95%, ≥96%, ≥97%, ≥98%, ≥99% or 100% sequence identity to SEQ ID NO: 182 and a light chain variable domain having ≥80%, ≥90%, ≥95%, ≥96%, ≥97%, ≥98%, ≥99% or 100% sequence identity to SEQ ID NO: 334. The antibody may comprise a heavy chain variable domain consisting of SEQ ID NO: 182 and a light chain variable domain consisting of SEQ ID NO: 334, i.e. the antibody is 165H318L.

In one embodiment, the first antibody may be 253 from Table 1 and the second antibody may be 318 from Table 1. Hence, an antibody of the invention may comprise a CDRH1, CDRH2 and CDRH3 having the amino acid sequences specified in SEQ ID NOs: 265, 266 and 267, respectively, and a CDRL1, CDRL2 and CDRL3 having the amino acid sequences specified in SEQ ID NOs: 338, 339 and 340, respectively. The antibody may comprise a heavy chain variable domain having ≥80%, ≥90%, ≥95%, ≥96%, ≥97%, ≥98%, ≥99% or 100% sequence identity to SEQ ID NO: 262 and a light chain variable domain having ≥80%, ≥90%, ≥95%, ≥96%, ≥97%, ≥98%, ≥99% or 100% sequence identity to SEQ ID NO: 334. The antibody may comprise a heavy chain variable domain consisting of SEQ ID NO: 262 and a light chain variable domain consisting of SEQ ID NO: 334, i.e. the antibody is 253H318L.

In one embodiment, the first antibody may be 318 from Table 1 and the second antibody may be 55 from Table 1. Hence, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 68, 69 and 70, respectively. The antibody may comprise a heavy chain variable domain having ≥80%, ≥90%, ≥95%, ≥96%, ≥97%, ≥98%, ≥99% or 100% sequence identity to SEQ ID NO: 332 and a light chain variable domain having ≥80%, ≥90%, ≥95%, ≥96%, ≥97%, ≥98%, ≥99% or 100% sequence identity to SEQ ID NO: 64. The antibody may comprise a heavy chain variable domain consisting of SEQ ID NO: 332 and a light chain variable domain consisting of SEQ ID NO: 64, i.e. the antibody is 318H55L.

In one embodiment, the first antibody may be 318 from Table 1 and the second antibody may be 165 from Table 1. Hence, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 188, 189 and 190, respectively. The antibody may comprise a heavy chain variable domain having ≥80%, ≥90%, ≥95%, ≥96%, ≥97%, ≥98%, ≥99% or 100% sequence identity to SEQ ID NO: 332 and a light chain variable domain having ≥80%, ≥90%, ≥95%, ≥96%, ≥97%, ≥98%, ≥99% or 100% sequence identity to SEQ ID NO: 184. The antibody may comprise a heavy chain variable domain consisting of SEQ ID NO: 332 and a light chain variable domain consisting of SEQ ID NO: 184, i.e. the antibody is 318H165L.

In one embodiment, the first antibody may be 318 from Table 1 and the second antibody may be 253 from Table 1. Hence, an antibody of the invention may comprise a CDRH1, CDRH2 and CDRH3 having the amino acid sequences specified in SEQ ID NOs: 335, 336 and 337, respectively, and a CDRL1, CDRL2 and CDRL3 having the amino acid sequences specified in SEQ ID NOs: 268, 269 and 270, respectively. The antibody may comprise a heavy chain variable domain having ≥80%, ≥90%, ≥95%, ≥96%, ≥97%, ≥98%, ≥99% or 100% sequence identity to SEQ ID NO: 332 and a light chain variable domain having ≥80%, ≥90%, ≥95%, ≥96%, ≥97%, ≥98%, ≥99% or 100% sequence identity to SEQ ID NO: 264. The antibody may comprise a heavy chain variable domain consisting of SEQ ID NO: 332 and a light chain variable domain consisting of SEQ ID NO: 264, i.e. the antibody is 318H253L.

### Antibody Conjugates

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Conjugates of the antibody and cytotoxic agent may be made using a variety of bifunctional protein-coupling agents known in the art.

An antibody, of the invention may be conjugated to a molecule that modulates or alters serum half-life. An antibody, of the invention may bind to albumin, for example in order to modulate the serum half-life. In one embodiment, an antibody of the invention will also include a binding region specific for albumin. In another embodiment, an antibody of the invention may include a peptide linker which is an albumin binding peptide. Examples of albumin binding peptides are included in WO2015/197772 and WO2007/106120 the entirety of which are incorporated by reference.

### Polynucleotides, Vectors and Host Cells

The invention also provides one or more isolated polynucleotides (e.g. DNA) encoding the antibody of the invention. In one embodiment, the polynucleotide sequence is collectively present on more than one polynucleotide, but collectively together they are able to encode an antibody of the invention. For example, the polynucleotides may encode the heavy and/or light chain variable regions(s) of an antibody of the invention. The polynucleotides may encode the full heavy and/or light chain of an antibody of the invention. Typically, one polynucleotide would encode each of the heavy and light chains.

Polynucleotides which encode an antibody of the invention can be obtained by methods well known to those skilled in the art. For example, DNA sequences coding for part or all of the antibody heavy and light chains may be synthesised as desired from the corresponding amino acid sequences.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

A polynucleotide of the invention may be provided in the form of an expression cassette, which includes control sequences operably linked to the inserted sequence, thus allowing for expression of the antibody of the invention in vivo. Hence, the invention also provides one or more expression cassettes encoding the one or more polynucleotides that encoding an antibody of the invention. These expression cassettes, in turn, are typically provided within vectors (e.g. plasmids or recombinant viral vectors). Hence, in one embodiment, the invention provides a vector encoding an antibody of the invention. In another embodiment, the invention provides vectors which collectively encode an antibody of the invention. The vectors may be cloning vectors or expression vectors. A suitable vector may be any vector which is capable of carrying a sufficient amount of genetic information, and allowing expression of a polypeptide of the invention.

The polynucleotides, expression cassettes or vectors of the invention are introduced into a host cell, e.g. by transfection. Hence, the invention also provides a host cell comprising the one or more polynucleotides, expression cassettes or vectors of the invention. The polynucleotides, expression cassettes or vectors of the invention may be introduced transiently or permanently into the host cell, allowing expression of an antibody from the one or more polynucleotides, expression cassettes or vectors. Such host cells include transient, or preferably stable higher eukaryotic cell lines, such as mammalian cells or insect cells, lower eukaryotic cells, such as yeast, or prokaryotic cells, such as bacteria cells. Particular examples of cells include mammalian HEK293, such as HEK293F, HEK293T, HEK293S or HEK Expi293F, CHO, HeLa, NS0 and COS cells, or any other cell line used herein, such as the ones used in the Examples. Preferably the cell line selected will be one which is not only stable, but also allows for mature glycosylation.

The invention also provides a process for the production of an antibody of the invention, comprising culturing a host cell containing one or more vectors of the invention under conditions suitable for the expression of the antibody from the one or more polynucleotides of the invention, and isolating the antibody from said culture.

### Combination of Antibodies

The inventors found that certain Table 1 antibodies are particularly effective when used in combination, e.g. to maximise therapeutic effects and/or increase diagnostic power. Useful combinations include the antibodies that do not cross-compete with one another and/or bind to non-overlapping epitopes, as exemplified in Tables 4 and 5.

Thus, the invention provides a combination of the antibodies of the invention, wherein each antibody is capable of binding to the spike protein of coronavirus SARS-CoV-2, wherein each antibody: (a) comprises at least three CDRs of any one of the 42 antibodies in Table 1; or (b) binds to the same epitope as or competes with antibody 159, or 384. In certain embodiments, the Table 1 antibodies may be:
a pair of antibodies listed in a row of Table 4;
a pair of antibodies listed in a row of Table 5;
a triplet of antibodies listed in a row of Table 5;
a pair of antibodies listed in a row of Table 4 and antibody 159;
a pair of antibodies listed in a row of Table 5 and antibody 159;
a triplet of antibodies listed in a row of Table 5 and antibody 159;
any two or more antibodies selected from the group consisting of: 384, 159, 253H55L, 253H165L, 253, 88, 40 and 316;
any two or more antibodies selected from the group consisting of: 253, 253H55L and 253H165L, 222, 318, 55 and 165;
any two or more antibodies selected from the group consisting of: 158H222L, 222, 150H222L, 384, 159, 253H55L, 253H165L, 253, 88, 40 and 316; or
any two or more antibodies selected from the group consisting of: 158H222L, 222, 150H222L, 253, 253H55L and 253H165L, 222, 318, 55 and 165.

In one embodiment, the invention provides a combination of any of the antibodies described in Table 1.

In one embodiment, the invention provides a combination of any of the antibodies disclosed herein, such as any of the antibodies listed in Table 1, 21, 22, 23, 24 and/or 25.

A combination of the antibodies of the invention may be useful as a therapeutic cocktail. Hence, the invention also provides a pharmaceutical composition comprising a combination of the antibodies of the invention, as explained further below.

A combination of the antibodies of the invention may be useful for diagnosis. Hence, the invention also provides a diagnostic kit comprising a combination of the antibodies of the invention. Also provided herein are methods of diagnosing a disease or complication associated with coronavirus infections in a subject, as explained further below.

### Pharmaceutical Composition

The invention provides a pharmaceutical composition comprising an antibody of the invention. The composition may comprise a combination (such as two, three or four) of the antibodies of the invention. The pharmaceutical composition may also comprise a pharmaceutically acceptable carrier.

The composition of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Suitable pharmaceutically acceptable carriers comprise aqueous carriers or diluents. Examples of suitable aqueous carriers include water, buffered water and saline.

Other suitable pharmaceutically acceptable carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional therapeutic agents, for example an anti-viral agent. The anti-viral agent may bind to coronavirus and inhibit viral activity. Alternatively, the anti-viral agent may not bind directly to coronavirus but still affect viral activity/infectivity. The anti-viral agent could be a further anti-coronavirus antibody, which binds somewhere on SARS-CoV-2 other than the spike protein. Examples of an anti-viral agent useful with the invention include Remdesivir, Lopinavir, ritonavir, APN01, and Favilavir.

The additional therapeutic agent may be an anti-inflammatory agent, such as a corticosteroid (e.g. Dexamethasone) or a non-steroidal anti-inflammatory drug (e.g. Tocilizumab).

The additional therapeutic agent may be an anti-coronavirus vaccine.

The pharmaceutical composition may be administered subcutaneously, intravenously, intradermally, intramuscularly, intranasally or orally.

Also within the scope of the invention are kits comprising antibodies or other compositions of the invention and instructions for use. The kit may further contain one or more additional reagents, such as an additional therapeutic or prophylactic agent as discussed herein.

### Methods and Uses of the Invention

The invention further relates to the use of the antibodies the combinations of the antibodies and the pharmaceutical compositions, described herein, e.g. in a method for treatment of the human or animal body by therapy, or in a diagnostic method. The method of treatment may be therapeutic or prophylactic.

For example, the invention relates to methods of treating coronavirus (e.g. SARS-CoV-2) infections, a disease or complication associated therewith, e.g. COVID-19. The method may comprise administering a therapeutically effective amount of an antibody, a combination of antibodies, or a pharmaceutical composition of the invention. The method may further comprise identifying the presence of coronavirus in a sample, e.g. SARS-CoV-2, from the subject. The invention also relates to an antibody, a combination of antibodies, or a pharmaceutical composition according to the invention for use in a method of treating coronavirus (e.g. SARS-CoV-2) infections, a disease or complication associated therewith, e.g. COVID-19.

The invention also relates to a method of formulating a composition for treating coronavirus (e.g. SARS-CoV-2) infections, a disease or complication associated therewith, e.g. COVID-19, wherein said method comprises mixing an antibody, a combination of antibodies, or a pharmaceutical composition according to the invention with an acceptable carrier to prepare said composition.

The invention also relates to the use of an antibody, a combination of antibodies, or a pharmaceutical composition according to the invention for the manufacture of a medicament for treating coronavirus (e.g. SARS-CoV-2) infections or a disease or complication associated therewith, e.g. COVID-19.

The invention also relates to preventing, treating or diagnosing coronavirus infections caused by any SARS-CoV-2 strain, as described herein. Coronavirus infections may be caused by any SARS-CoV-2 strain, including members of lineage A, A.1, A.2, A.3, A.5, B, B.1, B.1.1, B.2, B.3, B.4, B.1.1.7, B.1.351, P.1, B.1.617.2 or B.1.1.529. In particular, the invention relates to preventing, treating or diagnosing coronavirus infections caused by a SARS-CoV-2 strain from lineage B.1.1.7, B.1.351, P.1, B.1.617.2 or B.1.1.529. The invention also relates to preventing, treating or diagnosing coronavirus infections caused by a SARS-CoV-2 strain from lineage B.1.1.7, B.1.351, P.1, B.1.617.2, B.1.1.529, B.1.526.2, B.1.617.1, B.1.258, C.37, or C.36.3.

The invention also provides an antibody, a combination of antibodies, or a pharmaceutical composition of the invention for use in treating coronavirus infections, or a disease or complication associated therewith, caused by a SARS-CoV-2 strain comprising one or more mutations, e.g. in the spike protein, relative to the hCoV-19/Wuhan/WIV04/2019 (WIV04) (GISAID accession no. EPI_ISL_402124). In other words, the SARS-CoV-2 strain may be a modified hCoV-19/Wuhan/WIV04/2019 (WIV04) strain comprising one or more modifications, e.g. in the spike protein.

The mutations may be N501Y; residues 69-70 deleted, residue 144 deleted, A570D, D614G, P681H, T716I, S982A, and/or D1118H in the spike protein relative to the spike protein of hCoV-19/Wuhan/WIV04/2019 (WIV04). In particular, the SARS-CoV-2 strain comprises N501Y mutation in the spike protein. The SARS-CoV-2 strain may comprise all of the mutations in the spike protein listed above. The SARS-CoV-2 strain may be a member of the B.1.1.7 lineage.

For example, the SARS-CoV-2 strain may comprise deletion of residues 69-70 and N501Y in the spike protein relative to the spike protein in hCoV-19/Wuhan/WIV04/2019. Alternatively, the SARS-CoV-2 strain may comprise deletions of residues 69-70; deletions of residue 144; E484K, A570D, D614G, P681H, T716I, S982A, and D1118H in the spike protein.

The mutations may be: K417N, E484K, N501Y, L18F, D80G, D215G, 242-244 deletion, R246I, D614G, and/or A701V in the spike protein relative to the spike protein of hCoV-19/Wuhan/WIV04/2019 (WIV04). In particular, the SARS-CoV-2 strain comprises E484K mutation in the spike protein. The SARS-CoV-2 strain may comprise all of the mutations in the spike protein listed above. The SARS-CoV-2 strain may be a member of the B.1.351 lineage.

For example, the SARS-CoV-2 strain may comprise K417N, E484K, N501Y, D80G, D215G, deletion of residues 242-244, D614G, and/or A701V in the spike protein relative to the spike protein of hCoV-19/Wuhan/WIV04/2019 (WIV04). Alternatively, the SARS-CoV-2 strain may comprise deletion of residues 242-244 and N501Y in the spike protein. Alternatively, the SARS-CoV-2 strain may comprise deletion of residues 242-244 and E484K in the spike protein.

The mutations may be: K417T, E484K, N501Y, L18F, T20N, P26S, D138Y, R190S, H655Y, and/or T1027I in the spike protein relative to the spike protein of hCoV-19/Wuhan/WIV04/2019 (WIV04). In particular, the SARS-CoV-2 strain comprises E484K mutation in the spike protein. The SARS-CoV-2 strain may comprise all of the mutations in the spike protein listed above. The SARS-CoV-2 strain may be a member of the Y501.V2 lineage.

The mutations may be L18F, T20N, P26S, D138Y, R190S, K417T, E484K, N501Y D614G, H655Y, T1027I, and/or V1176F in the spike protein relative to the spike protein of hCoV-19/Wuhan/WIV04/2019 (WIV04). The SARS-CoV-2 strain may comprise all of the mutations in the spike protein listed above. The SARS-CoV-2 strain may be a member of the P.1 lineage.

The mutation may be a mutation (e.g. substitution) at position 417 in the spike protein relative to the spike protein of the hCoV-19/Wuhan/WIV04/2019 (WIV04) (GISAID accession no. EPI_ISL_402124), wherein the substitution is from the lysine residue to another amino acid residue, such as asparagine (N) or threonine (T).

The mutation may be a mutation (e.g. substitution) at position 501 in the spike protein relative to the spike protein of the hCoV-19/Wuhan/WIV04/2019 (WIV04) (GISAID accession no. EPI_ISL_402124), wherein the substitution is from the asparagine residue to another amino acid residue, such as tyrosine.

The mutation may be a mutation (e.g. substitution) at position 484 in the spike protein relative to the spike protein of the hCoV-19/Wuhan/WIV04/2019 (WIV04) (GISAID accession no. EPI_ISL_402124), wherein the substitution is from the glutamic acid residue to another amino acid residue, such as lysine.

The mutations may be mutation (e.g. substitution) at positions 417, 484 and 501 in the spike protein relative to the spike protein of the hCoV-19/Wuhan/WIV04/2019 (WIV04) (GISAID accession no. EPI_ISL_402124) as indicated above.

For example, the SARS-CoV-2 strain may comprise mutations at the positions 19, 142, 156, 157, 158, 452, 478, 614, 618 and/or 950 in the spike protein relative to the spike protein of the hCoV-19/Wuhan/WIV04/2019 (WIV04) (GISAID accession no. EPI_ISL_402124). The SARS-Cov-2 strain may comprise the substitutions T19R, G142D, R158G, L452R, T478K, D614G, P681R, D950N, e.g. B1.617.2 (delta) strain or a member of the lineage derived therefrom.

The mutation may be a mutation at position 452 in the spike protein relative to the spike protein of the hCoV-19/Wuhan/WIV04/2019 (WIV04) (GISAID accession no. EPI_ISL_402124). For example, the mutation may be a substitution from leucine (L) to another amino acid residue, such as arginine (R) or glutamine (Q). The SARS-Cov-2 strain may comprise the mutation L452R, e.g. a B.1.617.2 (delta) strain or a member of the lineage derived therefrom, a B.1.617.1 (kappa) strain or a member of the lineage derived therefrom, or a C.36.3 strain or a member of the lineage derived therefrom. The SARS-Cov-2 strain may comprise the mutation L452Q, e.g. a C.37 (lambda) strain or a member of the lineage derived therefrom.

The mutation may be a mutation at position 478 in the spike protein relative to the spike protein of the hCoV-19/Wuhan/WIV04/2019 (WIV04) (GISAID accession no. EPI_ISL_402124). For example, the mutation may be a substitution from threonine (T) to another amino acid residue, such as lysine (K). The SARS-Cov-2 strain may comprise the mutation T478K, e.g. a B.1.617.2 (delta) strain or a member of the lineage derived therefrom.

The mutation may be mutations at the positions 339, 371, 373, 375, 417, 440, 446, 477, 478, 484, 493, 496, 498, 501 and/or 505 in the spike protein relative to the spike protein of the hCoV-19/Wuhan/WIV04/2019 (WIV04) (GISAID accession no. EPI_ISL_402124). For example, the mutation may be a substitution from threonine (T) to another amino acid residue, such as lysine (K). The SARS-Cov-2 strain may comprise the substitutions G339D, S371L, S373P, S375F, K417N, N440K, G446S, S477N, T478K, E484A, Q493R, G496S, Q498R, N501Y, Y505H, e.g. a B.1.1.529 (omicron) strain or a member of the lineage derived therefrom.

Antibodies 58, 222, 253 and 253H/55L are particularly effective in neutralising a SARS-Cov-2 strain comprising mutations at the positions 339, 371, 373, 375, 417, 440, 446, 477, 478, 484, 493, 496, 498, 501, 505 in the spike protein relative to the spike protein of the hCoV-19/Wuhan/WIV04/2019 (WIV04). Hence, the invention may relate to these antibodies for use in treating, prevent, treating or diagnosing coronavirus infection caused by a SARS-Cov-2 strain comprising mutations at the positions 339, 371, 373, 375, 417, 440, 446, 477, 478, 484, 493, 496, 498, 501, 505 in the spike protein relative to the spike protein of the hCoV-19/Wuhan/WIV04/2019 (WIV04). Similarly, the invention relates to methods of using these antibodies and uses of these antibodies in treating, prevent, treating or diagnosing coronavirus infection caused by a SARS-Cov-2 strain mutations at the positions 339, 371, 373, 375, 417, 440, 446, 477, 478, 484, 493, 496, 498, 501, 505 in the spike protein relative to the spike protein of the hCoV-19/Wuhan/WIV04/2019 (WIV04).

The SARS-CoV-2 strain may comprise all of the mutations described herein.

The methods and uses of the invention may comprise inhibiting the disease state (such as COVID-19), e.g. arresting its development; and/or relieving the disease state (such as COVID-19), e.g. causing regression of the disease state until a desired endpoint is reached.

The methods and uses of the invention may comprise the amelioration or the reduction of the severity, duration or frequency of a symptom of the disease state (such as COVID-19) (e.g. lessen the pain or discomfort), and such amelioration may or may not be directly affecting the disease. The symptoms or complications may be fever, headache, fatigue, loss of appetite, myalgia, diarrhoea, vomiting, abdominal pain, dehydration, respiratory tract infections, cytokine storm, acute respiratory distress syndrome (ARDS) sepsis, and/or organ failure (e.g. heart, kidneys, liver, GI, lungs).

The methods and uses of the invention may lead to a decrease in the viral load of coronavirus (e.g. SARS-CoV-2), e.g. by ≥10%, ≥20%, ≥30%, ≥40%, ≥50%, ≥60%, ≥70%, ≥80%, ≥90%, or 100% compared to pre-treatment. Methods of determining viral load are well known in the art, e.g. infection assays.

The methods and uses of the invention may comprise preventing the coronavirus infection from occurring in a subject (e.g. humans), in particular, when such subject is predisposed to complications associated with coronavirus infection.

The invention also relates to identifying subjects that have a coronavirus infection, such as by SARS-CoV-2. For example, the methods and uses of the invention may involve identifying the presence of coronavirus (e.g. SARS-CoV-2), or a protein or a protein fragment thereof, in a sample. The detection may be carried out in vitro or in vivo. In certain embodiments, the invention relates to population screening.

The invention relates to identifying any SARS-CoV-2 strain, including members of lineage A, A.1, A.2, A.3, A.5, B, B.1, B.1.1, B.2, B.3, B.4, B.1.1.7, B.1.351, P.1, B.1.617.2 or B.1.1.529. In particular, the invention relates to identifying a SARS-CoV-2 strain from lineage B.1.1.7, B.1.351 or P.1. The invention also relates to identifying a SARS-CoV-2 strain from lineage B.1.1.7, B.1.351, P.1, B.1.617.2, B.1.1.529, B.1.526.2, B.1.617.1, B.1.258, C.37, or C.36.3. The various strains of SARS-CoV-2 are discussed in more detail above.

It has also been identified that many of the antibodies herein may cross-react with SARS-CoV-1. Accordingly, in one embodiment, the invention relates to identify the presence of SARS-CoV-1, e.g. for use in the diagnosis of SARS-CoV-1 infection, or a disease or complication associated therewith, using an antibody, a combination of antibodies, or a pharmaceutical composition according to the invention.

The invention may also relate to a method of identifying escape mutants of SARS-CoV-2, comprising contacting a sample with a combination of antibodies of the invention and identifying if each antibody binds to the virus. The term “escape mutants” refers to variants of SARS-CoV-2 comprising non-silent mutations that may affect the efficacy of existing treatments of SARS-CoV-2 infection. Typically, the non-silent mutations is on an epitope recognised by a prior art antibody and/or antibodies described herein that specifically binds to an epitope of SARS-CoV-2, e.g. on the spike protein of SARS-CoV-2. If the antibody does not bind to the target, it may indicate that the target comprises a mutation that may alter the efficacy of existing SARS-CoV-2 treatments.

The methods and uses of the invention may include contacting a sample with an antibody or a combination of the antibodies of the invention, and detecting the presence or absence of an antibody-antigen complex, wherein the presence of the antibody-antigen complex indicates that the subject is infected with SARS-CoV-2.

Methods of determining the presence of an antibody-antigen complex are known in the art. For example, in vitro detection techniques include enzyme linked immunosorbent assays (ELISAs), Western blots, immunoprecipitations, and immunofluorescence. In vivo techniques include introducing into a subject a labelled anti-analyte protein antibody. For example, the antibody can be labelled with a radioactive marker whose presence and location in a subject can be detected by standard imaging techniques. The detection techniques may provide a qualitative or a quantitative readout depending on the assay employed.

Typically, the invention relates to methods and uses for a human subject in need thereof. However, non-human animals such as rats, rabbits, sheep, pigs, cows, cats, or dogs is also contemplated.

The subject may be at risk of exposure to coronavirus infection, such as a healthcare worker or a person who has come into contact with an infected individual. A subject may have visited or be planning to visit a country known or suspected of having a coronavirus outbreak. A subject may also be at greater risk, such as an immunocompromised individual, for example an individual receiving immunosuppressive therapy or an individual suffering from human immunodeficiency syndrome (HIV) or acquired immune deficiency syndrome (AIDS).

The subject may be asymptomatic or pre-symptomatic.

The subject may be early, middle or late phase of the disease.

The subject may be in hospital or in the community at first presentation, and/or later times in hospital.

The subject may be male or female. In certain embodiments, the subject is typically male.

The subject may not have been infected with coronavirus, such as SARS-CoV-2.

The subject may have a predisposition to the more severe symptoms or complications associated with coronavirus infections. The method or use of the invention may comprise a step of identifying whether or not a patient is at risk of developing the more severe symptoms or complications associated with coronavirus.

In embodiments of the invention relating to prevention or treatment, the subject may or may not have been diagnosed to be infected with coronavirus, such as SARS-CoV-2.

The invention relates to analysing samples from subjects. The sample may be tissues, cells and biological fluids isolated from a subject, as well as tissues, cells and fluids present within a subject. The sample may be blood and a fraction or component of blood including blood serum, blood plasma, or lymph. Typically, the sample is from a throat swab, nasal swab, or saliva.

The antibody-antigen complex detection assays may be performed in situ, in which case the sample is a tissue section (fixed and/or frozen) of the tissue obtained from biopsies or resections from a subject.

In the embodiments of the invention where the antibodies pharmaceutical compositions and combinations are administered, they may be administered subcutaneously, intravenously, intradermally, orally, intranasally, intramuscularly or intracranially, Typically, the antibodies pharmaceutical compositions and combinations are administered intravenously or subcutaneously.

The dose of an antibody may vary depending on the age and size of a subject, as well as on the disease, conditions and route of administration. Antibodies may be administered at a dose of about 0.1 mg/kg body weight to a dose of about 100 mg/kg body weight, such as at a dose of about 5 mg/kg to about 10 mg/kg. Antibodies may also be administered at a dose of about 50 mg/kg, 10 mg/kg or about 5 mg/kg body weight.

A combination of the invention may for example be administered at a dose of about 5 mg/kg to about 10 mg/kg for each antibody, or at a dose of about 10 mg/kg or about 5 mg/kg for each antibody. Alternatively, a combination may be administered at a dose of about 5 mg/kg total (e.g. a dose of 1.67 mg/kg of each antibody in a three antibody combination).

The antibody or combination of antibodies of the invention may be administered in a multiple dosage regimen. For example, the initial dose may be followed by administration of a second or plurality of subsequent doses. The second and subsequent doses may be separated by an appropriate time.

As discussed above, the antibodies of the invention are typically used in a single pharmaceutical composition/combination (co-formulated). However, the invention also generally includes the combined use of antibodies of the invention in separate preparations/compositions. The invention also includes combined use of the antibodies with additional therapeutic agents, as described above.

Combined administration of the two or more agents and/or antibodies may be achieved in a number of different ways. In one embodiment, all the components may be administered together in a single composition. In another embodiment, each component may be administered separately as part of a combined therapy.

For example, the antibody of the invention may be administered before, after or concurrently with another antibody, or binding fragment thereof, of the invention. The particularly useful combinations are shown in Tables 4 and 5 for example.

For example, the antibody of the invention may be administered before, after or concurrently with an anti-viral agent or an anti-inflammatory agent.

In embodiments where the invention relates to detecting the presence of coronavirus, e.g. SARS-CoV-2, or a protein or a protein fragment thereof, in a sample, the antibody contains a detectable label. Methods of attaching a label to an antibody are known in the art, e.g. by direct labelling of the antibody by coupling (i.e., physically linking) a detectable substance to the antibody. Alternatively, the antibody may be indirect labelled, e.g. by reactivity with another reagent that is directly labelled. Examples of indirect labelling include detection of a primary antibody using a fluorescently-labelled secondary antibody and end-labelling of a DNA probe with biotin such that it can be detected with fluorescently-labelled streptavidin.

The detection may further comprise: (i) an agent known to be useful for detecting the presence of coronavirus, e.g. SARS-CoV-2, or a protein or a protein fragment thereof, e.g. an antibody against other epitopes of the spike protein, or other proteins of the coronavirus, such as an anti-nucleocapsid antibody; and/or (ii) an agent known to not be capable of detecting the presence of coronavirus, e.g. SARS-CoV-2, or a protein or a protein fragment thereof, i.e. providing a negative control.

In certain embodiments, the antibody is modified to have increased stability. Suitable modifications are explained above.

The invention also encompasses kits for detecting the presence of coronavirus, e.g. SARS-CoV-2, in a sample. For example, the kit may comprise: a labelled antibody or a combination of labelled antibodies of the invention; means for determining the amount of coronavirus, e.g. SARS-CoV-2, in a sample; and means for comparing the amount of coronavirus, e.g. SARS-CoV-2, in the sample with a standard. The labelled antibody or the combination of labelled antibodies can be packaged in a suitable container. The kit can further comprise instructions for using the kit to detect coronavirus, e.g. SARS-CoV-2, in a sample. The kit may further comprise other agents known to be useful for detecting the presence of coronavirus, as discussed above.

For example, the antibodies or combinations of antibodies of the invention are used in a lateral flow test. Typically, the lateral flow test kit is a hand-held device with an absorbent pad, which based on a series of capillary beds, such as pieces of porous paper, microstructured polymer, or sintered polymer. The test runs the liquid sample along the surface of the pad with reactive molecules that show a visual positive or negative result. The test may further comprise using other agents known to be useful for detecting the presence of coronavirus, e.g. SARS-CoV-2, or a protein or a protein fragment thereof, as discussed above, such as anti- an anti-nucleocapsid antibody.

### Other

It is to be understood that different applications of the disclosed antibodies combinations, or pharmaceutical compositions of the invention may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “an antibody” includes two or more “antibodies”.

Furthermore, when referring to “≥x” herein, this means equal to or greater than x. When referred to “≤x” herein, this means less than or equal to x.

When referring to sequence identity between two sequences, their sequences are compared. Sequences with identity share identical nucleotides at defined positions within the nucleic acid molecule. Thus, a first nucleic acid sequence sharing at least 70% nucleic acid sequence identity with a second sequence requires that, following alignment of the first nucleic acid sequence with the second sequence, at least 70% of the nucleotides in the first nucleic acid sequence are identical to the corresponding nucleotides in the second sequence.

Sequences are typically aligned for identity calculations using a mathematical algorithm, such as the algorithm of Karlin and Altschul (Proc. Natl. Acad. Sci. USA 87 (1990): 2264 2268), modified as in Karlin and Altschul (Proc. Natl. Acad. Sci. USA 90 (1993): 5873 5877). Such an algorithm is incorporated into the XBLAST programs of Altschul et al. (J. MoI. Biol. 215 (1990): 403 410). To obtain gapped alignments, Gapped BLAST can be utilized as described in Altschul et al. (Nucleic Acids Res. 25 (1997): 3389 3402). When utilizing BLAST and Gapped BLAST programs, the default parameters of the respective programs can be used.

The amino acid position numberings provided herein used the IMGT numbering system (http://www.imgt.org; Lefranc M P, 1997, J, Immunol. Today, 18, 509), although in some instances the KABAT numbering system or the absolute numbering of the amino acids based on the sequence listing may be used.

All publications, patents and patent applications cited herein, whether supra or infra, are hereby incorporated by reference in their entirety.

The following examples illustrate the invention.

### EXAMPLES

Antibodies are crucial to immune protection against SARS-CoV-2 with some in emergency use as therapeutics. As shown in the following Examples, the inventors identified 377 human monoclonal antibodies (mAbs) recognizing the viral spike, and focused on 80 which bind the receptor binding domain (RBD). By mapping antigenic sites using a unique computational methodology and comparing with inhibitory activity, the inventors show that binding sites are widely dispersed, but neutralizing epitopes highly focused. Nearly all highly potent neutralizing mAbs (IC50<0.1 μg/ml) block receptor interaction, although one binds a unique epitope in the N-terminal domain. Many mAbs use public V-genes and are close to germline, boding well for vaccine responses. 19 Fab-antigen structures, some as RBD complexed with two Fabs, reveal two novel modes of engagement for potently inhibitory mAbs. Several Fabs are glycosylated, enhancing neutralisation for three, for two of which the sugar contacts the antigen. The most potent mAbs protect, prophylactically or therapeutically, in animal models.

### Example 1. Characterization of mAbs

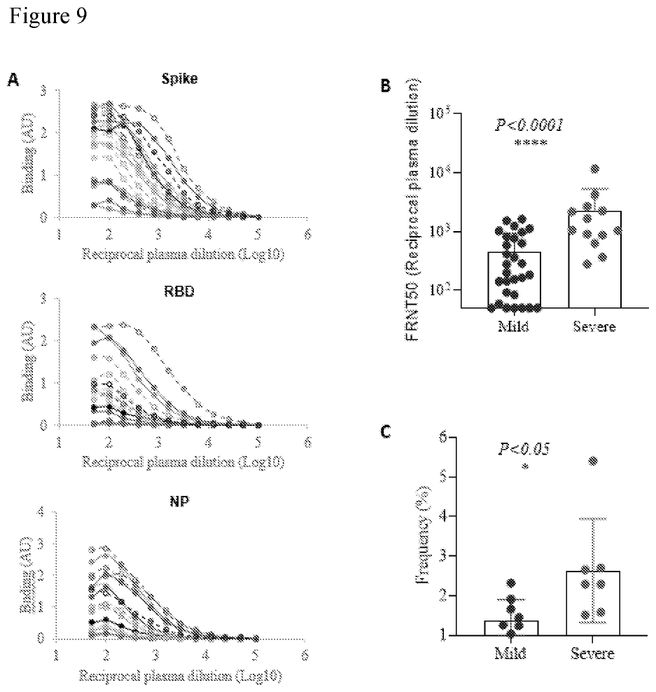

A cohort of 42 patients who had proven SARS-CoV-2 infection diagnosed by qRT-PCR (Table 2) was studied. ELISAs were performed against full-length stabilized S protein (Wuhan-Hu-1 strain, MN908947) where residues 986 and 987 in the linker between two helices in S2 were mutated to a Pro-Pro sequence to prevent the conversion to the post-fusion helical conformation (Walls et al., 2020; Wrapp et al., 2020), RBD (aa 330-532) or N protein (FIG. 9A). Antibody titres varied between patients, and there was a strong correlation between neutralisation titre or the level of anti-S expressing memory B cells with disease severity (Chen et al., 2020b) (FIG. 9B-C).

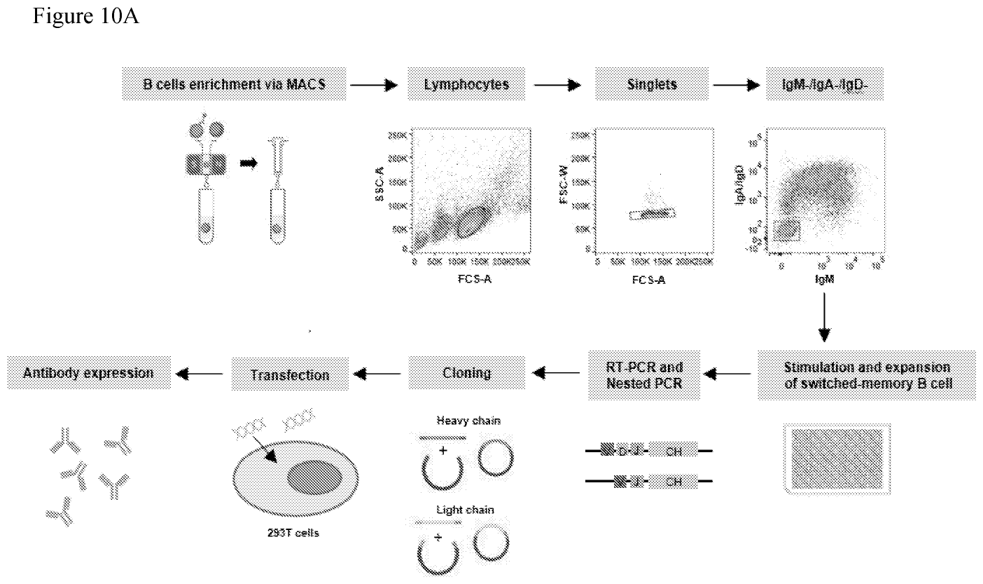

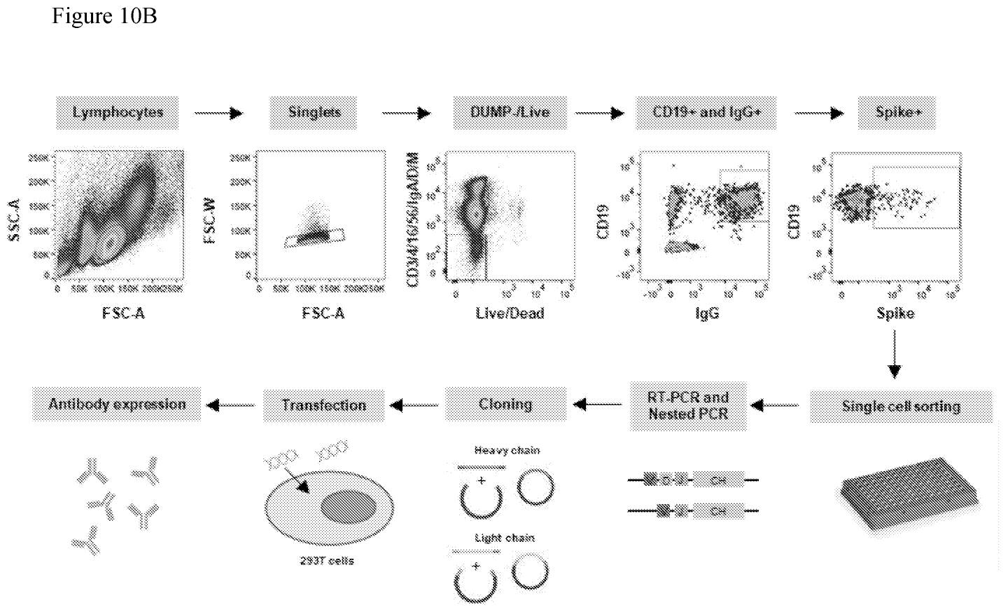

To generate mAbs, two strategies were used. First, IgG expressing B cells were sorted, 4 cells per well, cultured with IL-2, IL-21 and 3T3-msCD40L cells for 13-14 days, and supernatants were tested for reactivity to S protein; positive clones were identified by RT-PCR (FIG. 10A). In a second method, B cells were stained with labelled S or RBD and single positive cells were sorted and subjected to RT-PCR (FIG. 10B). Cell recovery was higher in the severe COVID-19 cases (FIG. 10C), and in total, mAbs from 16 patients (9 mild, 7 severe) were isolated.

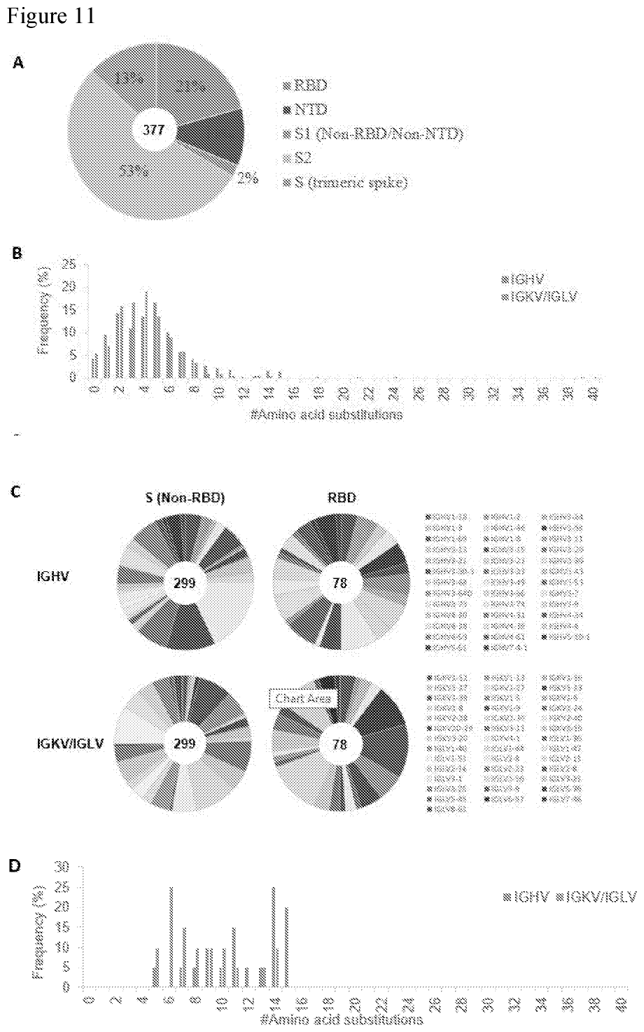

377 antibodies were produced, which reacted to full length S by ELISA. MAbs were further screened for reactivity to S1 (34%), S2 (53%), RBD (21%) and the NTD (11%), with the remaining 13% reactive only to full-length trimeric spike (FIG. 11A). Analysis of antibody sequences revealed low levels of somatic mutation of germline sequences for both heavy (mean 4.11±2.75 amino acids) and light chains (mean 4.10±2.84 amino acids) (FIG. 11B). In general, responses within and between individuals were highly polyclonal with diverse V-gene usage (FIG. 11C). Cross-reactivity of the 377 anti-S antibodies generated from SARS-CoV-2 patients to full-length S proteins from all human alpha and beta-coronaviruses was tested (FIG. 1A). Cross-reactivity was observed with SARS-CoV-1 (52%), MERS (7%), 0C43 (6%), HKU1 (7%), 229E (1%), and NL63 (1%). However, for antibodies recognising RBD, cross-reactivity was restricted to SARS-CoV-1, the RBD of which shares 74% sequence identity with SARS-CoV-2, much more than the other human CoVs (19-21%). Antibodies cross-reacting between the RBDs of SARS-CoV-2 and SARS-CoV-1 showed similarly low levels of germline mutation to the whole pool of S reactive antibodies. However, for antibodies cross-reacting between SARS-CoV-2 and the four seasonal coronaviruses there were more germline mutations particularly in the heavy chain (FIG. 11D). One plausible explanation for the increase in germline mutation in the cross-reactive clones is that they were selected from the memory pool of seasonal coronavirus-specific B cells, rather than generated de novo by SARS-CoV-2.

### Example 2. Neutralisation Activity of SARS-CoV-2 mAbs

The neutralizing activity of all 377 mAbs was investigated using a focus reduction neutralisation test (FRNT). Only 5% of non-RBD mAbs showed neutralizing activity (IC₅₀<10 μg/ml), whereas 60% of RBD-specific mAbs showed neutralizing activity (FIG. 1B).

In total, 19 of 80 anti-RBD antibodies yielded IC₅₀ levels of <0.1 μg/ml (FIG. 1C), defined herein as potent neutralizers. FRNT₅₀ values for a selection of antibodies is shown in Table 3. A number of antibodies outside the RBD had weak neutralizing activity (IC₅₀ values of 0.29-7.38 μg/ml). MAb 159, which binds to the NTD (see below), was one of the most potent inhibitory antibodies obtained with an IC₅₀ of 5 ng/ml.

The ability of anti-RBD mAbs to block interaction with ACE2 was measured using a competitive ELISA. For antibodies showing neutralisation, there was broad correlation between inhibitory potency and ACE2 blocking while NTD-binding mAb 159 did not block ACE2 binding (FIG. 1C).

To investigate the contribution of RBD binding antibodies to neutralisation in polyclonal serum, sera from 8 convalescent donors was immunodepleted with recombinant RBD; depletion of anti-RBD activity was confirmed by ELISA. Neutralisation assays were performed in RBD-depleted and mock-depleted samples and showed the major contribution made by anti-RBD antibodies (55-87% reduction) but also demonstrated that non-RBD antibodies have a significant role in the polyclonal neutralizing response to SARS CoV-2 (FIG. 1D).

### Example 3. Mapping the RBD Antigenic Surface

Pairwise competition between antibodies was measured using biolayer interferometry (BLI) in a 96-well plate format. 79 antibodies were used, and in total 4404 of the 6340 non-diagonal elements of the square competition matrix were populated (see Example 13).

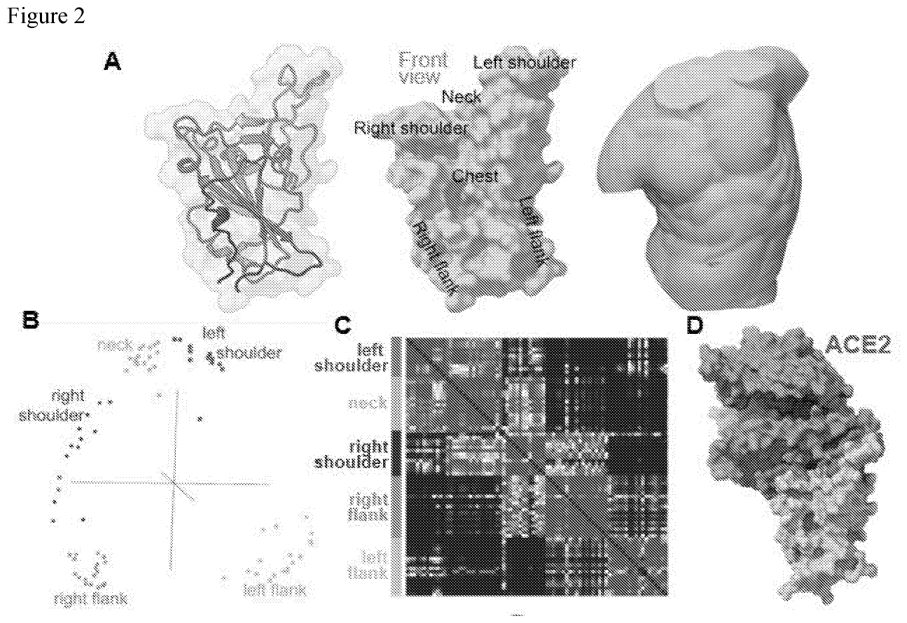

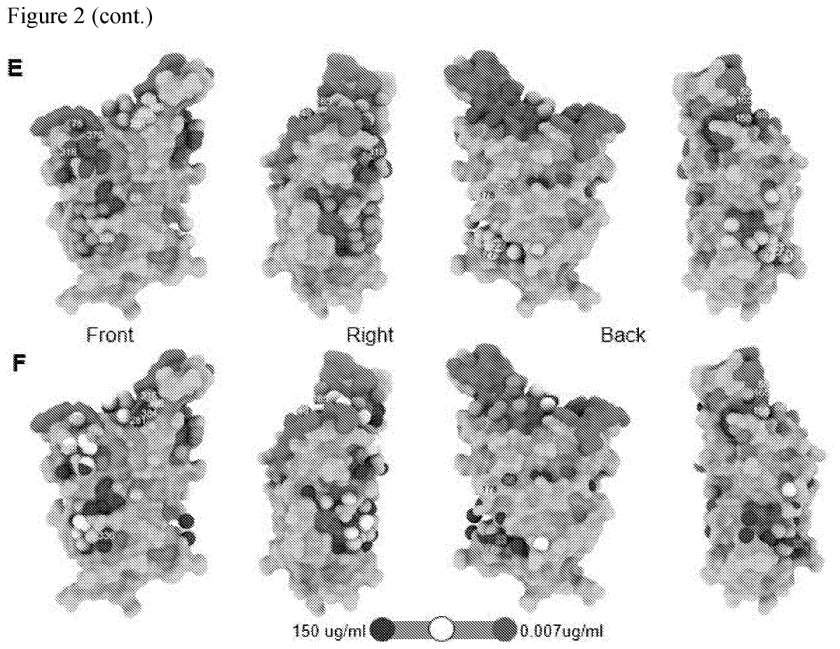

To facilitate interpretation of the results, a naming convention was used for the RBD by comparison with a torso (FIG. 2A). The predicted locations, covering most of the RBD surface, were classified into 5 groups using a clustering algorithm (Methods and cluster4× (Ginn, 2020)) (FIGS. 2B,C). The left flank cluster is distinct from the other 4 clusters which show marked competition at their boundaries and interact sequentially from the left shoulder, neck, right shoulder to right flank. Competition was strongest between the left shoulder and neck, although the neck and right shoulder groups also cross-compete strongly (FIG. 2C).

The ACE2 binding site is shown in FIG. 2D, and the positions of the 76 individual antibodies (plus externals) are depicted in FIG. 2E. The neck cluster is the site of attachment of a number of antibodies possessing the public IGVH3-53 V-region (Yuan et al., 2020b) and strongly overlaps the ACE2 binding site (FIG. 2D-E). The left flank cluster includes previously determined structures EY6A, CR3022 and H014, all of which are reported to show neutralizing activity, but do not compete with ACE2 binding (Yuan et al., 2020a; Huo et al., 2020; Zhou et al., 2020; Lv et al., 2020; Wrobel et al., 2020). Although the left flank is largely separated from the neck and shoulders, two mAbs (38, 178) nevertheless compete well and are situated closer to antibodies of the left shoulder, compared to more isolated antibodies (1, 22, 177) (FIG. 2E). Some regions of the RBD are notable for the lack of antibody binding. The right and left flank clusters both interact with the neck and shoulder clusters, but this does not produce a complete ‘belt’ of antibodies around the waist of the RBD. Antibodies are not seen against the N and C-termini, either because of incomplete presentation on the RBD or occlusion by other parts of the spike.

In FIG. 2F, neutralisation to antibody position is mapped on the RBD. There is generally good correlation between overlap with ACE2 footprint and neutralisation. However, there were notable examples of non-neutralizing antibodies that were good ACE2 blockers. From the competition data, it is possible to identify pairs of non-competing potently neutralizing mAbs and, if the potency threshold is relaxed, triplets (Tables 4 and 5). Such combinations may prove useful in therapeutic cocktails.

There are undoubtedly mechanisms of neutralisation other than just ACE2 blocking, for instance 159 binds the NTD, remote from the ACE2 binding site (see below). Interestingly, antibodies co-locating with known neutralizing/protecting antibodies EY6A/H014 and 5309 (Huo et al., 2020; Zhou et al., 2020; Lv et al., 2020) in the left and right flank clusters respectively did not show appreciable neutralisation in the assays.

### Example 4. Biophysical Characterisation of Selected Antibodies

The kinetics of RBD attachment for 20 potent RBD binders are shown in Table 3. K_{D} values for Fab fragments ranged from 0.7 to 7.6 nM and off-rates, potentially associated with therapeutic efficacy, were in the order of 1,000-10,000 s (Ylera et al., 2013). Expression levels, thermostability, mono-dispersity, and freeze-thaw robustness for 34 mAbs are shown in Table 6. All were stable at elevated temperatures with a first observed Tm at 65-80° C. (Walter et al., 2012) with more than 99% of the mass in a single species. Nearly all were resilient to 20 freeze-thaw cycles.

### Example 5. Structural Analysis of Potent Monoclonal Antibodies—Focusing on Limited Epitopes

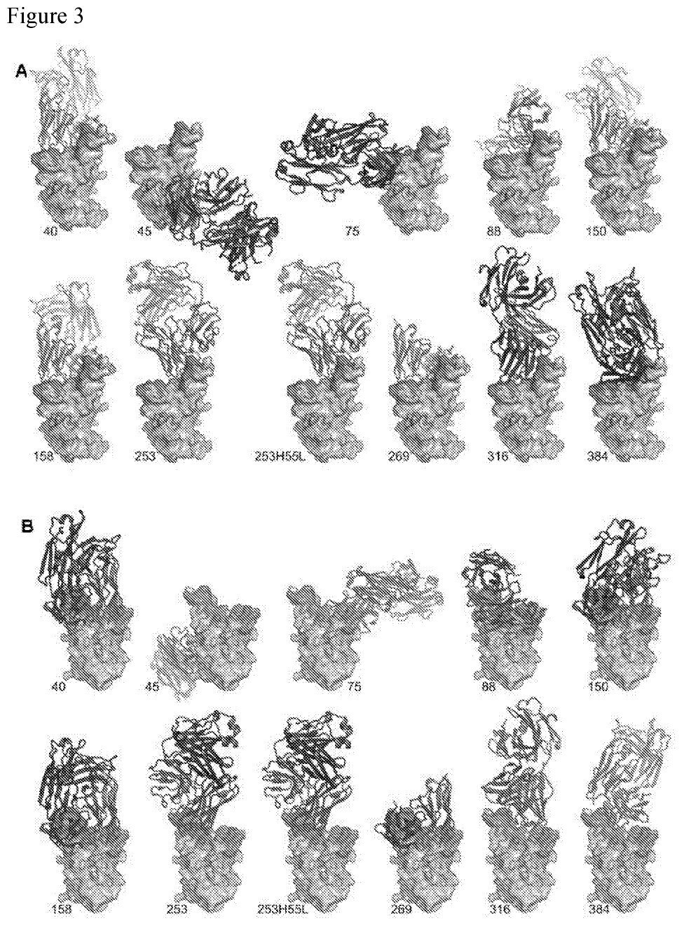

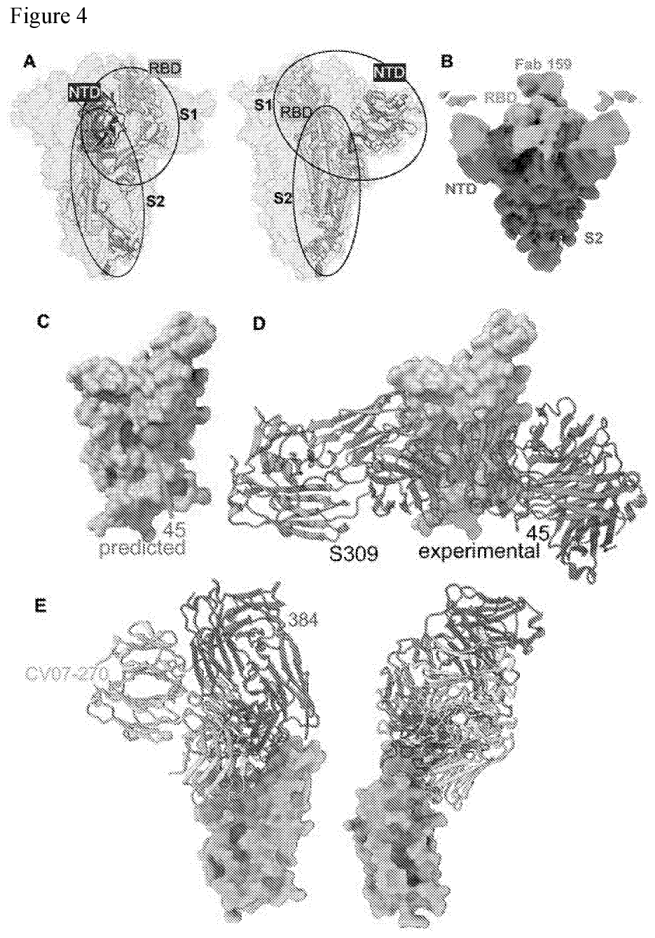

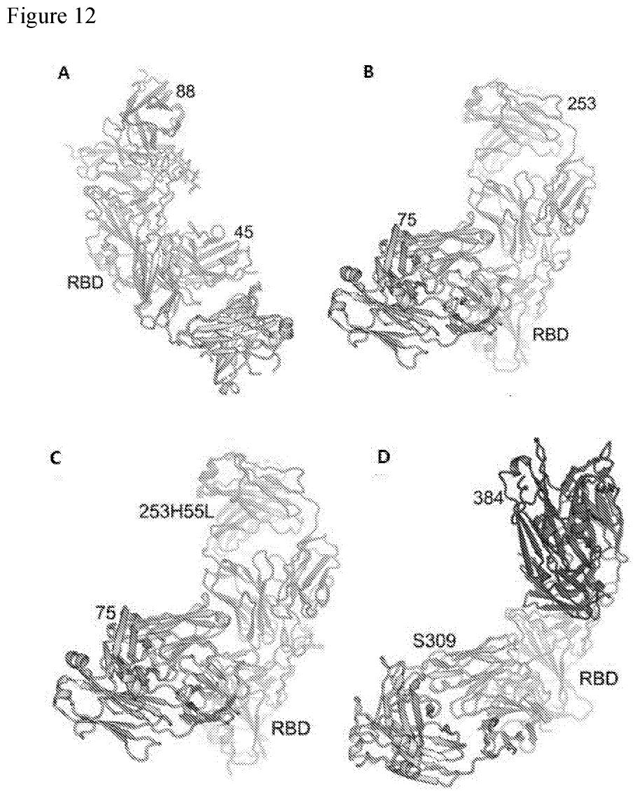

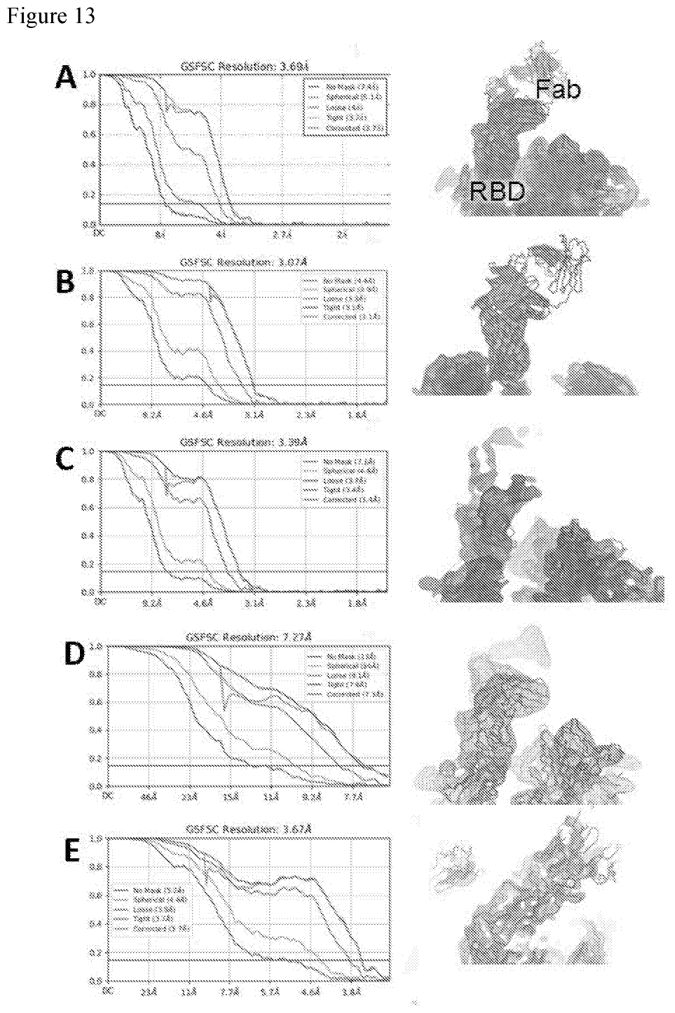

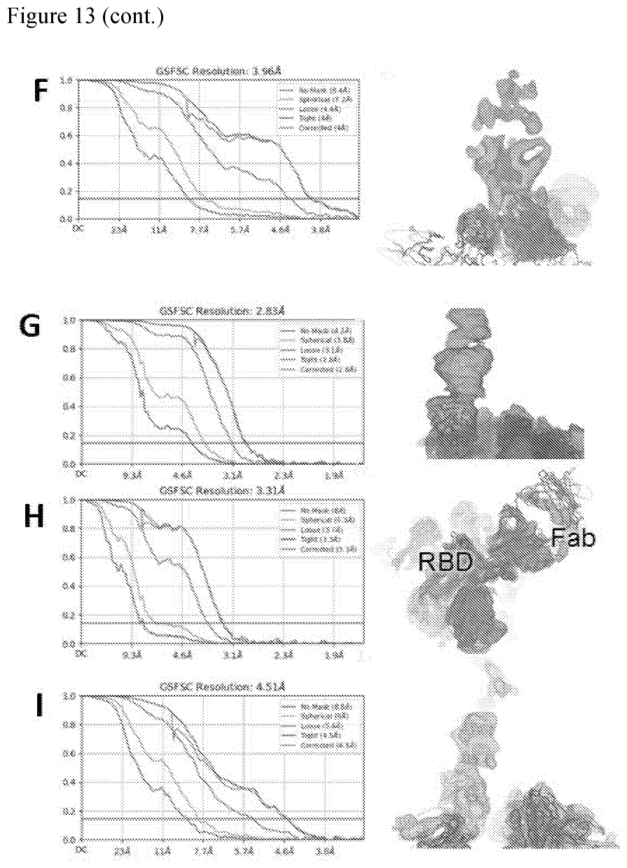

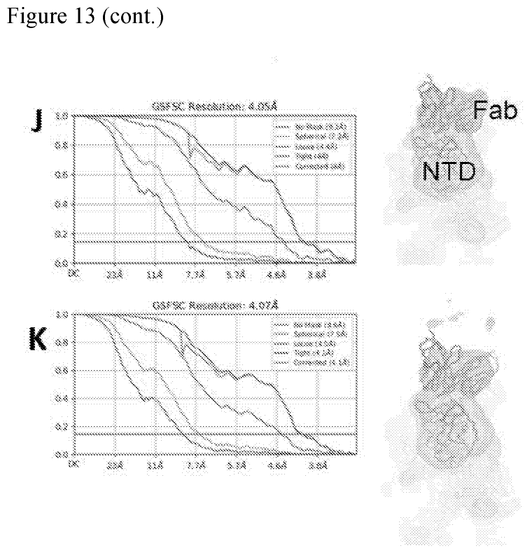

Based on the neutralisation data (Table 3), antibodies were sent for structural analysis. Structures of 19 complexes, usually of either one or two Fabs bound to the RBD alone (8, by crystallography) or of individual Fabs or mAbs bound to trimeric spike (11, by cryo-EM) were determined, these are presented in FIG. 3 (see also Methods, Tables 7 and 8, and FIG. 12, 13). The organisation of the spike is shown in FIG. 4A. Antibody 159 binds to the NTD (FIG. 4B), whereas all other antibodies studied bind to the RBD. The majority of the RBD binders (40, 150, 158 and 269) bind to a tightly defined site in the neck cluster, 253, 316 and 384 bind more towards the front of the left shoulder with 88 binding towards the back of the left shoulder (although the footprints overlap). Antibody binds at the right shoulder. The footprint of all of these antibodies overlap with that of ACE2 (FIG. 3).

By selecting antibodies that are potent neutralizers in the FRNT assay, a large number of high affinity antibodies were omitted. This can be seen for instance in antibody which had a K_{D} of 0.018 μg/ml. This mAb showed weak neutralisation (IC₅₀ 2/ml) and was predicted as mapping to the right flank (FIG. 4C). Structure determination of 45 in a ternary complex with potent neutralizer 88 and RBD, revealed binding in the predicted position, a site not reported previously, adjacent to potent neutralizer S309 (Pinto et al., 2020; Piccoli et al., 2020) (FIGS. 3, 4C, 4D), demonstrating the value of the predictive mapping in identifying novel epitopes.

### Example 6. Potent Antibody 384 Binds in a Previously Unreported Mode

Antibody 384 is the most potently neutralizing mAb reported here IC₅₀ 2 ng/ml. Its binding mode is unlike any other SARS-CoV-2 antibody reported to date. It approaches the binding site on the top of the neck and left shoulder from the front with a relatively small footprint of 630 Å² (460 Å² contributed by the heavy chain and 170 Å² by the light chain). Although the orientation of the bound 384 Fab is similar to a group of previously reported Fabs including CV07-270, p2b-2f6 and bd629 (Kreye et al., 2020; Ju et al., 2020; Du et al., 2020), it is shifted 20 Å towards the left shoulder such that it does not contact the right chest (FIGS. 3, 4E). Only CDRs H2 and H3 of the Fab 384 HC interact with the antigen (FIG. 5A). It is unusual in that the 18 residue long H3 of Fab 384 is bound across the top of the neck to reach the H3 binding site of a group of Fabs that have very short H3s including B38, CB6 and CC12.3 (discussed Example 7) (Wu et al., 2020b; Shi et al., 2020; Yuan et al., 2020b; Hurlburt et al., 2020; Wu et al., 2020a; Du et al., 2020; Clark et al., 2020) making hydrophobic interactions from F104 and L105 at the tip to L455 and F456 of the RBD (FIG. 5A). However, the main interactions that contribute to the binding affinity and orientation are with RBD residues 482-486 on top of the shoulder. W107 of H3 makes strong π-interactions with G485, Y59 of H2 contacts V483 and makes bifurcated H-bonds to the carbonyl oxygen of G482 and amino nitrogen of E484 which in turn salt-bridges with R52 and H-bonds to the side-chains of T57 and Y59 (FIG. 5A). E484-F486 also form a two-stranded antiparallel (3-sheet with residues A92-A94 of L3 and make stacking interactions from F486 to Y32 of L1. The preponderance of main-chain RBD interactions may confer resilience to mutational escape.

### Example 7. Repeated Usage of Heavy Chain V-Regions Demonstrates Potent Public Responses

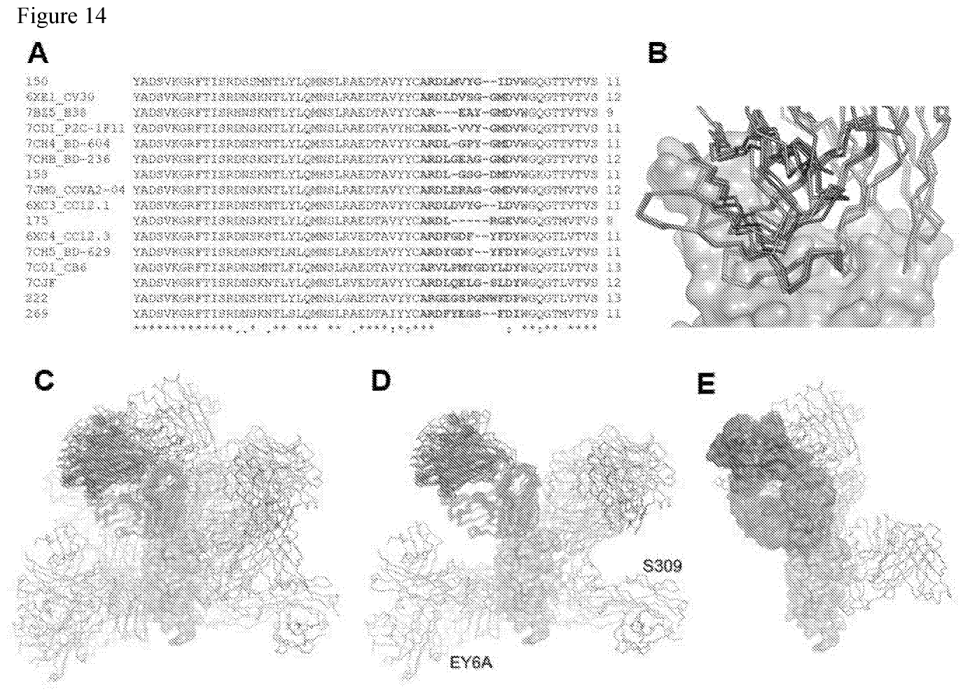

The potent neutralizers identified frequently use public HC V-regions (shared by most people, compared to private, patient specific responses). Five potent mAbs use IGVH3-53 (bearing 3-10 non-silent mutations) (FIG. 5B). Competition data showed that these all bind at a similar site. Structures for three members of the group were determined, 150, 158 and 269 (the others are 175 and 222) and found the mode of engagement of all three as almost identical (FIG. 3, 14B). These have shorter H3s (11 residues) and bind at the back of the neck with similar footprints of about 800 Å². The flat binding site of the RBD and the approach angle of the Fabs limit their H3 length and the number of contacts made with the RBD (FIG. 5C), however this is compensated for by the involvement of interactions from H1, H2 and all CDRs of the light chain. In the case of 158, four residues from H3 have direct contacts (≤4 Å) and make two hydrogen bonds to the RBD, contributing 190 Å² to the footprint. In contrast, 6 residues of H1 and 5 residues of H2 are involved in the interactions with RBD and together make 6 hydrogen bonds, whereas the three CDRs of the light chain contribute 6 residues and 5 hydrogen bonds to the binding (FIG. 5C). The H3 length matches that previously reported as optimal for this V-region (Yuan et al., 2020b), and indeed there are strong similarities in the H3 sequence of mAb 150 (SEQ ID NO: 433) and the previously reported mAb CC1.12 (Yuan et al., 2020b) (FIG. 14A). Thus H1 and H2 determine the mode of engagement, which is common to many previous studies of antibodies with this V-region (FIG. 14C) (Yuan et al., 2020b).

A second V-region which repeatedly confers potent (IC₅₀<0.1 μg/ml) neutralisation is IGVH1-58 (mAbs: 55, 165, 253 and 318 all of which are potent). These have even fewer non-silent mutations (2 to 5) and longer HC CDR3s (12-16 residues). Three antibodies (55, 165, 253) harbour a disulphide bond in their CDR3s, compete strongly with each other for binding and map to the neck epitope, but do not compete with mAb 318. In mAb 253 the disulphide brackets a glycosylation sequon (see below). The crystal structure of a complex including Fab 253 confirmed that it binds within the dominant neck epitope (FIG. 3). In contrast competition mapping indicates that Fab 318 binds at the right shoulder epitope (FIG. 2E). It appears that for this V-region the CDR3 is more critical to recognition and can switch binding to different epitopes on the same antigen. Remarkably, this does not preclude potent binding with near germline V-region sequences.

The final V-region with at least 2 potent neutralizers is IGHV3-66, which was found a total of 5 times with 2 potent neutralizers (282 and 40). These two (with rather few mutations from germline and CDR3 lengths 12 and 13 respectively) compete strongly. Once again, a complex structure was determined for one (Fab 40) and demonstrated that, as expected from the competition data, this antibody binds squarely in the dominant neck epitope, almost indistinguishable from those using IGHV3-53 (FIG. 5D). One IGHV3-66 mAb (398) has a much longer H3, 21 residues, and is predicted to bind on the edge of the neck epitope (FIG. 2E).

IGHV3.11 is found in the most potent neutralizer, 384 but is also used by CV07-270 (Kreye et al., 2020). CV07-270 is swung forward and sideways (compared to 384, FIG. 4E) so that it does not compete with ACE2 binding, suggesting that the potency of 384 derives from the extended H3 interaction which reaches across the ACE2 binding site.

Whilst IGHV3-30 is found in 11 RBD binders, none are potent neutralizers. The structure of two representatives was determined, 75 (in a ternary complex with 253) and 45 (in a ternary complex with 88) (Table 7). 75 binds on the right shoulder and overlaps the ACE2 binding site (FIG. 3), however the only HC-RBD contact is via the extended 20 residue H3 (FIG. 5E). H3 lengths for IGHV3-30 RBD binders vary from 12 to 20 residues, suggesting they bind at different sites, as confirmed by radically different binding of 45, with an H3 length of 14 residues, to the left flank (FIG. 3).

In summary, the major public V-regions used by potent antibodies generally target the neck epitope, often with a common mode of binding dictated by the V-region (although they can occasionally switch epitopes), but this is not true for weaker neutralizers. This likely explains the overwhelming representation of a common mode of binding at the neck epitope in the structures determined to date (FIG. 14C).

### Example 8. Light Chain Mixing can Increase Neutralisation Titre

For the three potent anti-RBD antibody clusters where >2 members shared the same IGVH (IGHV3-53, IGHV1-58 and IGHV3-66), a mixing experiment was performed, where each IGVH was matched with all the IGVL within that cluster (FIG. 6A). Chimeric antibodies were expressed and neutralizations were performed and compared with the original mAb clone. Unexpectedly, a 10-fold increase in neutralisation titres was found when the heavy chain of mAb 253 (IGVH1-58, IGVK3-20) was combined with the light chains of mAbs 55 and 165, which are also IGVH1-58, IGVK3-20 but contain the IGKJ1 region in contrast to IGKJ2 in mAb 253 (FIG. 6B). Remarkably the sole difference in contact residues is a Trp for Tyr substitution in mAbs 55 and 165 (FIG. 6C). Structural analyses of Fab-complexes with RBD reveals the large hydrophobic tryptophan side chain stabilising a hydrophobic region of the antibody and nestled against the key hydrophobic region (E484-F486) of the RBD used by many potent neutralizers, whilst the smaller tyrosine side chain makes fewer contacts.

In summary, the mapping method defines five binding clusters or epitopes. By analogy with a human torso four of these clusters form a continuous swathe running from the left shoulder to the neck, right shoulder and down the right flank of the torso whilst the fifth forms a more discrete site towards the left flank. These sites are widely distributed over the surface, however all but one of the 21 most potent (IC₅₀<0.1 μg/ml) neutralizing mAbs block receptor attachment to the neck. The single exception, mAb 159, binds the NTD and the mechanism of neutralisation is unclear, the lack of neutralisation by Fab 159 suggests that aggregation may play a role, however this domain is frequently associated with receptor-binding in other coronaviruses and 159 might conceivably interfere with co-receptor binding (Li, 2015).

There is now a substantial database of antibody/antigen complexes for the SARS-CoV-2 spike (84 PDB depositions as of 12 Dec. 2020, including nanobody structures). The number of unique structures is much smaller than this and the focus on potently neutralising public V-regions means that many of these have near identical binding modes (FIG. 14). A systematic analysis was performed using neutralisation and mapping to direct structure determination for 19 complexes by crystallography and cryoEM in order to dissect the high-resolution details of binding of the major classes of potent neutralizers. Highly potent ACE2 blocking mAbs map to two sites in the region of the neck and left shoulder, residues E484-F486 bridge the epitopes and are accessible to Fabs binding from a variety of different angles of attack. It is notable that mutation F486L, which would likely affect the binding of some of these antibodies, has been identified as a recurrent mutation associated with host-adaptation in mink (van Dorp et al., 2020).

### Example 9. The Role of N-Linked Glycan in Antibody Interaction

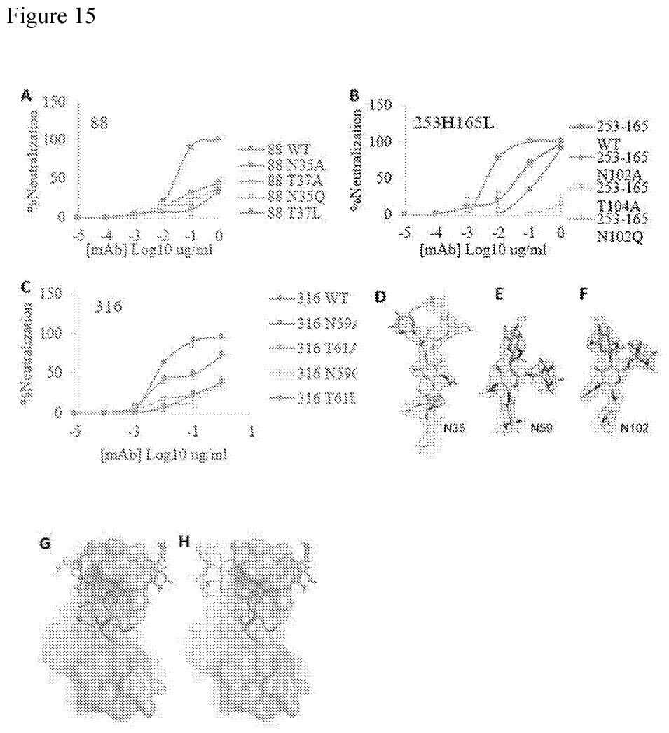

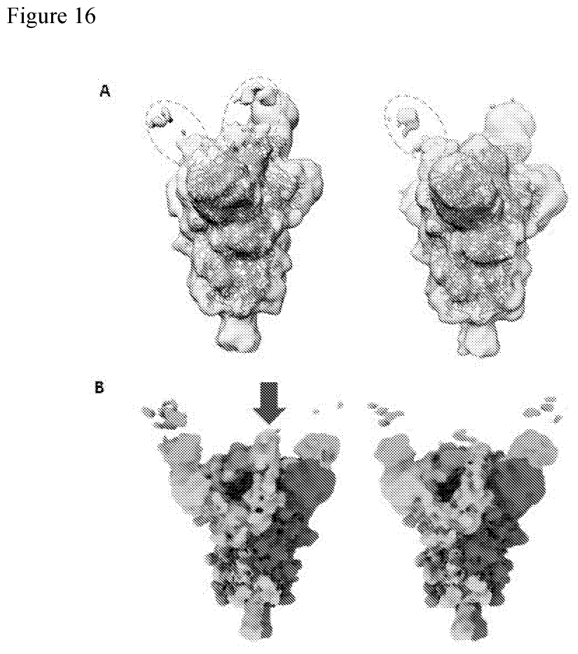

It is known that 15-25% of IgGs bear N-linked glycans in their variable regions, sometimes with impact on antigen binding. Of 80 RBD-binding antibodies described here, 14 (17.5%) contain glycosylation sequins arising from somatic mutations in their variable region. For 8 mAbs (1, 88, 132, 253, 263, 316, 337, 382) the sequins are in the HC and for they lie in a CDR. Several of the HC mutations, but none of the LC mutations, are in potently inhibitory antibodies (neutralisation IC₅₀<0.1 μg/ml). Two of these (88 and 316) could be de-glycosylated without denaturation, and BLI analysis showed that this had negligible effect on RBD/Fab affinities (K_{D}=0.8/1.2 nM and 1.0/2.0 nM, de-glycosylated/glycosylated respectively for 88 and 316), although the on-rate was a little faster in the absence of sugar (e.g. 3.8×10⁵ 1/Ms compared to 1.4×10⁵ 1/Ms for mAb 88. However mutations that eliminate glycosylation had a deleterious effect on neutralisation for these two and for the 253H165L chimera (FIG. 15). Structures were therefore determined for mAbs 88, 316 and 253 in complex with RBD and with spike (FIGS. 3, 6D, 15, Tables 7, 8).

Antibodies 88 and 316 contain glycosylation sites in H1 (N35) and H2 (N59) respectively. The crystal structure of the RBD-316 Fab complex at 2.3 Å resolution shows well-defined density for 3 glycans including an α1,6 linked fucose (FIGS. 6D and 15E). The structure of Fab 88 was determined in a ternary complex with 45 and RBD to 2.53 Å resolution (the ChCl domains of 88 were disordered but the VhVl domains had well defined density). Antibody 88 binds to the back of the neck whereas 316 binds to the top of the neck, orientated radically differently, however the H3s of the two Fabs overlap well (FIGS. 6D and S7). The glycans of Fab 88 surround the back of the left shoulder like a necklace and those of Fab 316 sit on the top of the same shoulder. Fab 88 has a footprint of 1110 Å² (390 Å², 420 Å² and 300 Å² from HC, LC and glycans, respectively), whereas Fab 316 has a footprint of 950 Å² (610 Å², 150 Å² and 190 Å² from HC, LC and glycans, respectively). As described above for mAb 384, residues E484-F486 of the RBD make extensive interactions in these antibodies with residues from the 3 CDRs of the HC and L1 and L3 of the LC, thus for 316 the side chain of E484 H-bonds to N52 and S55 of H2 and Y33 of H1, G485 contacts W50 of H2, and F486 makes strong ring stacking interactions with Y93 and W99 of L3 and Y34 of L1. This suggests E484-F486 constitutes a hot-spot of the epitope. These residues are accessible from a variety of different angles of attack, thus Fabs 384, 316 and 88 all interact with this region despite their markedly different poses on the RBD. In contrast, the H3 of 253 overlaps with the glycans of mAb 88 and the glycan of mAb 253 makes no direct interactions with the RBD (FIG. 6D).

In all cases the sugar is presented close to the top of the left shoulder, and in 2 out of 3 cases interacts directly but rather weakly with the antigen. The high frequency of sequon generation despite the rather few somatic mutations is intriguing and suggests positive selection.

Despite the most potently neutralizing mAbs being close to germline, somatic mutations introduce N-linked glycosylation sites into the variable region of 17.5% of the potent neutralizers. These can contribute to the interaction with the RBD, and although they appear to have relatively little effect on affinity they significantly enhance neutralisation.

### Example 10. Binding in the Context of the Trimeric Spike

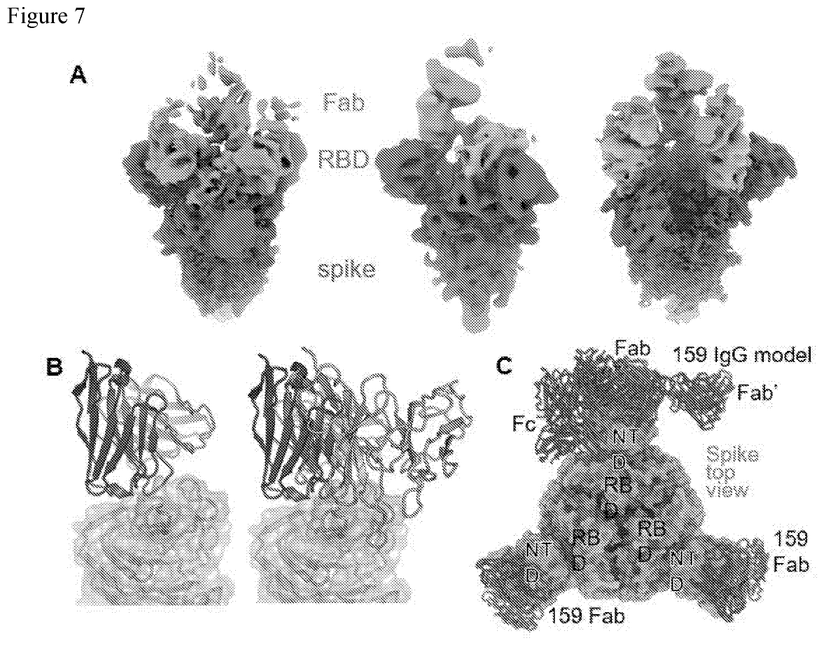

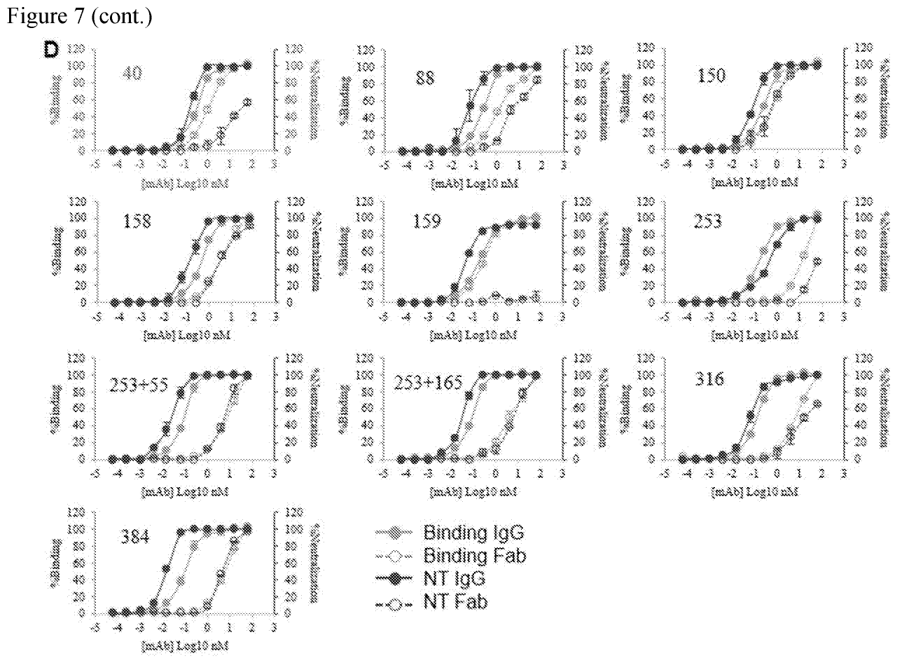

On isolated stabilised spikes the RBD is found in two orientations; ‘up’ and ‘down’ (Roy et al., 2020). Both of these form a family of conformations, up conformations vary by up to 20° (Zhou et al., 2020) and down can include a tighter packed ‘locked’ conformation (Ke et al., 2020; Toelzer et al., 2020; Carrique et al., 2020; Xiong et al., 2020). The structures seen by cryo-EM have the RBD in either the classic up or down conformation (see FIG. 7A), although antibody binding sometimes introduces small changes in the RBD orientation. The most common configuration observed for the spike construct used is 1 RBD-up and 2-down. ACE2 can only attach to the up conformation, which is assumed to be less stable, favouring conversion to the post-fusion state. In the structures, Fabs 40, 150, 158 and the chimeras 253H55L and 253H165L are seen binding to the spike in this one-up configuration. 253H55L also binds to the all-down configuration (1 Fab/trimer), as does Fab 316 (3 Fabs/trimer) and Fab 384 (1 Fab/trimer). In contrast, Fab 88 binds (3 Fabs/trimer) in the all-up configuration (Table 9 and FIG. 7A).

Fab 384 predominantly binds one RBD per trimer, although analysis of different particle classes revealed some weak density decorating the other RBDs, also in the down position, while a subtle movement can be seen between the RBDs of different classes (FIG. 16). This could be attributed to a more favourable RBD conformation that can only be sustained by one RBD at a time.

To visualize the binding of the highly potent mAb 159, it was necessary to incubate spike with 159 IgG (the Fab alone showed no binding). This revealed all three NTDs of the spike decorated by 159 with RBDs in either one-up or all-down configurations (FIG. 16). The 159 binding site is ˜15 Å from that of a previously reported NTD binder, 4A8 (Chi et al., 2020), in which the CDR-H3 binds on the side of the NTD between the 144-153 and 246-258 loops (FIG. 7B). The CDR-H3 of 159 is 11 residues shorter than that of 4A8 (Chi et al., 2020) and binds on the top centre of the NTD interacting with residues 144-147, 155-158, 250-253 and the N-terminus of NTD. All 3 CDRs of the heavy chain contribute to a foot-print of 515 Å² on the NTD, whereas the light chain has little contact with the NTD (35 Å²), similar to 4A8 (Chi et al., 2020) (FIG. 7B, C).

### Example 11. Valency of Interaction

Binding of full-length and Fab fragments to whole SARS-CoV-2 by ELISA was measured and compared these with neutralisation curves for a selection of antibodies for which structural information was available (FIG. 7D and Table 9). For the anti-NTD mAb-159 binding of full-length and Fab to virions were nearly identical, this is in-line with NTDs on a trimer being too far apart to allow bivalent engagement (118 Å) (FIG. 7C) and suggests that mAb-159 cannot reach between adjacent spike trimers at the virion surface. Interestingly, whilst IgG-159 is a potent neutralizer, Fab-159 has no neutralizing activity, suggesting that the Fc portion is crucial for activity, although the mechanism for this is not immediately apparent and does not involve blocking ACE2 interaction.

Loss in binding and neutralisation with Fabs compared to IgG is quite modest for mAb-88, which attaches in the all-up conformation (FIGS. 7D and 9), but much more marked for mAbs that bind the all-down form of the spike (253, 316, 384). Thus mAb-384 showed 79-fold less virus binding and a 486-fold loss of neutralisation activity when reduced to Fab, suggesting that both Fab arms are used when antibody interacts with virions and also highlights the exceptional K_{D} of Fab-159, 2.5 to 81-fold better than the other Fabs depicted in FIG. 7D and Table 9. Finally, the following formula was used to estimate the relationship between antibody binding and neutralisation: Percent occupancy=BMax*[Ab]/(Kd+[Ab]), where the BMax is percent maximal binding, [Ab] is the concentration of Ab required to reach 50% FRNT and Kd is the concentration of Ab required to reach half-maximal binding. mAb-384 can achieve NT50 with an estimated average occupancy of 12% of the maximum available antibody binding sites on each virion, perhaps in part due to the avidity conferred by bivalent attachment (Table 9). Bivalent attachment to the down conformation may also lock all three RBDs, ruling out attachment to ACE2. Some of the variation in the effects seen in FIG. 7D and Table 9 probably arises from the interplay between the angle and position of attack of the antibody arm to the RBD and the constraints on flexibility in the system.

A correlation was identified between Fab vs IgG binding/neutralisation and the mode of attachment to the prefusion spike as seen by cryoEM. Those antibodies which bind the spike in the down conformation appear to show a marked avidity boost to binding and neutralisation when Fab and full length IgG1 are compared (e.g. 316 and 384), suggesting that there is a relationship between the mode of attachment and neutralisation, as also seen from the potent neutralisation reported for antibodies that bind at the left and right flank (S309 and EY6A/H014 (Pinto et al., 2020; Zhou et al., 2020; Lv et al., 2020) epitopes that do not report strong neutralisation in the assay used herein.

### Example 12. In Vivo Efficacy

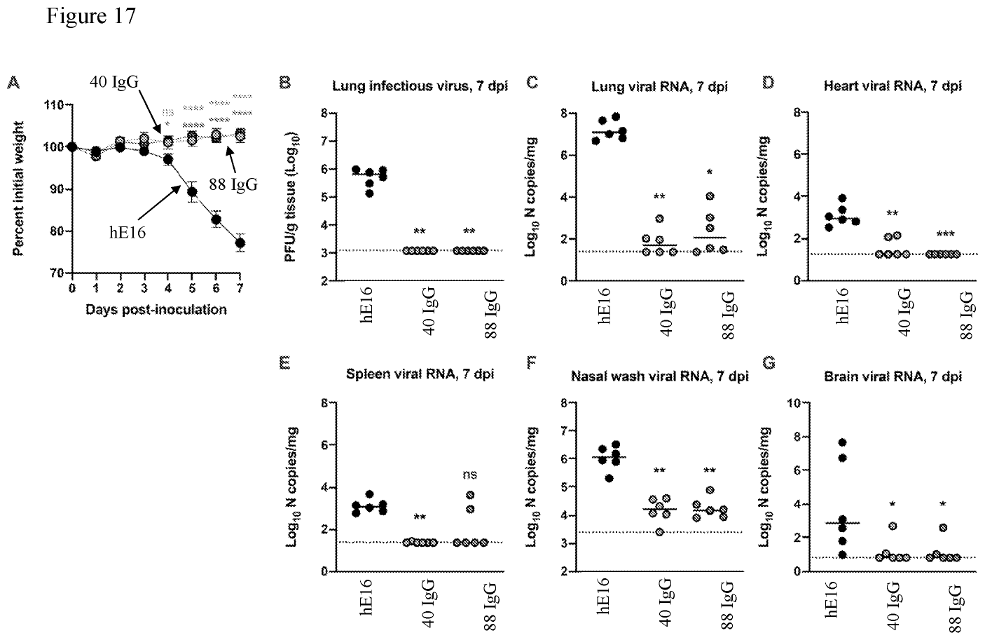

The efficacy of the most promising neutralizing human mAbs was determined in vivo. The K18-hACE2 transgenic mouse model of SARS-CoV-2 pathogenesis was used wherein human ACE2 expression is driven by an epithelial cell specific, cytokeratin-18 gene promoter (McCray et al., 2007; Winkler et al., 2020). In this model, SARS-CoV-2 infected animals develop severe pulmonary disease and high levels of viral infection in the lung that is accompanied by immune cell infiltration and tissue damage (Winkler et al., 2020). Initially, a single 250 μg (10 mg/kg) dose of mAbs 40 and 88 were administered as prophylaxis by intraperitoneal injection 1-day prior (D-1) to intranasal (i.n.) challenge with 10³ PFU of SARS-CoV-2. Passive transfer of mAb 40 or 88, but not an isotype control mAb (hE16), prevented SARS-CoV-2-induced weight loss (FIG. 17A). In the lung homogenates of antibody 40 and 88 treated animals, no infectious virus was detected at 7 days post infection (dpi), whereas substantial amounts were present in animals treated with the isotype control mAb (FIG. 17B). Consistent with these results, viral RNA levels were reduced by approximately 10,000-100,000-fold compared to isotype control mAb-treated animals (FIG. 17C). In peripheral organs, including the heart, spleen, or brain viral RNA levels were reduced or undetectable in mAb 40 or 88 treated animals (FIG. 17D-G). Moreover, levels of viral RNA at 7 dpi were markedly lower in the nasal washes of animals treated with mAbs 40 and 88 compared to the isotype control.

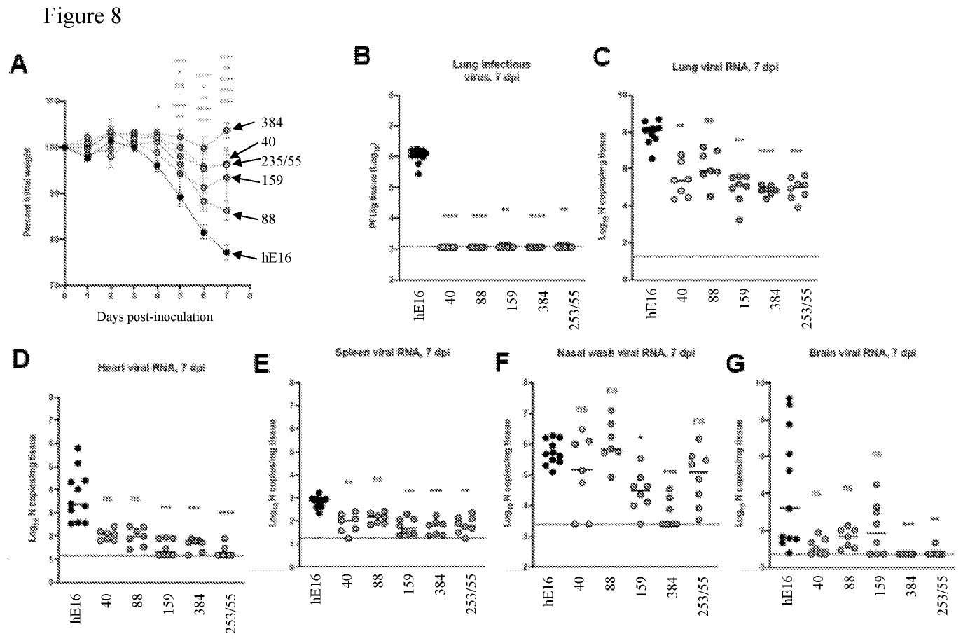

To further evaluate the in vivo potency of the mAbs, the therapeutic activity of a larger panel at 1 dpi (D+1) was assessed with 10³ PFU of SARS-CoV-2. Although varying degrees of protection were observed for individual mAbs, weight loss was significantly reduced in all animals treated with anti-SARS-CoV-2 mAbs at 6 and 7 dpi compared to the isotype control (FIG. 8A). Whereas the lungs of isotype control mAb-treated animals had infectious virus levels of ˜10⁶ PFU/g of tissue, infectious virus in animals treated with the mAbs 40, 88, 159, 384, or 253H55L was barely detected (FIG. 8B). Lung viral RNA levels at 7 dpi also were reduced in animals treated with mAbs 40, 159, 384, and 253H55L. mAb 88 displayed mean reductions of ˜100-fold (FIG. 8C). At sites of disseminated infection, notably the heart, spleen, and brain, all anti-SARS-CoV-2 mAbs showed protective activity although mAbs 384 and 253H55L conferred the greatest reductions in viral RNA levels (FIGS. 8D,E,G). In nasal washes, mAbs 159 and 384 showed the greatest ability to reduce viral RNA levels (FIG. 8F). Collectively, these data demonstrate several mAbs in the panel can reduce infection in the upper airway, lower airway, and at distant sites when administered after infection.

In summary, the most potent antibodies identified were demonstrated to protect in an animal model, when administered prophylactically and therapeutically. The competition mapping method devised suggests a series of combinations of antibodies with non-overlapping epitopes. These results might therefore contribute to immunotherapy.

### Example 13. Materials and Methods

Materials and methods for Examples 1 to 12.

### Trimeric Spike of SARS-CoV-2

To construct the expression plasmids for SARS-CoV-2 spike protein, a gene encoding residues 1-1208 of the spike ectodomain with a mutation at the furin cleavage site (residues 682-685) from RRAR (SEQ ID NO: 409) to GSAS (SEQ ID NO: 410), proline substitutions at residues 986 and 987, followed by the T4 fibritin trimerization domain, a HRV3C protease cleavage site, a twin Strep Tag and an 8XHisTag (SEQ ID NO: 411), was synthesized and optimized for mammalian expression (Wrapp et al., 2020). An optimized coding sequence was cloned into the mammalian expression vector pHLsec.

### Trimeric Spike of SARS-CoV, MERS-CoV, 0C63-CoV, HKU1-CoV, 229E-Cov, NL63-CoV

Expression plasmids were constructed using synthetic fragments coding for human codon-optimized spike glycoprotein sequences from CoV-229E (GenBank accession number NC_002645.1; amino acids 1-1113), CoV-HKU1 (GenBank accession number NC_006577.2; amino acids 1-1300), CoV-NL63 (GenBank accession number NC_005831.2; amino acids 1-1289), CoV-0C43 (GenBank accession number NC_006213.1; amino acids 1-1297), CoV-MERS (GenBank accession number AFS88936.1; amino acids 1-1291) (Zhao et al., 2013), CoV-SARS1 (GenBank accession number AY27874; amino acids 11-1195) (Simmons et al., 2004) and CoV-SARS2 (GenBank accession number MN908947; amino acids 1-1208). Fragments were cloned as previously reported by Wrapp et al. (Wrapp et al., 2020)

Mutations coding for stabilising proline residues and to eliminate putative furin cleavage sites were inserted in each sequence as follows: For CoV-229E, TI>PP (aa 871-872); for CoV-HKU1, RRKR (SEQ ID NO: 412)>GSAS (SEQ ID NO: 410) (aa 756-759) and AL>PP (aa 1071-1072); for CoV-NL63, RRSR (SEQ ID NO: 413)>GSAS (SEQ ID NO: 410) (aa 754-757) and SI>PP (aa 1052-1053); for CoV-0C43, AL>PP (aa 1070-1071); for CoV-MERS, RSVG (SEQ ID NO: 414)>ASVG (SEQ ID NO: 415) (aa 748), RSAR (SEQ ID NO: 416)>GSAS (SEQ ID NO: 410) (aa 884-887) and VL>PP 1060-1061; for CoV-SARS1, KV>PP (aa 968-969); for CoV-SARS2, RRAR (SEQ ID NO: 409)>GSAS (SEQ ID NO: 410) (aa 682-685) and KV>PP (aa 986-987). All sequences were verified by DNA sequencing.

DNA plasmids encoding the Strep-Tag-tagged spike proteins were transfected into HEK293T cells and incubated at 37° C. for 7 days. CoV spike protein trimers were affinity-purified. In the case of CoV-229E and CoV-NL63, the spike proteins were further purified by SEC.

Depletion of Anti-RBD Antibodies from Plasma Samples.

Nickel charged agarose beads were incubated overnight with His-tagged RBD. Beads incubated in the absence of RBD antigen were used as a beads-only, mock control. The beads were precleared with a pooled SARS-CoV-2 negative plasma. Beads were incubated with the human plasma samples of interest. The remaining depleted samples were collected, filter sterilized, and tested for complete depletion by RBD direct ELISA. ACE2 and RBD. Constructs are as described in Huo et al. 2020 (Huo et al., 2020) and production was as described in Zhou et al. 2020 (Zhou et al., 2020).

Isolation of Human Monoclonal Antibodies from Peripheral B Cells by Memory B Cell Stimulation

To generate human monoclonal antibodies from peripheral blood B cells, CD22+ B cells were isolated from PBMCs using CD22 Microbeads (130-046-401; Miltenyi Biotec). Pre-enriched B cells were stained with anti-IgM-APC, IgA-FITC and IgD-FITC. Double negative memory B cells (IgM-,IgA-/D-cells) were sorted by FACS and plated on 384-well plates at a density of 4 B cells per well. Cells were stimulated to proliferate and produce IgG by culturing with irradiated 3T3-msCD40L feeder cells (12535; NID AIDS Reagent Program), 100 U/ml IL-2 (200-02; Peprotech) and 50 ng/ml IL-21 (200-21; Peprotech) for 13-14 days. Supernatants were harvested from each well and screened for SARS-CoV-2 binding specificity by ELISA. Lysis buffer was added to positive wells containing SARS-CoV-2-specific B cells and immediately stored at −80° C. for future use in Ig gene amplification and cloning.

### Isolation of Spike and RBD-Specific Single B Cells by FACS

To isolate spike and RBD-specific B cells, PBMCs were sequentially stained with LIVE/DEAD Fixable Aqua dye (Invitrogen) followed by recombinant trimeric spike-twin-Strep or RBD-biotin. Cells were then stained with antibody cocktail consisting of CD3-FITC, CD14-FITC, CD56-FITC, CD16-FITC, IgM-FITC, IgA-FITC, IgD-FITC, IgG-BV786, CD19-BUV395 and Strep-MAB-DY549 (iba) or streptavidin-APC (Biolegend) to probe the Strep tag of spike or biotin of RBD. spike or RBD-specific single B cells were gated as CD19+, IgG+, CD3−, CD14−, CD56−, CD16−, IgM−, IgA−, IgD−, spike+ or RBD+ and sorted into each well of 96-well PCR plates containing RNase inhibitor (N2611; Promega). Plates were centrifuged briefly and frozen on dry ice before storage at −80° C. for future use in Ig gene amplification and cloning.

Cloning and Expression of SARS CoV2-Specific Human mAbs.

Genes encoding Ig VH, Ig Vκ and Vλ from positive wells were recovered using RT-PCR (210210; QIAGEN). Nested PCR (203205; Qiagen) was then performed to amplify genes encoding γ-chain, λ-chain and κ-chain with ‘cocktails’ of primers specific for human IgG. PCR products of genes encoding heavy and light chains were joined with the expression vector for human IgG1 or immunoglobulin κ-chain or λ-chain (gifts from H. Wardemann) by Gibson assembly. For the expression of antibodies, plasmids encoding heavy and light chains were co-transfected into the 293T cell line by the polyethylenimine method (408727; Sigma), and antibody-containing supernatants were harvested for further characterization.

### Construction of Fab Expression Plasmids

Heavy chain expression plasmids of specific antibodies were used as templates to amplify the first fragment, heavy chain vector include the variable region and CH1 until Kabat amino acid number 233. The second fragment of thrombin cleavage site and twin-Strep-tag with overlapping ends to the first fragment were amplified. The two fragments were ligated by Gibson assembly to make the Fab heavy chain expression plasmid.

Construction of scFv Antibody Plasmid

Heavy chain and light chain expression plasmids of specific antibodies were used as a template to amplify variable region gene of heavy and light chain respectively. Firstly, heavy chain gene products having the AgeI-SalII restriction enzyme sites were cloned into a scFv vector which is a modified human IgG expression vector which has a linker between the H chain and L chain genes followed by a thrombin cleavage site and twin-Strep-tags. Light chain gene products having NheI-NotI restriction enzyme site were cloned into scFv vector containing the heavy chain gene insert to produce scFv expression plasmids.

Fab and scFv Production and Purification

Protein production was done in HEK293T cells by transient transfection with polyethylenimine in FreeStyle 293 medium. For Fab antibody production, Fab heavy chain expression plasmids were co-transfected with the corresponding light chain. For scFv antibody production, scFv expression plasmid of specific antibody was used for transfection. After 5 days of culture at 37° C. and 5% CO2, culture supernatant was harvested and filtered using a 0.22 mm polyethersulfone (PES) filter. Fab and scFv antibody were purified by Strep-Tactin affinity chromatography (IBA lifescience) according to the Strep-Tactin XT manual.

### Determination of Plasma and Antibody Binding to Recombinant Protein by ELISA

MAXISORP immunoplates (442404; NUNC) were coated with 0.125 μg of StrepMAB-Classic were incubated with double strep-tag recombinant spike of SARS-CoV-2, SARS-CoV, MERS-CoV, 0C43-CoV, HKU1-CoV, 229E-CoV and NL43-CoV. Serially diluted plasma or mAbs were added, followed by ALP-conjugated anti-human IgG (A9544; Sigma). The reaction was developed by the addition of PNPP substrate and stopped with NaOH. To determine the binding to SARS-CoV-2 RBD, SARS-CoV-2 NP, SARS-CoV-2 spike S1 (40591-V08H; Sino Biological Inc) and SARS-CoV-2 spike S2 (40590-V08B; Sino Biological Inc), immunoplates were coated with Tetra-His antibody (34670; QIAGEN) followed by 5 μg/mL of His-tag recombinant SARS-CoV-2 RBD, SARS-CoV-2 NP, SARS-CoV-2 spike S1 and SARS-CoV-2 spike S2. The plasma endpoint titres (EPTs) were defined as reciprocal plasma dilutions that corresponded to two times the average OD values obtained with mock. EC50 of mAbs were evaluated using non-linear regression (curve-fit).

### Whole Virus ELISA

To determine the binding affinity of antibody to SARS-CoV-2 virus, virus was captured onto plates coated with mouse anti-SARS-CoV-2 spike (mAb31 with murine Fc) and then incubated with serial dilutions of SARS-CoV-2-specific human mAbs (full length IgG or Fab) followed by ALP-conjugated anti-human IgG (A8542, Sigma). The reaction was developed with PNPP substrate and stopped with NaOH. Results are expressed as the percentage of total binding.

### Focus Reduction Neutralisation Assay (FRNT)

The neutralisation potential of Ab was measured using a Focus Reduction Neutralisation Test (FRNT), where the reduction in the number of the infected foci is compared to a no antibody negative control well. Briefly, serially diluted Ab was mixed with authentic SARS-CoV-2/human/AUS/VIC01/2020 (Caly et al., 2020) and incubated for 1 hr at 37° C. The mixtures were then transferred to Vero cell monolayers and incubated for 2 hrs followed by the addition of 1.5% semi-solid carboxymethyl cellulose (CMC) overlay medium to each well to limit virus diffusion. A focus forming assay was then performed by staining Vero cells with human anti-NP mAb (mAb206) followed by peroxidase-conjugated goat anti-human IgG (A0170; Sigma). Finally, the foci (infected cells) were visualized by adding TrueBlue Peroxidase Substrate. The percentage of focus reduction was calculated and IC₅₀ was determined.

### NTD Binding Assay

MAbs were screened for binding to MDCK-SIAT1 cells expressing the N-terminal domain (NTD) of SARS-CoV-2 spike glycoprotein (MDCK-NTD, from Prof. Alain Townsend). In brief, MDCK-NTD cells were incubated overnight. mAbs supernatants from transfected 293T cells were added and incubated. A second antibody Goat anti-human IgG Fc specific-FITC (F9512, Sigma-Aldrich) was then added (50 μl per well) and incubated. After washing twice, the wells were fixed with 1% formaldehyde in PBS. The binding antibodies were detected by fluorescence intensities.

### ELISA Based ACE2 Binding Inhibition Assay

For the ACE2 competition ELISA, 250 ng of ACE2 protein was immobilized to a MAXIXORP immunoplate and the plates were blocked with 2% BSA in PBS. In the meantime, serially diluted Ab was mixed with recombinant RBD-mFc (40592-V05H; Sino Biological) and incubated for 1 h at 37° C. The mixtures were then transferred to the ACE2 coated plates and incubated for 1 h followed by goat anti-mouse IgG Fc-AP (Invitrogen #A16093) at 1:2000 dilution. The reaction was developed by the addition of PNPP substrate and stopped with NaOH. The absorbance was measured at 405 nm. The ACE2/RBD binding inhibition rate was calculated by comparing to antibody-free control well. IC₅₀ were determined using the probit program from the SPSS package.

### Spike Protein Production for Structural Analysis

The stable cell line generation vector pNeoSec was used for cloning of the SARS-Cov2 spike ectodomain comprising amino acids 27-1208 with mutations of the furin cleavage site (RRAR (SEQ ID NO: 409)>GSAS (SEQ ID NO: 410) at residues 682-685) and the PP (KV>PP at residues 986-987). At the N-terminus, there is a twin StrepII tag and at the C-terminus fused with a T4 fibritin trimerisation domain, an HRV 3C cleavage site and a His-8 tag. The human embryonic kidney (HEK) Expi293F cells (Thermo Fisher Scientific) were transfected with the construct together with a phiC31 integrase expression plasmid as described earlier (Zhao et al., 2014). The polyclonal G418 resistant (1 mg/ml) cell population were used for protein production. Expi293F cells were grown in adhesion in roller bottles with the high glucose DMEM (Sigma) with 2% FBS for 6 days at 30° C. The soluble spike protein was captured from the dialysed conditional media with prepacked 5 ml Columns of HisTrap excel (GE Healthcare Life Sciences). The protein was eluted in 300 mM imidazole containing phosphate-buffered saline (PBS) after a 20 mM imidazole PBS wishing step. The protein was further purified with a 16/600 Superdex 200 size exclusion chromatography with an acidic buffer (20 mM Acetate, 150 mM NaCl, pH 4.6) for the low pH spike incubations, or a neutral buffer (2 mM Tris, 150 mM NaCl, pH 7.5).

### Production of RBD for Structural Analysis

Stable HEK293 S cell line expressing His-tagged RBD was cultured in DMEM (high glucose, Sigma) supplemented with 10% FBS (Invitrogen), 1 mM glutamine and 1× non-essential amino acids at 37° C. Cells were transferred to roller bottles (Greiner) and cultured in DMEM supplemented with 2% FBS, 1 mM glutamine and 1× non-essential amino acids at 30° C. for 10 days for protein expression. For protein purification, the dialyzed media was passed through a 5 mL HisTrap Nickel column (GE Healthcare). The column was washed with buffer 20 mM Tris pH 7.4, 200 mM NaCl, 30 mM imidazole and RBD was eluted using buffer 20 mM Tris pH 7.4, 200 mM NaCl, 300 mM imidazole. A volume of 30 μl endoglycosidase H1 (˜1 mg ml⁻¹) was added to −30 mg RBD and incubated at room temperature for 2 h. Then the sample was further purified with a Superdex 75 HiLoad 16/600 gel filtration column (GE Healthcare) using 10 mM HEPES pH 7.4, 150 mM NaCl. Purified RBD was concentrated using a 10-kDa ultra centrifugal filter (Amicon) to 10.6 mg ml⁻¹ and stored at −80° C.

Preparation of Fabs from IgGs

Fab fragments were digested from purified IgGs with papain using a Pierce Fab Preparation Kit (Thermo Fisher), following the manufacturer's protocol.

### Physical Assays

Thermal stability was assessed using Thermofluor (DSF). Briefly, 3 μg of the Ab preparation was used in a 5011.1 reaction containing 10 mM HEPES pH 7.5, 100 mM NaCl, 3× SYPROorange (Thermo Fisher). Samples were heated from 25-97° C. in a RT-PCR machine (Agilent MX3005p) and the fluorescence monitored at 25° C. after every 1° C. of heating. Melting temperatures (Tm) were calculated by fitting of a 5-parameter sigmoid curve using the JTSA software (P. Bond, https://paulsbond.co.uk/jtsa). Polydispersity was assessed by DLS using 10 μg of the Ab preparation in an UNCLE instrument (Unchained Labs). Freeze thaw experiments on 4 of the mAbs were performed with material at 1 mg/ml by flash-freezing using LN2, thawing and centrifuging an aliquot (10 minutes at 20000 g) before measuring the absorbance at 280 nm of the soluble fraction.

### Crystallization

Purified RBD was combined separately with Strep-tagged Fab150, Fab58, scFv269 and Fab316 in a 1:1 molar ratio, with final concentrations of 13.2, 9.4, 12.7 and 13.0 mg ml⁻¹, separately. RBD was combined with Fab45 and Strep-tagged Fab88, Fab75 and Fab253, and Fab 75 and Strep-tagged chimeric Fab 253H55L in a 1:1:1 molar ratio all with a final concentration of 7 mg ml⁻¹, separately. Glycosylated RBD was combined with Fab 5309 (Pinto et al., 2020) and Fab384 in a 1:1:1 molar ratio with a final concentration of 8 mg ml⁻¹. Crystals of RBD-150 complex were formed in Molecular Dimensions Morpheus condition C2, containing 0.09 M of NPS (nitrate, phosphate and sulphate), 0.1 M MES/imidazole pH 6.5, 10% (w/v) PEG 8000 and 20% (v/v) ethylene glycol and crystals also formed in Hampton Research PEGRx condition D11, containing 0.1 M imidazole pH 7.0 and 12% (w/v) PEG 20000. Crystals of RBD-158 were obtained from Index condition C01, containing 3.5 M NaCOOH pH 7.0, while some crystals were formed in Proplex condition C1, containing 0.15 M (NH4)₂SO₄, 0.1 M Tris pH 8.0 and 15% (w/v) PEG 4000 and further optimized in 0.15 M (NH4)₂SO₄, 0.1 M Tris pH 7.6 and 14.6% (w/v) PEG 4000. Crystals of RBD-scFv269 complexed were obtained from Index condition F01, containing 0.2 M Proline, 0.1 M HEPES pH 7.5 and 10% (w/v) PEG 3350. Good crystals for the RBD-316 complex were obtained from Index condition G10, containing 0.2 M MgCl₂, 0.1 M bis-Tris pH 5.5 and 25% (w/v) PEG 3350. Crystals of RBD-45-88 complex were obtained from PEGRx condition G12, containing 10% (v/v) 2-Propanol, 0.1 M Sodium acetate trihydrate pH 4.0, 22% (w/v) PEG 6000. Crystals of RBD-75-253 complex were obtained from PEGRx condition D8, containing 0.1 M BIS-TRIS pH 6.5, 16% (w/v) PEG 10000. Crystals of RBD-75-253H55L were obtained from Index condition F5, containing 0.1 M ammonium acetate, 0.1 M bis-Tris pH 5.5 and 17% (w/v) PEG 10000. For the RBD-5309-384 ternary complex, good crystals were obtained from Morpheus condition H1, containing 0.1 M amino acids (Glu, Ala, Gly, Lys, Ser), 0.1 M MES/imidazole/pH 6.5, 10% (w/v) PEG 20000 and 20% (w/v) PEG MME 550.

### X-Ray Data Collection, Structure Determination and Refinement

Diffraction data were collected at 100 K at beamline I03 of Diamond Light Source, UK. The structures were determined by molecular replacement with PHASER (Liebschner et al., 2019) using search models of the RBD, VhVl and ChCl domains of a closely related Fab in sequence for each complex. The ChCl domains of Fab 88 in the RBD-88-45 complex are disordered. Data collection and structure refinement statistics are given in Table 7.

### Cryo-EM Grid Preparation

For all Fab or IgG-spike complexes, a 3 μL aliquot of S ˜0.6 μm (determined by OD) with Fab (1:6 molar ratio) was prepared, aspirated and almost immediately applied to a freshly glow-discharged Cu support Cflat 2/1-200 mesh holey carbon-coated grid (high intensity, 20 s, Plasma Cleaner PDC-002-CE, Harrick Plasma). Excess liquid was removed by blotting for 5-5.5 s with a force of −1 using vitrobot filter paper (grade 595, Ted Pella Inc.) at 4.5° C., 100% reported humidity before plunge freezing into liquid ethane using a Vitrobot Mark IV (Thermo Fisher).

### Cryo-EM Data Collection and Processing

40, 253H55L and 253H165L spike complexes:

Movies were collected in compressed tiff format on a Titan Krios G2 (Thermo Fisher) operating at 300 kV with a K3 detector (Gatan) in super resolution counting mode using a custom version of EPU 2.5 (Thermo Fisher). A defocus range of 0.8-2.6 μm was applied with a nominal magnification of ×105,000, corresponding to a calibrated pixel size of 0.83 Å/pixel and with a total dose of 43-47 e/Å².

Two-times binned movies were then motion corrected and aligned on the fly using Relion (3.1) scheduler (Zivanov et al., 2018) with a 5×5 patch based alignment. CTF-estimation of full-frame non-weighted micrographs was performed with the GCTF (1.06) (Zhang, 2016) module in cryoSPARC (v2.14.1-live) (Punjani et al., 2017).

88, 150, 158, 159IgG, 316 and 384 spike complexes:

Data for 88, 150, 158 were collected Titan Krios G2 (Thermo Fischer) operating at 300 kV with a K2 camera and a GIF Quantum energy filter (Gatan) with a 30 eV slit. For 159 (IgG), 384 and 316, data were collected as for 88, 150 and 158, except using a 20 keV slit. Rapid multi-shot data acquisition was set up using custom scripts with SerialEM (version 3.8.0 beta) (Mastronarde, 2005) at a nominal magnification of 165 kX, corresponding to a calibrated pixel size of 0.82 Å per pixel. A defocus range of −0.8 μm to −2.6 μm was used with a total dose of ˜45-57 e⁻/Å² applied across 40 frames. Motion and CTF correction of raw movies was performed on the fly using cryoSPARC live patch-motion and patch-CTF correction (Punjani et al., 2017).

40, 253H55L, 253H165L, 88, 150, 158, 159 IgG, 316 and 384 complexes:

Poor-quality images were discarded after manual inspection of CTF and motion estimations. Particles were then blob picked in cryoSPARC (Punjani et al., 2017) and initially extracted with four times binning. After inspection of 2D classes, classes of interest were selected to generate templates for complete particle picking. Binned particles were then subjected to one to three rounds of reference free 2D classification followed by 3D classification with an ab-initio derived model before further refinement and unbinning.

For both 150 and 158, two data separate data collections were set up on the same grid, and refined particle sets from each collection were separated by exposure groups before being combined. For 150, a total of 77,265 exposure-group split particles were initially combined (51,554 from 4726 movies and 25,711 from 2079 movies), re-classified into five classes, and the two best classes (42,655 particles) subjected to further non-uniform refinement, with obvious density for Fab bound to one RBD in an ‘up’ conformation. Notably, discarded classes included a high proportion of undecorated S (28,463 particles, 4.4 Å reported resolution at GSFSC=0.143, −43 Å² B-factor).

Classification using heterogeneous refinement in cryoSPARC was found to be generally poor, and, instead, 3D variability analysis was employed to try to better resolve full spike-Fab structures. Local refinements were also performed with masks focused around the Fab/RBD region (not reported here), but maps were still insufficient to clearly build a model at the RBD/Fab interface and far inferior to the crystallographic maps. 3D variability analysis was found to be essential for isolating the RBD up and RBD down conformations for 159-IgG. Results from this are presented for 159-IgG and 384. Briefly, data were separated into eight clusters using the 3D variability analysis module with a 6 Å resolution filter and a mask around the RBD/Fab region. Masks were generated by initially rigid body fitting a model of the spike and a Fab into a refined map in Chimera before selecting an area of the model including the RBD and fab and using the ‘color zone’ module to crop out this desired part of the map. The resulting map was smoothed with a Gaussian filter (Pettersen Ef Fau—Goddard et al., 2004), converted into a mask format using Relion3.1 ‘Mask Create’ before import into cryoSPARC. Resolution estimates were taken from Gold standard-FSC (FSC=0.143) reported in the local resolution module in cryoSPARC (Punjani et al., 2017).

### Competition Assay of Antibodies

Competition assay of anti-RBD antibodies was performed on a Fortebio Octet RED96e machine with Fortebio Anti-HIS (HIS2) Biosensors. 2 μg ml⁻¹ of His-tagged RBD dissolved in the running buffer (10 mM HEPES, pH 7.4 and 150 mM NaCl) was used as the ligand and was first immobilized onto the biosensors. The biosensors were then washed in the running buffer to remove unbound RBD. Each biosensor was dipped into different saturating antibodies (Ab1) to saturate the bound RBD, except one biosensor was into the running buffer in this step, acting as the reference. The concentration of saturating antibodies used was 15 μg ml⁻¹. Higher concentrations were applied if 15 μg ml⁻¹ was not enough to obtain saturating. Then all biosensors were washed with the running buffer again and dipped into wells containing the same competing antibody (Ab2). The concentration of competing antibodies used was 5 μg ml⁻¹. The Y-axis values of signals of different saturating antibodies in this step were divided by the value of the reference channel to get ratio results of different Ab1-Ab2 pairs. Ratio result close to 0 indicated total competition while 1 indicated no competition. In total, 50 IgGs and 4 Fabs (Fabs 40, EY6A (Zhou et al., 2020), FD5D (unpublished) and S309 (Pinto et al., 2020)) were used as the saturating antibodies and 80 IgGs as the competing antibodies.

### Competition Mapping of Antibodies

Gross binning of antibodies: Competition values were prepared for cluster analysis and binning by capping all competition values between 0 and 1. Competition values between antibodies i and j were averaged with the competition value for j and i when both were available. Cluster4× (Ginn, 2020) was used to cluster antibodies into three distinct groups using single value decomposition on the matrix of competition values.

Preparation of RBD surface and mesh: A surface of the receptor-binding domain was generated in PyMOL (The PyMOL Molecular Graphics System, Version 1.2r3pre, Schrödinger, LLC) from chain E of PDB code 6YLA. A mesh was generated and iteratively contracted and restrained to the surface of the RBD to provide a smoother surface on which to direct antibody refinement, reducing intricate surface features which could lead to unrealistic exploration of local minima.

Fixing positions of antibodies with known structure: In order to provide an objective position for those antibodies of known structure (FD5D (unpublished), EY6A (Zhou et al., 2020), S309 (Pinto et al., 2020) and mAb 40), to reflect the occluded region, all non-hydrogen antibody atoms were found within 20 Å of any RBD atom, and likewise all RBD atoms within 20 Å of an antibody atom. From each group, the atoms with the lowest sum-of-square-lengths from all other members were identified and the midpoint of these two atoms was locked to the nearest vertex on the mesh. Solvent molecules were ignored, but in the case of S309, the glyco-oligomer cofactor was included in the set of antibody atoms.

The target function: On an evaluation of the target function, either all unique pairs of antibodies were considered (all-pairs), or only unique pairs where one of the antibodies

$$\\ f ( x ) = \frac{{e}^{\frac{r - d}{2}}}{1 + {e}^{\frac{r - d}{2}}}$$

was fixed (fixed-pairs), depending on the stage of the minimisation protocol. Competition levels were estimated for each pair of antibodies as described by f(x) in Eq. 1 where r is the working radius of the antibody, set to 22 Å, accounting for the approximate antibody radius. The distance between the pair of antibodies at a given evaluation of the function is given by din Angstroms. The target function was the sum of squared differences between the competition estimation and the competition value from SPR data.

Obtaining a self-consistent set of refined antibody positions: Minimisation was carried out globally by 1000 macrocycles of Monte Carlo-esque sampling using LBFGS refinement. A random starting position for each antibody was generated by randomly assigning a starting vertex on the RBD mesh and the target function minimised for 20 cycles considering data points for pairs with at least one fixed antibody, followed by 40 cycles for all data points. Between each cycle, antibody positions were locked onto the nearest mesh vertex. Depending on the starting positions of antibodies, results were a mixture of well-refined and poorly refined solutions. Results were ordered in ascending target function scores. Positions of antibodies for each result was passed into cluster4× as dummy C-alpha positions (Ginn, 2020). A clear self-consistent solution was enriched in lower target function scores and separated using cluster4× for further analysis. From these, an average position for each antibody was locked to the nearest vertex on the mesh, and the RMSD calculated from all contributing antibody positions.

### Cells and Viruses (Mouse Experiments)

Vero CCL81 (American Type Culture Collection (ATCC)) and Vero-furin cells (Mukherjee et al., 2016) were cultured at 37° C. in Dulbecco's Modified Eagle medium (DMEM) supplemented with 10% foetal bovine serum (FBS), 10 mM HEPES, and 100 U/ml of penicillin—streptomycin. The 2019n-CoV/USA_WA1/2019 isolate of SARS-CoV-2 was obtained from the US Centers for Disease Control (CDC). Virus stocks were propagated by inoculating Vero CCL81 cells and collecting supernatant upon observation of cytopathic effect; debris was removed by centrifugation at 500×g for 5 min. Supernatant was aliquoted and stored at −80° C. All work with infectious SARS-CoV-2 was performed in Institutional Biosafety Committee approved BSL3 and A-BSL3 facilities at Washington University School of Medicine using appropriate positive pressure air respirators and protective equipment.

### Mouse Experiments

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anaesthesia that was induced and maintained with ketamine hydrochloride and xylazine, and all efforts were made to minimize animal suffering.

Heterozygous K18-hACE C57BL/6J mice (strain: 2B6.Cg-Tg(K18-ACE2)₂Prlmn/J) were obtained from The Jackson Laboratory. Seven to eight-week-old male and female animals were inoculated with 10³ PFU of SARS-CoV-2 via intranasal administration.

### Measurement of Viral Burden

Tissues were weighed and homogenized with zirconia beads in a MagNA Lyser instrument (Roche Life Science) in 1000 μL of DMEM supplemented to contain 2% heat-inactivated FBS. Tissue homogenates were clarified by centrifugation at 10,000 rpm for 5 min and stored at −80° C. RNA was extracted using the MagMax mirVana Total RNA isolation kit (Thermo Scientific) on a Kingfisher Flex extraction robot (Thermo Scientific). RNA was reverse transcribed and amplified using the TaqMan RNA-to-CT 1-Step Kit (ThermoFisher). Reverse transcription was carried out at 48° C. for 15 min followed by 2 min at 95° C. Amplification was accomplished over 50 cycles as follows: 95° C. for 15 s and 60° C. for 1 min. Copies of SARS-CoV-2 N gene RNA in samples were determined using a previously published assay (PubMed ID 32553273). Briefly, a TaqMan assay was designed to target a highly conserved region of the N gene (Forward primer: ATGCTGCAATCGTGCTACAA (SEQ ID NO: 417)); Reverse primer: GACTGCCGCCTCTGCTC (SEQ ID NO: 418); Probe: /56-FAM/TCAAGGAAC/ZEN/AACATTGCCAA/3IABkFQ/(SEQ ID NO: 419)). This region was included in an RNA standard to allow for copy number determination. The reaction mixture contained final concentrations of primers and probe of 500 and 100 nM, respectively.

Plaque assay. Vero-furin cells (Mukherjee et al., 2016) were seeded at a density of 2.5×10⁵ cells per well in flat-bottom 12-well tissue culture plates. The following day, medium was removed and replaced with 200 μL of 10-fold serial dilutions of the material to be titrated, diluted in DMEM+2% FBS. After incubation for 1 h at 37° C., 1 mL of methylcellulose overlay was added. Plates were incubated for 72 h, then fixed with 4% paraformaldehyde (final concentration) in phosphate-buffered saline for 20 min. Plates were stained with 0.05% (w/v) crystal violet in 20% methanol and washed twice with distilled, deionized water prior to plaque enumeration.

### Affinity Determination Using Biolayer Interferometry

Octet RED 96e (ForteBio) was used to determine the binding affinities of antibodies with RBD or spike. Anti-RBD IgGs were immobilized onto AR2G biosensors (ForteBio) while RBD was used as the analyte with serial dilutions. For IgG159, spike was immobilised onto AR2G biosensors with IgG159 acting as the analyte with serial dilutions. Kd values were calculated using Data Analysis HT 11.1 (ForteBio) with a 1:1 global fitting model.

### Example 14. Characterising the N501Y Mutation in the RBD

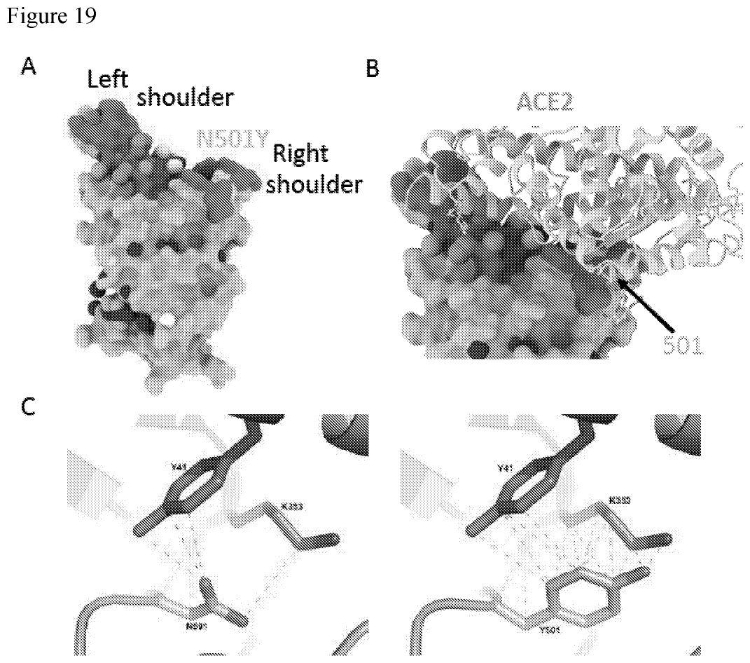

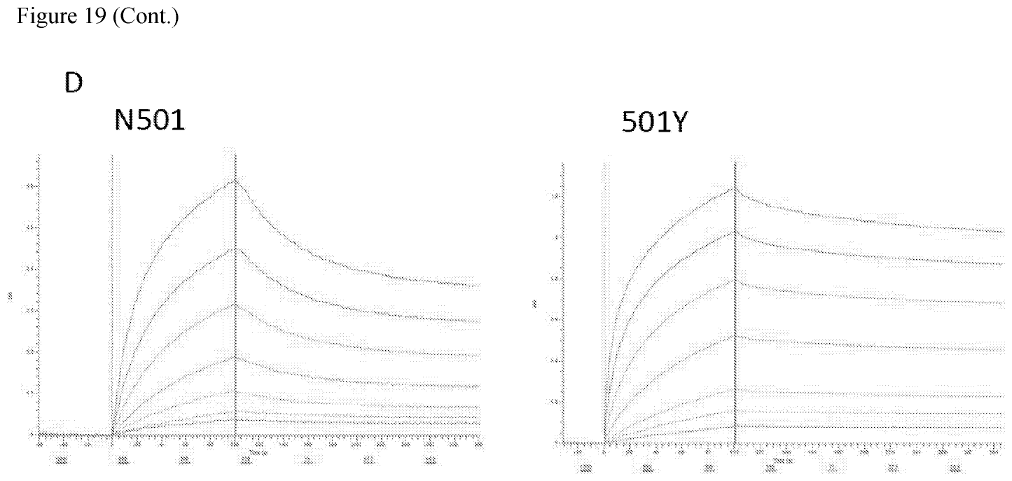

The RBD may be likened to a classical torso, in this analogy the shoulders and neck are involved in interactions with the ACE2 receptor (FIGS. 19A,B). In this context residue 501 lies within the footprint of the receptor on the right shoulder and is involved in hydrophobic interactions, especially with the side chains of residues Y41 and K353 of ACE2 with the 501 mutation from N to Y offering the opportunity for enhanced interactions (FIGS. 19 B,C).

### Effect on ACE2 Affinity

It has been reported that mutations at 501 can increase spike affinity for ACE2 (Starr et al., 2020; Gu et al., 2020), although these data are not for the mutation to Y. In contrast Zahradnik et al., (Zahradnik et al., 2021) report direct selection of N501Y when evolving the RBD to enhance affinity. The effect of this mutation on ACE2 binding by RBD was therefore investigated using biolayer interferometry (BLI) (FIG. 19D). The results indicate a marked (7-fold) increase in binding affinity due to a slower off-rate: WT RBD(501N)-ACE2: K_{D} 75.1 nM (Kₒₙ 3.88E4/Ms, K_{off} 2.92E-3/s), RBD(501Y)-ACE2: K_{D} 10.7 nM (Kₒₙ 6.38E4/Ms, K_{off} 6.85E-4/s). This is in-line with enhanced interactions of the tyrosine sidechain with the side chains of residues Y41 and K353 of ACE2 (FIG. 19C). In the context of a multivalent interaction at the cell surface this effect would be amplified. This alone might account for the selection of the N501Y mutation and an increase in transmission.

### Effect on Monoclonal Antibody Affinity

To investigate the effect of the N501Y mutation on antibody binding, the set of 377 monoclonal antibodies (80 of which mapped to the RBD) generated from SARS-CoV-2 cases infected during the first wave of the pandemic in the UK using samples collected before June 2020 were used. The potent neutralizers tended to have rather few somatic mutations (on average 5.33 and 4.33 amino acids in the heavy and light chains (HC, LC) respectively) and a number of public antibody responses (i.e. those using common V-region genes) including IGHV3-53 (5 potent mAbs), IGHV1-58 (4 potent mAbs) and IGHV3-66 (2 potent mAbs) were present in the collection of RBD specific mAbs.

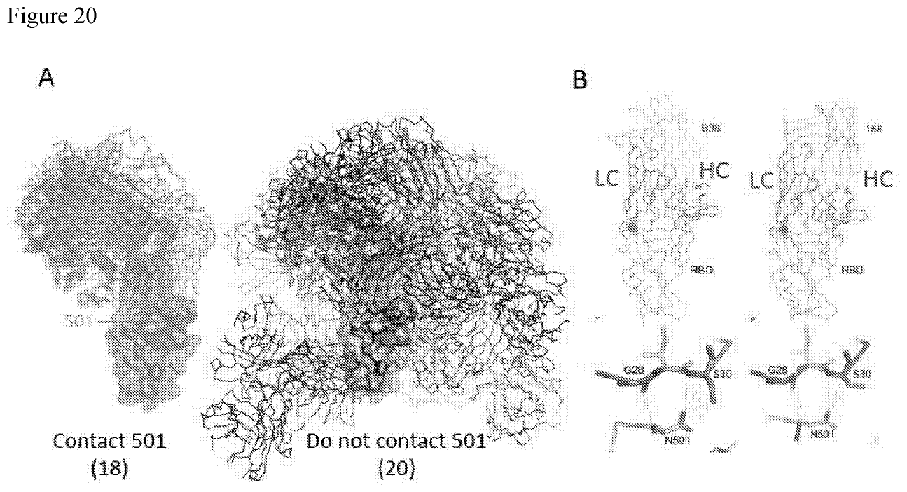

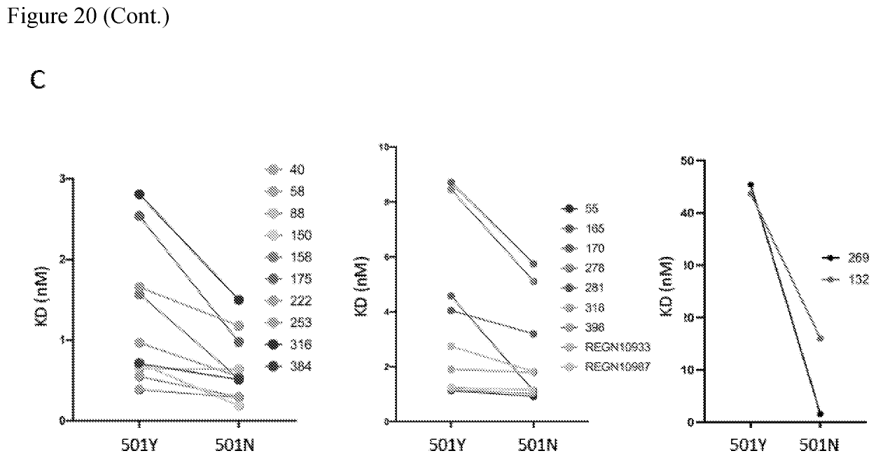

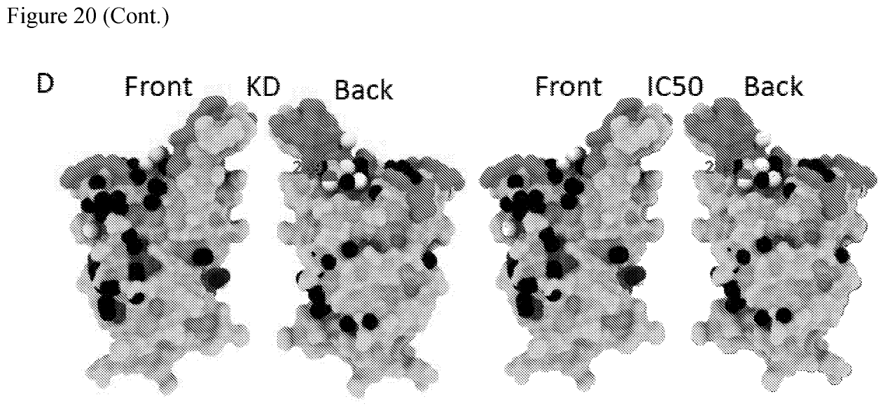

Analysis of the position of the N501Y change with respect to the binding of all structurally characterised potent monoclonal antibodies suggests that the binding of over half of the antibodies would be unaffected by the change (FIG. 20A). However, one class of public antibodies have attracted particular attention, those using IGHV3-53 (Yuan et al., 2020; Wu et al., 2020). These and the IGHV3-66 antibodies bind with N501Y beneath the light chain (LC) CDR1 region and may be expected to be affected by the mutation, since for them, unlike ACE2, the interaction with the asparagine is strongly favourable (FIG. 20 B).

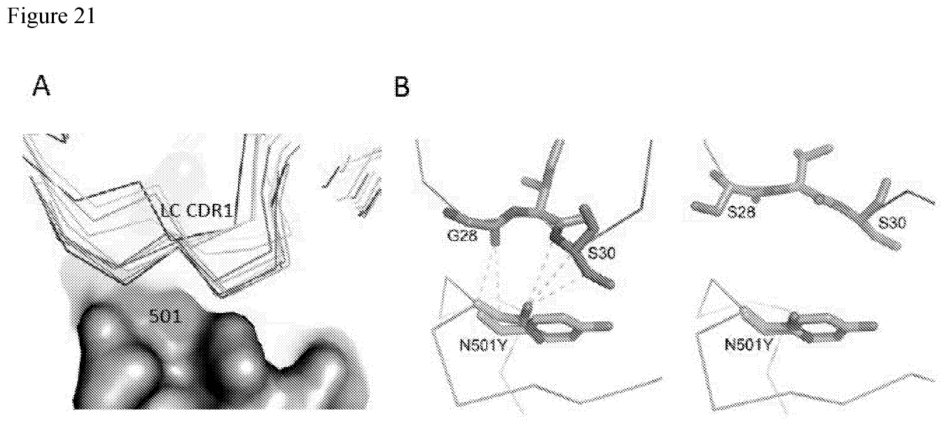

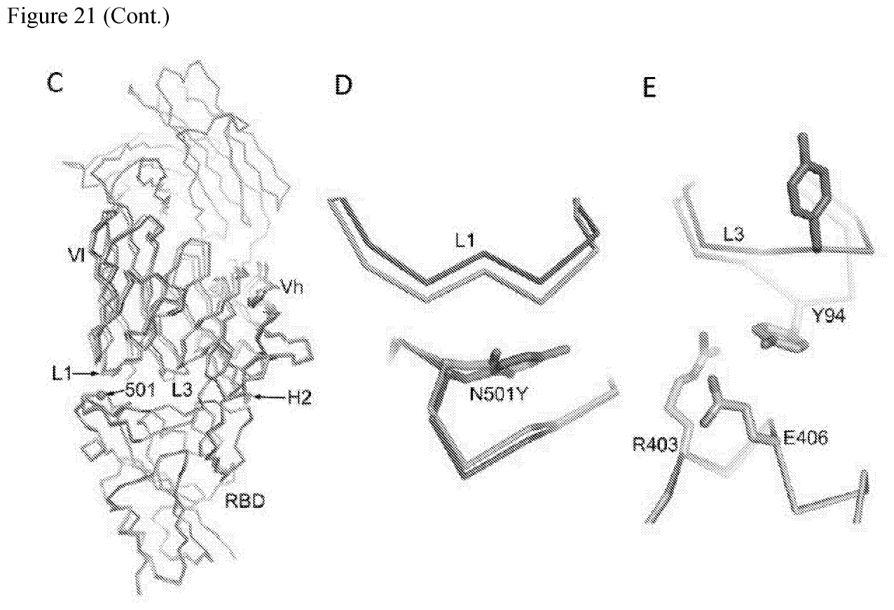

To examine the effects on antibody binding, BLI experiments were performed comparing the binding of potently neutralizing mAbs to RBDs containing 501Y and 501N (Example 17, FIG. 20 C). The results are mapped to the RBD in FIG. 20 D. There is little effect on many potent antibodies, for instance the IGVH1-58 antibodies: 55, 165, 253 and 318. There is a marked ˜3-fold effect for mAb 40 (IGHV3-66) and for most of the important IGHV3-53 antibodies (150, 158 and 175). However, there is a correlation between the LC for the IGHV3-53 antibodies and the magnitude of the effect, thus the common IGLV1-9 antibodies (mAbs 150 and 158) show a consistent reduction in affinity of roughly 3-fold (FIG. 21A). In contrast mAb 222 with IGLV3-20 shows no reduction. When modelled using the most similar light chain from the PDB, it does not contact residue 501 which explains this effect (FIG. 21B). mAb 269, however, appears hyper-sensitive to the mutation (30-fold effect). The structure of a single-chain Fv version of this antibody in complex with WT RBD (FIG. 21C) shows similar interactions to those observed for mAbs 150 and 158. In order to understand this further, the crystal structure of Fab 269 in complex with RBD harbouring 501Y was determined at 2.3 Å resolution (Example 17, Table 10). The result is shown in FIG. 21C. Essentially it seems that the mutation introduces a rather small displacement of the L1 loop (FIG. 21D) but there is a concomitant effect of the neighbouring L3 loop (FIG. 21E), with a significant switch in the position of Y94, abrogating contacts with residues R403 and E406 of the RBD. Finally, there was very little effect on either of the Regeneron antibodies currently in clinical trials, REGN10933 and REGN10987 (FIG. 20C)

### Example 15. Effect of B.1.1.7 Mutations on Neutralisation by Potent mAbs

Next, neutralization assays were performed with the potent mAb targeting the ACE2 interacting surface of RBD. Neutralizations were performed using focus reduction neutralization tests (FRNT) using viral strains Victoria and B.1.1.7 obtained from Public Health England (FIG. 22A, Table 11). For some antibodies (40, 88, 222, 316, 384, 398), FRNT 50 values between B.1.1.7 and Victoria strains were minimally affected (<2-fold difference). However, for others there was a fall in the neutralization titres for B.1.1.7, particularly pronounced for mAb 269, where neutralization was almost completely lost and mAb 278, which failed to reach 100% neutralization showing a maximum of only 78%. Comparing all of these result, an average 4.3-fold reduction in FRNT titres was found between the Victoria and B.1.1.7 strains (p<0.0001). Finally, 2 sets of monoclonal antibodies were looked at, which have reached late stage clinical trials for SARS-CoV-2: the Regeneron pair REGN10933 and REGN10987 and the AstraZeneca mAbs AZD1061, AZD8895 and AZD7442 (a combination of AZD1061 and AZD8895) (FIG. 22 B, Tables 11 and 12). The neutralization of REGN10987 was unaffected by B.1.1.7 while REGN10933 showed a slight reduction but still retained potent activity (FIG. 22 B, Tables 11 and 12). The neutralisation of the AZ antibodies was similarly little affected.

### Example 16. Neutralization Activity of Convalescent Plasma and Vaccine Sera

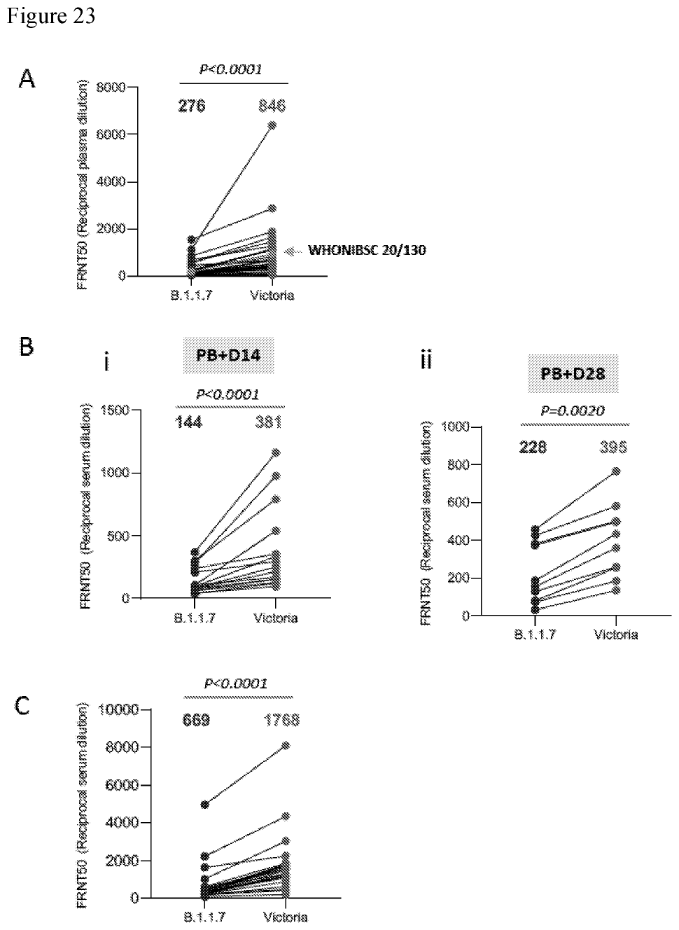

During the first wave of infection, before the emergence of B.1.1.7 strain, a number of samples from cases at convalescence (4-9 weeks following infection) for the generation of monoclonal antibodies were collected. Stored plasma from these cases was used in neutralization assays comparing Victoria and B.1.1.7 (FIG. 23A). 34 convalescent samples were analysed including the WHONIBSC 20/130 reference serum and although a few sera showed near identical FRNT 50 values, the mean FRNT₅₀ dilutions for the B.1.1.7 strain were 3-fold lower than those for the Victoria strain (p<0.0001).

Neutralization of the B.1.1.7 and Victoria strains were also assayed using serum obtained from recipients of the Oxford-AstraZeneca and Pfizer vaccines. For the AstraZeneca AZD1222 vaccine, serum was obtained at baseline and at 14 and 28 days following the second dose. For the Pfizer vaccine, serum was obtained 7-17 days following the second dose of vaccine which was administered 3 weeks after the first dose (participants were seronegative at entry). Neutralization assays against B.1.1.7 and Victoria strains showed a 1.7-fold (n=10 p=0.002) and 2.6-fold (n=15 p<0.0001) reduction in the neutralization titres between B.1.1.7 and Victoria strains for the AstraZeneca vaccine after 14 and 28 days following the second dose respectively (FIG. 23 B). For the Pfizer-BioNTech vaccine BNT162b2, the reduction was also 2.6-fold (n=25 p<0.0001) (FIG. 23 C).

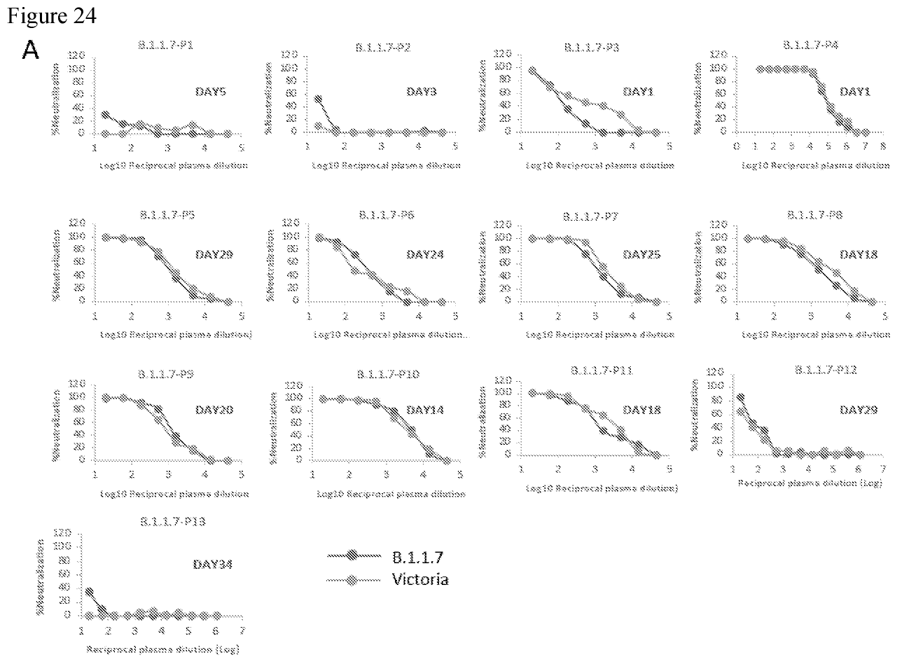

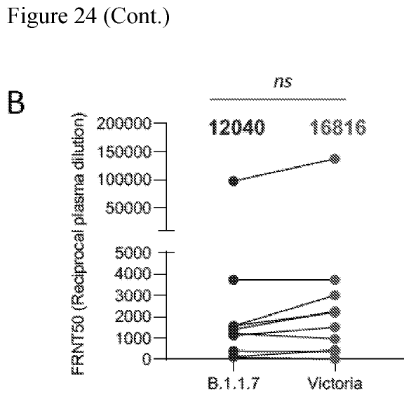

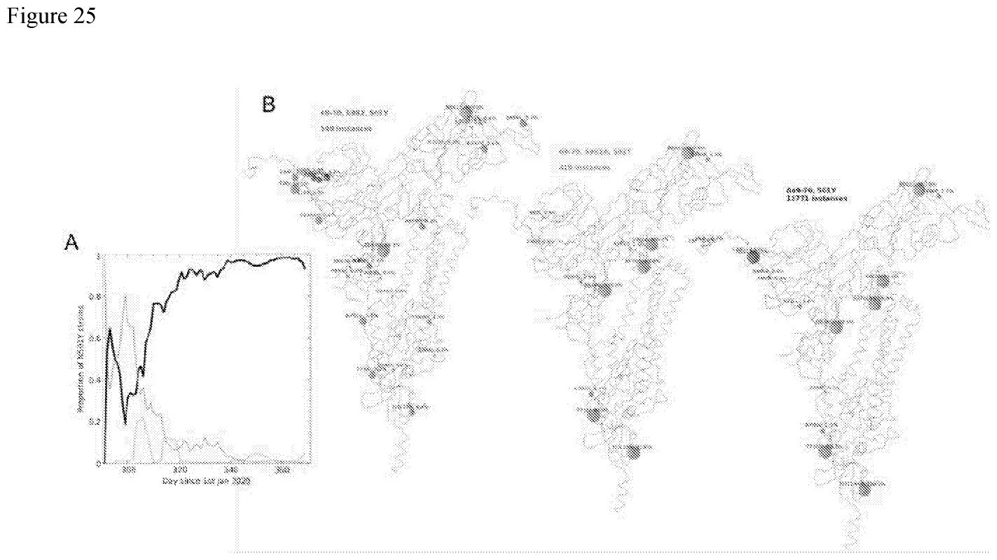

Finally, plasma from 13 patients infected with B.1.1.7 was obtained (all had spike gene dropout in viral PCR testing and 11 were verified by sequencing) at various time points following infection and compared neutralization between B.1.1.7 and Victorian strains (FIG. 24). At early time points neutralization titres were low or absent except in 1 case taken at day 1 of illness who showed identical neutralization of both viruses and was the highest titre of all the samples we have measured in this study at 1: 136884, we speculate that this may represent a reinfection with B.1.1.7. For these samples as a whole there was a no significant difference between the neutralization titres for the two viruses.

In conclusion, the neutralization assays on convalescent and vaccine serum revealed that the B.1.1.7 virus required higher concentration of serum to achieve neutralization, although there was no evidence that the B.1.1.7 virus could evade neutralization by serum raised to early SARS-CoV-2 strains or vaccines.

Neutralising responses against the Victoria virus are less effective against B.1.1.7 and that part of this effect is due to the N501Y mutation as demonstrated by the weaker binding of a number of antibodies to the RBD, where N501Y is the only difference. The reduced binding and neutralization was particularly marked for some, but not all, members of the public VH3-53 class of mAb where the light chain comes in close proximity to Y501. However, B.1.1.7 contains other mutations which may have a bearing on neutralization, in particular the deletions at 69-70 and 144 in the NTD. NTD binding antibodies, which do not block interaction with ACE2, have been described by a number of groups to be able to neutralize SARS-CoV-2 (Chi et al., 2020; Liu et al., 2020; Cerutti et al., 2021), with some antibodies showing IC50 values sub 10 ng/ml. In this study B.1.1.7 showed only a 5.7-fold reduction in the FRNT₅₀ for mAb 159 (FRNT₅₀ Victoria 11 ng/ml B.1.1.7 61 ng/ml) suggesting that despite the residue 144 deletion being on the edge of the footprint for this antibody the binding site has not been completely disrupted.

The level of expression of ACE2 has been shown to correlate with likelihood of infection by SARS-CoV-1 (Jia et al., 2005) and the higher affinity for ACE2 of SARS-CoV-2 has been imputed to underlie its greater transmission. It is reasonable to assume that a further increase in affinity will increase the likelihood of the stochastic events of virus attachment resulting in localisation for sufficient time to trigger, perhaps by the recruitment of additional receptors, internalisation of the virus. As noted by Zahradnik, J. et al. (Zahradnik et al., 2021) in a situation where public health measures reduce RO to below 1 there will be selective pressure to increase receptor affinity.

This increase in transmission is compounded by the reduction in neutralization potency of antibodies generated by prior infection. Modification of the ACE2 binding surface of the RBD would be predicted to directly disrupt the binding of antibodies that lose affinity to the mutated residues. However, antibodies that neutralize by ACE2 competition, even if not directly affected by the mutation will have to compete with ACE2 for binding to the RBD, and mutations of RBD that increase the affinity of ACE2 will tip the equilibrium away from mAb/RBD interaction toward RBD/ACE2 making the virus more difficult to neutralize.

Mutation at position 484 of the Spike likely has a similar dual effect and Zahradnik, J. et al. report that further affinity increase in ACE2 binding is possible. Although most effort has been directed at generating antibodies that neutralize by blocking ACE2 binding, other mechanisms are possible (Huo et al., 2020; Zhou et al., 2020) and indeed partial or non-neutralising antibodies may confer protection (Dunand et al., 2016). Such antibodies would likely be unaffected by mutations in the ACE2 binding site and they deserve more thorough investigation since they would form excellent components in therapeutic cocktails. In addition, natural exposure and vaccination may confer protective immunity against symptomatic and severe COVID-19 via memory T cell responses (Sariol and Perlman, 2020; Altmann and Boyton, 2020).

The recent description of a number of virus variants which appear to have developed independently is a cause for concern as it may signal the emergence of strains able to evade vaccine induced antibody responses. There is now an imperative to closely survey the emergence of novel SARS-CoV2 strains on a global basis and to quickly understand the consequences for immune escape. There is a need to define correlates of protection from SARS-CoV-2 and also to understand how T cells contribute to protection in addition to the antibody response. It is also imperative to understand whether the newly emerging strains including B.1.1.7, 501Y.V2 and P.1 are leading to more severe disease and whether they can evade natural or vaccine induced immune responses (Zhu et al., 2021).

### Example 17. Materials and Methods

For examples 14 to 16.

### COG-UK Sequence Analysis

All COG-UK sequences were downloaded on 24^{th} Jan. 2020, and the translated protein sequences were roughly to the wild-type reference from start and stop codons between nucleotides 21000-25000, and filtered on the mutation 501Y. Sequence alignment was carried out, and identified mutations were plotted as red balls (single point mutations) or black balls (deletions) on the modelled C-alpha positions of the Spike structure, size proportional to the logarithm of the number of mutations. Residues which mutated at an incidence greater than 0.3% compared to the wild-type were labelled explicitly.

### Cloning of Native RBD, RBD N501Y and ACE2

The constructs of native RBD and ACE2 are the same as in Zhou et al., (Zhou et al., 2020). To clone RBD N501Y, a construct of native RBD was used as the template and two primers of RBD (Forward primer 5′-CTACGGCTTTCAGCCCACATACGGTGTGGGCTACCAGCCTT-3′ (SEQ ID NO: 420) and reverse primer 5′-AAGGCTGGTAGCCCACACCGTATGTGGGCTGAAAGCCGTAG-3′ (SEQ ID NO: 421)) and two primers of pNEO vector (Forward primer 5′-CAGCTCCTGGGCAACGTGCT-3′ (SEQ ID NO: 422) and reverse primer 5′-CGTAAAAGGAGCAACATAG-3′ (SEQ ID NO: 423)) were used to do PCR. Amplified DNA fragments were digested with restriction enzymes AgeI and KpnI and then ligated with digested pNEO vector. This construct encodes exactly the same protein as native RBD except the N501Y mutation.

### Protein Production

Protein expression and purification were performed as described in Zhou et al. (Zhou et al., 2020).

### Preparation of 269 Fab

Fab fragments of 269 antibody was digested and purified using Pierce Fab Preparation Kit, following the manufacturer's protocol.

### Crystallization

269 Fab was mixed with RBD N501Y in a 1:1 molar ratio with a final concentration of 9.9 mg ml⁻¹. After incubated at room temperature for 30 min, the sample was used for initial screening of crystals in Crystalquick 96-well X plates (Greiner Bio-One) with a Cartesian Robot using the nanoliter sitting-drop vapor-diffusion method as previously described (Walter et al., 2003). Crystals for the complex were obtained from a Molecular Dimensions Proplex screen, condition B10 containing 0.15 M ammonium sulfate, 0.1 M MES pH 6.0 and 15% PEG 4000.

### Biolayer Interferometry

BLI experiments were run on an Octet Red 96e machine (Fortebio). To measure the binding affinities of monoclonal antibodies with native RBD and RBD N501Y, RBD and RBD N501Y were immobilized onto AR2G biosensors (Fortebio) separately. monoclonal antibodies were used as analytes. To measure the binding affinities of native RBD and RBD N501Y with ACE2, native RBD and RBD N501Y were immobilized onto AR2G biosensors separately. ACE2 with serial dilutions was used as analytes. Data were recorded using software Data Acquisition 11.1 (Fortebio) and analysed using software Data Analysis HT 11.1 (Fortebio) with a 1:1 fitting model.

### X-Ray Data Collection, Structure Determination and Refinement

Crystals were mounted in loops and dipped in solution containing 25% glycerol and 75% mother liquor for a second before being frozen in liquid nitrogen prior to data collection. Diffraction data were collected at 100 K at beamline I03 of Diamond Light Source, UK. Diffraction images of 0.1° rotation were recorded on an Eiger2 XE 16M detector (exposure time of either 0.007 s per image, beam size 80×20 μm, 100% beam transmission and wavelength of 0.9763 Å). Data were indexed, integrated and scaled with the automated data processing program Xia2-dials (Winter, 2010; Winter et al., 2018). The data set of 720° was collected from 2 frozen crystal to 2.19 Å resolution.

The crystal belongs to space group C2 with unit cell dimensions a=195.1, b=85.0 Å, c=57.9 Å and 0=100.6°. The structure was determined by molecular replacement with PHASER (McCoy et al., 2007) using search models of SARS-CoV-2 RBD/COVOX-scFv269 complex (PDB ID, 7BEM) and the ChCl domains of SARS-CoV-2 RBD/COVOX-158 complex (PDB ID, 7BEK). There is one N501Y RBD/COVOX-269 Fab complex in the crystal asymmetric unit and a solvent content of ˜51%. Cyclic model rebuilding with COOT (Emsley and Cowtan, 2004) and refinement with PHENIX (Liebschner et al., 2019) resulted in the current structure with R_{work}=0.197 and R_{free}=0.222 for all data to 2.19 Å resolution.

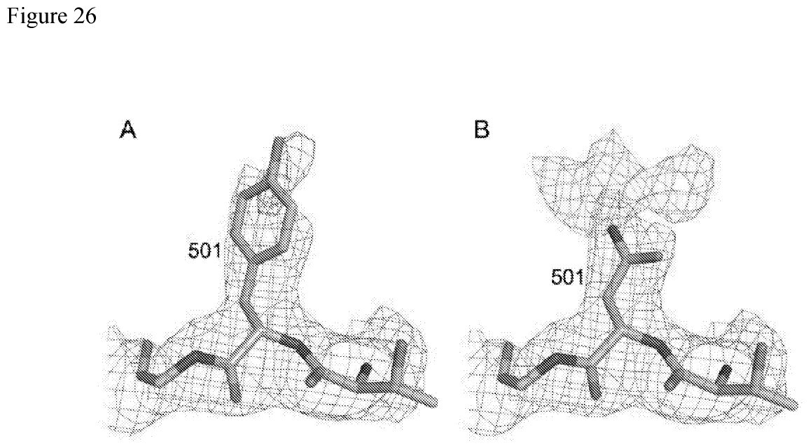

Electron density for the side chain of Y501 is weak. However, when the structure was refined with an asparagine at 501, there is strong, but dispersed positive density around the side chain, suggesting the presence of a flexible tyrosine residue (FIG. 26). Mass spectrometry and biolayer interferometry data confirm it is indeed a tyrosine at 501.

Data collection and structure refinement statistics are given in Table 10. Structural comparisons used SHP (Stuart et al., 1979), residues forming the RBD/Fab interface were identified with PISA (Krissinel and Henrick, 2007), figures were prepared with PyMOL (The PyMOL Molecular Graphics System, Version 1.2r3pre, Schrödinger, LLC).

### Viral Stocks

SARS-CoV-2/human/AUS/VIC01/2020 (Caly et al., 2020) and SAR-CoV-2/B.1.1.7, provided by Public Health England, were grown in Vero (ATCC CCL-81) cells. Cells were infected with the SARS-CoV-2 virus at multiplicity of infection of 0.0001. Virus containing supernatant was harvested when 80% CPE was observed and spun at 2000 rpm at 4° C. before being stored at −80° C. Viral titres were determined by a focus-forming assay on Vero cells. Both Victoria passage 5 and B.1.1.7 stocks passage 2, were sequence verified to contain the expected spike protein sequence and no changes to the furin cleavage sites.

### Focus Reduction Neutralization Assay (FRNT)

The neutralization potential of Ab was measured using a Focus Reduction Neutralization Test (FRNT), where the reduction in the number of the infected foci is compared to a no antibody negative control well. Briefly, serially diluted Ab or plasma was mixed with SARS-CoV-2 strain Victoria or B.1.1.7 and incubated for 1 hr at 37° C. The mixtures were then transferred to 96-well, cell culture-treated, flat-bottom microplate containing confluent Vero cell monolayers in duplicate and incubated for further 2 hrs followed by the addition of 1.5% semi-solid carboxymethyl cellulose (CMC) overlay medium to each well to limit virus diffusion. A focus forming assay was then performed by staining Vero cells with human anti-NP mAb (mAb206) followed by peroxidase-conjugated goat anti-human IgG (A0170; Sigma). Finally, the foci (infected cells) approximately 100 per well in the absence of antibodies, were visualized by adding TrueBlue Peroxidase Substrate. Virus-infected cell foci were counted on the classic AID EliSpot reader using AID ELISpot software. The percentage of focus reduction was calculated and IC₅₀ was determined using the probit program from the SPSS package.

### Pfizer Vaccine

Pfizer vaccine serum was obtained 7-17 days following the second dose of vaccine which was administered 3 weeks after the first dose (participants were to the best of their knowledge seronegative at entry).

The study was approved by the Oxford Translational Gastrointestinal Unit GI Biobank Study 16/YH/0247 [research ethics committee (REC) at Yorkshire & The Humber-Sheffield]. The study was conducted according to the principles of the Declaration of Helsinki (2008) and the International Conference on Harmonization (ICH) Good Clinical Practice (GCP) guidelines. Written informed consent was obtained for all patients enrolled in the study. Vaccines were Health Care Workers, based at Oxford University Hospitals NHS Foundation Trust, not known to have prior infection with SARS-COV-2. Each received two doses of COVID-19 mRNA Vaccine BNT162b2, 30 micrograms, administered intramuscularly after dilution as a series of two doses (0.3 mL each) 18-28 days apart. The mean age of vaccines was 43 years (range 25-63), 11 male and 14 female.

### Astrazeneca-Oxford Vaccine Study Procedures and Sample Processing

Full details of the randomized controlled trial of ChAdOx1 nCoV-19 (AZD1222), were previously published (PMID: 33220855/PMID: 32702298). These studies were registered at ISRCTN (U.S. Pat. Nos. 15,281,137 and 89,951,424) and ClinicalTrials.gov (NCT04324606 and NCT04400838). Written informed consent was obtained from all participants, and the trial is being done in accordance with the principles of the Declaration of Helsinki and Good Clinical Practice. The studies were sponsored by the University of Oxford (Oxford, UK) and approval obtained from a national ethics committee (South Central Berkshire Research Ethics Committee, reference 20/SC/0145 and 20/SC/0179) and a regulatory agency in the United Kingdom (the Medicines and Healthcare Products Regulatory Agency). An independent DSMB reviewed all interim safety reports. A copy of the protocols was included in previous publications (PMID: 33220855/PMID: 32702298).

Data from vaccinated volunteers who received two vaccinations are included in this paper. Vaccine doses were either 5×10¹⁰ viral particles (standard dose; SD/SD cohort n=21) or half dose as their first dose (low dose) and a standard dose as their second dose (LD/SD cohort n=4). The interval between first and second dose was in the range of 8-14 weeks. Blood samples were collected and serum separated on the day of vaccination and on pre-specified days after vaccination e.g. 14 and 28 days after boost.

### Example 18. Mutational Changes in B.1.351

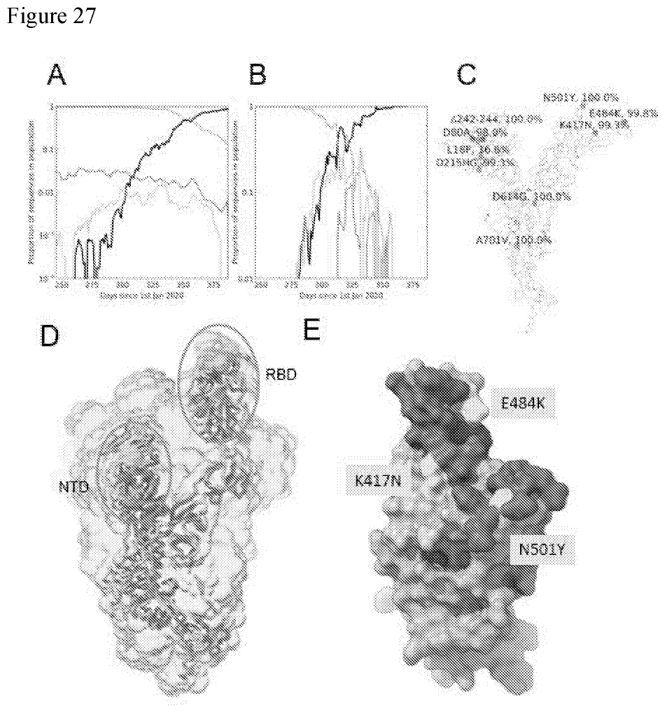

A number of isolates of B.1.351 have been described, all of which have the key mutations K417N, E484K and N501Y in the RBD. Tegally et al. (Tegally et al., 2021) reported an isolate containing 10 changes relative to the Wuhan sequence, L18F, D80A, D215G, L242-244 deleted, R246I, K417N, E484K, N501Y, D614G, A701V. Sequencing of the strain used in this report, from a case in the UK, shows only 8 changes and lacks L18F and R246I compared to the Tegaly et al. isolate. Coronavirus genome sequences were analysed in both the UK, acquired from the COG-UK database (Tatusov et al., 2000), and South Africa, acquired from GISAID (https://www.gisaid.org). It appears that B.1.1.7 and B.1.351 quickly become overwhelmingly dominant in the UK and South Africa respectively. In the evolution of both the B.1.1.7 variant in the UK, and the B.1.351 variant in South Africa, a substantial population of NTD-deletion-only mutants (469-70 in B.1.1.7 and 4242-244 in B.1.153) and 501Y-only mutants were observed in both countries preceding the rising dominance of strains harbouring both deletions and 501Y (FIG. 27 A, B). Counts of both ‘single-mutant’ variants have since waned. The characteristic mutations for B.1.351 as found in South Africa are shown (FIGS. 27 C,D,E). In addition, as of 2^{nd} Feb. 2021 in the COG-UK database, 21 of the B.1.1.7 sequences were observed to have independently acquired the 484K (but not the 417N) mutation found in the B.1.351 variant, and 90 sequences display these mutations in the background of B.1.351 (as defined by bearing the characteristic 4242-244 NTD deletion).

### Example 19. Neutralization of B.1.351 by Convalescent Plasma

Plasma was collected from a cohort of infected patients during the first wave of SARS-CoV-2 infection in the UK. Samples were collected from convalescent cases 4-9 weeks following infection in June 2020, before the emergence of B.1.1.7. Also included is a recent collection of plasma from patients infected with B.1.1.7.

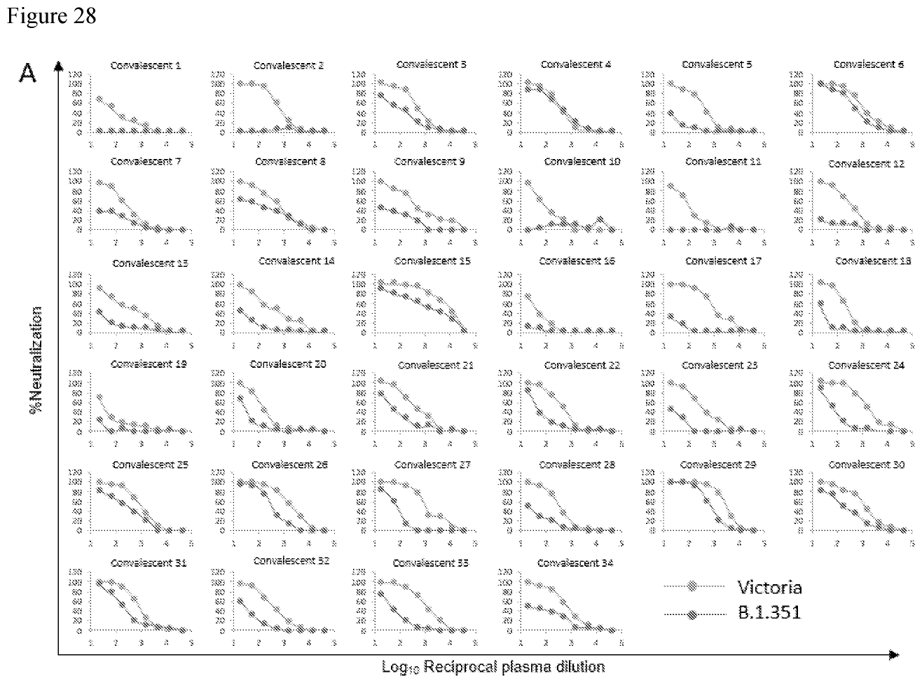

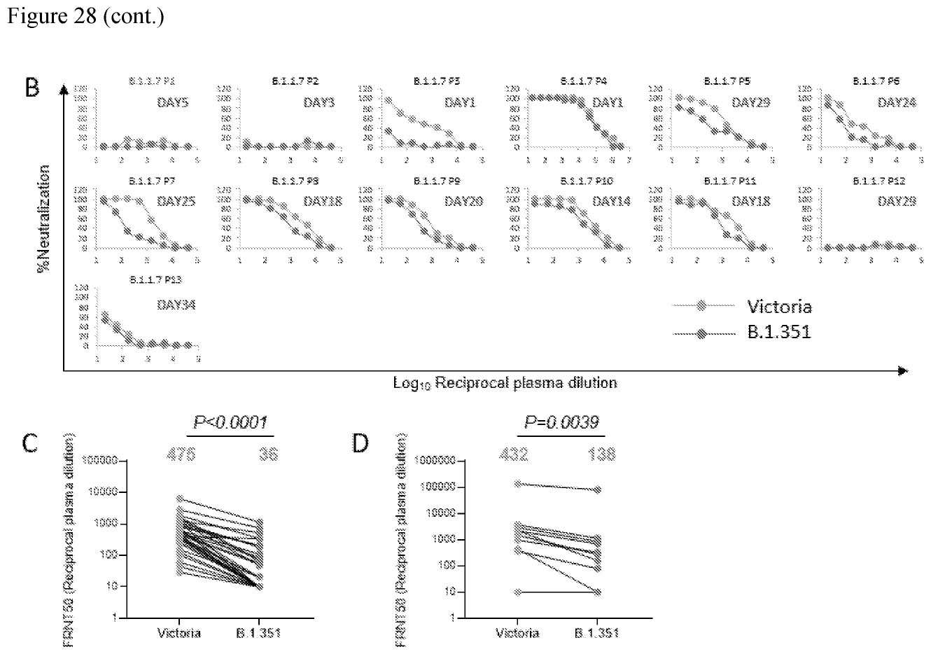

Neutralization titres against Victoria, an early Wuhan related strain of SARS-CoV-2 (Seemann et al., 2020), were compared to B.1.351 using a focus reduction neutralization test (FRNT). For the early convalescent samples (n=34), neutralization titres against B.1.351 were on average 13.3-fold reduced compared to Victoria (p=<0.0001) (FIG. 28A, Table 14). A few convalescent samples e.g. 4, 6, 15 retained good neutralization of B.1.351, but for most, titres were considerably reduced and significantly, 18/34 samples failed to reach 50% neutralization at a plasma dilution of 1:20 with a number showing a near total reduction of neutralization activity. Overall in the 34 convalescent plasma samples there was a 13.3-fold (geometric mean) reduction in neutralization titre between Victoria and B.1.351 p<0.0001 (FIG. 28 C).

Neutralization was also performed using plasma recently collected, at different time points, from patients suffering from B.1.1.7 (n=13), all of these cases had S-gene knock out on diagnostic PCR (Thermo Fisher TaqPath, characteristic of B.1.1.7) and 11 had viral sequencing confirming B.1.1.7 (FIG. 28B Table 14). Neutralization titres were low at early time points for both Victoria and B.1.351, but in one case (B.1.1.7 P4), a sample taken 1 day following admission to hospital, showed a very high titre against Victoria (1:136,884) and B.1.351 (1:81,493) and this may represent a reinfection with B.1.1.7. Overall there was a 3.1-fold (geometric mean) reduction in titres between Victoria and B.1.351 in sera from patients infected with B.1.1.7 (FIG. 28D).

### Example 20. Neutralization of B.1.351 by Vaccine Serum

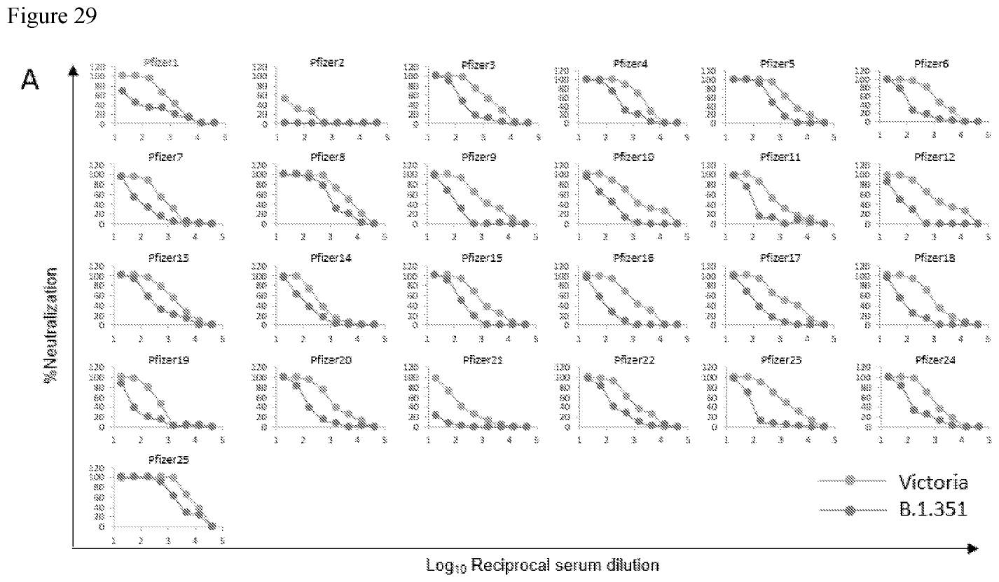

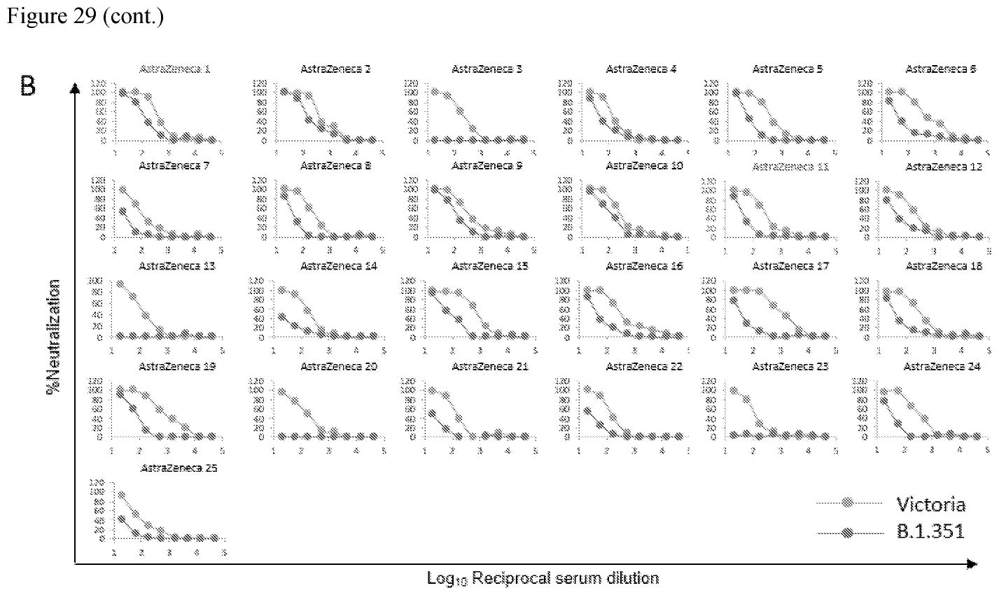

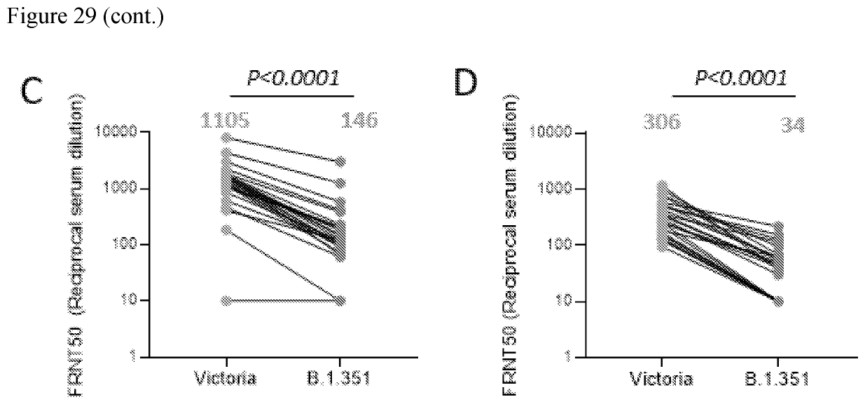

Neutralization was measured of Victoria and B.1.351 was using vaccine serum obtained from individuals vaccinated with the Pfizer-BioNTech vaccine BNT162b2 and Oxford-AstraZeneca AZD1222 vaccine. For Pfizer-BioNTech, vaccinated serum was obtained from healthcare workers (n=25), 4-17 days following the second dose of vaccine, administered 3 weeks after the first dose (FIG. 29A and Table 15). For the AstraZeneca vaccine, samples (n=25), were obtained 14 or 28 days following the second vaccine dose, with a dosing interval of 8-14 weeks (FIG. 29B Table 15). For the Pfizer-BioNTech vaccine serum, geometric mean titres for B.1.351 were 7.6-fold lower than Victoria (p=<0.0001) (FIG. 29C) and for the Oxford-AstraZeneca vaccine serum geometric mean B.1.351 titres were 9-fold lower than Victoria (p<0.0001) (FIG. 29D and Table 15).

The Pfizer-BioNTech vaccine serum induced 3.6-fold higher neutralization titres against the Victoria strain than the Oxford-AstraZeneca vaccine (p=<0.0001). Although the overall reduction of titres was quite similar, 7.6-fold vs 9-fold respectively, because the AstraZeneca titres started from a lower base more of the samples failed to reach 50% FRNT titres against B.1.351 (9/25) than for the Pfizer vaccine (2/25).

### Example 21. Neutralization of B.1.351 by Monoclonal Antibodies

The pool of 377 human monoclonal antibodies directed to the spike protein was raised from convalescent samples obtained from patients infected during the first wave of SARS-CoV-2 in the UK. The 20 most potent mAb (FRNT₅₀ titres <50 μg/ml), (19 anti-RBD and 1 anti-NTD) and performed neutralization assays against the UK B.1.1.7 strain, the Victoria strain and B.1.351 strains (FIG. 22A, Tables 12 and 13). Data against the Victoria and B.1.351 strains are also shown in FIG. 30 & Table 16.

The effects on mAb neutralization were severe, 14/20 antibodies had >10-fold fall in neutralization titres, with most of these showing a complete knock out of activity. This is in line with the key role of K417, E484 and N501, in particular E484, in antibody recognition of the ACE2 interacting surface of the RBD described below and FIG. 31A-G.

Interestingly, the single potent NTD binding antibody included in these analyses mAb 159, also showed a complete knock out of activity against B.1.351 which contains deletion of amino acids 242-244 in the NTD part of the epitope for mAb 159. As can be seen from FIGS. 31 H,I, the RBD loop 246-253 interacts with the heavy chain of mAb 159 and also that of 4A8, the only other potent neutralising NTD binder with a structure reported (Chi et al., 2020). The 242-244 deletion will undoubtedly alter the presentation of this loop compromising binding to these mAbs. Binding at this so-called ‘supersite’ has been reported as of potential therapeutic relevance (McCallum et al., 2021). The B.1.1.7, B.1.351 and P.1 lineages have all converged with either deletions or systematic changes in the NTD. Although P.1 does not harbour NTD deletions, the changes L18F, T20N and P26S (Faria et al., 2021) would be expected to impact markedly on binding at the NTD epitope. Since these convergent features may not have arisen by selective pressure from antibody responses it seems likely there is an underlying biological driver still to be discovered, like the increased receptor binding and potential increased transmissibility imparted by the RBD mutations, which may cause this epitope to be extremely susceptible to mutation and escape from antibody binding.

### Example 22. Neutralization of B.1.351 by Monoclonal Antibodies in Late Stage Clinical Trials

A number of monoclonal antibodies are in late stage clinical trials as therapy or prophylaxis against SARS-CoV2. Regeneron and AstraZeneca use cocktails of 2 monoclonal antibodies to give resistance to mutational escape of viruses. Neutralization assays were performed with the Regeneron pair REGN10933 and REGN10987 and the AstraZeneca pair of mAb AZD106 and AZD8895 and (FIGS. 22B, 30B). The neutralization of REGN10987 was unaffected by B.1.351, while REGN10933 was severely impaired (317-fold) (FIG. 30B). Neutralisation by the AZ pair of antibodies was little affected on B.1.351 compared to Victoria.

Table 12 shows that many of the most-potent mAbs against the Victoria strain retained high potency against the B.1.1.7 strain. In particular, mAbs 40, 55, 58, 222, 281, 316, 384, 394 and 398 maintained strong potency against B.1.1.7. A number of the most-potent mAbs also retained high potency against the South African strain (B.1.351). In particular, mAbs 55, 58, 150, 165, 222, 253, 278 and 318 retained strong potency against B.1.351.

### Example 23. Understanding the Abrogation of Neutralisation: ACE2 Binding to B.1.351 RBD

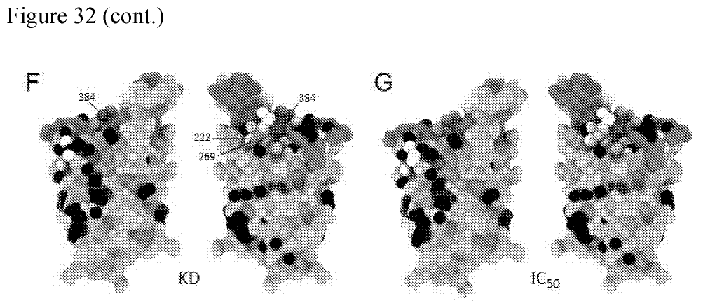

The triple mutation K417N, E484R and N501Y is characteristic of the B.1.351 RBD. These residues are situated within the ACE2 footprint (FIG. 27 E) and in vitro evolution to optimise the affinity for ACE2 has suggested that they confer higher affinity for the receptor (Starr et al., 2020; Zahradnik et al., 2021). To investigate this effect, the kinetics of binding of soluble ACE2 to recombinant RBD was measured by biolayer interferometry (BLI), (FIG. 32A,B). As expected the affinity for B.1.351 RBD is high, in fact 19-fold higher than for the Victoria RBD and 2.7-fold higher than for B.1.1.7. The KD is 4.0 nM, Kon 4.78E4/Ms and Koff 1.93E-4/s, thus the off-rate is approximately 1.5 hours, this will further exacerbate the decline in potency observed in neutralisation assays, since antibody of lower affinity will struggle to compete with ACE2 unless they have a very slow off-rate or show an avidity effect to block attachment. Thus, while all of the set of potent RBD binders have an affinity higher than that between ACE2 and Victoria or B.1.1.7 RBD (KDs 75.1 and 10.7 nM respectively) five of the 19 have lower or equal affinity than for ACE2 and B.1.351 RBD. A small further increase in affinity (eg 2-fold) would beat almost all the antibodies.

### Example 24. Dissection of Impact of RBD Mutations on RBD Binding

To understand the order of magnitude of the abrogation in neutralisation of more than two thirds of the 19 potent mAbs that bind the RBD, the K_{D} for binding to recombinant RBD was measured by BLI, (FIGS. 32 C,D Table 16). Whereas for the Fabs tested against Victoria, 17 had KDs below 4 nM (the affinity of ACE2 for B.1.351), against B.1.351 this reduced to 4 (or 2 if the engineered light chain versions of 253 are removed with 7 Fabs failing to achieve near μM affinity. These results broadly follow the neutralisation results (compare panels C and D of FIG. 32, and see Table 16), suggesting that the observed pattern of effects on neutralisation is largely due to the amino acid substitutions in the RBD, K417N, E484K and N501Y.

The basis of these effects may be understood in the context of an anatomical description of the RBD, in terms of a human torso we have defined four almost contiguous structural epitopes, left shoulder, neck, right shoulder and right flank, with a separate left flank epitope (FIG. 32 E). In this context, the ACE2 binding site extends across the neck and both shoulders. N501Y is on the right shoulder, K417N at the back of the neck and E484R on the left shoulder. Although the three mutations are nominally in different epitopes the overlapping nature of these epitopes means that the residues are sufficiently close that more than one might directly affect the binding of any one antibody. In addition, there may be allosteric effects (the structural equivalent of epistasis in genetics) whereby effects may extend over some distance. The combination of this, with the observation that only a relatively small fraction of the footprint residues are critical to binding, accounts for the distinction between structural epitopes (footprints) and functional epitopes (Cunningham and Wells, 1993). Despite these caveats the majority of the effects observed are directly explicable by reference to prior structural knowledge.

Many of the reported Fab/SARS-CoV-2 RBD complexes are for antibodies which use the public HC V-region IGHV3-53 (Yuan et al., 2020) and these are well represented in the set by five antibodies that are potent against the Victoria virus. Four of these, 150, 158, 175 and 269, have their neutralization and binding abilities severely compromised or abolished, while 222 is an exception, since its binding is unaffected by the B.1.351 variant (FIGS. 32 F,G). The family of IGHV3-53 antibodies bind at the same epitope at the back of the neck of the RBD with very similar approach orientations also shared by the IGHV3-66 Fabs. The majority of these make direct contacts to K417 and N501, but none of them contact E484. The rather short HC CDR3s of these Fabs are usually positioned directly above K417, making hydrogen bonds or salt bridges as well as hydrophobic interactions, while N501 interacts with the LC CDR-1 loop (FIG. 31). However mAb 150 is a little different, forming both a salt-bridge between K417 and the LC CDR3 D92 and a H-bond between N501 and S30 in the LC CDR1 (FIG. 31B), whereas 158 is more typical, making a hydrogen bond from the carbonyl oxygen of G100 of the HC CDR3 and K417 and hydrophobic contacts from S30 of the LC CDR1 to N501. It would therefore be expected that the combined effects of the K417N and N501Y mutations would severely compromise the binding of most IGHV3-53 and IGHV3-66 class mAbs. However one member of this class, 222, is unaffected by either the B.1.1.7 or B.1.351 variant.

Fab 88 binds RBD at the back of the left shoulder, residues G104 and K108 of the HC CDR3 contact E484 meanwhile the LC CDR2 makes extensive hydrophobic interactions and a main chain hydrogen bond from Y51 and a salt bridge from D53 to K417 (FIG. 31 A). The change of charge at E484 from negative to positive and shortening of the residue 417 side chain from K to N would be expected to abolish all these interactions, explaining the several hundred-fold loss in K_{D}. 384 is one of the most potent neutralizing mAbs we have found against the Victoria virus. This mAb approaches the binding site from the front of the left shoulder, burying 82% of the solvent accessible area of E484 by hydrogen bonding with Y50, T57 and Y59 as well as making a salt bridge with R52 of the HC CDR2 (FIG. 31 D), explaining the catastrophic impact of the E484K mutation on binding (Table 16).

MAb 222 was not the only antibody to show resilience to B.1.351. The FRNT₅₀ titres for mAbs 55, 165, 253 and 318 were also relatively equal between Victoria and B.1.351 indicating that their epitopes are not perturbed by the K417N, E484K and N501Y mutations. Antibodies 55, 165 and 253 are related to each other and it is shown that combining the light chains of 55 or 165 with the heavy chain of 253 leads to a >1 log increase in neutralization titres. The Chimeras 253H/55L and 253H/165L can both neutralize B.1.351 with FRNT₅₀ titres of 9 and 13 ng/ml respectively. Structures of 253 and these chimera Fabs with either RBD or Spike show that they bind almost identically to the same epitope and don't contact any of the three mutation site residues, correlating well with the neutralization and BLI binding data (FIG. 31 C).

### Example 25. Methods for Examples 18 to 24

### Viral Stocks

SARS-CoV-2/human/AUS/VIC01/2020 (Caly et al., 2020) and SARS-CoV-2/B.1.1.7, provided by Public Health England, were both grown in Vero (ATCC CCL-81) cells. Cells were infected with the SARS-CoV-2 virus using an MOI of 0.0001. Virus containing supernatant was harvested at 80% CPE and spun at 2000 rpm at 4° C. before storage at −80° C. Viral titres were determined by a focus-forming assay on Vero cells. Both Victoria passage 5 and B.1.157 passage 5 stocks were sequenced to verify that they contained the expected spike protein sequence and no changes to the furin cleavage sites. The B1.351 virus used in these studies contained the following mutations: D80A, D215G, L242-244 deleted, K417N, E484K, N501Y, D614G, A701V.

### Bacterial Strains and Cell Culture

Vero (ATCC CCL-81) cells were cultured at 37° C. in Dulbecco's Modified Eagle medium (DMEM) high glucose (Sigma-Aldrich) supplemented with 10% fetal bovine serum (FBS), 2 mM GlutaMAX (Gibco, 35050061) and 100 U/ml of penicillin-streptomycin. Human mAbs were expressed in HEK293T cells cultured in UltraDOMA PF Protein-free Medium (Cat #12-727F, LONZA) at 37° C. with 5% CO2.

E. coli DH5α bacteria were used for transformation of plasmid pNEO-RBD K417N, E484K, N501Y. A single colony was picked and cultured in LB broth with 50 μg mL⁻ Kanamycin at 37° C. at 200 rpm in a shaker overnight. HEK293T (ATCC CRL-11268) cells were cultured in DMEM high glucose (Sigma-Aldrich) supplemented with 10% FBS, 1% 100× Mem Neaa (Gibco) and 1% 100× L-Glutamine (Gibco) at 37° C. with 5% CO₂. To express RBD, RBD K417N, E484K, N501Y and ACE2, HEK293T cells were cultured in DMEM high glucose (Sigma) supplemented with 2% FBS, 1% 100× Mem Neaa and 1% 100× L-Glutamine at 37° C. for transfection.

### Participants

Participants were recruited through three studies: Sepsis Immunomics [Oxford REC C, reference:19/SC/0296]), ISARIC/WHO Clinical Characterisation Protocol for Severe Emerging Infections [Oxford REC C, reference 13/SC/0149] and the Gastrointestinal illness in Oxford: COVID substudy [Sheffield REC, reference: 16/YH/0247]. Diagnosis was confirmed through reporting of symptoms consistent with COVID-19 and a test positive for SARS-CoV-2 using reverse transcriptase polymerase chain reaction (RT-PCR) from an upper respiratory tract (nose/throat) swab tested in accredited laboratories. A blood sample was taken following consent at least 14 days after symptom onset. Clinical information including severity of disease (mild, severe or critical infection according to recommendations from the World Health Organisation) and times between symptom onset and sampling and age of participant was captured for all individuals at the time of sampling.

Sera from Pfizer Vaccinees

Pfizer vaccine serum was obtained 7-17 days following the second dose of vaccine which was administered 3 weeks after the first dose (participants were to the best of their knowledge seronegative at entry).

The study was approved by the Oxford Translational Gastrointestinal Unit GI Biobank Study 16/YH/0247 [research ethics committee (REC) at Yorkshire & The Humber-Sheffield]. The study was conducted according to the principles of the Declaration of Helsinki (2008) and the International Conference on Harmonization (ICH) Good Clinical Practice (GCP) guidelines. Written informed consent was obtained for all patients enrolled in the study. Vaccinees were Health Care Workers, based at Oxford University Hospitals NHS Foundation Trust, not known to have prior infection with SARS-COV-2. Each received two doses of COVID-19 mRNA Vaccine BNT162b2, 30 micrograms, administered intramuscularly after dilution as a series of two doses (0.3 mL each) 18-28 days apart. The mean age of vaccines was 43 years (range 25-63), 11 male and 14 female.

### AstraZeneca-Oxford Vaccine Study Procedures and Sample Processing

Full details of the randomized controlled trial of ChAdOx1 nCoV-19 (AZD1222), were previously published (PMID: 33220855/PMID: 32702298). These studies were registered at ISRCTN (U.S. Pat. Nos. 15,281,137 and 89,951,424) and ClinicalTrials.gov (NCT04324606 and NCT04400838). A copy of the protocols was included in previous publications (PMID: 33220855/PMID: 32702298).

Data from vaccinated volunteers who received two vaccinations are included in this paper. Vaccine doses were either 5×10¹⁰ viral particles (standard dose; SD/SD cohort n=21) or half dose as their first dose (low dose) and a standard dose as their second dose (LD/SD cohort n=4). The interval between first and second dose was in the range of 8-14 weeks. Blood samples were collected and serum separated on the day of vaccination and on pre-specified days after vaccination e.g. 14 and 28 days after boost.

### COG-UK Sequence Analysis

COG-UK sequences from the 2nd Feb. 2021 (Tatusov et al., 2000), and GISAID sequences (https://www.gisaid.org/) from South Africa from 30th Jan. 2021 were downloaded and the protein sequence for the Spike protein was obtained after nucleotide 21000, followed by sequence alignment and recognition of mutations. The B.1.351 variant was filtered using selection criteria 501Y and 4242. The B.1.1.7 variant was filtered using selection criteria 501Y and 469. The structural locations of mutations were modelled as red (single point mutations), black (deletions) or blue (additions) on the Spike structure with the size proportional to the logarithm of the incidence, and those mutations over 5% incidence in the population were explicitly labelled.

### Focus Reduction Neutralization Assay (FRNT)

The neutralization potential of Ab was measured using a Focus Reduction Neutralization Test (FRNT), where the reduction in the number of the infected foci is compared to a no antibody negative control well. Briefly, serially diluted Ab or plasma was mixed with SARS-CoV-2 strain Victoria or B.1.351 and incubated for 1 hr at 37° C. The mixtures were then transferred to 96-well, cell culture-treated, flat-bottom microplates containing confluent Vero cell monolayers in duplicate and incubated for a further 2 hrs followed by the addition of 1.5% semi-solid carboxymethyl cellulose (CMC) overlay medium to each well to limit virus diffusion. A focus forming assay was then performed by staining Vero cells with human anti-NP mAb (mAb206) followed by peroxidase-conjugated goat anti-human IgG (A0170; Sigma). Finally, the foci (infected cells) approximately 100 per well in the absence of antibodies, were visualized by adding TrueBlue Peroxidase Substrate. Virus-infected cell foci were counted on the classic AID EliSpot reader using AID ELISpot software. The percentage of focus reduction was calculated and IC₅₀ was determined using the probit program from the SPSS package.

### Cloning of Native RBD, ACE2 and RBD K417N, E484K, N501Y

The constructs of native RBD and ACE2 are the same as in Zhou et al., (Zhou et al., 2020). A further construct comprising K417N, E484K, N501Y was generated using PCR, which is exactly the same protein as native RBD except the K417N, E484K and N501Y mutations, as confirmed by sequencing.

### Protein Production

Protein production was as described in Zhou et al. (Zhou et al., 2020).

### Bio-Layer Interferometry

BLI experiments were run on an Octet Red 96e machine (Fortebio). To measure the binding affinities of monoclonal antibodies and ACE2 with native RBD and RBD K417N, E484K, N501Y, each RBD was immobilized onto an AR2G biosensor (Fortebio). Monoclonal antibodies were used as analytes or serial dilutions of ACE2 were used as analytes. All experiments were run at 30° C. Data were recorded using software Data Acquisition 11.1 (Fortebio) and Data Analysis HT 11.1 (Fortebio) with a 1:1 fitting model used for analysis.

### Example 26. Mutational Changes in P.1

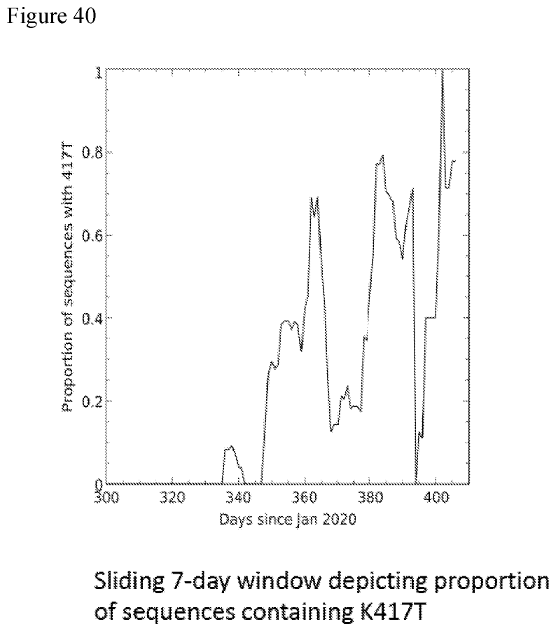

P.1 was first reported in December 2020 from Manaus in Amazonas province of Northern Brazil (Faria et al., 2021). A large first wave of infection was seen in Manaus in March-June 2020 and by October around 75% of individuals from the region are estimated to have been infected, representing a very high attack rate. A second large wave of infection began in December 2020 leading to further hospitalizations. This second wave corresponded with the rapid emergence of P.1, not seen before December when it was found in 52% of cases, rising to 85% by January 2021 (FIG. 40).

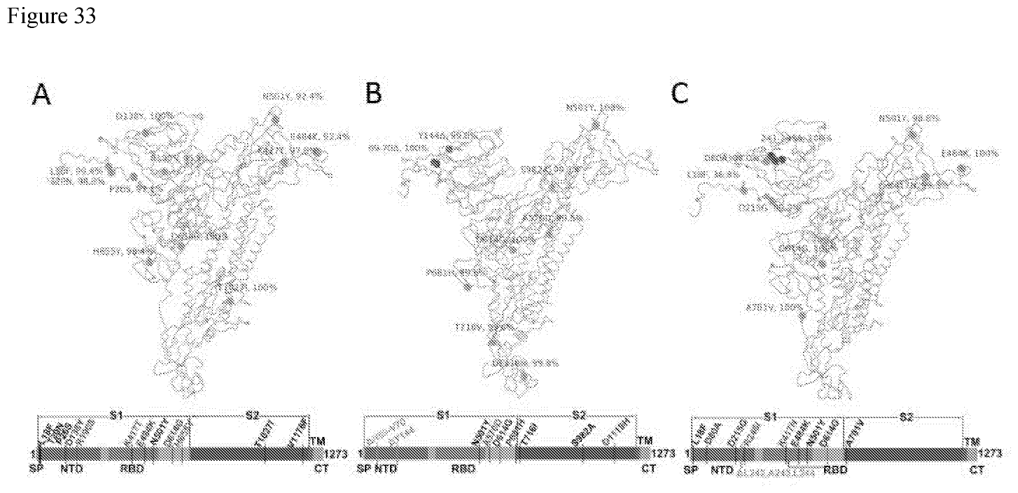

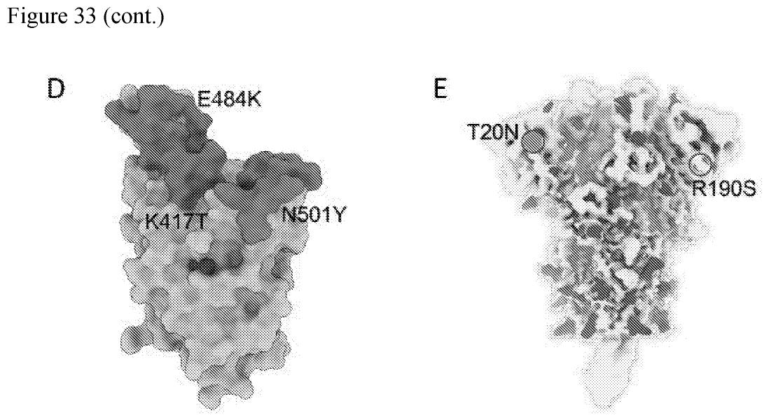

P.1 contains multiple changes compared to B.1.1.28 and P.2 which had been previously circulating in Brazil (Faria et al., 2021). Compared to the Wuhan sequence P.1 contains the following mutations: L18F, T20N, P26S, D138Y, R190S in the NTD, K417T, E484K, N501Y in the RBD, D614G and H655Y at the C-terminus of S1 and T1027I, V1176F in S2. The position of the changes seen in P.1 compared with those found in B.1.1.7 and B.1.351 together with a representation on where they occur on the full spike protein and RBD are shown in FIG. 33. Mutations K417T, E484K, N501Y in the ACE2 interacting surface are of the greatest concern because of their potential to promote escape from the neutralizing antibody response which predominately targets this region (FIG. 33D). The COVID-19 genomics UK (COG-UK) (Tatusov et al., 2000) and the global initiative on sharing avian influenza data (GISAID) (https://www.gisaid.org) databases were searched. A small number of sequences including the K417T mutation, inclusive of the P.1 lineage, have been observed in sequencing from Japan, France, Belgium, Italy, the Netherlands and Colombia (FIG. 40).

It is noteworthy that P.1, B.1.1.7 and B.1.351 have accrued multiple mutations in the NTD, in B.1.1.7 there are two deletions Δ69-70 and Δ 144, in B.1.351 four amino acid changes and the Δ242-244 deletion, while in P.1 there are 6 amino acid changes in the NTD but no deletions. Of note, two of the NTD changes in P.1 introduce N-linked glycosylation sequons T20N (TRT to NRT) and R190S (NLR to NLS, FIG. 33E). The NTD, in the absence of these changes, reasonably well populated with glycosylation sites, indeed it has been suggested that a single bare patch surrounded by N-linked glycans attached at N17, N74, N122, and N149 defines a ‘supersite’ limiting where neutralizing antibodies can attach to the NTD (Cerutti et al., 2021). Residue 188 is somewhat occluded whereas residue 20 is highly exposed, is close to the site of attachment of neutralizing antibody 159 and impinges on the proposed NTD supersite.

### Example 27. The Effects of RBD Mutations on ACE2 Affinity

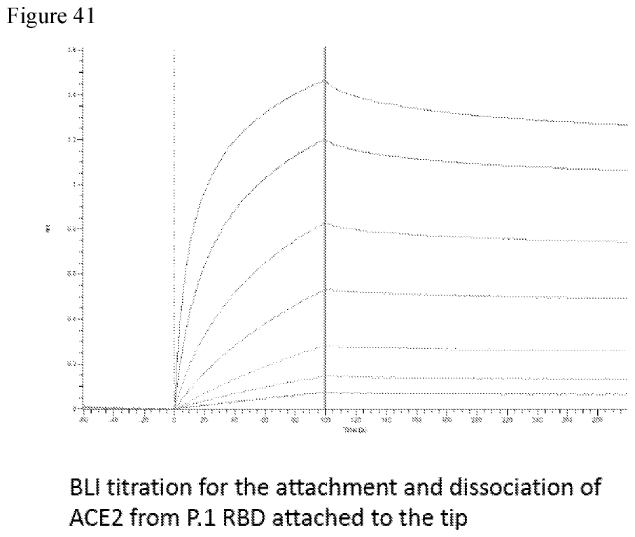

The affinity of RBD/ACE2 interaction for Wuhan, B.1.1.7 (N501Y) and B.1.351 (K417N, E484K, N501Y) RBDs is measured in earlier examples. N501Y increased affinity 7-fold and the combination of 417, 484 and 501 mutations further increased affinity (19-fold compared to Wuhan). Here, the P.1 RBD (K417T, E484K, N501Y) was expressed. The K_{D} for the P.1/ACE2 interaction is 4.8 nM with Kon=1.08E5/Ms, Koff=5.18E-4/s (FIG. 41, Methods), showing that binding to P.1 is essentially indistinguishable from B.1.351 (4.0 nM).

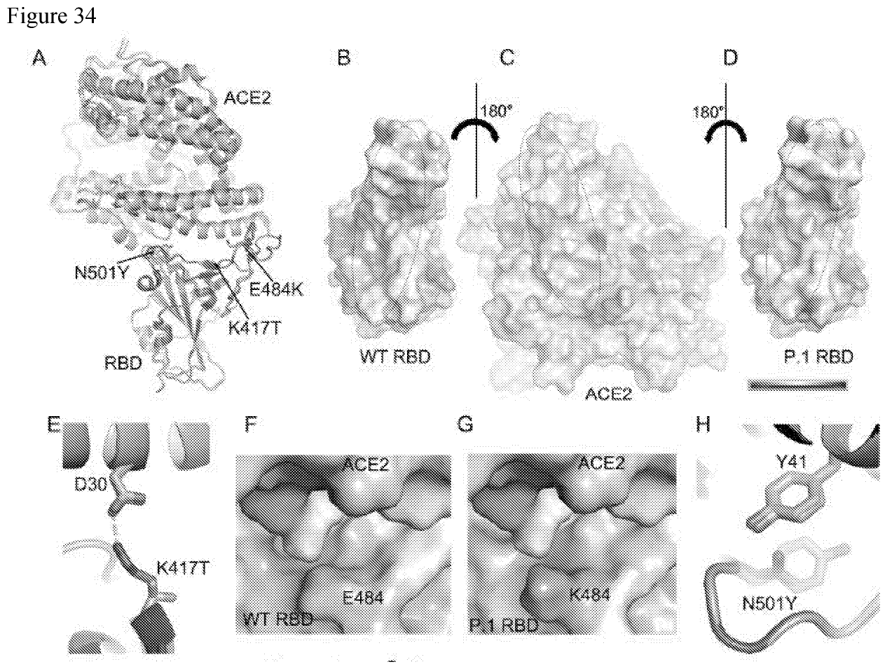

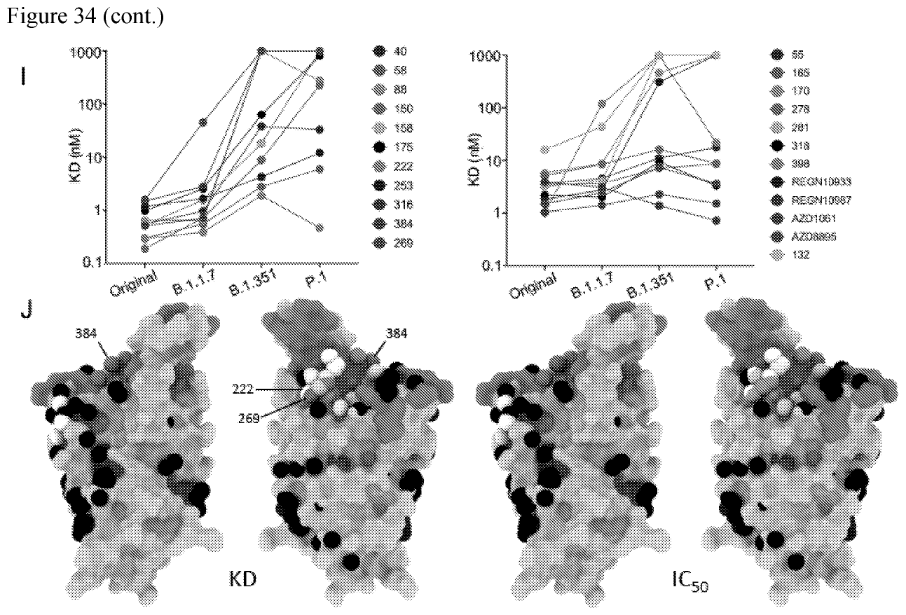

To better understand RBD-ACE2 interactions, the crystal structure of the RBD-ACE2 complex was determined at 3.1 Å resolution (Example 35, Table 17). The mode of RBD-ACE2 engagement is essentially identical for P.1 and the original Wuhan RBD sequence (FIG. 34A). The RMS deviation between the 791 Ca positions is 0.4 Å, similar to the experimental error in the coordinates, and the local structure around each of the three mutations is conserved. Nevertheless, calculation of the electrostatic potential of the contact surfaces reveals a marked change, with much greater complementarity for the P.1 RBD consistent with higher affinity. (FIG. 34B,C,D).

Residue 417 lies at the back of the RBD neck and in the original SARS-CoV-2 is a lysine residue which forms a salt-bridge with D30 of ACE2 (FIG. 34E). The threonine of P.1 RBD no longer forms this interaction and the gap created is open to solvent, so there is no obvious reason why the mutation would increase affinity for ACE2, and this is consistent with directed evolution studies (Zahradnik et al., 2021) where this mutation was rarely selected in RBDs with increased affinity for ACE2.

Residue 484 lies atop the left shoulder of the RBD and neither the original Glu nor the Lys of P1 make significant contact with ACE2, nevertheless the marked change in charge substantially improves the electrostatic complementarity (FIG. 34F,G), consistent with increased affinity.

Residue 501 lies on the right shoulder of the RBD and the change from a relatively short Asn sidechain to the large aromatic Tyr allows for favourable ring stacking interactions consistent with increased affinity (FIG. 34H).

### Example 28. Binding and Neutralisation of P.1 RBD by Potent Human Monoclonal Antibodies

From the panel of 20 potent antibodies which have focus reduction neutralization 50% (FRNT₅₀) values <100 ng/ml, 19 of these mAbs have an epitope on the RBD and all of these block ACE2/RBD interaction, whilst mAb 159 binds the NTD. Biolayer interferometry (BLI) was used to measure the affinity of the RBD-binding antibodies and found that compared to Victoria (SARS-CoV-2/human/AUS/VIC01/2020), an early isolate of SARS-CoV-2, which has a single change S247R in S compared to the Wuhan strain (Seemann et al., 2020; Caly et al., 2020). Monoclonal antibody binding was significantly impacted with a number showing complete knock-out of activity (FIG. 34I). The results with P.1 showed a greater impact compared to B.1.1.7 but similar to B.1.351 (Zhou et al., 2021), this is expected since both contain mutation of the same 3 residues in the RBD, only differing at position 417, K417N in B.1.351 and K417T in P.1. The localization of the impact on binding is shown in FIG. 34J and reflects direct interaction with mutated residues. Of note is mAb 222 which maintains binding potency across all variants despite adjacency to mutated residues, as discussed in the below examples.

### Example 29. Neutralization of P.1 by Potent Human Monoclonal Antibodies

Using the same set of 20 potent antibodies, neutralization was measured by a focus reduction neutralization test (FRNT) and compared with neutralization of Victoria and variants B.1.1.7 and B.1.351. Compared to Victoria neutralization by the monoclonal antibodies was significantly impacted by P.1, with 12/20 showing >10-fold reduction in FRNT₅₀ titre and a number showing complete knock out of activity (FIG. 35; Table 18). The results with P.1 showed a greater impact compared to B.1.1.7 but were similar to those with B.1.351 (Zhou et al., 2021). There is good correlation between the negative impact on neutralization and on RBD-affinity (FIG. 34J).

### Example 30. Reduced Neutralization of P.1 by Monoclonal Antibodies being Developed for Clinical Use

A number of potent neutralizing antibodies are being developed for clinical use either therapeutically of prophylactically (Ku et al., 2021; Baum et al., 2020; Kemp et al., 2021). Neutralization assays were performed against P.1 using antibodies 5309 Vir (Pinto et al., 2020), AZD8895 and AZD1061 AstraZeneca, REGN10987 and REGN10933 Regeneron, LY-CoV555 and LY-CoV16 Lilly and ADG10, ADG20 and ADG30 from Adagio (FIG. 35B). The affinity of binding to P.1 RBD was also investigated by BLI for the Regeneron and AstraZeneca antibodies and the results (FIG. 34I) parallel closely the neutralization results. Neutralization of both Lilly antibodies was severely impacted with LY-CoV16 and LY-CoV555 showing almost complete loss of neutralization of P.1 and B.1.351 while LY-CoV16 also showed marked reduction in neutralization of B.1.1.7. There was also escape from neutralization of P.1 by REGN10933 and a modest reduction in neutralization of P.1 by AZD8895. The three Adagio antibodies neutralized all variants with all reaching a plateau at 100% neutralization and ADG30 showed a slight increase of neutralization of P.1. S309 Vir was largely unaffected although for several viruses, including P.1, the antibody failed to completely neutralize, conceivably reflecting incomplete glycosylation at N343, since the sugar interaction is key to binding of this antibody N343 (Pinto et al., 2020). The escape from REGN10933 and LY-CoV555 mirrors that of other potent antibodies (including antibodies 316 and 384) which make strong interactions with residues 484-486 and are severely compromised by the marked change E484K, whereas LY-CoV016, an IGHV3-53 mAb, is affected by changes at 417 and 501. The abrogation of the Lilly Ly-CoV-16 and LyCoV-555 antibodies reflects the observation of Starr et al. (Starr et al., 2021) (Greaney et al., 2021) that LY-CoV555 is sensitive to mutation at residue 384 and LY-CoV16 is sensitive to changes at 417.

### Example 31. Reduced Neutralization by an NTD-Binding Antibody

The neutralization titre of NTD-binding mAb159, was 133-fold reduced on P.1 compared to Victoria with only 64% neutralization at 10 μg/ml (FIG. 35A). Although P.1 does not harbour deletions in the NTD like B.1.1.7 (469-70, 4144) or B.1.351 (4242-244), it is clear that the 6 NTD mutations in P.1 (L18F, T20N, P26S, D138Y, R190S) disrupt the epitope for mAb159 (FIG. 36A). It is possible that the failure of this antibody to achieve complete neutralization could be due to partial glycosylation at residue 20, which is some 16 Å from bound Fab 159, however the L18F mutation is even closer and likely to diminish affinity (FIG. 36A). Since it has been proposed that there is a single supersite for potent NTD binding antibodies, the binding of many of these is expected to be affected (Cerutti et al., 2021).

### Example 32. Reduced Neutralization by VH3-53 Public Antibodies

Five of the potent monoclonal antibodies used herein (150, 158, 175, 222 and 269), belong to the VH3-53 family and a further 2 (out of 5 of this family) belong to the almost identical VH3-66, and the following discussion applies also to these antibodies. The binding sites for these have been described in the earlier examples. The large majority of these antibodies attach to the RBD in a very similar fashion. These motifs recur widely, VH3-53 are the most prevalent deposited sequences and structures for SARS-CoV-2 neutralizing antibodies. Their engagement with the RBD is dictated by CDR-H1 (SEQ ID NOs: 449, 452, 455, 458 and 461) and CDR-H2 (SEQ ID NOs: 450, 453, 456, 459 and 462) whilst the CDR-H3 (SEQ ID NOs: 451,454,457,460 and 463) is characteristically short and makes rather few interactions (Yuan et al., 2020; Barnes et al., 2020). The structures of mAbs 150, 158 and 269 have been solved (FIG. 36B) which show that whilst there are no contacts with residue 484, there are interactions of CDR-H3 with K417 and CDR-L1 with N501, meaning that binding and neutralization by VH3-53 antibodies would be predicted to be compromised by the N501Y change in variant viruses B.1.1.7, B.1.351 and P.1, whilst the additional change at 417 in P.1 (K417T) and B.1.351 (K417N) might be expected to have an additive effect.

Neutralization of P.1 by 175 and 158 is severely impacted and neutralization of P.1 by 269 is almost completely lost. However, for 150 P.1 neutralization is less compromised than for B.1.351 (Zhou et al., 2021), whilst for 222 neutralization is completely unaffected by the changes in P.1 and indeed all variants (FIG. 35A).

The affinity of 222 was measured for both P.1 (KD=1.92±0.01 nM) and Wuhan RBD (KD=1.36±0.08 nM) showing no reduction in the strength of interaction despite the changes occurring in the putative binding site for P.1 (Table 18).

To understand how 222 is able to still neutralize P.1, the crystal structures of six ternary complexes of 222 in complex with the RBDs was solved for (i) the original virus, and bearing mutations (ii) K417N; (iii) K417T; (iv) N501Y; the 417, 484 and 501 changes characteristic of B.1.351 (v) and P.1 (vi). All crystals also contained a further Fab, EY6A as a crystallization chaperone (Zhou et al., 2020), were isomorphous and the resolution of the structures ranged from 1.95 to 2.67 Å, FIG. 36C,D, Example Table 17. As expected, the structures are highly similar with the binding pose of 222 being essentially identical in all structures (pairwise RMSD in Ca atoms between pairs of structures are ˜0.2-0.3 Å for all residues in the RBD and Fv region of mAb 222, FIG. 36D).

In the original virus residue 417 makes a weak salt bridge interaction with heavy chain CDR3 residue E99. Mutation to either Asn or Thr abolishes this and there is little direct interaction, although there are weak (˜3.5 Å) contacts to heavy chain Y52 and light chain Y92 (FIG. 36E). However, a buffer molecule/ion rearranges to form bridging interactions and this may mitigate the loss of the salt bridge, in addition the original salt bridge is weak and its contribution to binding may be offset by the loss of entropy in the lysine sidechain. CDR-H3 of 222 (SEQ ID NO: 457), at 13 residues is slightly longer than found in the majority of potent VH3-53 antibodies, however this seems unlikely to be responsible for the resilience of 222, rather it seems that there is little binding energy in general from the CDR3-H3, since most of the binding energy contribution of the heavy chain comes from CDR-H1 (SEQ ID NO: 455) and CDR-H2 (SEQ ID NO: 456) which do not interact with RBD residue 417, meaning that many VH3-53 antibodies are likely to be resilient to the common N/T mutations (FIG. 36B).

Residue 501 makes contact with CDR-L1 of mAb 222 (SEQ ID NO: 468) (FIG. 36D,F), however the interaction, with P30 is probably slightly strengthened by the N501Y mutation which provides a stacking interaction with the proline, conferring resilience. This is in contrast to the situation with most other VH3-53 antibodies where direct contacts confer susceptibility to escape by mutation to Tyr (FIGS. 34I,J and 35A).

### Example 33. The 222 Light Chain can Rescue Neutralization by Other VH3-53 mAbs

Reasoning that the relative robustness of mAb 222 to common variants (P.1, B.1.1.7 and B.1.351) compared to other VH3-53 antibodies stems from the choice of light chain we modelled the 222LC with the heavy chains of other VH3-53 antibodies to see if they might be compatible (FIG. 36G). Unexpectedly, it appeared that there would likely be no serious steric clashes. This contrasted with the numerous clashes seen when we docked the light chains of other VH3-53 antibodies onto the heavy chain of 222 (FIG. 36G,H). This suggests that the 222 light chain might be an almost universal light chain for these 3-53 antibodies and could confer resilience to P.1, B.1.1.7 and B.1.351 variants. This led us to create chimeric antibodies containing the 222LC combined with the HC of the other VH3-53 mAbs 150, 158, 175 and 269. In all cases, chimeric antibodies expressed well and neutralization assays were performed against Victoria, B.1.1.7, B.1.351 and P.1 viruses (FIG. 37). For B.1.1.7 neutralization of 150HC/222LC, 158HC/222LC and 269HC/222LC was restored to near the level seen on Victoria, whilst 175HC/222LC could not fully neutralize B.1.1.7. For B.1.351 and P.1 the activity of mAbs 150 and 158 was restored in chimeras containing the 222LC, with the 150HC/222LC showing 50-fold greater potency against B.1.351 (Ing vs 350 ng/ml) and 13-fold greater potency against P.1 (3 ng vs 40 ng/ml) than native 150. With an FRNT₅₀ of 3 ng/ml 150HC/222LC was the most potent antibody tested against P.1.

A number of public antibody responses (antibodies derived from public v-genes) have been reported for SARS-CoV-2, principal amongst these being VH3-53/VH3-66 and VH1-58 (Yuan et al., 2020; Barnes et al., 2020). Mixing heavy and light chains from antibodies within VH1-58 can increase the neutralization titre by 20-fold from the parent antibodies (chimera of 253HC with 55LC or 165LC). Here it is shown that chimeras created amongst the VH3-53 antibodies using the 222LC are able to confer broad neutralization to antibodies which have reduced neutralization capacity against the viral variants. Furthermore, the chimera of 150HC with 222LC achieved 13 and 3-fold increases in neutralization titre compared to the parental 150 and 222 mAb respectively. Due to the similarities between VH3-53 and VH3-66, chimeras between heavy chain and light chains of such antibodies are also expected to lead to an increase in neutralisation titres in a similar fashion. Creation of such antibody chimeras amongst other anti-SARS-CoV2 antibodies may similarly lead to the discovery of more antibodies with enhanced activity.

### Example 34. Neutralization of P.1 by Convalescent Plasma and Vaccine Serum

As described in earlier examples, convalescent plasma samples were collected from a cohort of volunteers who had suffered from SARS-CoV-2 infection evidenced by a positive diagnostic PCR. Samples were collected during the convalescent phase, 4-9 weeks following infection, all samples were taken during the first wave of infection in the UK, prior June 2020 and well before the emergence of the B.1.1.7 variant. Plasma was also collected from volunteers recently infected with B.1.1.7 as demonstrated by viral sequencing or S gene drop out from the diagnostic PCR.

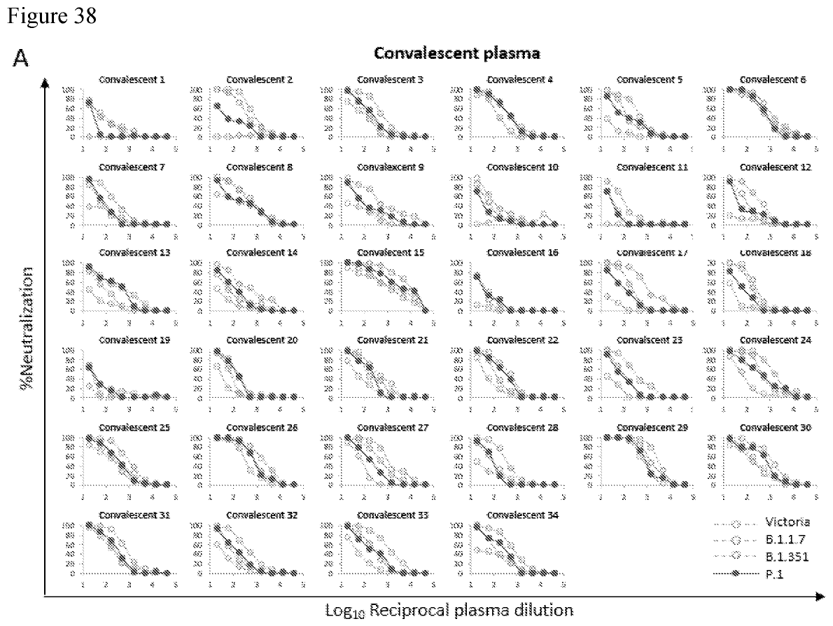

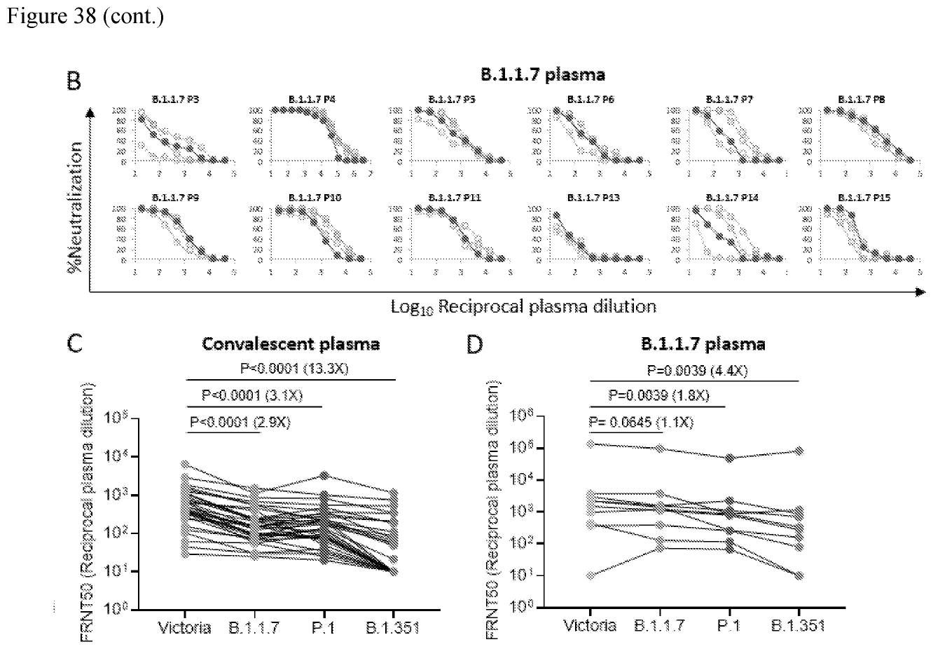

Neutralization of P.1 was assessed by FRNT on 34 convalescent samples (FIG. 38A; Table 19A). P.1 neutralization curves are displayed alongside neutralization curves for Victoria, together with B.1.1.7 and B.1.351. P.1 geometric mean neutralization titres were reduced 3.1-fold compared to Victoria (p<0.0001). This reduction was similar to B.1.1.7 (2.9-fold) and considerably less than B.1.351 (13.3-fold) (FIG. 38C). When using plasma from individuals infected with B.1.1.7 we saw only modest (1.8-fold p=0.0039) reductions in neutralization comparing P.1 with Victoria (FIGS. 38B and D Table 19B).

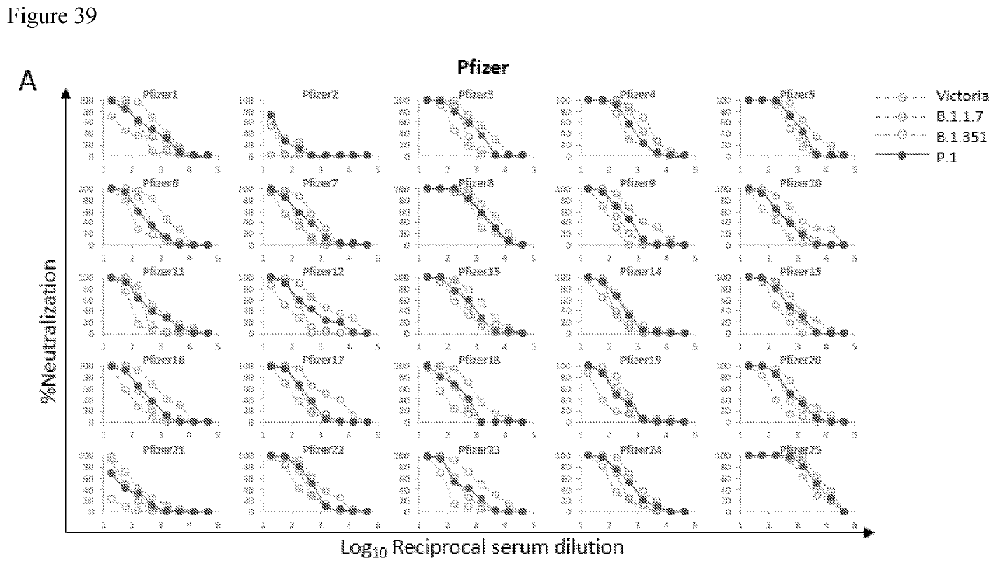

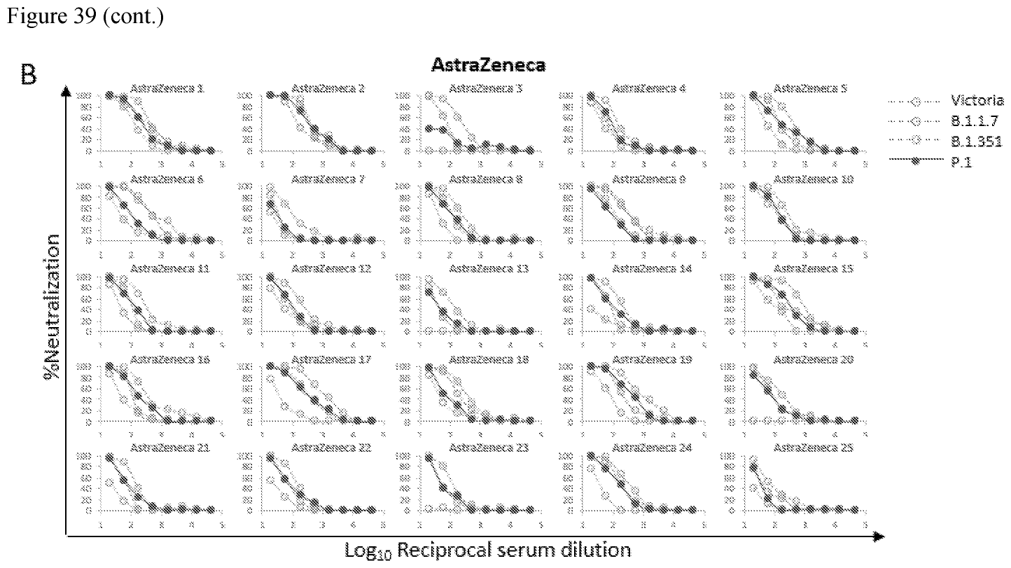

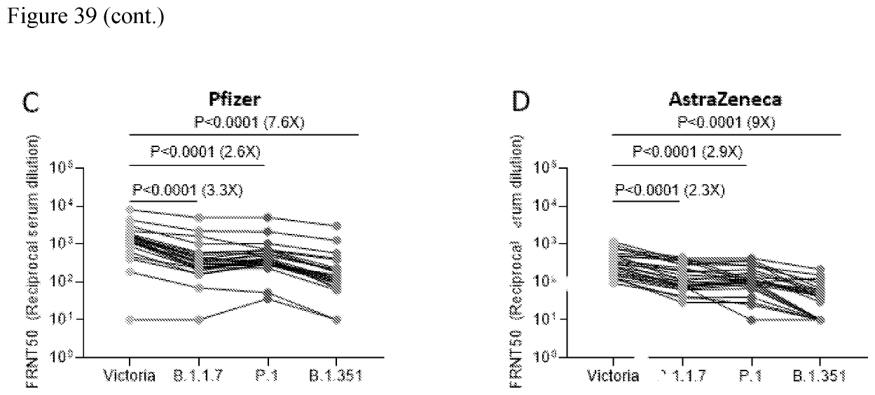

Neutralization assays were next performed using serum collected from individuals who had received either the BNT162b2 Pfizer-BioNTech or ChAdOx1 nCoV-19 Oxford-AstraZeneca vaccine FIG. 39. For the Pfizer BioNTech vaccine serum was collected 4-14 days following the second dose of vaccine administered three weeks after the first dose (n=25). For the Oxford-AstraZeneca vaccine serum was taken 14 or 28 days following the second dose which was administered 8-14 weeks following the first dose (N=25). Geometric mean neutralization titres against P.1 were reduced 2.6-fold (p<0.0001) relative to the Victoria virus for the Pfizer-BioNTech vaccine serum FIGS. 39A,C and 2.9-fold (P<0.0001) for the Oxford-AstraZeneca vaccine FIG. 39B,D Table 20.

Neutralization titres against P.1 were similar to those against B.1.1.7 and only a minority of samples failed to reach 100% neutralization at 1:20 dilution of serum, considerably better than neutralization of B.1.351, where titres were reduced 7.6-fold and 9-fold for the BNT162b2 Pfizer and ChAdOx1 nCoV-19 AstraZeneca vaccines respectively.

The reason for the differences in neutralization of B.1.351 and P.1 by immune serum are not immediately clear, but may reflect the difference in the mutations introduced outside the RBD. In addition to mAb 159, a number of potent neutralizing mAbs have been reported that map to the NTD (Cerutti et al., 2021), and this domain has multiple mutations in all three major variant strains: B.1.1.7 has two deletions, B.1.351 has a deletion and four substitutions and P.1 has 6 amino acid substitutions, including the creation of two N-linked glycan sequons (FIG. 33A-C). Comparison of neutralization of pseudoviruses expressing only the three RBD mutations (K417N E484K N501Y) of B.1.351 with pseudovirus expressing the full suite of mutations in B.1.351 spike show that the non-RBD changes substantially increase escape from neutralization (Wibmer et al., 2021; Wang et al., 2021). The changes in the NTD of the major variants are far less consistent than those found in the RBD, and there are no strong trends in electrostatic properties (FIG. 33A-C). It therefore remains unclear what the drivers are for these changes, although one or more of immune escape, co-receptor binding, and modulation of RBD dynamics affecting presentation of the receptor binding site are plausible. Nonetheless, it seems likely that these changes are largely responsible for the non-RBD component of neutralization variation between strains.

### Example 35. Materials and Methods for Examples 26 to 34

### Viral Stocks

SARS-CoV-2/human/AUS/VIC01/2020 (Caly et al., 2020), SARS-CoV-2/B.1.1.7 and SARS-CoV-2/B.1.351 were provided by Public Health England, P.1 from a throat swab from Brazil were grown in Vero (ATCC CCL-81) cells. Cells were infected with the SARS-CoV-2 virus using an MOI of 0.0001. Virus containing supernatant was harvested at 80% CPE and spun at 3000 rpm at 4° C. before storage at −80° C. Viral titres were determined by a focus-forming assay on Vero cells. Victoria passage 5, B.1.1.7 passage 2 and B.1.351 passage 4 stocks were sequenced to verify that they contained the expected spike protein sequence and no changes to the furin cleavage sites. The P.1 virus used in these studies contained the following mutations: L18F, T20N, P26S, D138Y, R190S, K417T, E464K, N501Y, D614G, H655Y, T1027I, V1176F. Passage 1 P.1 virus was sequence confirmed and contained no changes to the furin cleavage site.

### Bacterial Strains and Cell Culture

Vero (ATCC CCL-81) cells were cultured at 37° C. in Dulbecco's Modified Eagle medium (DMEM) high glucose (Sigma-Aldrich) supplemented with 10% fetal bovine serum (FBS), 2 mM GlutaMAX (Gibco, 35050061) and 100 U/ml of penicillin-streptomycin. Human mAbs were expressed in HEK293T cells cultured in UltraDOMA PF Protein-free Medium (Cat #12-727F, LONZA) at 37° C. with 5% CO₂. E. coli DH5α bacteria were used for transformation of plasmids encoding wt and mutated RBD proteins. A single colony was picked and cultured in LB broth with 50 μg mL⁻¹ Kanamycin at 37° C. at 200 rpm in a shaker overnight. HEK293T (ATCC CRL-11268) cells were cultured in DMEM high glucose (Sigma-Aldrich) supplemented with 10% FBS, 1% 100× Mem Neaa (Gibco) and 1% 100× L-Glutamine (Gibco) at 37° C. with 5% CO₂. To express RBD, RBD K417T, E484K, N501Y, RBD K417N, RBD K417T, RBD E484K and ACE2, HEK293T cells were cultured in DMEM high glucose (Sigma) supplemented with 2% FBS, 1% 100× Mem Neaa and 1% 100× L-Glutamine at 37° C. for transfection.

### Participants

Participants were recruited through three studies: Sepsis Immunomics [Oxford REC C, reference:19/SC/0296]), ISARIC/WHO Clinical Characterisation Protocol for Severe Emerging Infections [Oxford REC C, reference 13/SC/0149] and the Gastrointestinal illness in Oxford: COVID sub study [Sheffield REC, reference: 16/YH/0247]. Diagnosis was confirmed through reporting of symptoms consistent with COVID-19 and a test positive for SARS-CoV-2 using reverse transcriptase polymerase chain reaction (RT-PCR) from an upper respiratory tract (nose/throat) swab tested in accredited laboratories. A blood sample was taken following consent at least 14 days after symptom onset. Clinical information including severity of disease (mild, severe or critical infection according to recommendations from the World Health Organisation) and times between symptom onset and sampling and age of participant was captured for all individuals at the time of sampling.

P.1 virus from throat swabs. The International Reference Laboratory for Coronavirus at FIOCRUZ (WHO) as part of the national surveillance for coronavirus had the approval of the FIOCRUZ ethical committee (CEP 4.128.241) to continuously receive and analyze samples of COVID-19 suspected cases for virological surveillance. Clinical samples (throat swabs) containing P.1 were shared with Oxford University, UK under the MTA IOC FIOCRUZ 21-02.

Sera from Pfizer Vaccinees

Pfizer vaccine serum was obtained 7-17 days following the second dose of the BNT162b2 vaccine. Vaccinees were Health Care Workers, based at Oxford University Hospitals NHS Foundation Trust, not known to have prior infection with SARS-CoV-2 and were enrolled in the OPTIC Study as part of the Oxford Translational Gastrointestinal Unit GI Biobank Study 16/YH/0247 [research ethics committee (REC) at Yorkshire & The Humber-Sheffield]. The study was conducted according to the principles of the Declaration of Helsinki (2008) and the International Conference on Harmonization (ICH) Good Clinical Practice (GCP) guidelines. Written informed consent was obtained for all patients enrolled in the study. Each received two doses of COVID-19 mRNA Vaccine BNT162b2.30 micrograms, administered intramuscularly after dilution as a series of two doses (0.3 mL each) 18-28 days apart. The mean age of vaccines was 43 years (range 25-63), 11 male and 14 female.

### AstraZeneca-Oxford Vaccine Study Procedures and Sample Processing

Full details of the randomized controlled trial of ChAdOx1 nCoV-19 (AZD1222), were previously published (PMID: 33220855/PMID: 32702298). These studies were registered at ISRCTN (U.S. Pat. Nos. 15,281,137 and 89,951,424) and ClinicalTrials.gov (NCT04324606 and NCT04400838). Written informed consent was obtained from all participants, and the trial is being done in accordance with the principles of the Declaration of Helsinki and Good Clinical Practice. The studies were sponsored by the University of Oxford (Oxford, UK) and approval obtained from a national ethics committee (South Central Berkshire Research Ethics Committee, reference 20/SC/0145 and 20/SC/0179) and a regulatory agency in the United Kingdom (the Medicines and Healthcare Products Regulatory Agency). An independent DSMB reviewed all interim safety reports. A copy of the protocols was included in previous publications (PMID: 33220855/PMID: 32702298).

Data from vaccinated volunteers who received two vaccinations are included in this paper. Vaccine doses were either 5×10 10 viral particles (standard dose; SD/SD cohort n=21) or half dose as their first dose (low dose) and a standard dose as their second dose (LD/SD cohort n=4). The interval between first and second dose was in the range of 8-14 weeks. Blood samples were collected and serum separated on the day of vaccination and on pre-specified days after vaccination e.g. 14 and 28 days after boost.

### Focus Reduction Neutralization Assay (FRNT)

The neutralization potential of Ab was measured using a Focus Reduction Neutralization Test (FRNT), where the reduction in the number of the infected foci is compared to a negative control well without antibody. Briefly, serially diluted Ab or plasma was mixed with SARS-CoV-2 strain Victoria or P.1 and incubated for 1 hr at 37° C. The mixtures were then transferred to 96-well, cell culture-treated, flat-bottom microplates containing confluent Vero cell monolayers in duplicate and incubated for a further 2 hrs followed by the addition of 1.5% semi-solid carboxymethyl cellulose (CMC) overlay medium to each well to limit virus diffusion. A focus forming assay was then performed by staining Vero cells with human anti-NP mAb (mAb206) followed by peroxidase-conjugated goat anti-human IgG (A0170; Sigma). Finally, the foci (infected cells) approximately 100 per well in the absence of antibodies, were visualized by adding TrueBlue Peroxidase Substrate. Virus-infected cell foci were counted on the classic AID EliSpot reader using AID ELISpot software. The percentage of focus reduction was calculated and IC50 was determined using the probit program from the SPSS package.

### Cloning of ACE2 and RBD Proteins

The constructs of EY6A Fab, 222 Fab, ACE2, WT RBD, B.1.1.7 and B.1.351 mutant RBD are the same as described in earlier examples. To clone RBD K417T and RBD K417N, primers of RBD K417T (forward primer 5′-GGGCAGACCGGCACGATCGCCGACTAC-3′ (SEQ ID NO: 424) and reverse primer5′-GTAGTCGGCGATCGTGCCGGTCTGCCC (SEQ ID NO: 425)) and primers of RBD K417N (forward primer 5′-CAGGGCAGACCGGCAATATCGCCGACTACAATTAC-3′ (SEQ ID: 426) and reverse primer 5′-GTAATTGTAGTCGGCGATATTGCCGGTCTGCCCTG-3′ (SEQ ID NO: 427)) were used separately, together with two primers of pNEO vector (Forward primer 5′-CAGCTCCTGGGCAACGTGCT-3′ (SEQ ID NO: 422) and reverse primer 5′-CGTAAAAGGAGCAACATAG-3′ (SEQ ID NO: 423)) to do PCR, with the plasmid of WT RBD as the template. To clone P.1 RBD, the construct of B.1.351 RBD was used as the template and the primers of RBD K417T and of pNEO vector mentioned above were used to do PCR. Amplified DNA fragments were digested with restriction enzymes AgeI and KpnI and then ligated with digested pNEO vector. All constructs were verified by sequencing.

### Protein Production

Protein production was as described in Zhou et al. (Zhou et al., 2020). Briefly, plasmids encoding proteins were transiently expressed in HEK293T (ATCC CRL-11268) cells. The conditioned medium was dialysed and purified with a 5-ml HisTrap nickel column (GE Healthcare) and further polished using a Superdex 75 HiLoad 16/60 gel filtration column (GE Healthcare).

### Bio-Layer Interferometry

BLI experiments were run on an Octet Red 96e machine (Fortebio). To measure the binding affinity of ACE2 with P.1 RBD and affinities of monoclonal antibodies and ACE2 with native RBD and, RBD K417N, RBD K417T, RBD E484K and RBD K417T E484K N501Y, each P.1 RBD, each RBD was immobilized onto an AR2Gbiosensor (Fortebio). Monoclonal antibodies were used as analytes or serial dilutions of ACE2 were used as analytes. All experiments were run at 30° C. Data were recorded using software Data Acquisition 11.1 (Fortebio) and Data Analysis HT 11.1 (Fortebio) with a 1:1 fitting model used for the analysis.

### Antibody Production

AstraZeneca and Regeneron antibodies were provided by AstraZeneca, Vir, Lilly and Adagio antibodies were provided by Adagio. For the chimeric antibodies heavy and light chains of the indicated antibodies were transiently transfected into 293Y cells and antibody purified from supernatant on protein A.

### Crystallisation

ACE2 was mixed with P.1 RBD in a 1:1 molar ratio to a final concentration of 12.5 mg ml⁻¹. EY6A Fab, 222 Fab and WT or mutant RBD were mixed in a 1:1:1 molar ratio to a final concentration of 7.0 mg ml 1. All samples were incubated at room temperature for 30 min. Most crystallization experiments was set up with a Cartesian Robot in Crystalquick 96-well X plates (Greiner Bio-One) using the nanoliter sitting-drop vapor-diffusion method, with 100 nl of protein plus 100 nl of reservoir in each drop, as previously described (Water et al., 2003). Crystallization of B.1.1.7 RBD/EY6A/222 complex was set up by hand pipetting, with 500 nl of protein plus 500 nl of reservoir in each drop. Good crystals of EY6A Fab and 222 Fab complexed with WT, K417T, K417N, B.1.1.7, B.1.351 or P.1 RBD were all obtained from Hampton Research PEGRx 2 screen, condition 35, containing 0.15 M Lithium sulfate, 0.1 M Citric acid pH 3.5, 18% w/v PEG 6,000. Crystals of P.1 RBD/ACE2 complex were formed in Hampton Research PEGRx 1 screen, condition 38, containing 0.1 M Imidazole pH 7.0.

### X-Ray Data Collection, Structure Determination and Refinement

Crystals of ternary complexes of WT and mutant RBD/EY6A and 222 Fabs were mounted in loops and dipped in solution containing 25% glycerol and 75% mother liquor for a second before being frozen in liquid nitrogen prior to data collection. No cryo-protectant was used for the P.1. RBD/ACE2 crystals. Diffraction data were collected at 100 K at beamline I03 of Diamond Light Source, UK. All data (except some of the WT RBD-EY6A-222 Fab complex images) were collected as part of an automated queue system allowing unattended automated data collection (https://www.diamond.ac.uk/Instruments/Mx/I03403-Manual/Unattended-Data-Collections.html). Diffraction images of 0.1° rotation were recorded on an Eiger2 XE 16M detector (exposure time of either 0.004 or 0.006 s per image, beam size 80×20 μm, 100% beam transmission and wavelength of 0.9763 Å). Data were indexed, integrated and scaled with the automated data processing program Xia2-dials (Winter, 2010; Winter et al., 2018). A data set of 1080° was collected from 3 positions of a frozen crystal for the WT RBD-EY6A-222 Fab complex. 720° of data was collected for each of the B.1.1.7, P.1 and B.1.351 mutant RBD/EY6A and 222 Fab complexes (each from 2 crystals), and 360° for each of the K417N and K417T RBD with EY6A and 222 Fabs, and ACE2 complexes was collected from a single crystal.

Structures of WT RBD-EY6A-222 and the P.1 RBD-ACE2 complexes were determined by molecular replacement with PHASER (McCoy et al., 2007) using search models of SARS-CoV-2 RBD-EY6A-H4 (PDB ID 6ZCZ) (Zhou et al., 2020) and RBD-158 (PDB ID, 7BEK) complexes, and a RBD and ACE2 complex (PDB ID, 6LZG (Wang et al., 2020)), respectively. Model rebuilding with COOT (Emsley and Cowtan, 2004) and refinement with PHENIX (Liebschner et al., 2019) were done for all the structures. The ChCl domains of EY6A are flexible and have poor electron density. Data collection and structure refinement statistics are given in Table 51. Structural comparisons used SHP (Stuart et al., 1979), residues forming the RBD/Fab interface were identified with PISA (Krissinel and Henrick, 2007) and figures were prepared with PyMOL (The PyMOL Molecular Graphics System, Version 1.2r3pre, Schrödinger, LLC).

### Quantification and Statistical Analysis

Statistical analyses are reported in the results and figure legends. Neutralization was measured by FRNT. The percentage of focus reduction was calculated and IC50 was determined using the probit program from the SPSS package. The Wilcoxon matched-pairs signed rank test was used for the analysis and two-tailed P values were calculated and geometric mean values. BLI data were analysed using Data Analysis HT 11.1 (Fortebio) with a 1:1 fitting model.

### Example 36. Cross-Reactivity of mAbs

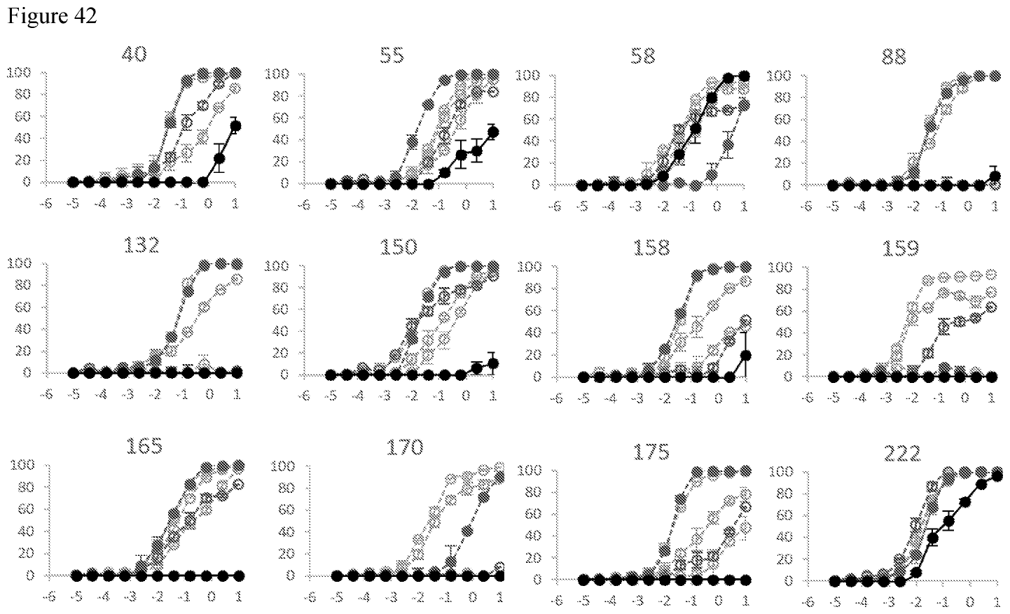

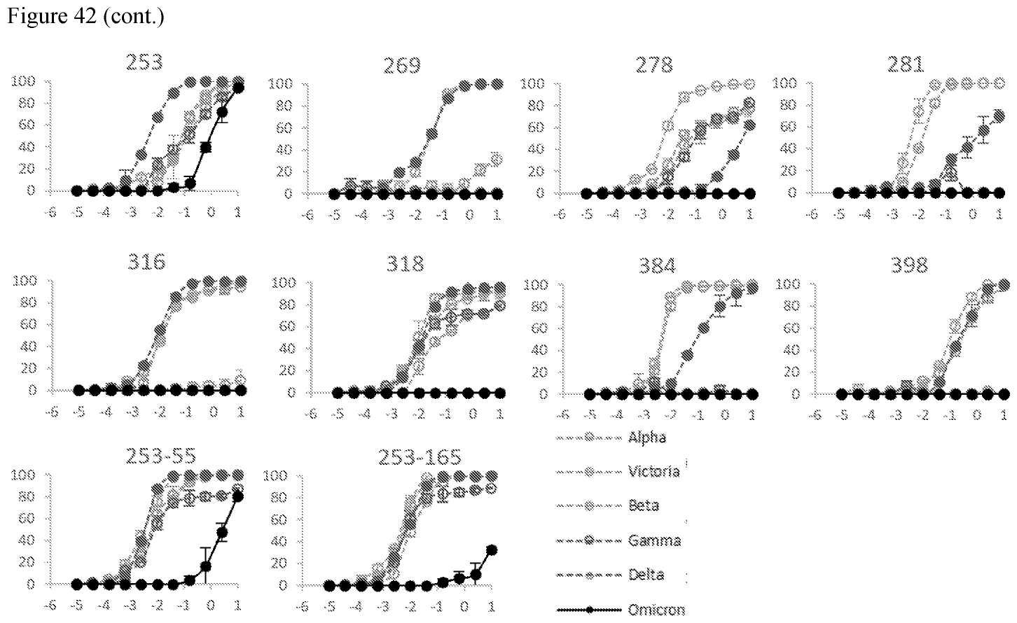

Live virus neutralization assays were performed using the following viruses, containing the indicated changes in the RBD: Victoria, an early Wuhan related strain, Alpha (N501Y), Beta (K417N, E484K, N501Y), Gamma (K417T, E484K, N501Y), Delta (L452R, T478K), and Omicron (G339D, S371L, S373P, S375F, K417N, N440K, G446S, S477N, T478K, E484A, Q493R, G496S, Q498R, N501Y, Y505H)(FIG. 42, Table 26).

Mabs 58, 222, 253, 253H55L, from early pandemic samples, show neutralization of Omicron. In particular, Mabs 58 and 222 retain potent neutralisation of omicron. Mab 222 potently neutralizes all strains tested. Mab 58 potently neutralizes all strains except for Delta.

### Example 37. Further Neutralisation Data of Selected Antibodies Against SARS-CoV-2 Antibodies

Further neutralisation experiments were carried out to determine the neutralisation of SARS-CoV-2 variants by selected antibodies. As discussed in the detailed description above, antibodies derived from the same heavy chain V-gene may swap light chains to result in an antibody comprising the heavy chain variable region of a first antibody and a light chain variable region of a second antibody, and such new antibodies may have improved neutralisation and/or other characteristics when compared to the ‘parent’ antibodies.

Tables 21 to 25 provide examples of such antibodies that may be created by swapping the light chain between antibodies derived from the same heavy chain V-gene.

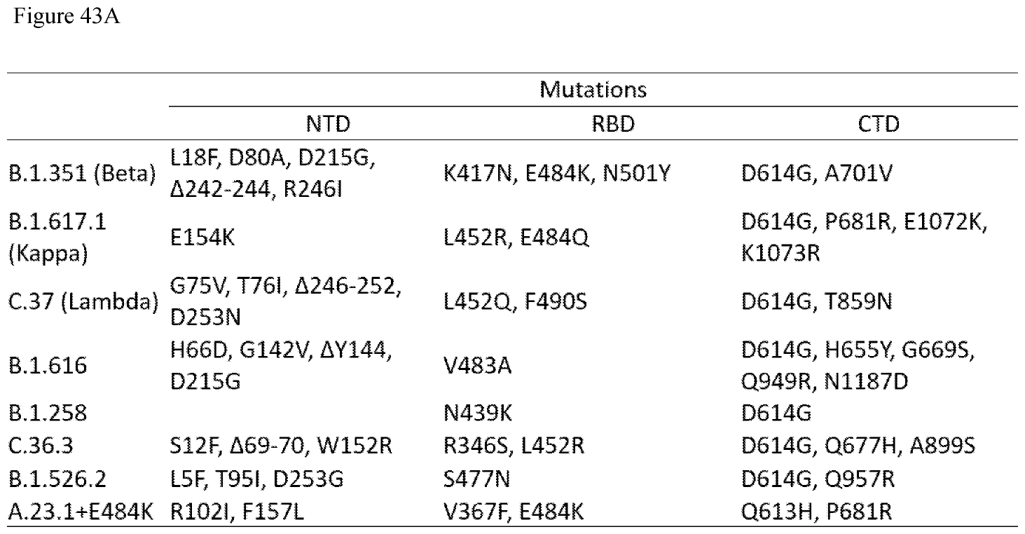

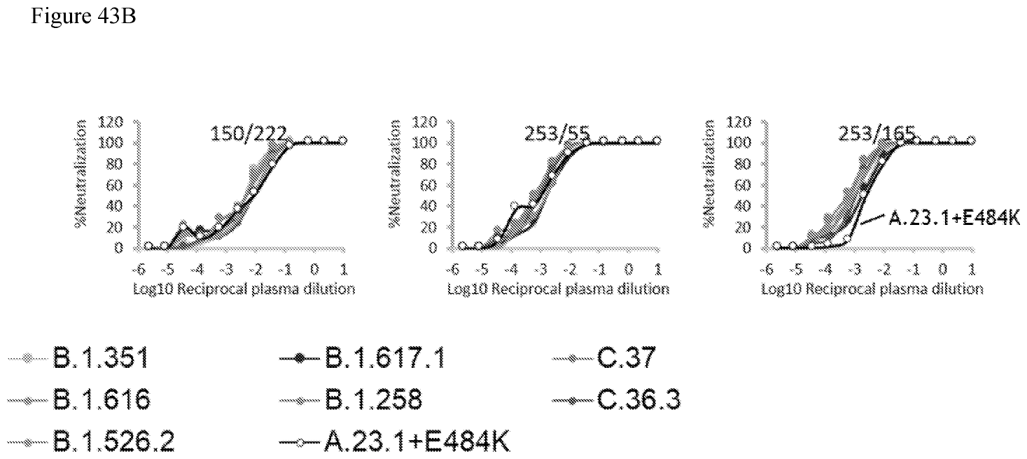

Tables 27 to 28 provide further neutralisation data for selected antibodies and antibodies created by swapping the light chain between antibodies derived from the same heavy chain V-gene. Almost all the antibodies created by swapping the light chain between antibodies derived from the same heavy chain V-gene exhibit improved neutralisation when compared to the ‘parent’ antibodies. The data in FIG. 43A describes the mutations in the NTD, RBD and CTD of the spike protein of SARS-CoV-2 variants when compared with the Wuhan SARS-CoV-2 spike protein sequence. The data in FIG. 43B correspond to the data shown in Table 28.

### Tables

TABLE 1
SEQ ID NOs of selected antibodies

| | Heavy | Heavy | Light | Light | | | | | | |
|---|---|---|---|---|---|---|---|---|---|---|
| | Chain | Chain | Chain | Chain | | | | | | |
| Antibody | protein | nucleotide | protein | nucleotide | | | | | | |
| number | sequence | sequence | sequence | sequence | CDRH1 | CDRH2 | CDRH3 | CDRL1 | CDRL2 | CDRL3 |
| 2 | 2 | 1 | 4 | 3 | 5 | 6 | 7 | 8 | 9 | 10 |
| 22 | 12 | 11 | 14 | 13 | 15 | 16 | 17 | 18 | 19 | 20 |
| 40 | 22 | 21 | 24 | 23 | 25 | 26 | 27 | 28 | 29 | 30 |
| 44 | 32 | 31 | 34 | 33 | 35 | 36 | 37 | 38 | 39 | 5 |
| 45 | 42 | 41 | 44 | 43 | 45 | 46 | 47 | 48 | 49 | 50 |
| 54 | 52 | 51 | 54 | 53 | 55 | 56 | 57 | 58 | 59 | 60 |
| 55 | 62 | 61 | 64 | 63 | 65 | 66 | 67 | 68 | 69 | 70 |
| 58 | 72 | 71 | 74 | 73 | 75 | 76 | 77 | 00 | 79 | 80 |
| 61 | 82 | 81 | 84 | 83 | 85 | 86 | 87 | 88 | 89 | 90 |
| 75 | 92 | 91 | 94 | 93 | 95 | 96 | 9 | 98 | 99 | 100 |
| 88 | 102 | 101 | 104 | 103 | 105 | 106 | 107 | 108 | 109 | 110 |
| 111 | 112 | 111 | 114 | 113 | 115 | 116 | 117 | 118 | 119 | 120 |
| 132 | 122 | 121 | 124 | 123 | 125 | 126 | 127 | 128 | 129 | 130 |
| 140 | 132 | 131 | 134 | 133 | 135 | 136 | 137 | 138 | 139 | 140 |
| 148 | 142 | 141 | 144 | 143 | 145 | 146 | 147 | 148 | 149 | 150 |
| 150 | 152 | 151 | 154 | 153 | 155 | 156 | 157 | 158 | 159 | 160 |
| 158 | 162 | 161 | 164 | 163 | 165 | 166 | 167 | 168 | 169 | 170 |
| 159 | 172 | 171 | 174 | 173 | 175 | 176 | 177 | 178 | 179 | 180 |
| 165 | 182 | 181 | 184 | 183 | 185 | 186 | 187 | 188 | 189 | 190 |
| 170 | 192 | 191 | 194 | 193 | 195 | 196 | 197 | 198 | 199 | 200 |
| 175 | 202 | 201 | 204 | 203 | 205 | 206 | 207 | 208 | 209 | 210 |
| 177 | 212 | 211 | 214 | 213 | 215 | 216 | 217 | 218 | 219 | 220 |
| 181 | 222 | 221 | 224 | 223 | 225 | 226 | 227 | 228 | 229 | 230 |
| 182 | 232 | 231 | 234 | 233 | 235 | 236 | 237 | 238 | 239 | 240 |
| 183 | 242 | 241 | 244 | 243 | 245 | 246 | 247 | 248 | 249 | 250 |
| 222 | 252 | 251 | 254 | 253 | 255 | 256 | 257 | 258 | 259 | 260 |
| 253 | 262 | 261 | 264 | 263 | 265 | 266 | 267 | 268 | 269 | 270 |
| 253H55L | 262 | 261 | 64 | 63 | 265 | 266 | 267 | 68 | 69 | 70 |
| 253H165L | 262 | 261 | 184 | 183 | 265 | 266 | 267 | 188 | 189 | 190 |
| 269 | 272 | 271 | 274 | 273 | 275 | 276 | 277 | 278 | 279 | 280 |
| 278 | 282 | 281 | 284 | 283 | 285 | 286 | 287 | 288 | 289 | 290 |
| 281 | 292 | 291 | 294 | 293 | 295 | 296 | 297 | 298 | 299 | 300 |
| 282 | 302 | 301 | 304 | 303 | 305 | 306 | 307 | 308 | 309 | 310 |
| 285 | 312 | 311 | 314 | 313 | 315 | 316 | 317 | 318 | 319 | 320 |
| 316 | 322 | 321 | 324 | 323 | 325 | 326 | 327 | 328 | 329 | 330 |
| 318 | 332 | 331 | 334 | 333 | 335 | 336 | 337 | 338 | 339 | 340 |
| 334 | 342 | 341 | 344 | 343 | 345 | 346 | 347 | 348 | 349 | 350 |
| 361 | 352 | 351 | 354 | 353 | 355 | 356 | 357 | 358 | 359 | 360 |
| 382 | 362 | 361 | 364 | 363 | 365 | 366 | 367 | 368 | 369 | 370 |
| 384 | 372 | 371 | 374 | 373 | 375 | 376 | 377 | 378 | 379 | 380 |
| 394 | 382 | 381 | 384 | 383 | 385 | 386 | 387 | 388 | 389 | 390 |
| 398 | 392 | 391 | 394 | 393 | 395 | 396 | 397 | 398 | 399 | 400 |

TABLE 2
Summary of SARS-CoV-2-infected patients enrolled in the study.

| Participants | | |
|---|---|---|
| Female | 16 | |
| Male | 26 | |
| Average Age (y) | 55.4 (IQR 47-61) | |
| Days post-symptom onset | 45.5 (IQR 40-53) | |
| Disease severity | | |
| Asymptomatic | 1 | (2.4%) |
| Mild | 28 | (66.6%) |
| Severe | 12 | (28.6%) |
| Critical | 1 | (2.4%) |

TABLE 3
Neutralisation and biolayer interferometry (BLI) measurements
of affinity (KD) and on (Ka) and off (Kdis) rates for selected
mAbs (except for 159 BLI measurements are for Fabs)

| | Neutralisation IC₅₀ | KD (nM) | Ka (1/Ms) | Kdis (1/s) |
|---|---|---|---|---|
| IgG | (ug/ml)# | $ | $ | $ |
| 45 | 2.005* | 5.9 | 7.8E04 | 4.6E−04 |
| 55 | 0.037 ± 0.008 | 3.6 | 1.1E05 | 3.9E−04 |
| 58 | 0.046 ± 0.016 | 3.4 | 1.0E05 | 3.6E−04 |
| 132 | 0.054 ± 0.014 | 3.1 | 1.1E05 | 3.3E−04 |
| 148 | 6.734* | 4.0 | 7.6E04 | 3.0E−04 |
| 150 | 0.023 ± 0.003 | 0.9 | 2.1E05 | 1.9E−04 |
| 158 | 0.034 ± 0.003 | 0.7 | 1.2E05 | 8.4E−05 |
| 165 | 0.021 ± 0.006 | 4.8 | 7.0E04 | 3.3E−04 |
| 170 | 0.025* | 2.7 | 1.7E05 | 4.5E−04 |
| 175 | 0.033 ± 0.009 | 2.4 | 2.8E05 | 6.5E−04 |
| 222 | 0.016 ± 0.003 | 3.0 | 1.2E05 | 3.5E−04 |
| 253 | 0.040 ± 0.007 | 4.5 | 1.4E05 | 6.1E−04 |
| 253H55L | 0.004 ± 0.000 | 1.5 | 3.0E05 | 4.5E−04 |
| 253H165L | 0.003 ± 0.000 | 3.1 | 1.2E05 | 3.9E−04 |
| 269 | 0.036 ± 0.009 | 2.2 | 1.8E05 | 3.9E−04 |
| 278 | 0.009 ± 0.002 | 3.0 | 2.6E05 | 7.8E−04 |
| 281 | 0.005 ± 0.001 | 1.0 | 2.3E05 | 2.3E−04 |
| 282 | 0.073 ± 0.009 | 1.5 | 1.6E05 | 2.4E−04 |
| 316 | 0.010 ± 0.002 | 2.0 | 3.5E05 | 7.1E−04 |
| 318 | 0.015 ± 0.002 | 7.6 | 6.7E04 | 5.1E−04 |
| 384 | 0.002 ± 0.001 | 2.7 | 1.1E05 | 2.9E−04 |
| 159 (anti-spike) | 0.005 ± 0.001 | 1.1 | 5.7E05 | 6.3E−04 |

#Neutralisation activity of selected antibodies against SARS-CoV-2 were determined by FRNT. Data are from 3 independent experiments, each with duplicate wells and the data are shown as mean ± s.e.m.
*FRNT was performed once with duplicate well.
$ Determined for Fab fragments

TABLE 4
Pairs of non-competing antibodies from Cluster
Analysis with potency threshold of <0.1 ug/ml IC₅₀
max 20% competition
<0.1 ug/ml IC₅₀

| | |
|---|---|
| 55 | 58 |
| 55 | 278 |
| 58 | 88 |
| 58 | 132 |
| 58 | 150 |
| 58 | 158 |
| 58 | 165 |
| 58 | 175 |
| 58 | 222 |
| 58 | 282 |
| 88 | 278 |
| 132 | 278 |
| 150 | 278 |
| 158 | 278 |
| 165 | 278 |
| 175 | 278 |
| 222 | 278 |
| 278 | 282 |

TABLE 5
Pairs of non-competing antibodies from Cluster
Analysis with potency threshold of <1 ug/ml IC₅₀
max 20% competition
<1 ug/ml IC₅₀

| | | |
|---|---|---|
| 40 | 318 | |
| 40 | 334 | |
| 40 | 382 | |
| 55 | 58 | 61 |
| 55 | 61 | 278 |
| 55 | 61 | 318 |
| 55 | 61 | 334 |
| 55 | 61 | 361 |
| 55 | 61 | 382 |
| 58 | 61 | 165 |
| 58 | 61 | 175 |
| 58 | 88 | |
| 58 | 132 | |
| 58 | 150 | |
| 58 | 158 | |
| 58 | 222 | |
| 58 | 282 | |
| 61 | 165 | 278 |
| 61 | 165 | 318 |
| 61 | 165 | 334 |
| 61 | 165 | 361 |
| 61 | 165 | 382 |
| 61 | 170 | 334 |
| 61 | 175 | 278 |
| 61 | 175 | 318 |
| 61 | 175 | 334 |
| 61 | 175 | 361 |
| 61 | 175 | 382 |
| 61 | 181 | 334 |
| 61 | 182 | 334 |
| 61 | 183 | 334 |
| 61 | 281 | 334 |
| 61 | 316 | 334 |
| 61 | 334 | 384 |
| 61 | 334 | 394 |
| 61 | 334 | 398 |
| 88 | 278 | |
| 88 | 318 | |
| 88 | 334 | |
| 88 | 361 | |
| 88 | 382 | |
| 132 | 278 | |
| 132 | 318 | |
| 132 | 334 | |
| 132 | 361 | |
| 132 | 382 | |
| 150 | 278 | |
| 150 | 318 | |
| 150 | 334 | |
| 150 | 361 | |
| 150 | 382 | |
| 158 | 278 | |
| 158 | 318 | |
| 158 | 334 | |

TABLE 6
Stability tests for selected antibodies using thermofluor and dynamic light
scattering (DLS).

| | | | | | | | Fraction | |
|---|---|---|---|---|---|---|---|---|
| | Thermofluor (reporter dye) | | | | DLS @ 20 C | | soluble after | |
| | | Prop | | Prop | Pk1 | | N freeze- | |
| Antibody | | n of | | n of | est | Pk1 | thaw cycles | |
| id | Tm 1 | melt | Tm 2 | melt | MW | % mass | N = 5 | N = 20 |
| 2 | 78.4 | | | | 146.25 | 100 | | |
| 22 | 80.7 | | | | 177.55 | 99.98 | | |
| 40 | 73.8 | | | | 215.20 | 100.00 | 0.9 | 0.8 |
| 44 | 77.6 | 0.9 | 89.1 | 0.1 | 685.57* | 100.00 | | |
| 45 | 75.9 | 0.3 | 86.6 | 0.8 | 146.17 | 100.00 | 1.0 | 0.8 |
| 54 | 71.9 | 0.9 | 87.2 | 0.1 | 177.31 | 99.71 | | |
| 55 | 75.9 | 0.4 | 86.7 | 0.7 | 215.20 | 99.83 | 1.0 | 0.9 |
| 58 | 80.6 | | | | 213.93 | 99.98 | 0.9 | 0.9 |
| 61 | 75.9 | 0.9 | 87.1 | 0.1 | 146.17 | 100.00 | | |
| 75 | 75.3 | 0.4 | 84.1 | 0.7 | 215.08 | 100.00 | 0.9 | 1.0 |
| 88 | 73.3 | | | | 213.93 | 100.00 | 1.0 | 0.9 |
| 111 | 80.1 | | | | 214.23 | 100.00 | | |
| 132 | 69.1 | 0.7 | 79.7 | 0.3 | 146.25 | 99.96 | 1.0 | 0.6 |
| 140 | 79.0 | | | | 260.07 | 99.90 | | |
| 148 | 75.4 | 0.3 | 85.4 | 0.7 | 213.93 | 99.98 | | |
| 150 | 71.1 | 0.9 | 89.1 | 0.1 | 176.08 | 100.00 | 1.0 | 0.7 |
| 158 | 76.4 | 0.9 | 88.4 | 0.1 | 213.93 | 99.91 | 1.0 | 0.9 |
| 165 | 78.2 | | | | 177.31 | 100.00 | 1.0 | 1.0 |
| 170 | 64.9 | 0.8 | 76.1 | 0.2 | Out of | 99.16 | 0.9 | 1.0 |
| | | | | | range | | | |
| 175 | 74.3 | | | | 215.08 | 100.00 | 1.0 | 0.9 |
| 177 | 74.3 | | | | 176.08 | 99.93 | | |
| 222 | 76.7 | | | | 214.05 | 100.00 | 0.9 | 0.9 |
| 253 | 75.9 | | | | 146.17 | 100.00 | 1.0 | 0.9 |
| 269 | 73.7 | | | | 214.90 | 100.00 | 0.9 | 0.8 |
| 278 | 76.2 | 1.0 | 88.0 | 0.1 | 316.65 | 100.00 | 1.0 | 0.8 |
| 281 | 70.5 | | | | 214.23 | 100.00 | 0.9 | 0.8 |
| 282 | 70.8 | 0.9 | 88.3 | 0.1 | 215.08 | 99.99 | 0.9 | 0.9 |
| 316 | 74.9 | | | | 465.66 | 100.00 | 0.9 | 0.9 |
| 318 | 72.1 | 0.9 | 86.7 | 0.1 | 144.84 | 100.00 | | |
| 384 | 74.9 | 0.9 | 88.0 | 0.1 | 146.17 | 100.00 | 1.0 | 0.8 |
| 253H165L | 74.5 | 0.6 | 86.4 | 0.4 | 176.08 | 100.00 | 0.9 | 0.7 |
| 253H55L | 74.2 | 0.6 | 87.3 | 0.4 | 175.84 | 100.00 | 0.9 | 0.9 |

TABLE 7
X-ray data collection and refinement statistics (molecular replacement).

| | | | | | | RBD- | | | |
|---|---|---|---|---|---|---|---|---|---|
| | RBD-88- | | RBD-158 | RBD-158 | RBD-253- | 253H55L- | RBD- | | RBD-384- |
| | 45 | RBD-150 | (form 1) | (form 2) | 75 | 75 | scFV269 | RBD-316 | S309 |
| Data | | | | | | | | | |
| collection | | | | | | | | | |
| Space | C2ª | C222₁ | C222₁ | C222₁ | P2₁2₁2₁ª | P2₁ | P6₂22 | P2₁2₁2 | P2₁2₁2₁ |
| group | | | | | | | | | |
| Cell | 180.1, | 81.4, | 53.2, | 83.0, | 93.2, | 93.4, | 173.6, | 104.0, | 108.8, |
| dimensions | 140.7, | 150.7, | 232.3, | 149.4, | 149.8, | 150.1, | 173.6, | 150.9, | 113.2, |
| (Å)^{a, b, c} | 131.0 | 145.5 | 135.2 | 145.5 | 229.1 | 116.1 | 120.6 | 46.0 | 302.8 |
| α, β, γ (°) | 90, 124.6, | 90, 90, 90 | 90, 90, 90 | 90, 90, 90 | 90, 90, 90 | 90, 92.0, | 90, 90, | 90, 90, 90 | 90, 90, 90 |
| | 90 | | | | | 90 | 120 | | |
| Resolution | 59-2.53 | 52-2.30 | 53-2.42 | 51-2.04 | 59-2.50 | 66-3.19 | 57-2.52 | 52-2.30 | 63-2.61 |
| (Å) | (2.57- | (2.34- | (2.46- | (2.08- | (2.54- | (3.25- | (2.56- | (2.34- | (2.65- |
| | 2.53)^{b} | 2.30) | 2.42) | 2.04) | 2.50) | 3.19) | 2.52) | 2.30) | 2.61) |
| ^{R}merge | 0.189 (—) | 0.192 | 0.250 (—) | 0.118 (—) | 0.283 (—) | 0.412 (—) | 0.432 (—) | 0.107 (—) | 0.112 (—) |
| | | (0.990) | | | | | | | |
| ^{R}pim | 0.079 | 0.039 | 0.051 | 0.033 | 0.056 | 0.169 | 0.051 | 0.032 | 0.032 |
| | (1.101) | (0.362) | (0.781) | (1.296) | (1.526) | (1.189) | (2.921) | (1.042) | (0.982) |
| I/σ(I) | 5.6 (0.3) | 12.5 (0.5) | 7.6 (0.4) | 13.1 (0.5) | 7.3 (0.3) | 4.0 (0.5) | 9.2 (0.2) | 14.2 (0.6) | 13.1 (0.5) |
| CC_{1/2} | 0.995 | 0.997 | 0.993 | 0.999 | 0.999 | 0.977 | 0.999 | 0.998 | 0.998 |
| | (0.326) | (0.651) | (0.363) | (0.345) | (0.623) | (0.280) | (0.627) | (0.223) | (0.344) |
| Completeness | 97.8 | 84.4 | 98.5 | 99.9 | 100 | 100 | 99.9 | 85.7 | 100 |
| (%) | (75.6) | (42.8) | (85.2) | (97.2) | (99.5) | (99.5) | (96.3) | (40.2) | (99.1) |
| Redundancy | 6.6 (5.0) | 22.0 (8.2) | 23.7 (13.5) | 13.4 (9.9) | 26.6 (24.8) | 6.9 (7.1) | 73.4 (54.1) | 11.0 (4.9) | 13.3 (12.3) |
| Refinement | | | | | | | | | |
| Resolution | 49-2.53 | 43-2.30 | 53-2.42 | 43-2.04 | 58-2.50 | 58-3.19 | 47-2.52 | 45-2.30 | 63-2.61 |
| (Å) | | | | | | | | | |
| No. | 81142/ | 30799/ | 30107/ | 54733/ | 105179/ | 50411/ | 33910/ | 26846/ | 108562/ |
| reflections | 4196 | 1567 | 1610 | 2897 | 5570 | 2657 | 1753 | 1392 | 5577 |
| R_{work}/R_{free} | 0.250/ | 0.197/ | 0.195/ | 0.194/ | 0.242/ | 0.232/ | 0.217/ | 0.218/ | 0.206/ |
| | 0.285 | 0.236 | 0.229 | 0.216 | 0.284 | 0.274 | 0.248 | 0.233 | 0.241 |
| No. atoms | | | | | | | | | |
| Protein | 13221 | 4787 | 4777 | 4816 | 16052 | 16152 | 3235 | 4768 | 16682 |
| Ligand/ion | 158 | 47 | 68 | 98 | 187 | 177 | 47 | 104 | 234 |
| B factors | | | | | | | | | |
| (Å²) | | | | | | | | | |
| Protein | 70 | 49 | 58 | 63 | 73 | 90 | 91 | 60 | 98 |
| Ligand/ion | 86 | 84 | 82 | 98 | 97 | 114 | 139 | 74 | 124 |
| r.m.s. | | | | | | | | | |
| deviations | | | | | | | | | |
| Bond | 0.003 | 0.005 | 0.003 | 0.005 | 0.002 | 0.002 | 0.003 | 0.002 | 0.003 |
| lengths | | | | | | | | | |
| (Å) | | | | | | | | | |
| Bond | 0.7 | 0.8 | 0.6 | 0.8 | 0.6 | 0.5 | 0.6 | 0.5 | 0.6 |
| angles | | | | | | | | | |
| (°) | | | | | | | | | |

^{a}Presence of translational NCS.
^{b}Values in parentheses are for highest-resolution shell

TABLE 8
Cryo-EM data collection, refinement and validation statistics for spike/COVOX-
Fab(IgG) complexes.
Numbers in brackets refer to the 30° tilted dataset that was merged with the 0° data.
Square brackets provide values for C1 symmetry. *, Rigid body refinement only.

| | | | | 158* | | |
|---|---|---|---|---|---|---|
| | | | 150 | (EMD- | 316 | 384 |
| | 40 (EMD- | 88 | (EMD- | ID, | (EMD-ID, | (EMD-ID, |
| | ID, PDB | (EMD-ID, | ID, PDB | PDB | PDB | PDB |
| | XXX) | PDB XXX) | XXX) | XXX) | XXX) | XXX) |
| Data collection & processing | | | | | | |
| Voltage (kV) | | | | | 300 | |
| Electron exposure (e⁻/Å²) | 43.1 | 57.6 | 48.8 | 45.3 | 46.0 | 47.7 |
| Defocus range (μm) | | | | | −0.8 to −2.6 | |
| Pixel size [super res] (Å) | 0.83 [0.415] | 0.82 | 0.82 | 0.8 | 0.82 | 0.8 |
| Symmetry imposed | C1 | C3 | C1 | C1 | C3 | C1 |
| Particles in final | 39186 | 56686 | 93555 | 18768 | 162,905 | 73,158 |
| reconstruction (no.) | | | | | | |
| Map resolution (Å) | 3.7 | 3.6 | 3.4 | 7.3 | 3.6 | 3.5 |
| FSC threshold | 0.143 | 0.143 | 0.143 | 0.143 | 0.143 | 0.143 |
| Refinement | | | | | | |
| Initial model (PDB ID) | 6Z97 | 6ZDH | 6Z97 | 6Z97 | 6VXX | 6VXX |
| RBD conformation | One-up | All-up | One-up | One-up | All-down | All-down |
| Model res. (Masked) (Å) | 3.8 | 3.3 | 3.8 | 7.9 | 3.8 | 3.7 |
| FSC threshold | 0.5 | 0.5 | 0.5 | 0.5 | 0.5 | 0.5 |
| Sharpening B (Å²) | −83 | −56 | −45 | −369 | −90 | −48 |
| Model composition | | | | | | |
| Non-H atoms | 23467 | 24303 | 23467 | 25152 | 29655 | 26133 |
| Protein residues | 2975 | 3006 | 2975 | 3197 | 3699 | 3237 |
| Ligands | 49 | 60 | 49 | 49 | 69 | 63 |
| B factors (Å²) | | | | | | |
| Protein | 124 | 79 | 102 | 390 | 112 | 89 |
| Ligand | 120 | 110 | 91 | 414 | 195 | 121 |
| R.m.s. deviations | | | | | | |
| Bond lengths (Å) | 0.003 | 0.019 | 0.009 | 0.026 | 0.006 | 0.007 |
| Bond angles (°) | 0.5 | 1.0 | 0.8 | 1.6 | 0.9 | 0.9 |
| Validation | | | | | | |
| MolProbity score | 1.44 | 1.54 | 1.68 | 2.09 | 1.53 | 1.50 |
| Clashscore | 3.81 | 4.84 | 7.07 | 17.0 | 5.17 | 4.21 |
| Poor rotamers (%) | 0.97 | 0.23 | 1.01 | 1.09 | 0.66 | 1.13 |
| Ramachandran plot | | | | | | |
| Flavored (%) | 96.0 | 95.8 | 95.9 | 95.2 | 96.3 | 96.2 |
| Allowed (%) | 3.9 | 3.6 | 4.1 | 4.5 | 3.6 | 3.6 |
| Disallowed (%) | 0 | 0.6 | 0 | 0.3 | 0.2 | 0.2 |

| | | | | 159 | 159* |
|---|---|---|---|---|---|
| | 253H55L | 253H55L | 253H165L* | (EMD- | (EMD- |
| | (EMD-ID, | (EMD-ID, | (EMD-ID, | ID, PDB | ID, PDB |
| | PDB XXX) | PDB XXX) | PDB XXX) | XXX) | XXX) |
| Data collection & processing | | | | | |
| Voltage (kV) | | | | | |
| Electron exposure (e⁻/Å²) | 47.2 | 47.2 | 44.7 | 50.5 | 50.5 |
| Defocus range (μm) | | | | | |
| Pixel size [super res] (Å) | 0.83 [0.415] | 0.83 [0.415] | 0.83 [0.415] | 0.82 | 0.82 |
| Symmetry imposed | C1 | C1 | C1 | C3 | C1 |
| Particles in final | 206,548 | 47,242 | 31,477 | 13638 | 27880 |
| reconstruction (no.) | | | | | |
| Map resolution (Å) | 2.8 | 3.3 | 4.6 [5.1] | 4.1 [4.3] | 4.2 [4.3] |
| FSC threshold | 0.143 | 0.143 | 0.143 | 0.143 | 0.143 |
| Refinement | | | | | |
| Initial model (PDB ID) | 6Z97 | 6VXX | 6Z97 | PDB mix | PDB mix |
| RBD conformation | One-up | All-down | One-up | All-down | One-up |
| Model res. (Masked) (Å) | 3.1 | 3.7 | 5.0 | 5.0 | 6.7 |
| FSC threshold | 0.5 | 0.5 | 0.5 | 0.5 | 0.5 |
| Sharpening B (Å²) | −61 | −37 | −133 | −61 | −49 |
| Model composition | | | | | |
| Non-H atoms | 23790 | 23694 | 23467 | 31035 | 31035 |
| Protein residues | 3018 | 2916 | 2975 | 3879 | 3879 |
| Ligands | 47 | 63 | 49 | 54 | 54 |
| B factors (Å²) | | | | | |
| Protein | 106 | 193 | 231 | 126 | 137 |
| Ligand | 106 | 213 | 239 | 198 | 206 |
| R.m.s. deviations | | | | | |
| Bond lengths (Å) | 0.009 | 0.009 | 0.011 | 0.005 | 0.010 |
| Bond angles (°) | 0.8 | 0.9 | 0.8 | 0.7 | 0.9 |
| Validation | | | | | |
| MolProbity score | 2.01 | 1.55 | 1.76 | 1.56 | 1.78 |
| Clashscore | 6.35 | 7.15 | 8.72 | 5.14 | 8.35 |
| Poor rotamers (%) | 2.54 | 0.23 | 0.97 | 0.80 | 0.86 |
| Ramachandran plot | | | | | |
| Flavored (%) | 94.9 | 97.2 | 95.9 | 95.8 | 95.3 |
| Allowed (%) | 4.7 | 2.6 | 4.4 | 4.1 | 4.4 |
| Disallowed (%) | 0.5 | 0.2 | 0.1 | 0.1 | 0.3 |

TABLE 9
Affinity (KD), neutralisation potency (IC₅₀) and % occupancy against
SARS-CoV-2 of full-length IgG and Fab for ten selected antibodies
were determined. Data are from 2 independent experiments, each
with duplicate wells and the data are shown as mean ± s.e.m.

| | Binding | Neutralisation | |
|---|---|---|---|
| Antibody | KD (nM) | IC₅₀ (nM) | % Occupancy |
| 40 IgG | 0.33 ± 0.04 | 0.16 ± 0.03 | 33.3 ± 1.3 |
| 40 Fab | 1.23 ± 0.19 | 45.68 ± 7.15 | 97.4 ± 0.0 |
| 88 IgG | 0.25 ± 0.01 | 0.07 ± 0.04 | 21.9 ± 9.5 |
| 88 Fab | 1.21 ± 0.14 | 6.95 ± 0.07 | 85.2 ± 1.6 |
| 150 IgG | 0.24 ± 0.01 | 0.08 ± 0.02 | 25.1 ± 5.7 |
| 150 Fab | 0.75 ± 0.10 | 0.61 ± 0.23 | 43.2 ± 6.6 |
| 158 IgG | 0.50 ± 0.02 | 0.13 ± 0.02 | 20.5 ± 2.6 |
| 158 Fab | 3.73 ± 0.56 | 4.12 ± 0.04 | 52.7 ± 3.5 |
| 159 IgG | 0.19 ± 0.02 | 0.08 ± 0.01 | 30.0 ± 5.2 |
| 159 Fab | 0.29 ± 0,02 | N/A | N/A |
| 253 IgG | 0.17 ± 0.01 | 0.36 ± 0.02 | 67.9 ± 2.3 |
| 253 Fab | 23.38 ± 3.84 | N/A | N/A |
| 253H55L IgG | 0.09 ± 0.01 | 0.02 ± 0.00 | 19.6 ± 2.1 |
| 253H55L Fab | 10.19 ± 0.56 | 5.08 ± 0.47 | 33.2 ± 0.8 |
| 253H165L IgG | 0.09 ± 0.01 | 0.03 ± 0.00 | 24.5 ± 0.6 |
| 253H165L Fab | 5.02 ± 1.40 | 5.22 ± 1.18 | 51.3 ± 1.4 |
| 316 IgG | 0.13 ± 0.00 | 0.08 ± 0.02 | 35.4 ± 6.0 |
| 316 Fab | 8.76 ± 0.64 | 27.64 ± 9.47 | 74.0 ± 8.1 |
| 384 IgG | 0.10 ± 0.03 | 0.01 ± 0.00 | 12.1 ± 2.3 |
| 384 Fab | 7.86 ± 0.96 | 4.86 ± 0.16 | 38.4 ± 3.7 |

TABLE 10
X-ray data collection and refinement statistics
Data collection

| | |
|---|---|
| Data set | RBDN501Y/COVOX-269 |
| Space group | C2 |

Cell dimensions (Å)

| a, b, c (Å) | 195.1, 85.0, 57.9 | |
|---|---|---|
| a, b, g (°) | 90, 100.6, 90 | |
| Resolution (Å) | 96-2.19 | (2.23-2.19) |
| Unique reflections | 48007 | (2371) |
| Rmerge | 0.177 | (—) |
| Rpim | 0.050 | (0.988) |
| CC½ | 0.997 | (0.468) |
| <I>/<σI> | 8.4 | (0.2) |
| Completeness (%) | 99.9 | (98.5) |
| Redundancy | 13.0 | (9.1) |

Refinement

| | |
|---|---|
| Resolution (Å) | 56.9-2.19 |
| No. reflections | 41325/2137 |
| Rwork /Rfree | 0.197/0.222 |
| No. atoms | 4948 |
| Average B-factors (Å2) | 68 |

R.m.s. deviations

| | |
|---|---|
| Bond lengths (Å) | 0.003 |
| Bond angles (°) | 0.6 |

Numbers in brackets refer to the highest resolution shell of data.

TABLE 11
Neutralization of selected antibodies against
SARS-CoV-2 strains Victoria and B.1.1.7

| | IC50 (ug/ml) ^{#} | | B.1.1.7/Victoria |
|---|---|---|---|
| mAb | B.1.1.7 | Victoria | Ratio ^{$} |
| 40 | 0.035 ± 0.008 | 0.026 ± 0.007 | 1.36 |
| 55 | 0.348 ± 0.044 | 0.095 ± 0.015 | 3.68 |
| 58 | 0.116 ± 0.029 | 0.041 ± 0.003 | 2.81 |
| 88 | 0.058 ± 0.008 | 0.033 ± 0.001 | 1.78 |
| 132 | 0.337 ± 0.048 | 0.048 ± 0.000 | 7.03 |
| 150 | 0.139 ± 0.019 | 0.012 ± 0.000 | 11.96 |
| 158 | 0.254 ± 0.109 | 0.031 ± 0.004 | 8.33 |
| 159 | 0.061 ± 0.020 | 0.011 ± 0.000 | 5.71 |
| 165 | 0.212 ± 0.004 | 0.034 ± 0.004 | 6.31 |
| 170 | 0.105 ± 0.050 | 0.025 ± 0.004 | 4.22 |
| 175 | 0.575 ± 0.280 | 0.026 ± 0.000 | 22.51 |
| 222 | 0.014 ± 0.002 | 0.019 ± 0.000 | 0.74 |
| 253 | 0.126 ± 0.018 | 0.055 ± 0.008 | 2.28 |
| 269 | >10 | 0.030 ± 0.000 | N/A |
| 278 | 0.307 ± 0.149 | 0.014 ± 0.007 | 22.46 |
| 281 | 0.012 ± 0.000 | 0.005 ± 0.001 | 2.50 |
| 316 | 0.024 ± 0.005 | 0.018 ± 0.007 | 1.37 |
| 318 | 0.185 ± 0.037 | 0.029 ± 0.008 | 6.30 |
| 384 | 0.005 ± 0.002 | 0.004 ± 0.001 | 1.13 |
| 398 | 0.180 ± 0.001 | 0.091 ± 0.004 | 1.98 |
| REGN10933 | 0.014 ± 0.002 | 0.004 ± 0.002 | 3.31 |
| REGN10987 | 0.028 ± 0.003 | 0.032 ± 0.007 | 0.86 |
| AZD1061 | 0.012 ± 0.002 | 0.013 ± 0.003 | 0.93 |
| AZD8895 | 0.011 ± 0.002 | 0.005 ± 0.001 | 2.17 |
| AZD7442 | 0.007 ± 0.001 | 0.009 ± 0.000 | 0.77 |

^{#} Neutralization activity of selected antibodies against SARS-CoV-2 strain Victoria and B.1.1.7 were determined by FRNT. Data are from 2 independent experiments, each with duplicate wells and the data are shown as mean ± s.e.m.
^{$} IC50 ratio between strains B.1.1.7 and Victoria

TABLE 12
Neutralization and RBD binding of selected antibodies against SARS-CoV-2
strains Victoria and B.1.1.7

| | IC50 (ug/ml)^{#} | | | | | | | |
|---|---|---|---|---|---|---|---|---|
| | | | B.1.1.7/ | | | | | |
| | | | Victoria | KD (nM)^{€} | | Immunoglobulin gene usage | | |
| mAb | B.1.1.7 | Victoria | ratio^{$} | RBD-N501Y | Native RBD | IGHV | K/λ | IGLV |
| 40 | 0.035 ± 0.008 | 0.026 ± 0.007 | 1.36 | 0.97 ± 0.006 | 0.55 ± 0.003 | 3-66 | K | 1-33 or 1D-33 |
| 55 | 0.348 ± 0.044 | 0.095 ± 0.015 | 3.68 | 4.55 ± 0.19 | 3.61 ± 0.15 | 1-58 | K | 3-20 |
| 58 | 0.116 ± 0.029 | 0.041 ± 0.003 | 2.81 | 0.55 ± 0.004 | 0.30 ± 0.003 | 3-9 | λ | 3-21 |
| 88 | 0.058 ± 0.008 | 0.033 ± 0.001 | 1.78 | 0.65 ± 0.005 | 0.64 ± 0.004 | 4-61 | λ | 1-36 |
| 132 | 0.337 ± 0.048 | 0.048 ± 0.000 | 7.03 | 43.7 ± 0.43 | 16.0 ± 0.09 | 4-34 | λ | 7-46 |
| 150 | 0.139 ± 0.019 | 0.012 ± 0.000 | 11.96 | 0.72 ± 0.004 | 0.19 ± 0.002 | 3-53 | K | 1-9 |
| 158 | 0.254 ± 0.109 | 0.031 ± 0.004 | 8.33 | 1.57 ± 0.01 | 0.53 ± 0.0003 | 3-53 | K | 1-9 |
| 159 | 0.061 ± 0.020 | 0.011 ± 0.000 | 5.71 | N/A | N/A | 3-30 | K | 3-20 |
| 165 | 0.212 ± 0.004 | 0.034 ± 0.004 | 6.31 | 3.64 ± 0.14 | 3.49 ± 0.15 | 1-58 | K | 3-20 |
| 170 | 0.105 ± 0.050 | 0.025 ± 0.004 | 4.22 | 8.71 ± 0.13 | 5.75 ± 0.07 | 5-51 | K | 2D-29 |
| 175 | 0.575 ± 0.280 | 0.026 ± 0.000 | 22.51 | 2.54 ± 0.02 | 0.97 ± 0.005 | 3-53 | K | 1-33 or 1D-33 |
| 222 | 0.014 ± 0.002 | 0.019 ± 0.000 | 0.74 | 0.39 ± 0.005 | 0.29 ± 0.003 | 3-53 | K | 3-20 |
| 253 | 0.126 ± 0.018 | 0.055 ± 0.008 | 2.28 | 19.5 ± 0.20 | 13.9 ± 0.48 | 1-58 | K | 3-20 |
| 269 | >10 | 0.030 ± 0.000 | N/A | 45.4 ± 1.42 | 1.59 ± 0.009 | 3-53 | K | 1-9 |
| 278 | 0.307 ± 0.149 | 0.014 ± 0.007 | 22.46 | 8.45 ± 0.06 | 5.11 ± 0.03 | 1-18 | K | 1-39 or 1D-39 |
| 281 | 0.012 ± 0.000 | 0.005 ± 0.001 | 2.50 | 4.05 ± 0.02 | 3.20 ± 0.01 | 3-7 | K | 2-24 |
| 316 | 0.024 ± 0.005 | 0.018 ± 0.007 | 1.37 | 2.81 ± 0.03 | 1.50 ± 0.01 | 1-2 | λ | 2-8 |
| 318 | 0.185 ± 0.037 | 0.029 ± 0.008 | 6.30 | 1.99 ± 0.02 | 2.23 ± 0.02 | 1-58 | K | 3-20 |
| 384 | 0.005 ± 0.002 | 0.004 ± 0.001 | 1.13 | 0.71 ± 0.006 | 0.51 ± 0.004 | 3-31 | K | 1-27 |
| 398 | 0.180 ± 0.001 | 0.091 ± 0.004 | 1.98 | 1.18 ± 0.009 | 1.02 ± 0.007 | 3-66 | λ | 2-8 |
| REGN10933 | 0.014 ± 0.002 | 0.004 ± 0.002 | 3.31 | 2.75 ± 0.04 | 1.82 ± 0.03 | | | |
| REGN10987 | 0.028 ± 0.003 | 0.032 ± 0.007 | 0.86 | 3.09 ± 0.06 | 3.85 ± 0.08 | | | |
| AZD1061 | 0.012 ± 0.002 | 0.013 ± 0.003 | 0.93 | 1.41 ± 0.02 | 1.04 ± 0.01 | | | |
| AZD8895 | 0.011 ± 0.002 | 0.005 ± 0.001 | 2.17 | 2.44 ± 0.04 | 1.49 ± 0.02 | | | |

^{#}Neutralization activity of selected antibodies against SARS-CoV-2 strain Victoria and B.1.1.7 were determined by FRNT. Data are from 2 independent experiments, each with duplicate wells and the data are shown as mean ± s.e.m.
^{$}IC50 ratio between strains B.1.1.7 and Victoria.
^{€}KD values for Fab association with N501-RBD and Y501-RBD measured by BLI are shown mAb159 binds to the NTD and was not tested.
VH and VL gene usage for monoclonal antibodies is indicated on the right.

TABLE 13
RBD/Fab complexes analysed for N501 contact

| Fabs contact N501 (≤4 Å) | | Fabs that do not contact N501 (≤4 Å) | |
|---|---|---|---|
| Name | PDB ID | Name | PDB ID |
| B38 | 7BZ5 | CV07-270 | 6XKP |
| CC12.1 | 6XC3 | P2B-2F6 | 7BWJ |
| CC12.3 | 6XC4 | P2C-1A3 | 7CDJ |
| BD604 | 7CH4 | BD368 | 7CHE |
| BD629 | 7CH5 | P17 | 7CWO |
| BD236 | 7CHB | COVA2 | 7JMP |
| COVA2-04 | 7JMO | COVA1 | 7JMW |
| C102 | 7K8M | 52 | 7KZ9 |
| C1A-B12 | 7KFV | 298 | 7KZ9 |
| C1A-B3 | 7KFW | CR3022 | 6YLA |
| C1A-C2 | 7KFX | REGN10933 | 6XDG |
| C1A-F10 | 7KFY | REGN10987 | 6XDG |
| STE90-C11 | 7B3O | CV30 | 6XE1 |
| Fab-7CJF | 7CJF | CB6 | 7C01 |
| 40 | 7ND3 | P2C-1F11 | 7CDI |
| 150 | 7ND5 | EY6A | 6ZCZ |
| 158 | 7ND6 | S309 | 7BEP |
| 269 | 7NEH | 88 | 7BEL |
| | | 253 | 7BEN |
| | | 384 | 7BEP |

TABLE 14
FRNT50 titres against Victoria and B.1.351 strains (A) 34
convalescent plasma (B) Plasma from 13 patients infected with B.1.1.7.
Table 14 A. Neutralization of SARS-CoV-2 convalescent plasma against
SARS-CoV-2 strains Victoria and B.1.351

| | | | Victoria/ |
|---|---|---|---|
| Convalescent | FRNT50 (Reciprocal plasma dilution)# | | B.1.351 |
| plasma | Victoria | B.1.351 | ratio$ |
| Convalescent 1 | 61 | <20 | N/A |
| Convalescent 2 | 689 | <20 | N/A |
| Convalescent 3 | 526 | 88 | 5.99 |
| Convalescent 4 | 409 | 339 | 1.20 |
| Convalescent 5 | 369 | <20 | N/A |
| Convalescent 6 | 1270 | 522 | 2.43 |
| Convalescent 7 | 274 | <20 | N/A |
| Convalescent 8 | 633 | 111 | 5.73 |
| Convalescent 9 | 667 | <20 | N/A |
| Convalescent 10 | 124 | <20 | N/A |
| Convalescent 11 | 102 | <20 | N/A |
| Convalescent 12 | 339 | <20 | N/A |
| Convalescent 13 | 331 | <20 | N/A |
| Convalescent 14 | 438 | <20 | N/A |
| Convalescent 15 | 6397 | 1147 | 5.58 |
| Convalescent 16 | 44 | <20 | N/A |
| Convalescent 17 | 1115 | <20 | N/A |
| Convalescent 18 | 242 | <20 | N/A |
| Convalescent 19 | 29 | <20 | N/A |
| Convalescent 20 | 154 | 21 | 7.44 |
| Convalescent 21 | 487 | 58 | 8.46 |
| Convalescent 22 | 438 | 53 | 8.24 |
| Convalescent 23 | 381 | <20 | N/A |
| Convalescent 24 | 1647 | 72 | 22.89 |
| Convalescent 25 | 913 | 202 | 4.51 |
| Convalescent 26 | 1880 | 348 | 5.41 |
| Convalescent 27 | 1464 | 67 | 21.90 |
| Convalescent 28 | 361 | <20 | N/A |
| Convalescent 29 | 2859 | 748 | 3.82 |
| Convalescent 30 | 1109 | 184 | 6.01 |
| Convalescent 31 | 811 | 210 | 3.87 |
| Convalescent 32 | 395 | <20 | N/A |
| Convalescent 33 | 1144 | 48 | 23.74 |
| Convalescent 34 | 676 | <20 | N/A |

TABLE 14
B. Neutralization of SARS-CoV-2 convalescent plasma
against SARS-CoV-2 strains Victoria and B.1.351

| | | FRNT50 | | Victoria/ |
|---|---|---|---|---|
| Kent | Day after | (Reciprocal plasma dilution) | | B.1.351 |
| plasma | admission | Victoria | B.1.351 | ratio |
| B.1.1.7 P1 | 5 | <20 | <20 | N/A |
| B.1.1.7 P2 | 3 | <20 | <20 | N/A |
| B.1.1.7 P3 | 1 | 440 | <20 | N/A |
| B.1.1.7 P4 | 1 | 136884 | 81493 | 1.7 |
| B.1.1.7 P5 | 29 | 1506 | 278 | 5.4 |
| B.1.1.7 P6 | 24 | 370 | 80 | 4.6 |
| B.1.1.7 P7 | 25 | 2250 | 159 | 14.2 |
| B.1.1.7 P8 | 18 | 2999 | 867 | 3.5 |
| B.1.1.7 P9 | 20 | 970 | 336 | 2.9 |
| B.1.1.7 P10 | 14 | 3735 | 1150 | 3.2 |
| B.1.1.7 P11 | 18 | 2193 | 705 | 3.1 |
| B.1.1.7 P12 | 29 | <20 | <20 | N/A |
| B.1.1.7 P13 | 34 | <20 | <20 | N/A |

TABLE 15
FRNT50 titres against Victoria and B.1.351 strains
(A) Serum from 25 recipients of Pfizer-BioNTech
vaccine. (B) Oxford-AstraZeneca vaccine.

| | | FRNT50 (Reciprocal plasma | | Victoria/ |
|---|---|---|---|---|
| Vaccine | Day Post- | dilution) | | B.1.351 |
| samples | boost | Victoria | B.1.351 | ratio |
| Pfizer1 | 7 | 1149 | 73 | 15.7 |
| Pfizer2 | 7 | <20 | <20 | N/A |
| Pfizer3 | 7 | 1727 | 230 | 7.5 |
| Pfizer4 | 8 | 2234 | 420 | 5.3 |
| Pfizer5 | 7 | 3016 | 577 | 5.2 |
| Pfizer6 | 7 | 1521 | 152 | 10.0 |
| Pfizer7 | 7 | 609 | 109 | 5.6 |
| Pfizer8 | 7 | 4340 | 1255 | 3.5 |
| Pfizer9 | 7 | 1467 | 102 | 14.4 |
| Pfizer10 | 7 | 1757 | 124 | 14.2 |
| Pfizer11 | 7 | 860 | 121 | 7.1 |
| Pfizer12 | 7 | 1749 | 66 | 26.6 |
| Pfizer13 | 7 | 1851 | 385 | 4.8 |
| Pfizer14 | 7 | 407 | 122 | 3.3 |
| Pfizer15 | 8 | 1285 | 202 | 6.4 |
| Pfizer16 | 8 | 1286 | 91 | 14.1 |
| Pfizer17 | 8 | 1810 | 143 | 12.7 |
| Pfizer18 | 8 | 1198 | 93 | 12.9 |
| Pfizer19 | 8 | 466 | 61 | 7.6 |
| Pfizer20 | 8 | 1539 | 178 | 8.7 |
| Pfizer21 | 9 | 184 | <20 | N/A |
| Pfizer22 | 11 | 1061 | 212 | 5.0 |
| Pfizer23 | 12 | 1658 | 100 | 16.6 |
| Pfizer24 | 12 | 1155 | 192 | 6.0 |
| Pfizer25 | 15 | 8092 | 3006 | 2.7 |
| AstraZeneca 1 | 28 | 495 | 155 | 3.2 |
| AstraZeneca 2 | 28 | 580 | 217 | 2.7 |
| AstraZeneca 3 | 28 | 253 | <20 | N/A |
| AstraZeneca 4 | 28 | 183 | 62 | 3.0 |
| AstraZeneca 5 | 28 | 432 | 62 | 7.0 |
| AstraZeneca 6 | 28 | 764 | 54 | 14.3 |
| AstraZeneca 7 | 28 | 133 | <20 | N/A |
| AstraZeneca 8 | 28 | 257 | 41 | 6.3 |
| AstraZeneca 9 | 28 | 501 | 128 | 3.9 |
| AstraZeneca 10 | 28 | 357 | 116 | 3.1 |
| AstraZeneca 11 | 14 | 334 | 45 | 7.5 |
| AstraZeneca 12 | 14 | 250 | 51 | 4.9 |
| AstraZeneca 13 | 14 | 122 | <20 | N/A |
| AstraZeneca 14 | 14 | 212 | <20 | N/A |
| AstraZeneca 15 | 14 | 789 | 94 | 8.4 |
| AstraZeneca 16 | 14 | 538 | 57 | 9.5 |
| AstraZeneca 17 | 14 | 1159 | 36 | 32.5 |
| AstraZeneca 18 | 14 | 353 | 44 | 8.1 |
| AstraZeneca 19 | 14 | 975 | 69 | 14.0 |
| AstraZeneca 20 | 14 | 169 | <20 | N/A |
| AstraZeneca 21 | 14 | 155 | <20 | N/A |
| AstraZeneca 22 | 14 | 152 | <20 | N/A |
| AstraZeneca 23 | 14 | 126 | <20 | N/A |
| AstraZeneca 24 | 14 | 293 | 30 | 9.7 |
| AstraZeneca 25 | 14 | 94 | <20 | N/A |

TABLE 16
FRNT50 titres against Victoria and B.1.351 strains (A) 22 human monoclonal
antibodies. (B) Two Regeneron and 2 AstraZeneca monoclonal antibodies. The Mann-
Whitney U test was used for the analysis and two-tailed P values were calculated, data are
shown as mean ± s.e.m.

| | | | B.1.351/ | | | | | |
|---|---|---|---|---|---|---|---|---|
| | IC50 (ug/ml) | | Victoria | KD (nM) | | Immunoglobulin gene usage | | |
| mAb | Victoria | B.1.351 | ratio | Native RBD | RBD B.1.351 | IGHV | K/λ | IGLV |
| 40 | 0.026 ± 0.007 | 0.738 ± | 28.6 | 0.55 ± 0.003 | 33.2 ± 0.88 | 3-66 | K | 1-33 or |
| | | 0.311 | | | | | | 1D-33 |
| 55 | 0.095 ± 0.015 | 0.127 ± | 1.3 | 3.61 ± 0.15 | 17.6 ± 0.57 | 1-58 | K | 3-20 |
| | | 0.014 | | | | | | |
| 58 | 0.041 ± 0.003 | 0.136 ± | 3.3 | 0.30 ± 0.003 | 5.96 ± 0.12 | 3-9 | λ | 3-21 |
| | | 0.010 | | | | | | |
| 88 | 0.033 ± 0.001 | >10 | N/A | 0.64 ± 0.004 | 277.3 ± 7.17 | 4-61 | λ | 1-36 |
| 132 | 0.048 ± 0.000 | >10 | N/A | 16.0 ± 0.09 | No signal | 4-34 | λ | 7-46 |
| 150 | 0.012 ± 0.000 | 0.350 ± | 30.0 | 0.19 ± 0.002 | 227.7 ± 8.58 | 3-53 | K | 1-9 |
| | | 0.010 | | | | | | |
| 158 | 0.031 ± 0.004 | >10 | N/A | 0.53 ± 0.0003 | 976.5 ± 30.7 | 3-35 | K | 1-9 |
| 159 | 0.011 ± 0.000 | >10 | N/A | N/A | N/A | 3-30 | K | 3-20 |
| 165 | 0.034 ± 0.004 | 0.054 ± | 1.6 | 3.49 ± 0.15 | 8.57 ± 0.20 | 1-58 | K | 3-20 |
| | | 0.013 | | | | | | |
| 170 | 0.025 ± 0.004 | >10 | N/A | 5.75 ± 0.07 | 1207 ± 74.94 | 5-51 | K | 2D-29 |
| 175 | 0.026 ± 0.000 | >10 | N/A | 0.97 ± 0.005 | 1350 ± 45.9 | 3-53 | K | 1-33 or |
| | | | | | | | | 1D-33 |
| 222 | 0.019 ± 0.000 | 0.017 ± | 0.9 | 0.29 ± 0.003 | 0.47 ± 0.09 | 3-53 | K | 3-20 |
| | | 0.005 | | | | | | |
| 253 | 0.055 ± 0.008 | 0.109 ± | 2.0 | 13.9 ± 0.48 | 12.1 ± 0.25 | 1-58 | K | 3-20 |
| | | 0.055 | | | | | | |
| 269 | 0.030 ± 0.000 | >10 | N/A | 1.59 ± 0.009 | >1000 | 3-53 | K | 1-9 |
| 278 | 0.014 ± 0.007 | 0.160 ± | 11.7 | 5.11 ± 0.03 | 8.84 ± 0.16 | 1-18 | K | 1-39 or |
| | | 0.018 | | | | | | 1D-39 |
| 281 | 0.005 ± 0.001 | >10 | N/A | 3.20 ± 0.01 | 79540 ± 170 | 3-7 | K | 2-24 |
| 316 | 0.018 ± 0.007 | >10 | N/A | 1.50 ± 0.01 | No signal | 1-2 | λ | 2-8 |
| 318 | 0.029 ± 0.008 | 0.019 ± | 0.7 | 2.23 ± 0.02 | 3.26 ± 0.04 | 1-58 | K | 3-20 |
| | | 0.008 | | | | | | |
| 384 | 0.004 ± 0.001 | >10 | N/A | 0.51 ± 0.004 | No signal | 3-11 | K | 1-27 |
| 398 | 0.091 ± 0.004 | >10 | N/A | 1.02 ± 0.007 | 21.4 ± 0.20 | 3-66 | λ | 2-8 |
| 253- | 0.003 ± 0.000 | 0.009 ± | 2.7 | 0.88 ± 0.02 | 3.43 ± 0.06 | 1-58 | 3-20 | 3-20 |
| 55 | | 0.002 | | | | | | |
| 253- | 0.003 ± 0.000 | 0.013 ± | 3.6 | 0.73 ± 0.01 | 3.10 ± 0.05 | 1-58 | 3-20 | 3-20 |
| 165 | | 0.003 | | | | | | |

TABLE 16 B

| | | | | KD (nM) | |
|---|---|---|---|---|---|
| | IC50 (ug/ml) | | B.1.351/Victoria | Native | RBD |
| mAb | Victoria | B.1.351 | ratio | RBD | B.1.351 |
| REGN | 0.032 ± 0.007 | 0.007 ± 0.001 | 0.2 | 3.85 ± 0.08 | 0.72 ± 0.02 |
| R10987 | | | | | |
| REGN | 0.004 ± 0.002 | 3.284 ± 2.014 | 773.7 | 1.82 ± 0.03 | No signal |
| R10933 | | | | | |
| AZD1061 | 0.013 ± 0.003 | 0.014 ± 0.002 | 1.1 | 1.04 ± 0.01 | 1.52 ± 0.02 |
| AZD8895 | 0.005 ± 0.001 | 0.046 ± 0.031 | 8.9 | 1.49 ± 0.02 | 3.63 ± 0.06 |

TABLE 17
Data Collection and refinement statistics of RBD complexes

| | | K417N-RBD- | K417T-RBD- | B.1.1.7-RBD- |
|---|---|---|---|---|
| Structure | RBD-EYGA-222 | EY6A-222 | EYA-222 | EY6A-222 |

Data collection

| | | | | |
|---|---|---|---|---|
| Space group | P2₁2₁2₁ | P2₁2₁2₁ | P2₁2₁2₁ | P2₁2₁2₁ |

Cell dimensions

| a, b, c (Å) | 54.4, 120.1, 211.7 | | 54.8, 122.5, 214.2 | | 54.8, 122.7, 213.9 | | 54.3, 120.9, 210.2 | |
|---|---|---|---|---|---|---|---|---|
| a, b, g (°) | 90, 90, 90 | | 90, 90, 90 | | 90, 90, 90 | | 90, 90, 90 | |
| Resolution (Å) | 120-2.25 | (2.29-2.25)^{a} | 62-2.24 | (2.28-2.24) | 81-1.95 | (1.98-1.95) | 79-2.40 | (2.44-2.40) |
| R_{merge} | 0.438 | (—) | 0.185 | (—) | 0.120 | (0.990) | 0.257 | (—) |
| Rₚᵢₘ | 0.073 | (0.767) | 0.053 | (0.805) | 0.034 | (0.972) | 0.051 | (0.547) |
| I/s(I) | 4.3 | (0.3) | 5.7 | (0.3) | 12.0 | (0.4) | 7.7 | (0.6) |
| CC_{1/2} | 0.991 | (0.32) | 0.992 | (0.328) | 0.996 | (0.354) | 0.995 | (0.584) |
| Completeness (%) | 99.8 | (95.8) | 100 | (99.1) | 100 | (99.6) | 100 | (98.5) |
| Redundancy | 34.8 | (17.3) | 13.3 | (13.3) | 13.4 | (13.2) | 26.6 | (25.8) |

Refinement

| | | | | |
|---|---|---|---|---|
| Resolution (Å) | 106-2.25 | 62-2.24 | 81-1.95 | 79-2.40 |
| No. reflections | 61666/3310 | 65884/3438 | 100792/5255 | 52327/2727 |
| R_{work}/R_{free} | 0.207/0.247 | 0.221/0.251 | 0.223/0.246 | 0.225/0.247 |

No. atoms

| | | | | |
|---|---|---|---|---|
| Protein | 8014 | 8013 | 8017 | 8018 |
| | 386 | 423 | 389 | 298 |

Ligand/ion/water
B factors (Å²)

| | | | | |
|---|---|---|---|---|
| Protein | 67 | 84 | 78 | 76 |
| | 60 | 67 | 50 | 67 |

Ligand/ion/water
r.m.s. deviations

| | | | | |
|---|---|---|---|---|
| Bond lengths (Å) | 0.002 | 0.002 | 0.002 | 0.003 |
| Bond angles (°) | 0.5 | 0.5 | 0.5 | 0.6 |

| | |
|---|---|
| P.1-RBD-EY6A- | B.1.351-RBD- |

| | | | |
|---|---|---|---|
| Structure | 222 | EY6A-222 | P.1-RBD-ACE2 |

Data collection

| | | | |
|---|---|---|---|
| Space group | P2₁2₁2₁ | P2₁2₁2₁ | P4₁2₁2 |

Cell dimensions

| | | | |
|---|---|---|---|
| a, b, c (Å) | 54.7, 122.8, 212.3 | 54.2, 120.4, 211.6 | 103.5, 103.5, 225.9 |
| a, b, g (°) | 90, 90, 90 | 90, 90, 90 | 90, 90, 90 |

| | | | | | | |
|---|---|---|---|---|---|---|
| Resolution (Å) | 80-2.67 | (2.72-2.67) | 61-2.50 | (2.54-2.50) | 70-3.14 | (3.27-3.14) |
| R_{merge} | 0.365 | (—) | 0.401 | (—) | 0.500 | (—) |
| Rₚᵢₘ | 0.072 | (1.176) | 0.080 | (1.086) | 0.100 | (1.087) |
| I/s(I) | 7.0 | (0.4) | 4.6 | (0.3) | 5.3 | (0.5) |
| CC_{1/2} | 0.997 | (0.344) | 0.992 | (0.360) | 0.992 | (0.306) |
| Completeness (%) | 100 | (97.7) | 99.9 | (94.2) | 100 | (98.1) |
| Redundancy | 26.3 | (27.5) | 26.3 | (27.1) | 25.7 | (27.1) |

Refinement

| | | | |
|---|---|---|---|
| Resolution (Å) | 80-2.67 | 61-2.50 | 70-3.14 |
| No. reflections | 39534/2103 | 45980/2393 | 20278/1904 |
| R_{work}/R_{free} | 0.224/0.255 | 0.211/0.255 | 0.229/0.276 |

No. atoms

| | | | |
|---|---|---|---|
| Protein | 8034 | 8017 | 6407 |
| | 182 | 145 | 71 |

Ligand/ion/water
B factors (Å²)

| | | | |
|---|---|---|---|
| Protein | 93 | 78 | 91 |
| | 84 | 87 | 107 |

Ligand/ion/water
r.m.s. deviations

| | | | |
|---|---|---|---|
| Bond lengths (Å) | 0.002 | 0.002 | 0.003 |
| Bond angles (°) | 0.5 | 0.5 | 0.5 |

Values in parentheses are for highest-resolution shell.

TABLE 18
Neutralization and RBD binding of selected antibodies against
SARS-CoV-2 strains Victoria, B.1.1.7, B.1.351 and P.1.

| | IC50 (ug/ml) | | | |
|---|---|---|---|---|
| mAb | Victoria | B.1.1.7 | B.1.351 | P.1 |
| 40 | 0.026 ± 0.007 | 0.035 ± 0.008 | 0.738 ± 0.311 | 0.153 ± 0.037 |
| 55 | 0.095 ± 0.015 | 0.348 ± 0.044 | 0.127 ± 0.014 | 0.306 ± 0.046 |
| 58 | 0.041 ± 0.003 | 0.116 ± 0.029 | 0.136 ± 0.010 | 0.236 ± 0.075 |
| 88 | 0.033 ± 0.001 | 0.058 ± 0.008 | >10 | >10 |
| 132 | 0.048 ± 0.000 | 0.337 ± 0.048 | >10 | >10 |
| 150 | 0.012 ± 0.000 | 0.139 ± 0.019 | 0.350 ± 0.010 | 0.040 ± 0.003 |
| 158 | 0.031 ± 0.004 | 0.254 ± 0.109 | >10 | >10 |
| 159 | 0.011 ± 0.000 | 0.061 ± 0.020 | >10 | 1.434 ± 0.804 |
| 165 | 0.034 ± 0.004 | 0.212 ± 0.004 | 0.054 ± 0.013 | 0.241 ± 0.030 |
| 170 | 0.025 ± 0.004 | 0.105 ± 0.050 | >10 | >10 |
| 175 | 0.026 ± 0.000 | 0.575 ± 0.280 | >10 | 3.881 ± 0.738 |
| 222 | 0.019 ± 0.000 | 0.014 ± 0.002 | 0.017 ± 0.005 | 0.008 ± 0.003 |
| 253 | 0.055 ± 0.008 | 0.126 ± 0.018 | 0.109 ± 0.055 | 0.137 ± 0.005 |
| 269 | 0.030 ± 0.000 | >10 | >10 | >10 |
| 278 | 0.014 ± 0.007 | 0.307 ± 0.149 | 0.160 ± 0.018 | 0.245 ± 0.042 |
| 281 | 0.005 ± 0.001 | 0.012 ± 0.000 | >10 | >10 |
| 316 | 0.018 ± 0.007 | 0.024 ± 0.005 | >10 | >10 |
| 318 | 0.029 ± 0.008 | 0.185 ± 0.037 | 0.019 ± 0.008 | 0.083 ± 0.032 |
| 384 | 0.004 ± 0.001 | 0.005 ± 0.002 | >10 | >10 |
| 398 | 0.091 ± 0.004 | 0.180 ± 0.001 | >10 | >10 |
| 253-55 | 0.003 ± 0.000 | 0.008 ± 0.002 | 0.009 ± 0.002 | 0.026 ± 0.006 |
| 253-165 | 0.003 ± 0.000 | 0.006 ± 0.000 | 0.013 ± 0.003 | 0.019 ± 0.000 |
| AZD1061 | 0.013 ± 0.003 | 0.013 ± 0.003 | 0.012 ± 0.002 | 0.014 ± 0.002 |
| AZD8895 | 0.005 ± 0.001 | 0.005 ± 0.001 | 0.011 ± 0.002 | 0.046 ± 0.031 |
| REGN10987 | 0.032 ± 0.007 | 0.032 ± 0.007 | 0.028 ± 0.003 | 0.007 ± 0.001 |
| REGN10933 | 0.004 ± 0.002 | 0.004 ± 0.002 | 0.014 ± 0.002 | 3.284 ± 2.014 |
| ADG10 | 0.006 ± 0.000 | 0.006 ± 0.000 | 0.010 ± 0.001 | 0.011 ± 0.001 |
| ADG20 | 0.004 ± 0.001 | 0.004 ± 0.001 | 0.006 ± 0.000 | 0.01 ± 0.001 |
| ADG30 | 0.007 ± 0.002 | 0.007 ± 0.002 | 0.016 ± 0.001 | 0.029 ± 0.003 |
| LY-CoV555 | 0.006 ± 0.002 | 0.006 ± 0.002 | 0.009 ± 0.000 | >10 |
| LY-CoV16 | 0.034 ± 0.007 | 0.034 ± 0.007 | 3.225 ± 1.030 | >10 |
| S309 | 0.040 ± 0.005 | 0.040 ± 0.005 | 0.078 ± 0.069 | 0.082 ± 0.002 |
| 222H + 222L | 0.017 ± 0.001 | 0.011 ± 0.002 | 0.016 ± 0.001 | 0.009 ± 0.000 |
| 150H + 222L | 0.016 ± 0.003 | 0.010 ± 0.001 | 0.007 ± 0.001 | 0.003 ± 0.000 |
| 158H + 222L | 0.033 ± 0.003 | 0.014 ± 0.001 | 0.056 ± 0.015 | 0.019 ± 0.000 |
| 175H + 222L | >10 | 0.399 ± 0.012 | >10 | >10 |
| 269H + 222L | 0.552 ± 0.085 | 0.024 ± 0.001 | >10 | >10 |

| | FRNT₅₀ ratio | | | Immunoglobulin gene usage | | |
|---|---|---|---|---|---|---|
| mAb | B.1.1.7/Victoria | B.1.351/VictoriaP.1/Victoria | | IGHV | K/λ | IGLV |
| 40 | 1.4 | 28.6 | 5.9 | 3-66 | K | 1-33 or 1D-33 |
| 55 | 3.7 | 1.3 | 3.2 | 1-58 | K | 3-20 |
| 58 | 2.8 | 3.3 | 5.7 | 3-9 | λ | 3-21 |
| 88 | 1.8 | >307.6 | >307.6 | 4-61 | λ | 1-36 |
| 132 | 7.0 | >208.4 | >208.4 | 4-34 | λ | 7-46 |
| 150 | 12.0 | 30.0 | 3.4 | 3-53 | K | 1-9 |
| 158 | 8.3 | >327.5 | >327.5 | 3-53 | K | 1-9 |
| 159 | 5.7 | >928.4 | 133.2 | 3-30 | K | 3-20 |
| 165 | 6.3 | 1.6 | 7.2 | 1-58 | K | 3-20 |
| 170 | 4.2 | >402.2 | >402.2 | 5-51 | K | 2D-29 |
| 175 | 22.5 | >391.5 | 151.9 | 3-53 | K | 1-33 or 1D-33 |
| 222 | 0.7 | 0.9 | 0.4 | 3-53 | K | 3-20 |
| 253 | 2.3 | 2.0 | 2.5 | 1-58 | K | 3-20 |
| 269 | >337.5 | >337.5 | >337.5 | 3-53 | K | 1-9 |
| 278 | 22.5 | 11.7 | 17.9 | 1-18 | K | 1-39 or 1D-39 |
| 281 | 2.5 | >2026.3 | >2026.3 | 3-7 | K | 2-24 |
| 316 | 1.4 | >563.6 | >563.6 | 1-2 | λ | 2-8 |
| 318 | 6.3 | 0.7 | 2.8 | 1-58 | K | 3-20 |
| 384 | 1.1 | >2398.4 | >2398.4 | 3-11 | K | 1-27 |
| 398 | 2.0 | >110.2 | >110.2 | 3-66 | λ | 2-8 |
| 253-55 | 2.3 | 2.7 | 8.1 | 1-58 | K | 3-20 |
| 253-165 | 1.8 | 3.6 | 5.6 | 1-58 | K | 3-20 |
| AZD1061 | 0.9 | 1.1 | 0.5 | | | |
| AZD8895 | 2.2 | 8.9 | 8.8 | | | |
| REGN10987 | 0.9 | 0.2 | 0.4 | | | |
| REGN10933 | 3.3 | 773.7 | 1455.2 | | | |
| ADG10 | 1.8 | 1.9 | 0.5 | | | |
| ADG20 | 1.4 | 2.5 | 2.2 | | | |
| ADG30 | 2.5 | 4.4 | 0.3 | | | |
| LY-CoV555 | 1.5 | >1545.3 | >1545.3 | | | |
| LY-CoV16 | 1.5 | >291.2 | >291.2 | | | |
| S309 | 1.5 | 2.0 | 1.9 | | | |
| 222H + 222L | 0.6 | 1.0 | 0.5 | 3-53 | K | 3-20 |
| 150H + 222L | 0.6 | 0.4 | 0.2 | 3-53 | K | 3-20 |
| 158H + 222L | 0.4 | 1.7 | 0.6 | 3-53 | K | 3-20 |
| 175H + 222L | <0.04 | N/A | N/A | 3-53 | K | 3-20 |
| 269H + 222L | 0.04 | >18.1 | >18.1 | 3-53 | K | 3-20 |

| | KD (nM) | | | | |
|---|---|---|---|---|---|
| mAb | Native RBD | K417T | K417N | E484K | RBD P.1 |
| 40 | 1.33 ± 0.03 | 38.9 ± 0.89 | 56.7 ± 1.18 | 7.31 ± 0.03 | 38.2 ± 0.95 |
| 55 | 2.66 ± 0.05 | 4.60 ± 0.12 | 4.21 ± 0.09 | 3.82 ± 0.02 | 11.1 ± 0.37 |
| 58 | 1.46 ± 0.04 | 1.83 ± 0.02 | 2.75 ± 0.03 | 0.97 ± 0.04 | 2.84 ± 0.03 |
| 88 | 2.90 ± 0.03 | Knocked out | Knocked out | 5.37 ± 0.06 | Knocked out |
| 132 | 2.93 ± 0.10 | 1846 ± 364 | 2495 ± 1095 | 29.8 ± 0.36 | 1338 ± 452 |
| 150 | 0.40 ± 0.01 | 1.64 ± 0.02 | 3.35 ± 0.03 | 1.83 ± 0.03 | 8.90 ± 0.13 |
| 158 | 1.89 ± 0.03 | 4.83 ± 0.06 | 9.35 ± 0.11 | 3.03 ± 0.10 | 18.2 ± 0.31 |
| 159 | | | | | |
| 165 | 2.15 ± 0.03 | 2.62 ± 0.06 | 3.29 ± 0.05 | 3.49 ± 0.03 | 7.10 ± 0.15 |
| 170 | 5.23 ± 0.02 | 6.24 ± 0.04 | 6.95 ± 0.05 | Knocked out | 463.4 ± 25.4 |
| 175 | 1.36 ± 0.01 | 3.17 ± 0.02 | 8.60 ± 0.04 | 10.8 ± 0.07 | 68.3 ± 0.50 |
| 222 | 1.36 ± 0.08 | 3.96 ± 0.11 | 5.16 ± 0.12 | 2.25 ± 0.04 | 1.92 ± 0.01 |
| 253 | 1.15 ± 0.03 | 5.99 ± 0.11 | 6.11 ± 0.11 | 2.66 ± 0.03 | 4.25 ± 0.11 |
| 269 | 0.76 ± 0.02 | 6.69 ± 0.04 | 8.88 ± 0.04 | 1.29 ± 0.03 | Knocked out |
| 278 | 4.16 ± 0.03 | 5.21 ± 0.03 | 6.37 ± 0.03 | 6.95 ± 0.27 | 16.2 ± 0.17 |
| 281 | 0.97 ± 0.03 | 0.68 ± 0.02 | 4.42 ± 0.03 | 3.54 ± 0.08 | Knocked out |
| 316 | 4.81 ± 0.05 | 4.96 ± 0.06 | 5.17 ± 0.08 | Knocked out | Knocked out |
| 318 | 4.43 ± 0.04 | 4.96 ± 0.04 | 5.35 ± 0.04 | 6.05 ± 0.02 | 9.30 ± 0.03 |
| 384 | 1.19 ± 0.02 | 1.30 ± 0.03 | 1.80 ± 0.03 | 1.75 ± 0.04 | Knocked out |
| 398 | 4.63 ± 0.04 | 10.4 ± 0.10 | 13.6 ± 0.24 | Knocked out | Knocked out |
| 253-55 | 2.18 ± 0.03 | 2.30 ± 0.03 | 4.65 ± 0.02 | 6.33 ± 0.04 | 11.0 ± 0.22 |
| 253-165 | 3.38 ± 0.02 | 1.99 ± 0.02 | 2.44 ± 0.02 | 1.41 ± 0.03 | 6.02 ± 0.08 |
| AZD1061 | 5.13 ± 0.07 | 8.00 ± 0.12 | 6.14 ± 0.08 | 7.66 ± 0.05 | 2.27 ± 0.03 |
| AZD8895 | 2.18 ± 0.04 | 2.96 ± 0.05 | 3.56 ± 0.06 | 8.50 ± 0.05 | 7.35 ± 0.15 |
| REGN10987 | 1.35 ± 0.03 | 1.61 ± 0.03 | 1.84 ± 0.04 | 1.39 ± 0.05 | 1.38 ± 0.02 |
| REGN10933 | 0.97 ± 0.02 | 1.99 ± 0.02 | 1.70 ± 0.01 | 1.92 ± 0.02 | 308.7 ± 10.0 |
| ADG10 | | | | | |
| ADG20 | | | | | |
| ADG30 | | | | | |
| LY-CoV555 | | | | | |
| LY-CoV16 | | | | | |
| S309 | | | | | |
| 222H + 222L | | | | | |
| 150H + 222L | | | | | |
| 158H + 222L | | | | | |
| 175H + 222L | | | | | |
| 269H + 222L | | | | | |

Where the table is blank, no data has been obtained for these points.

TABLE 19A
TABLE 19. FRNT50 titres against Victoria and
P.1 strains (A) 34 convalescent plasma (B) Plasma
from 13 patients infected with B.1.1.7.

| Convalescent | FRNT50 (Reciprocal plasma dilution) | | Victoria/P.1 |
|---|---|---|---|
| plasma | Victoria | P.1 | ratio |
| Convalescent 1 | 61 | 25 | 2.5 |
| Convalescent 2 | 689 | 49 | 14.2 |
| Convalescent 3 | 526 | 173 | 3.0 |
| Convalescent 4 | 409 | 356 | 1.1 |
| Convalescent 5 | 369 | 105 | 3.5 |
| Convalescent 6 | 1270 | 556 | 2.3 |
| Convalescent 7 | 274 | 80 | 3.4 |
| Convalescent 8 | 633 | 223 | 2.8 |
| Convalescent 9 | 667 | 126 | 5.3 |
| Convalescent 10 | 124 | 34 | 3.7 |
| Convalescent 11 | 102 | 30 | 3.4 |
| Convalescent 12 | 339 | 74 | 4.6 |
| Convalescent 13 | 331 | 240 | 1.4 |
| Convalescent 14 | 438 | 95 | 4.6 |
| Convalescent 15 | 6397 | 3261 | 2.0 |
| Convalescent 16 | 44 | 39 | 1.1 |
| Convalescent 17 | 1115 | 87 | 12.8 |
| Convalescent 18 | 242 | 64 | 3.8 |
| Convalescent 19 | 29 | 20 | 1.4 |
| Convalescent 20 | 154 | 136 | 1.1 |
| Convalescent 21 | 487 | 165 | 3.0 |
| Convalescent 22 | 438 | 241 | 1.8 |
| Convalescent 23 | 381 | 83 | 4.6 |
| Convalescent 24 | 1647 | 390 | 4.2 |
| Convalescent 25 | 913 | 322 | 2.8 |
| Convalescent 26 | 1880 | 825 | 2.3 |
| Convalescent 27 | 1464 | 206 | 7.1 |
| Convalescent 28 | 361 | 81 | 4.4 |
| Convalescent 29 | 2859 | 1010 | 2.8 |
| Convalescent 30 | 1109 | 477 | 2.3 |
| Convalescent 31 | 811 | 274 | 3.0 |
| Convalescent 32 | 395 | 130 | 3.0 |
| Convalescent 33 | 1144 | 207 | 5.5 |
| Convalescent 34 | 676 | 201 | 3.4 |

TABLE 19B

| | Day post | FRNT50 | | Victoria/ |
|---|---|---|---|---|
| B.1.1.7 | symptom | (Reciprocal plasma dilution) | | P.1 |
| plasma | onset | Victoria | P.1 | ratio |
| B.1.1.7 P3 | 11 | 440 | 116 | 3.8 |
| B.1.1.7 P4 | 18 | 136884 | 48440 | 2.8 |
| B.1.1.7 P5 | 45 | 1506 | 787 | 1.9 |
| B.1.1.7 P6 | 31 | 370 | 259 | 1.4 |
| B.1.1.7 P7 | 33 | 2250 | 267 | 8.4 |
| B.1.1.7 P8 | 25 | 2999 | 2261 | 1.3 |
| B.1.1.7 P9 | 26 | 970 | 861 | 1.1 |
| B.1.1.7 P10 | 18 | 3735 | 861 | 4.3 |
| B.1.1.7 P11 | 24 | 2193 | 1116 | 2.0 |
| B.1.1.7 P13 | 29 | <20 | 67 | <0.3 |
| B.1.1.7 P14 | 21 | 1700 | 168 | 10.1 |
| B.1.1.7 P15 | 16 | 168 | 414 | 0.4 |

TABLE 20
FRNT50 titres against Victoria and P.1 strains (A) Serum
from 25 recipients of Pfizer-BioNTech vaccine. (B) Serum
from 26 recipients of Oxford-AstraZeneca vaccine.

| | Day | FRNT50 | | Victoria/ |
|---|---|---|---|---|
| Vaccine | Post- | (Reciprocal serum dilution) | | P.1 |
| samples | boost | Victoria | P.1 | ratio |
| Pfizer1 | 7 | 1149 | 396 | 2.9 |
| Pfizer2 | 7 | <20 | 36 | <0.6 |
| Pfizer3 | 7 | 1727 | 698 | 2.5 |
| Pfizer4 | 8 | 2234 | 712 | 3.1 |
| Pfizer5 | 7 | 3016 | 1033 | 2.9 |
| Pfizer6 | 7 | 1521 | 302 | 5.0 |
| Pfizer7 | 7 | 609 | 294 | 2.1 |
| Pfizer8 | 7 | 4340 | 2119 | 2.0 |
| Pfizer9 | 7 | 1467 | 361 | 4.1 |
| Pfizer10 | 7 | 1757 | 343 | 5.1 |
| Pfizer11 | 7 | 860 | 424 | 2.0 |
| Pfizer12 | 7 | 1749 | 452 | 3.9 |
| Pfizer13 | 7 | 1851 | 669 | 2.8 |
| Pfizer14 | 7 | 407 | 294 | 1.4 |
| Pfizer15 | 8 | 1285 | 571 | 2.3 |
| Pfizer16 | 8 | 1286 | 311 | 4.1 |
| Pfizer17 | 8 | 1810 | 304 | 6.0 |
| Pfizer18 | 8 | 1198 | 282 | 4.3 |
| Pfizer19 | 8 | 466 | 229 | 2.0 |
| Pfizer20 | 8 | 1539 | 693 | 2.2 |
| Pfizer21 | 9 | 184 | 52 | 3.5 |
| Pfizer22 | 11 | 1061 | 491 | 2.2 |
| Pfizer23 | 12 | 1658 | 355 | 4.7 |
| Pfizer24 | 12 | 1155 | 569 | 2.0 |
| Pfizer25 | 15 | 8092 | 5029 | 1.6 |
| AstraZeneca 1 | 28 | 495 | 265 | 1.9 |
| AstraZeneca 2 | 28 | 580 | 429 | 1.4 |
| AstraZeneca 3 | 28 | 253 | <20 | >12.6 |
| AstraZeneca 4 | 28 | 183 | 102 | 1.8 |
| AstraZeneca 5 | 28 | 432 | 215 | 2.0 |
| AstraZeneca 6 | 28 | 764 | 111 | 6.9 |
| AstraZeneca 7 | 28 | 133 | 29 | 4.5 |
| AstraZeneca 8 | 28 | 257 | 116 | 2.2 |
| AstraZeneca 9 | 28 | 501 | 97 | 5.2 |
| AstraZeneca 10 | 28 | 357 | 133 | 2.7 |
| AstraZeneca 11 | 14 | 334 | 115 | 2.9 |
| AstraZeneca 12 | 14 | 250 | 94 | 2.7 |
| AstraZeneca 13 | 14 | 122 | 40 | 3.1 |
| AstraZeneca 14 | 14 | 212 | 110 | 1.9 |
| AstraZeneca 15 | 14 | 789 | 281 | 2.8 |
| AstraZeneca 16 | 14 | 538 | 181 | 3.0 |
| AstraZeneca 17 | 14 | 1159 | 359 | 3.2 |
| AstraZeneca 18 | 14 | 353 | 85 | 4.1 |
| AstraZeneca 19 | 14 | 975 | 382 | 2.6 |
| AstraZeneca 20 | 14 | 169 | 74 | 2.3 |
| AstraZeneca 21 | 14 | 155 | 87 | 1.8 |
| AstraZeneca 22 | 14 | 152 | 98 | 1.5 |
| AstraZeneca 23 | 14 | 126 | 67 | 1.9 |
| AstraZeneca 24 | 14 | 293 | 151 | 1.9 |
| AstraZeneca 25 | 14 | 94 | 25 | 3.8 |

TABLE 21
Examples of mixed chain antibodies generated from antibodies
derived from the same germline heavy chain IGHV3-53.

light

| chain (L) of | Heavy chain (H) | | | | |
|---|---|---|---|---|---|
| antibody | 150H | 158H | 175H | 222H | 269H |
| 150L | — | 158H 150L | 175H 150L | 222H 150L | 269H 150L |
| 158L | 150H 158L | — | 175H 158L | 222H 158L | 269H 158L |
| 175L | 150H 175L | 158H 175L | — | 222H 175L | 269H 175L |
| 222L | 150H 222L | 158H 222L | 175H 222L | — | 269H 222L |
| 269L | 150H 269L | 158H 269L | 175H 269L | 222H 269L | — |

TABLE 22
Examples of mixed chain antibodies generated from antibodies
derived from the germline heavy chain IGHV3-53 or IGHV3-66.

| Heavy | | | | | | | |
|---|---|---|---|---|---|---|---|
| chain | | | | | | | |
| (H)/light | | | | | | | |
| chain (L) | | | | | | | |
| of antibody | 150H | 158H | 175H | 222H | 269H | 40H | 398H |
| 150L | — | 158H | 175H | 222H | 269H | 40H | 398H |
| | | 150L | 150L | 150L | 150L | 150L | 150L |
| 158L | 150H | — | 175H | 222H | 269H | 40H | 398H |
| | 158L | | 158L | 158L | 158L | 158L | 158L |
| 175L | 150H | 158H | — | 222H | 269H | 40H | 398H |
| | 175L | 175L | | 175L | 175L | 175L | 175L |
| 222L | 150H | 158H | 175H | — | 269H | 40H | 398H |
| | 222L | 222L | 222L | | 222L | 222L | 222L |
| 269L | 150H | 158H | 175H | 222H | — | 40H | 398H |
| | 269L | 269L | 269L | 269L | | 269L | 269L |
| 40L | 150H | 158H | 175H | 222H | 269H | — | 398H |
| | 40L | 40L | 40L | 40L | 40L | | 40L |
| 398L | 150H | 158H | 175H | 222H | 269H | 40H | — |
| | 398L | 398L | 398L | 398L | 398L | 398L | |

TABLE 23
Examples of mixed chain antibodies generated from antibodies
derived from the same germline light chain IGKV3-20.

| Heavy | | | | | | |
|---|---|---|---|---|---|---|
| chain | | | | | | |
| (H)/light | | | | | | |
| chain (L) | 55H | 159H | 165H | 222H | 253H | 318H |
| 55L | — | 159H | 165H | 222H | 253H | 318H |
| | | 55L | 55L | 55L | 55L | 55L |
| 159L | 55H | — | 165H | 222H | 253H | 318H |
| | 159L | | 159L | 159L | 159L | 159L |
| 165L | 55H | 159H | — | 222H | 253H | 318H |
| | 165L | 165L | | 165L | 165L | 165L |
| 222L | 55H | 159H | 165H | — | 253H | 318H |
| | 222L | 222L | 222L | | 222L | 222L |
| 253L | 55H | 159H | 165H | 222H | — | 318H |
| | 253L | 253L | 253L | 253L | | 253L |
| 318L | 55H | 159H | 165H | 222H | 253H | — |
| | 318L | 318L | 318L | 318L | 318L | |

TABLE 24
Examples of mixed chain antibodies generated from antibodies
derived from the same germline light chain IGκV1-9.

| light | |
|---|---|
| chain (L) of | Heavy chain (H) |

| antibody | 150H | 158H | 269H |
|---|---|---|---|
| 150L | — | 158H 150L | 269H 150L |
| 158L | 150H 158L | — | 269H 158L |
| 269L | 150H 269L | 158H 269L | — |

TABLE 25
Examples of the mixed chain antibodies generated from antibodies
derived from the same germline heavy chain IGHV1-58.

light

| chain (L) of | Heavy chain (H) | | | |
|---|---|---|---|---|
| antibody | 55H | 165H | 253H | 318H |
| 55L | — | 165H 55L | 253H 55L | 318H 55L |
| 165L | 55H 165L | — | 253H 165L | 318H 165L |
| 253L | 55H 253L | 165H 253L | — | 318H 253L |
| 318L | 55H 318L | 165H 318L | 253H 318L | — |

TABLE 26
IC50 titres of selected antibodies against SARS-CoV-2 variants

| | IC50 (ug/ml) | | | | | |
|---|---|---|---|---|---|---|
| mAb | Victoria | Alpha | Beta | Gamma | Delta | Omicron |
| 40 | 0.026 ± | 0.035 ± | 0.738 ± | 0.153 ± | 0.029 ± | 7.989 ± |
| | 0.007 | 0.008 | 0.311 | 0.037 | 0.010 | 2.011 |
| 55 | 0.095 ± | 0.348 ± | 0.127 ± | 0.306 ± | 0.016 ± | 7.12 ± |
| | 0.015 | 0.044 | 0.014 | 0.046 | 0.005 | 2.880 |
| 58 | 0.041 ± | 0.116 ± | 0.136 ± | 0.236 ± | 6.434 ± | 0.141 ± |
| | 0.003 | 0.029 | 0.010 | 0.075 | 2.623 | 0.063 |
| 88 | 0.033 ± | 0.058 ± | >10 | >10 | 0.039 ± | >10 |
| | 0.001 | 0.008 | | | 0.007 | |
| 132 | 0.048 ± | 0.337 ± | >10 | >10 | 0.051 ± | >10 |
| | 0.000 | 0.048 | | | 0.013 | |
| 150 | 0.012 ± | 0.139 ± | 0.350 ± | 0.040 ± | 0.020 ± | >10 |
| | 0.000 | 0.019 | 0.010 | 0.003 | 0.001 | |
| 158 | 0.031 ± | 0.254 ± | >10 | >10 | 0.026 ± | >10 |
| | 0.004 | 0.109 | | | 0.002 | |
| 159 | 0.011 ± | 0.061 ± | >10 | 1.434 ± | >10 | >10 |
| | 0.000 | 0.020 | | 0.804 | | |
| 165 | 0.034 ± | 0.212 ± | 0.054 ± | 0.241 ± | 0.027 ± | >10 |
| | 0.004 | 0.004 | 0.013 | 0.030 | 0.006 | |
| 170 | 0.025 ± | 0.105 ± | >10 | >10 | 0.841 ± | >10 |
| | 0.004 | 0.050 | | | 0.103 | |
| 175 | 0.026 ± | 0.575 ± | >10 | 3.881 ± | 0.017 ± | >10 |
| | 0.000 | 0.280 | | 0.738 | 0.003 | |
| 222 | 0.019 ± | 0.014 ± | 0.017 ± | 0.008 ± | 0.018 ± | 0.240 ± |
| | 0.000 | 0.002 | 0.005 | 0.003 | 0.001 | 0.122 |
| 253 | 0.055 ± | 0.126 ± | 0.109 ± | 0.137 ± | 0.005 ± | 1.063 ± |
| | 0.008 | 0.018 | 0.055 | 0.005 | 0.001 | 0.367 |
| 269 | 0.030 ± | >10 | >10 | >10 | 0.021 ± | >10 |
| | 0.000 | | | | 0.004 | |
| 278 | 0.014 ± | 0.307 ± | 0.160 ± | 0.245 ± | 7.374 ± | >10 |
| | 0.007 | 0.149 | 0.018 | 0.042 | 1.397 | |
| 281 | 0.005 ± | 0.012 ± | >10 | >10 | 1.494 ± | >10 |
| | 0.001 | 0.000 | | | 0.302 | |
| 316 | 0.018 ± | 0.024 ± | >10 | >10 | 0.008 ± | >10 |
| | 0.007 | 0.005 | | | 0.001 | |
| 318 | 0.029 ± | 0.185 ± | 0.019 ± | 0.083 ± | 0.018 ± | >10 |
| | 0.008 | 0.037 | 0.008 | 0.032 | 0.003 | |
| 384 | 0.004 ± | 0.005 ± | >10 | >10 | 0.108 ± | >10 |
| | 0.001 | 0.002 | | | 0.035 | |
| 398 | 0.091 ± | 0.180 ± | >10 | >10 | 0.237 ± | >10 |
| | 0.004 | 0.001 | | | 0.038 | |
| 253- | 0.003 ± | 0.008 ± | 0.009 ± | 0.026 ± | 0.003 ± | 2.945 ± |
| 55 | 0.000 | 0.002 | 0.002 | 0.006 | 0.000 | 1.283 |
| 253- | 0.003 ± | 0.006 ± | 0.013 ± | 0.019 ± | 0.007 ± | >10 |
| 165 | 0.000 | 0.000 | 0.003 | 0.000 | 0.002 | |

TABLE 27
IC50 titres of selected antibodies against SARS-CoV-2 variants
The following table shows 50% Focus Reduction Neutralization Titres (FRNT50) for the
indicated monoclonal antibodies against the indicated viruses. Supp stands for a tissue
culture supernatant as opposed to other antibodies where purified antibody was used in the
assay. The chimeric antibodies where the heavy chain (HC) from one antibody is combined
with the light chain (LC) of another antibody are indicated as follows 150HC/222LC
represents the heavy chain from antibody 150 combined with the light chain of antibody
222.

| | IC50 (ug/ml) | | | | | | |
|---|---|---|---|---|---|---|---|
| | | B.1.1.7 | B.1.351 | P.1 | B.1.617.2 | B.1.1.7 ± | |
| mAbs | Victoria | (Alpha) | (Beta) | (Gamma) | (Delta) | E484Q | B.1.525 |
| 150H/222L | 0.013 | | 0.003 | | 0.01 | | |
| 150 | 0.012 ± | 0.139 ± | 0.350 ± | 0.040 ± | 0.020 ± | 0.075 | 0.007 |
| | 0.000 | 0.019 | 0.010 | 0.003 | 0.001 | | |
| 158 | 0.031 ± | 0.254 ± | >10 | >10 | 0.026 ± | 0.094 | 0.022 |
| | 0.004 | 0.109 | | | 0.002 | | |
| 175 | 0.026 ± | 0.575 ± | >10 | 3.881 ± | 0.017 ± | 5.082 | 0.027 |
| | 0.000 | 0.280 | | 0.738 | 0.003 | | |
| 222 | 0.019 ± | 0.014 ± | 0.017 ± | 0.008 ± | 0.018 ± | 0.018 | 0.022 |
| | 0.000 | 0.002 | 0.005 | 0.003 | 0.001 | | |
| 269 | 0.030 ± | >10 | >10 | >10 | 0.021 ± | >10 | 0.040 |
| | 0.000 | | | | 0.004 | | |
| 253H/55L | 0.003 ± | 0.008 ± | 0.009 ± | 0.026 ± | 0.003 ± | 0.389 | |
| | 0.000 | 0.002 | 0.002 | 0.006 | 0.000 | | |
| 253H/165L | 0.003 ± | 0.006 ± | 0.013 ± | 0.019 ± | 0.007 ± | 0.135 | |
| | 0.000 | 0.000 | 0.003 | 0.000 | 0.002 | | |
| 55 | 0.095 ± | 0.348 ± | 0.127 ± | 0.306 ± | 0.016 ± | 0.692 | 0.090 |
| | 0.015 | 0.044 | 0.014 | 0.046 | 0.005 | | |
| 165 | 0.034 ± | 0.212 ± | 0.054 ± | 0.241 ± | 0.027 ± | 1.288 | 0.122 |
| | 0.004 | 0.004 | 0.013 | 0.030 | 0.006 | | |
| 253 | 0.055 ± | 0.126 ± | 0.109 ± | 0.137 ± | 0.005 ± | 0.675 | 0.164 |
| | 0.008 | 0.018 | 0.055 | 0.005 | 0.001 | | |
| 318 | 0.029 ± | 0.185 ± | 0.019 ± | 0.083 ± | 0.018 ± | 0.176 | 0.015 |
| | 0.008 | 0.037 | 0.008 | 0.032 | 0.003 | | |

*no data yet available for the blank boxes

TABLE 28
IC50% values for neutralization of a panel of pseudoviral constructs containing
the indicated mutations in the spike protein when compared with the Wuhan SARS-CoV-2
spike protein sequence
IC50 values are shown for the indicated antibodies. The mixed chain antibodies where the
heavy chain (HC) from one antibody is combined with the light chain (LC) of another
antibody are indicated as follows: 150HC/222LC represents the heavy chain from antibody
150 combined with the light chain of antibody 222.

| | IC50 (ng/ml) | | | | | | | |
|---|---|---|---|---|---|---|---|---|
| | B.1.351 | | | | | | | A.23.1 + |
| | K417N, | B.1.617.1 | C.37 | | | C.36.3 | | E484K |
| | E484K, | L452R, | L452Q, | B.1.616 | B.1.258 | R346S, | B.1.526.2 | V367F, |
| — | N501Y | E484Q | F490S | V483A | N439K | L452R | S477N | E484K |
| 150HC/ | 3 | 4.13 | 2.77 | 2.96 | 2.39 | 5.95 | 4.43 | 3.87 |
| 222LC | | | | | | | | |
| 253HC/ | 1 | 1 | 0.62 | 0.58 | 1 | 0.72 | 1 | 1 |
| 55LC | | | | | | | | |
| 253HC/ | 1.2 | 1 | 1 | 0.51 | 0.54 | 0.94 | 0.73 | 2.88 |
| 165LC | | | | | | | | |

1. An antibody capable of binding to the spike protein of coronavirus SARS-CoV-2, wherein the antibody:
(a) comprises at least three CDRs of antibody 222, or of any one of the 41 antibodies in Table 1; and/or
(b) binds to the same epitope as or competes with antibody 159, 45 or 384.

2. The antibody according to claim 1, comprising:
(a) at least four, five, or all six CDRs of an antibody in Table 1;
(b) a heavy chain variable domain having at least 80% sequence identity to the heavy chain variable domain of an antibody in Table 1;
(c) a light chain variable domain having at least 80% sequence identity to the light chain variable domain of an antibody in Table 1; and/or
(d) a heavy chain variable domain and a light chain variable domain having at least 80% identity to the heavy chain variable domain and light chain domain, respectively, of an antibody in Table 1.

3. The antibody of claim 1 or claim 2, wherein the antibody in Table 1 is:
(a) 222, 253H55L, 253H165L, 318, 253, 55, 165, 384, 159, 88, 40 or 316; or
(b) 58, 222, 253 or 253H55L.

4. The antibody according to any one of the preceding claims, comprising:
(a) a CDRH1, CDRH2, CDRH3, CDRL1, CDRL2 and CDRL3 having an amino acid sequence set forth in SEQ ID NOs: 265, 266, 267, 68, 69 and 70, respectively;
(b) a CDRH1, CDRH2, CDRH3, CDRL1, CDRL2 and CDRL3 having an amino acid sequence set forth in SEQ ID NOs: 265, 266, 267, 188, 189 and 190, respectively;
(c) a CDRH1, CDRH2, CDRH3, CDRL1, CDRL2 and CDRL3 having an amino acid sequence set forth in SEQ ID NOs: 255 to 260, respectively;
(d) a CDRH1, CDRH2, CDRH3, CDRL1, CDRL2 and CDRL3 having an amino acid sequence set forth in SEQ ID NOs: 335 to 340, respectively;
(e) a CDRH1, CDRH2, CDRH3, CDRL1, CDRL2 and CDRL3 having an amino acid sequence set forth in SEQ ID NOs: 65 to 70, respectively;
(f) a CDRH1, CDRH2, CDRH3, CDRL1, CDRL2 and CDRL3 having an amino acid sequence set forth in SEQ ID NOs: 185 to 190, respectively;
(g) a CDRH1, CDRH2, CDRH3, CDRL1, CDRL2 and CDRL3 having an amino acid sequence set forth in SEQ ID NOs: 265 to 270, respectively;
(h) a CDRH1, CDRH2 and CDRH3 having the amino acid sequences set forth in SEQ ID NOs: 175 to 177, respectively;
(i) a CDRH2, CDRH3, CDRL1 and CDRL3 having an amino acid sequence set forth in SEQ ID NOs: 376, 377, 378 and 380, respectively;
(j) a CDRH1, CDRH2, CDRH3, CDRL1, CDRL2 and CDRL3 having an amino acid sequence set forth in SEQ ID NOs: 105 to 110, respectively; or
(k) a CDRH1, CDRH2, CDRH3, CDRL1, CDRL2 and CDRL3 having an amino acid sequence set forth in SEQ ID NOs: 25 to 30, respectively.

5. The antibody according to claim 1 or 2, comprising a heavy chain variable domain amino acid sequence having at least 80% sequence identity to the heavy chain variable domain from a first antibody in Table 1, and a light chain variable domain amino acid sequence having at least 80% sequence identity to the light chain variable domain from a second antibody in Table 1, wherein the first and second antibodies derive from the same germline heavy chain v-region, optionally wherein the heavy chain v-region is IGHV3-53, IGHV1-58 or IGHV3-66; for example wherein:
(a) the first antibody is 150 and the second antibody is 222;
(b) the first antibody is 253 and the second antibody is 55;
(c) the first antibody is 253 and the second antibody is 165; or
(d) the second antibody is 222.

6. The antibody according to any one of the preceding claims, wherein the antibody binds to an epitope that is:
(a) defined by residues 144-147, 155-158 and 250-253 of the N-terminal domain of the spike protein of coronavirus SARS-CoV-2; or
(b) defined by residues F104, L105, L455, F456 and G482 to F486 of the receptor binding domain of the spike protein of coronavirus SARS-CoV-2.

7. The antibody according claim 1 or claim 2, wherein the antibody in Table 1 is 1, 88, 132, 253, 263, 316, 337 or 382, and wherein the N-glycosylation sequon of the antibody in Table 1 is retained.

8. The antibody according to any one of the preceding claims, which is a full-length antibody, e.g. comprising a IgG1 constant region.

9. The antibody according to one of the preceding claims, which comprises an Fc region comprising at least one modification such that serum half-life is extended.

10. A combination of antibodies comprising two or more antibodies according to any one of claims 1 to 9.

11. The combination of antibodies according to claim 10, comprising two, three or four antibodies according to any one of claims 1 to 9.

12. The combination of antibodies according to claim 10 or claim 11, comprising:
(a) a combination of two antibodies listed in a row of Table 4;
(b) a combination of two antibodies, listed in a row of Table 5;
(c) a combination of three antibodies listed in a row of Table 4;
(d) a combination of two antibodies listed in a row of Table 4, and antibody 159;
(e) a combination of two antibodies listed in a row of Table 5, and antibody 159; or
(f) a combination of three antibodies listed in a row of Table 5, and antibody 159.

13. One or more polynucleotides encoding the antibody according to any one of claims 1 to 9, one or more vectors comprising said polynucleotides, or a host cell comprising said vectors.

14. A method for producing an antibody that is capable of binding to the spike protein of coronavirus SARS-CoV-2, comprising culturing the host cell of claim 13 and isolating the antibody from said culture.

15. A pharmaceutical composition comprising: (a) the antibody according to any one of claims 1 to 9, or the combination of antibodies according to any one of claims 10 to 12, and (b) at least one pharmaceutically acceptable diluent or carrier.

16. The antibody according to any one of claims 1 to 9, the combination according to any one of claims 10 to 12, or the pharmaceutical composition according to claim 15, for use in a method for treatment of the human or animal body by therapy.

17. The antibody according to any one of claims 1 to 9, the combination according to any one of claims 10 to 12, or the pharmaceutical composition according to claim 15, for use in a method of treating or preventing coronavirus infection, or a disease or complication associated with coronavirus infection.

18. A method of treating a subject comprising administering a therapeutically effective amount of the antibody according to any one of claims 1 to 9, the combination according to any one of claims 10 to 12, or the pharmaceutical composition according to claim 15, to said subject.

19. The method according to claim 17 or 18, wherein the method is for treating SARS-CoV-2 infection, or a disease or complication associated therewith.

20. A method of identifying the presence of coronavirus, or a protein or a protein fragment thereof, in a sample, comprising:
(i) contacting the sample with the antibody according to any one of claims 1 to 9, or the combination according to any one of claims 10 to 12, and
(ii) detecting the presence or absence of an antibody-antigen complex,
wherein the presence of the antibody-antigen complex indicates the presence of coronavirus, or a protein or a protein fragment thereof, in the sample.

21. The method of claim 20, wherein the antibody is antibody 45 or the combination comprises antibody 45.

22. A method of treating or preventing coronavirus infection, or a disease or complication associated therewith, in a subject, comprising identifying the presence of coronavirus according to the method of claim 20 or claim 21 in a sample, and treating the subject with an anti-viral drug or an anti-inflammatory agent.

23. Use of the antibody according to any one of claims 1 to 9, the combination according to any one of claims 10 to 12, or the pharmaceutical composition according to claim 15, for: (i) preventing, treating and/or diagnosing coronavirus infection, or a disease or complication associated therewith, or (ii) identifying the presence of coronavirus, or a protein or a protein fragment thereof, in a sample.

24. The use of the antibody according to any one of claims 1 to 9, the combination according to any one of claims 10 to 12, or the pharmaceutical composition according to claim 15, for the manufacture of a medicament for treating or preventing coronavirus infection, or a disease or complication associated therewith.

25. The antibody according to any one of claims 1 to 9, the combination according to any one of claims 10 to 12, or the pharmaceutical composition according to claim 15, for use in a method of preventing, treating or diagnosing coronavirus infections caused by a SARS-CoV-2 strain comprising substitution at positions 417, 484 and/or 501 in the spike protein relative to the spike protein of the hCoV-19/Wuhan/WIV04/2019 strain, e.g. it is a member of lineage B.1.1.7, B.1.351, P.1, or B.1.1.529.

26. A method of preventing, treating or diagnosing coronavirus infections caused by a SARS-CoV-2 strain in a subject, wherein the method comprises administering the antibody according to any one of claims 1 to 9, the combination according to any one of claims 10 to 12, or the pharmaceutical composition according to claim 15, to the subject, wherein the SARS-CoV-2 strain comprises mutation at positions 417, 484 and/or 501 in the spike protein relative to the spike protein of the hCoV-19/Wuhan/WIV04/2019 strain, e.g. it is a member of lineage B.1.1.7, B.1.351, P.1, or B.1.1.529.

27. The use of the antibody according to any one of claims 1 to 9, the combination according to any one of claims 10 to 12, or the pharmaceutical composition according to claim 15, for the manufacture of a medicament for preventing, treating or diagnosing coronavirus infections caused by a SARS-CoV-2 strain comprising substitution at positions 417, 484 and/or 501 in the spike protein relative to the spike protein of the hCoV-19/Wuhan/WIV04/2019 strain, e.g. it is a member of lineage B.1.1.7, B.1.351, P.1, or B.1.1.529.
